(19) United States
(12) Patent Application Publication
(10) Pub. No.: US 2026/0069707 A1
(43) Pub. Date: Mar. 12, 2026

(54) COMBINATION THERAPY WITH ANTIBODY-DRUG CONJUGATES AND HYALURONIDASES

(71) Applicant: HALOZYME, INC., San Diego, CA (US)

(72) Inventors: Robert CONNOR, San Diego, CA (US); David W. KANG, San Diego, CA (US); Tara NEKOROSKI, San Diego, CA (US); Michael LABARRE, San Diego, CA (US); Ryan NOLAN, San Diego, CA (US); Nathan NOLL, San Diego, CA (US); Peter CHANG, San Diego, CA (US); Chris WAHL, San Diego, CA (US); Manuel SANCHEZ-FELIX, San Diego, CA (US)

(73) Assignee: HALOZYME, INC., San Diego, CA (US)

(21) Appl. No.: 19/324,546

(22) Filed: Sep. 10, 2025

Related U.S. Application Data
(63) Continuation of application No. PCT/US2025/018582, filed on Mar. 5, 2025.
(60) Provisional application No. 63/561,701, filed on Mar. 5, 2024.
(60) Provisional application No. 63/673,280, filed on Jul. 19, 2024.
(60) Provisional application No. 63/826,202, filed on Jun. 18, 2025.

(51) Int. Cl. A61K 47/68; A61K 38/47; C07K 16/30; C07K 16/32

(52) U.S. Cl. CPC A61K 47/6851; A61K 38/47; A61K 47/68037; C07K 16/30; C07K 16/32; C12Y 302/01035; C07K 2317/94

(57) ABSTRACT

Provided herein are combination dosing regimens comprising administering an antibody-drug conjugate (ADC) and a hyaluronidase. In embodiments, the hyaluronidase is a soluble hyaluronidase. In embodiments, the ADC and hyaluronidase are administered subcutaneously. Pharmaceutical compositions comprising an ADC and a hyaluronidase are also provided. In embodiments, the dosing regimens and the pharmaceutical compositions are used in a method of treating or preventing cancer, a cardiometabolic disease/disorder, an inflammatory disease/disorder, or an autoimmune disease/disorder in a patient in need thereof.

### CROSS-REFERENCE TO RELATED APPLICATIONS

This application is a continuation-in-part of International Patent Application No. PCT/US2025/018582, filed Mar. 5, 2025, which claims priority to U.S. Provisional Patent Application No. 63/561,701, filed Mar. 5, 2024, and U.S. Provisional Patent Application No. 63/673,280, filed Jul. 19, 2024, each of which is incorporated by reference herein in its entirety.

This application claims priority to U.S. Provisional Patent Application No. 63/826,202, filed Jun. 18, 2025, which is incorporated by reference herein in its entirety.

### FIELD OF THE INVENTION

The present disclosure relates to compositions, combination dosing regimen comprising administering an antibody-drug conjugate and hyaluronidase, and methods of treating or preventing diseases using such compositions and dosing regimen.

### INCORPORATION BY REFERENCE OF SEQUENCE LISTING

An electronic version of the Sequence Listing is filed herewith, the contents of which are incorporated by reference in their entirety. The electronic file was created on Sep. 9, 2025, is 33,496 bytes in size, and is titled 20250910_SeqListing.xml.

### BACKGROUND

Patient surveys have shown that the majority of patients would prefer to receive a subcutaneous injection rather than taking intravenous infusions. Currently, all available antibody-drug conjugates are administered via intravenous administration.

A safety concern when treating patients with injectable suspensions is injection site reactions. Combining an antibody-drug conjugate with other drug products can alter the injection site reaction profile of an antibody-drug conjugate. Combining an antibody-drug conjugate with other drug products also may alter the pharmacokinetic (PK) profile of an antibody-drug conjugate.

Subcutaneous (SC) administration offers several advantages over intravenous (IV) delivery that enhance both patient and physician experience. From a patient perspective, SC dosing is generally more convenient, less invasive, and can be administered outside of clinical settings, reducing the need for frequent hospital visits. This shift enables less frequent dosing schedules, improving adherence and quality of life. For physicians, SC formulations simplify treatment logistics and reduce the burden on infusion resources and may lower the overall treatment cost.

There is a need in the art for subcutaneously administered injectable antibody-drug conjugates wherein the administration yields minimal discomfort for the patient while having an improved therapeutic window. The present disclosure addresses this unmet need.

### SUMMARY

In one aspect, the present disclosure provides a combination dosing regimen, comprising: (i) subcutaneously administering to a patient in need thereof a composition comprising a soluble hyaluronidase comprising a sequence of amino acids that has at least 85%, 86%, 87%, 88%, 89%, 90%, 91%, 92%, 93%, 94%, 95%, 96%, 97%, 98% or 99% sequence identity to a sequence of amino acids that contains at least amino acids 36-464 of SEQ ID NO:1 and retains hyaluronidase activity; and an antibody-drug conjugate; or (ii) subcutaneously administering to a patient in need thereof a first composition comprising a soluble hyaluronidase comprising a sequence of amino acids that has at least 85%, 86%, 87%, 88%, 89%, 90%, 91%, 92%, 93%, 94%, 95%, 96%, 97%, 98% or 99% sequence identity to a sequence of amino acids that contains at least amino acids 36-464 of SEQ ID NO: 1 and retains hyaluronidase activity; and subcutaneously administering to the patient in need thereof a second composition comprising an antibody-drug conjugate; wherein the soluble hyaluronidase is subcutaneously administered to the patient in an amount sufficient to obtain at least 50% bioavailability of the antibody-drug conjugate compared to the bioavailability obtained when the antibody-drug conjugate is administered intravenously. In one embodiment, the soluble hyaluronidase and the antibody-drug conjugate are subcutaneously administered to the patient in an amount sufficient to obtain from at least 50% to 225%, 75% to 225%, 75% to 200%, 100% to 200%, 100% to 175%, 125% to 175%, 125% to 150%, or 90% to 110% of the bioavailability of the antibody-drug conjugate compared to the bioavailability obtained when the antibody-drug conjugate is administered intravenously at a therapeutically effective dose. In one embodiment, the combination dosing regimen comprises subcutaneously administering to a subject an antibody-drug conjugate (ADC) and a soluble hyaluronidase; wherein subcutaneous administration of the ADC with the soluble hyaluronidase provides: a maximum blood concentration (Cmax) of ADC that is about 20% to about 60%, optionally about 20% to about 40%, of the Cmax obtained via intravenous (IV) administration of an equivalent dose of the ADC; and an area under the concentration-time curve (AUC) in blood of ADC that is about 50% to about 90%, optionally about 50% to about 80%, of the AUC obtained via IV administration of an equivalent dose of the ADC. In one embodiment, the ADC comprises an antibody and a payload conjugated to the antibody via a cleavable linker, wherein upon cleavage of the cleavable linker, free antibody and payload are released. In one embodiment, subcutaneous administration of the ADC with the soluble hyaluronidase provides a Cmax of free antibody that is about 20% to 60% of the Cmax achieved by IV administration of an equivalent dose of the ADC. In one embodiment, subcutaneous administration of the ADC with the soluble hyaluronidase provides a Cmax of the ADC that is about 20% to 55% of the Cmax achieved by IV administration of an equivalent dose of the ADC. In one embodiment, subcutaneous administration of the ADC with the soluble hyaluronidase provides a Cmax of free payload that is about 30% to about 80% of the Cmax achieved by IV administration of an equivalent dose of the ADC. In one embodiment, subcutaneous administration of the ADC with the soluble hyaluronidase provides a Cmax of total payload that is about 30% to about 80% of the Cmax achieved by IV administration of an equivalent dose of the ADC. In one embodiment, subcutaneous administration of the ADC with the soluble hyaluronidase provides an AUC in blood of the ADC that is about 50% to 90% of the blood AUC achieved by IV administration of an equivalent dose of the ADC. In one embodiment, subcutaneous administration of the ADC with the soluble hyaluronidase provides an AUC in blood of total antibody that is about 50% to 90% of the blood AUC achieved by IV administration of an equivalent dose of the ADC. In one embodiment, subcutaneous administration of the ADC with the soluble hyaluronidase provides an AUC in blood of free payload that is about 50% to 90% of the blood AUC achieved by IV administration of an equivalent dose of the ADC. In one embodiment, the IV administration of the equivalent dose of the ADC does not comprise soluble hyaluronidase. In one embodiment, subcutaneous administration of the ADC with the soluble hyaluronidase achieves a weekly average AUC in blood of the ADC that is about 100 μg/mL*day to 230 μg/mL*day. In one embodiment, subcutaneous administration of the ADC with the soluble hyaluronidase achieves a Cmax of the ADC that is about 20 μg/mL to 150 μg/mL. In one embodiment, subcutaneous administration of the ADC with the soluble hyaluronidase achieves a Cmax of the ADC that is about 25 μg/mL to 100 μg/mL. In one embodiment, the dose delivered subcutaneously is 25% to 400% of the dose administered intravenously. In one embodiment, the dose delivered subcutaneously is 100 to 350% of the dose administered intravenously. In one embodiment, the dose delivered subcutaneously is 150% to 300% of the dose administered intravenously. In one embodiment, the dose delivered subcutaneously is 200% to 250% of the dose administered intravenously. In one embodiment, the dose delivered subcutaneously is 150% to 200% of the dose administered intravenously. In one embodiment, the dose delivered subcutaneously is 200% of the dose administered intravenously. In one embodiment, the dose delivered subcutaneously is 100% to 150% of the dose administered intravenously.

In another aspect, the present disclosure provides a combination dosing regimen comprising subcutaneously administering to a subject an antibody-drug conjugate (ADC) and a soluble hyaluronidase; wherein the dosing regimen is characterized by: a higher dose of the ADC being delivered subcutaneously than an a therapeutically effective dose of the ADC when administered intravenously; the subcutaneous administration of the ADC yields a blood area under the concentration-time curve (AUC) that is equal to or higher than the AUC obtained from an intravenously administered therapeutically effective dose of the ADC; and the subcutaneous administration of the ADC yields a maximum blood concentration (Cmax) that is equal to or lower than the Cmax obtained from an intravenously administered therapeutically effective dose of the ADC. In one embodiment, the ADC comprises an antibody and a payload conjugated to the antibody via a cleavable linker, wherein upon cleavage of the cleavable linker, free antibody and payload are released. In one embodiment, the AUC and Cmax are for the total antibody. In one embodiment, the AUC and Cmax are for the free antibody. In one embodiment, the AUC and Cmax are for the free payload. In one embodiment, the AUC and Cmax are for the total payload. In one embodiment, the AUC is for the ADC and Cmax is for the total payload. In one embodiment, the intravenously administered therapeutically effective dose of the ADC does not comprise soluble hyaluronidase. In one embodiment, subcutaneous administration of the ADC with the soluble hyaluronidase achieves a weekly average AUC in blood of the ADC that is about 120 μg/mL*day to 680 μg/mL*day. In one embodiment, subcutaneous administration of the ADC with the soluble hyaluronidase achieves a Cmax of the ADC that is about 25 μg/mL to 410 μg/mL. In one embodiment, subcutaneous administration of the ADC with the soluble hyaluronidase achieves a Cmax of the ADC that is about 35 μg/mL to 330 μg/mL. In one embodiment, the dose delivered subcutaneously is delivered more frequently than the therapeutically effective dose delivered intravenously. In one embodiment, the dose delivered subcutaneously is 25% to 400% of the dose administered intravenously. In one embodiment, the dose delivered subcutaneously is 100 to 350% of the dose administered intravenously. In one embodiment, the dose delivered subcutaneously is 150% to 300% of the dose administered intravenously. In one embodiment, the dose delivered subcutaneously is 200% to 250% of the dose administered intravenously. In one embodiment, the dose delivered subcutaneously is 150% to 200% of the dose administered intravenously. In one embodiment, the dose delivered subcutaneously is 200% of the dose administered intravenously. In one embodiment, the dose delivered subcutaneously is 100% to 150% of the dose administered intravenously.

In yet another aspect, the present disclosure provides a combination dosing regimen described herein wherein the subcutaneous administration of the ADC in combination with the soluble hyaluronidase results in: greater therapeutic efficacy of the ADC in the subject relative to intravenous administration of the ADC; and/or increased overall survival in the subject relative to intravenous administration of the antibody drug conjugate; and/or increased objective response rate of the ADC in the subject relative to intravenous administration of the antibody drug conjugate; and/or produces greater complete response of the ADC in the subject relative to intravenous administration of the ADC; and/or increased progression-free survival of the ADC in the subject relative to intravenous administration of the ADC; and/or decreased time to treatment failure of the ADC in the subject relative to intravenous administration of the ADC; and/or increased duration of response of the ADC in the subject relative to intravenous administration of the ADC. In one embodiment, the increased survival is disease free. In one embodiment, the intravenous administration of the ADC is without the soluble hyaluronidase.

In yet another aspect, the present disclosure provides a combination dosing regimen described herein, wherein the subcutaneous administration of the ADC in combination with the soluble hyaluronidase results in: reduced toxicity of the ADC in the subject relative to intravenous administration of the ADC; and/or reduced adverse events of the ADC in the subject relative to intravenous administration of the ADC. In one embodiment, the intravenous administration of the ADC is without the soluble hyaluronidase. In one embodiment, the adverse events are selected from hypersensitivity and infusion-related reactions. In one embodiment, the adverse events are selected from itching, redness, rash, hives, fever, chills, back or belly pain, muscle or joint pain, fast heartbeat, and nausea or vomiting, and severe anaphylactic reactions, that could include signs and symptoms of cardiac arrest, hypotension, wheezing, angioedema, swelling, pneumonitis, and skin reactions. In one embodiment, the adverse events are selected from cytopenia, neutropenia, thrombocytopenia, anemia, leukopenia and lymphocytopenia. In one embodiment, the adverse events are selected from diarrhea, constipation, abdominal pain, gastroenteritis, nausea, vomiting, decreased appetite, mucositis and stomatitis. In one embodiment, the adverse events are selected from rash, pruritic, edema, dry skin and alopecia. In one embodiment, the adverse events are selected from back pain and arthralgia. In one embodiment, the adverse event is neuropathy. In one embodiment, the adverse event is interstitial lung disease.

In yet another aspect, the present disclosure provides a combination dosing regimen described herein, wherein the subcutaneous administration of the ADC in combination with the soluble hyaluronidase results in: an increased blood haemoglobin in the subject relative to intravenous administration of the ADC; and/or an increased blood albumin in the subject relative to intravenous administration of the ADC; and/or an increased creatinine clearance in the subject relative to intravenous administration of the ADC; and/or decreased blood alkaline phosphatase in the subject relative to intravenous administration of the ADC; and/or increased blood magnesium in the subject relative to intravenous administration of the ADC; and/or increased blood potassium in the subject relative to intravenous administration of the ADC; and/or increased blood sodium in the subject relative to intravenous administration of the ADC. In one embodiment, the ADC comprises an antibody and a payload conjugated to the antibody via a cleavable linker, wherein upon cleavage of the cleavable linker, free antibody and payload are released. In one embodiment, the intravenous administration of the ADC does not comprise the soluble hyaluronidase.

In yet another aspect, the present disclosure provides a combination dosing regimen comprising: subcutaneously administering to a subject an antibody-drug conjugate (ADC) comprising an antibody that specifically targets Trop 2; and a soluble hyaluronidase, wherein subcutaneous administration of the ADC with the soluble hyaluronidase provides a lower maximum blood concentration (Cmax) of ADC compared to intravenous (IV) administration of an equivalent dose of the ADC. In one embodiment, the ADC comprises a topoisomerase I inhibitor payload conjugated to the antibody via a cleavable linker. In one embodiment, the topoisomerase I inhibitor payload is a chemotherapy drug. In one embodiment, the chemotherapy drug is SN-38. In one embodiment, the ADC is sacituzumab govitecan. In one embodiment, subcutaneous administration of the combination of the ADC with the soluble hyaluronidase provides a maximum blood concentration (Cmax) of ADC that is 20% to 50% of the Cmax achieved by intravenous (IV) administration of an equivalent dose of the ADC. In one embodiment, subcutaneous administration of the combination of the ADC with the soluble hyaluronidase provides an area under the concentration-time curve (AUC) of ADC that is about 40% to about 60% of the AUC obtained via intravenous (IV) administration of an equivalent dose of the ADC. In one embodiment, the intravenous administration of an equivalent dose of the ADC is without the soluble hyaluronidase.

In yet another aspect, the present disclosure provides a combination dosing regimen comprising: subcutaneously administering to a subject an antibody-drug conjugate (ADC) comprising an antibody that specifically targets HER2 and a soluble hyaluronidase; wherein subcutaneous administration of the ADC with the soluble hyaluronidase provides a lower maximum blood concentration (Cmax) of ADC compared to intravenous (IV) administration of an equivalent dose of the ADC. In one embodiment, the ADC comprises a topoisomerase I inhibitor payload conjugated to the antibody via a cleavable linker. In one embodiment, the topoisomerase I inhibitor payload is a chemotherapy drug. In one embodiment, the chemotherapy drug is exatecan derivative DXd. In one embodiment, the ADC is trastuzumab deruxtecan. In one embodiment, subcutaneous administration of the combination of the ADC with the soluble hyaluronidase provides a maximum blood concentration (Cmax) of ADC that is about 15% to about 45% of the Cmax achieved by intravenous (IV) administration of an equivalent dose of the ADC. In one embodiment, subcutaneous administration of the combination of the ADC with the soluble hyaluronidase provides an area under the concentration-time curve (AUC) of ADC that is about 60% to about 90% of the AUC obtained via intravenous (IV) administration of an equivalent dose of the ADC. In one embodiment, the intravenous administration of an equivalent dose of the ADC is without the soluble hyaluronidase.

In yet another aspect, the present disclosure provides a combination dosing regimen described herein, wherein: the free payload weekly average AUC of the ADC administered subcutaneously with the soluble hyaluronidase is 80% to 140%, optionally 100% to 125%, of the equivalent dose of free payload weekly average AUC of the ADC administered subcutaneously without soluble hyaluronidase; and/or the weekly average AUC of the ADC administered subcutaneously with the soluble hyaluronidase is 110% to 170%, optionally 125% to 160%, of the equivalent dose total ADC weekly average AUC of the ADC administered subcutaneously without the soluble hyaluronidase. In one embodiment of the combination dosing regimen described herein, the free payload weekly average AUC of the ADC administered subcutaneously with the soluble hyaluronidase is 80% to 140%, optionally 100% to 125%, of the equivalent dose of free payload weekly average AUC of the ADC administered subcutaneously without soluble hyaluronidase. In one embodiment of the combination dosing regimen described herein, the weekly average AUC of the ADC administered subcutaneously with the soluble hyaluronidase is 110% to 170%, optionally 125% to 160%, of the equivalent dose total ADC weekly average AUC of the ADC administered subcutaneously without the soluble hyaluronidase. In one embodiment of the combination dosing regimen described herein, subcutaneous administration the combination of the ADC with the soluble hyaluronidase provides improved local tolerability in the subject relative to subcutaneous administration of an equivalent dose of the ADC without the soluble hyaluronidase, evidenced at the injection site by reduced erythema, swelling, pain, subcutaneous cytotoxicity, necrosis, lesions, ulcers, pruritis, infection, rash, or dry skin. In one embodiment of the combination dosing regimen described herein, subcutaneous administration of the combination of the ADC with the soluble hyaluronidase provides a reduced incidence of adverse events in the subject compared to intravenous administration of the ADC. In one embodiment of the combination dosing regimen described herein, the soluble hyaluronidase is administered concurrently with the ADC. In one embodiment of the combination dosing regimen described herein, the soluble hyaluronidase is co-formulated with the ADC. In one embodiment of the combination dosing regimen described herein, the soluble hyaluronidase and the ADC are lyophilized together and reconstituted prior to administration to the subject. In one embodiment of the combination dosing regimen described herein, the soluble hyaluronidase and the ADC are lyophilized separately and reconstituted prior to administration to the subject. In one embodiment of the combination dosing regimen described herein, the ADC is lyophilized and reconstituted in a solution comprising the soluble hyaluronidase prior to administration to the subject. In one embodiment of the combination dosing regimen described herein, co-administration of the ADC with the soluble hyaluronidase subcutaneously reduces injection time by at least 10% compared to ADC administered alone either subcutaneously or intravenously. In one embodiment of the combination dosing regimen described herein, the soluble hyaluronidase is administered within about 60 minutes prior to administration of the ADC. In one embodiment of the combination dosing regimen described herein, the soluble hyaluronidase is administered within about 30 minutes prior to administration of the ADC. In one embodiment of the combination dosing regimen described herein, the soluble hyaluronidase is administered within about 15 minutes prior to administration of the ADC. In one embodiment of the combination dosing regimen described herein, the combination dosing regimen is administered on a dosing schedule of once or twice weekly. In one embodiment of the combination dosing regimen described herein, the combination dosing regimen is administered on a dosing schedule of once every 2-4 weeks. In one embodiment of the combination dosing regimen described herein, the combination dosing regimen is administered on a dosing schedule of once every 2-6 weeks. In one embodiment of the combination dosing regimen described herein, the combination dosing regimen is administered on a dosing schedule of once every 2-8 weeks. In one embodiment of the combination dosing regimen described herein, the combination dosing regimen is administered on a dosing schedule of once every two (2) to twelve (12) weeks. In one embodiment of the combination dosing regimen described herein, the dosing schedule is maintained for at least two cycles. In one embodiment of the combination dosing regimen described herein, the dosing schedule is maintained for at least four cycles. In one embodiment of the combination dosing regimen described herein, the dosing schedule is maintained for at least six cycles. In one embodiment of the combination dosing regimen described herein, the dosing schedule is maintained for at least eight cycles. In one embodiment of the combination dosing regimen described herein, the ADC is administered at a dose of 0.1 mg/kg to 50 mg/kg. In one embodiment of the combination dosing regimen described herein, the ADC is administered at a dose of 0.1 mg/kg to 10 mg/kg. In one embodiment of the combination dosing regimen described herein, the ADC is administered at a dose of 0.1 mg/kg to 5 mg/kg. In one embodiment of the combination dosing regimen described herein, the ADC is administered at a dose of 1 mg/kg to 6 mg/kg. In one embodiment of the combination dosing regimen described herein, the soluble hyaluronidase is administered at a dose of 2,000 to 100,000 U, optionally at a dose of 2,000 to 25,000 U. In one embodiment of the combination dosing regimen described herein, the soluble hyaluronidase is administered at a dose of about 2,000 U. In one embodiment of the combination dosing regimen described herein, the soluble hyaluronidase is administered at a dose of about 4,000 U. In one embodiment of the combination dosing regimen described herein, the soluble hyaluronidase is administered at a dose of about 6,000 U. In one embodiment of the combination dosing regimen described herein, the soluble hyaluronidase comprises a soluble human hyaluronidase. In one embodiment of the combination dosing regimen described herein, the soluble hyaluronidase comprises a recombinant soluble human hyaluronidase. In one embodiment of the combination dosing regimen described herein, the soluble hyaluronidase comprises a sequence of amino acids that has at least 85%, 86%, 87%, 88%, 89%, 90%, 91%, 92%, 93%, 94%, 95%, 96%, 97%, 98% or 99% sequence identity to a sequence of amino acids that contains at least amino acids 36-464 of SEQ ID NO: 1 and retains hyaluronidase activity.

In yet another aspect, the present disclosure provides a pharmaceutical composition for subcutaneous administration, the pharmaceutical composition comprising: an antibody-drug conjugate (ADC) comprising an antibody and a payload conjugated to the antibody via a cleavable linker; and a soluble hyaluronidase. In one embodiment, the antibody binds to Trop-2, HER-2, B7-H3, EGFR, DLL3, HER-3, CDH17, folate receptor alpha, Nectin-4, CLDN18.2, c-MET, NaPI2b, CEACAM5, PSMA, CLDN6, FGFR2b, ROR1, CD33, CD30, CD22, CD79b, CD19, integrin beta-6, or Tissue Factor. In one embodiment, the antibody binds to more than one antigen. In one embodiment, the payload is a topoisomerase inhibitor, optionally a topoisomerase I inhibitor. In one embodiment, the payload is a topoisomerase I inhibitor selected from A-1743332 (Adizutecan), AMDCPT, ATI020, AZ14170132 (AZ′0132) (Samrotecan), AZ14170133, BCPT02, Belotecan and Analogues, BLD1102, Bultecan, C24, Camptothecin, CPT-113, CPT116, CPT2, D2102, Deruxtecan, DDDXd, DXd/DX8951 (MAAA-1181a), Dxh, Ed-04, Exatecan, FL-118, GS-P-000, HC74, HS-9265/SHR9265/Rezetecan, Irinotecan (CPT-11), JS-1, KL610023, LD-38, LDX2, Masetecan, MH30010008, Mtoxin (MF-6), NT1, P1003, P1021 (Drozuntecan), PBX-7, PBX-7016, PY-4car2, PY-4car2, QLS6916, SC3386, SN-38, T01, Tavatecan, Topotecan, VIP126, YL0010014, YL0014, ZD06519, or a derivative or analogue of any one thereof; optionally wherein the payload is selected from SN-38, deruxtecan, exatecan, topotecan, camptothecin, or a derivative of any one thereof. In one embodiment, the topoisomerase I inhibitor is SN-38. In one embodiment, the topoisomerase I inhibitor is deruxtecan. In one embodiment, the cleavable linker is a chemically cleavable linker. In one embodiment, the cleavable linker is selected from an acid labile linker, an enzyme cleavable linker, a reducible disulfide linker, a glutathione-sensitive linker, an Fe (II)-responsive linker, an oxidation labile/ROS (reactive oxygen species) sensitive linker, a photo-responsive linker, a bioorthogonal linker, or a combination thereof. In one embodiment, the acid labile linker is a hydrazone linker or a CL2A linker. In one embodiment, the cleavable linker is a hydrazone linker. In one embodiment, the cleavable linker is cleaved in the acidic environment of endosomes or lysosomes. In one embodiment, the ADC has a drug antibody ratio of 2-16. In one embodiment, the ADC has a drug-antibody ratio of 2-8. In one embodiment, the ADC has a drug-antibody ratio of 2-4. In one embodiment, the ADC is sacituzumab govitecan. In one embodiment, the ADC is trastuzumab deruxtecan. In one embodiment, the pharmaceutical composition further comprises one or more excipients selected from 2-(N-morpholino) ethane sulfonic acid (MES), citric acid monohydrate, dextran, d-mannitol, glacial acetic acid, histidine, histidine hydrochloride monohydrate, L-histidine, L-histidine hydrochloride monohydrate, L-histidine monohydrochloride, polysorbate, sodium acetate, sodium chloride, sodium citrate dihydrate, sodium hydroxide, sodium phosphate dibasic anhydrous, sodium phosphate monobasic monohydrate, sodium succinate, succinic acid, sucrose, trehalose, trehalose dihydrate, or tromethamine. In one embodiment, the pharmaceutical composition further comprises one or more buffers selected from histidine, MES, citrate, acetate, phosphate, or TRIS. In one embodiment, the pharmaceutical composition further comprises one or more stabilizers selected from trehalose, sucrose, mannitol, sorbitol, glycine, or arginine. In one embodiment, the pharmaceutical composition further comprises one or more surfactants selected from polysorbate 20, polysorbate 80, poloxamer 188, or sodium deoxycholate. In one embodiment, the pharmaceutical composition further comprises one or more tonicity-adjusting agents selected from sodium chloride, potassium chloride, calcium chloride, or glycerol. In one embodiment, the pharmaceutical composition further comprises one or more antioxidants selected from methionine, cysteine, ascorbic acid, a tocopherol, or BHT. In one embodiment, the pharmaceutical composition further comprises one or more preservatives selected from benzyl alcohol, phenol, m-cresol, or a paraben. In one embodiment, the pharmaceutical composition further comprises a combination of a buffer, a stabilizer, a surfactant, and a tonicity-adjusting agent. In one embodiment, the soluble hyaluronidase comprises a soluble human hyaluronidase. In one embodiment, the soluble hyaluronidase comprises a recombinant soluble human hyaluronidase. In one embodiment, the soluble hyaluronidase comprises a sequence of amino acids that has at least 85%, 86%, 87%, 88%, 89%, 90%, 91%, 92%, 93%, 94%, 95%, 96%, 97%, 98% or 99% sequence identity to a sequence of amino acids that contains at least amino acids 36-464 of SEQ ID NO:1 and retains hyaluronidase activity.

### BRIEF DESCRIPTION OF DRAWINGS

The following detailed description of embodiments of the hyaluronidase formulations for high volume administration, will be better understood when read in conjunction with the appended drawings of exemplary embodiments.

FIG. 1 is a chart of mean injection time (seconds±SEM) of Trodelvy and Trodelvy+rHuPH20.

FIG. 2 is a chart of mean (mg±SEM) and individual weights of back-leakage.

FIG. 3 is a chart of individual swelling volumes (mL) after SC Injection of Trodelvy and Trodelvy+rHuPH20—caliper measurement.

FIG. 4 is a chart of individual swelling area (cm) after SC injection of Trodelvy and Trodelvy+rHuPH20—caliper measurement.

FIG. 5 is a chart of individual swelling height (mm) after SC injection of Trodelvy and Trodelvy+rHuPH20—caliper measurement.

FIGS. 6A and 6B are composite 3D images of the minipigs by treatment. FIG. 6A: Composite images of minipigs treated with Trodelvy. FIG. 6B: Composite 3D images of minipigs treated with Trodelvy+rHuPH20.

FIG. 7 is a chart of individual bleb volume (mL) after SC injection of Trodelvy and Trodelvy+rHuPH20—3D imaging.

FIG. 8 is a chart of individual bleb area (cm²) after SC injection of Trodelvy and Trodelvy+rHuPH20—3D imaging.

FIG. 9 is a chart of individual bleb height (mm) after SC injection of Trodelvy and Trodelvy+rHuPH20—3D imaging.

FIG. 10 is a chart of the qualitative assessment of post-injection swelling size.

FIG. 11 is a chart of the qualitative assessment of post-injection induration (firmness).

FIGS. 12A-12I are images of the histological staining of the minipigs taken from injection sites and naïve skin. FIG. 12A provides an image of the histological staining of minipig AID #4493 after injection with Trodelvy. FIG. 12B provides an image of the histological staining of minipig AID #4493 after injection with Trodelvy+rHuPH20. FIG. 12C provides an image of the histological staining of the naïve skin of minipig AID #4493. FIG. 12D provides an image of the histological staining of minipig AID #4498 after injection with Trodelvy. FIG. 12E provides an image of the histological staining of minipig AID #4498 after injection with Trodelvy+rHuPH20. FIG. 12 F provides an image of the histological staining of the naïve skin of minipig AID #4498. FIG. 12G provides an image of the histological staining of minipig AID #4593 after injection with Trodelvy. FIG. 12H provides an image of the histological staining of minipig AID #4593 after injection with Trodelvy+rHuPH20. FIG. 12I provides an image of the histological staining of the naïve skin of minipig AID #4593.

FIG. 13 is a chart of mean injection time (seconds±SEM) of ADC and ADC+rHuPH20.

FIG. 14 is a chart of mean (mg±SEM) and individual weights of back-leakage of Trodelvy and Trodelvy+rHuPH20.

FIGS. 15A-15C show swelling amounts after SC injection of ADC and ADC+rHuPH20. FIG. 15A is a chart of individual swelling volumes (mL) after SC injection of ADC and ADC+rHuPH20 taken by caliper measurement. FIG. 15B is a chart of individual swelling area (cc) after SC injection of ADC and ADC+rHuPH20 taken by caliper measurement. FIG. 15C is a chart of individual swelling height (mm) after SC injection of ADC and ADC+rHuPH20 taken by caliper measurement.

FIG. 16 shows a composite of 3D images of injection sites by treatment and AID #.

FIGS. 17A-17C show bleb size after SC injection of ADC and ADC+rHuPH20. FIG. 17A is a chart of individual bleb volume (mL) after SC injection of ADC and ADC+rHuPH20 measured by 3D imaging. FIG. 17B is a chart of individual bleb area (cm²) after SC injection of ADC and ADC+rHuPH20 measured by 3D imaging. FIG. 17C is a chart of individual bleb height (mm) after SC injection of ADC and ADC+rHuPH20 measured by 3D imaging.

FIG. 18 is a chart of a qualitative assessment of post-injection swelling size measured by swelling score.

FIG. 19 is a chart showing qualitative assessment of post-injection induration (firmness) measured by induration score.

FIGS. 20A-20F show injection sites post-injection for different treatments and AID #. FIG. 20A shows injection sites post dosing with ADC for AID #4493L. FIG. 20B shows injection sites post dosing with ADC+rHuPH20 for AID #4493R. FIG. 20C shows injection sites post dosing with ADC for AID #4498R. FIG. 20D shows injection sites post dosing with ADC+rHuPH20 for AID #4498L. FIG. 20E shows injection sites post dosing with ADC for AID #4593L. FIG. 20F shows injection sites post dosing with ADC+rHuPH20 for AID #4593R.

FIGS. 21A-21I show histological staining of tissues taken from injection sites post-injection for different treatments and AID #. FIG. 21A shows H&E staining of AID #4493L: ADC. FIG. 21B shows H&E staining of AID #4493R: ADC+rHuPH20. FIG. 21C shows H&E staining of AID #4493: naïve skin. FIG. 21D shows H&E staining of AID #4498R: ADC. FIG. 21E shows H&E staining of AID #4498L: ADC+rHuPH20. FIG. 21F shows H&E staining of AID #4498: naïve skin. FIG. 21G shows H&E staining of AID #4593L: ADC. FIG. 21H shows H&E staining of AID #4593R: ADC+rHuPH20. FIG. 21I shows H&E staining of AID #4593: naïve skin.

FIG. 22 shows injection sites Q1-Q8 before injection.

FIG. 23 shows close-up views of injection sites Q1-Q8 before injection.

FIG. 24 shows injection site Q7 (ADC) and Q2 (ADC+rHuPH20): 0-0.5h.

FIG. 25 shows injection site Q3 (ADC) and Q6 (ADC+rHuPH20): 0-1h.

FIG. 26 shows injection site Q5 (ADC) and Q4 (ADC+rHuPH20): 0-2h.

FIG. 27 shows injection site Q1 (ADC) and Q8 (ADC+rHuPH20): 0-4h.

FIGS. 28A-28J show histological staining of tissue samples taken from injection sites. FIG. 28A shows H&E staining of injection Site #7—ADC—0.5 hour exposure. FIG. 28B shows H&E Staining of Injection Site #2—ADC+rHuPH20—0.5 hour exposure. FIG. 28C shows H&E Staining of Injection Site #3—ADC—1 hour exposure. FIG. 28D shows H&E Staining of Injection Site #6—ADC+rHuPH20—1 hour exposure. FIG. 28E shows H&E Staining of Injection Site #5—ADC—2 hour exposure. FIG. 28F shows H&E Staining of Injection Site #4—ADC+rHuPH20—2 hour exposure. FIG. 28G shows H&E Staining of Injection Site #1—ADC—4 hour exposure. FIG. 28H shows H&E Staining of Injection Site #8—ADC+rHuPH20—4 hour exposure. FIG. 28I shows H&E Staining of Injection Site #9—Naïve skin. FIG. 28J shows H&E Staining of Injection Site #10—Naïve skin.

FIG. 29 is a chart of post-injection back-leakage of ADC and ADC+rHuPH20.

FIGS. 30-30B show bleb size post-injection of ADC and ADC+rHuPH20. FIG. 30A is a chart of bleb volume of ADC and ADC+rHuPH20. FIG. 30B shows a chart of bleb height of ADC and ADC+rHuPH20.

FIG. 31 shows a comparison of injection site of ADC and ADC+rHuPH20 from 0-2h.

FIGS. 32A-32D compare swelling and induration post-injection with ADC and ADC+rHuPH20. FIG. 32A is a chart comparison of post-injection swelling of ADC and ADC+rHuPH20 at T0. FIG. 32B is a chart comparison of post-injection induration of ADC and ADC+rHuPH20 at T0. FIG. 32C is a chart comparison of post-injection swelling of ADC and ADC+rHuPH20 from T0 to T2h. FIG. 32D is a chart comparison of post-injection induration of ADC and ADC+rHuPH20 from T0 to T2h.

FIG. 33 is a chart comparing post-injection back-leakage of ADC and ADC+rHuPH20.

FIGS. 34A and 34B show comparisons of bleb size post-injection with ADC and ADC+rHuPH20. FIG. 34A is a chart comparing post-injection bleb volume of ADC and ADC+rHuPH20. FIG. 34B is a chart comparing post-injection bleb height of ADC and ADC+rHuPH20.

FIGS. 35A-35D compare swelling and induration post-injection post-injection with ADC and ADC+rHuPH20. FIG. 35A is chart comparing post-injection swelling at T0. FIG. 35B is chart comparing post-injection induration at T0. FIG. 35C is chart comparing post-injection swelling from T0 to T2h. FIG. 35D is chart comparing post-injection induration from T0 to T2h.

FIGS. 36A-36A show a comparison of delivery time and back leakage of injection with ADC and ADC+rHuPH20. FIG. 36A is a chart comparing delivery time of ADC v. ADC+rHuPH20. FIG. 36B is a chart comparing back leakage of ADC v. ADC+rHuPH20.

FIGS. 37A-37C show a comparison of bleb size post-injection with ADC and ADC+rHuPH20. FIG. 37A is a chart comparing bleb volume post-injection with ADC v. ADC+rHuPH20. FIG. 37B is a chart comparing bleb area post-injection with ADC v. ADC+rHuPH20. FIG. 37C is a chart comparing bleb height post-injection with ADC v. ADC+rHuPH20.

FIGS. 38A-38B compare swelling and induration post-injection with ADC and ADC+rHuPH20. FIG. 38A is a chart comparing post-injection swelling post-injection with ADC and ADC+rHuPH20. FIG. 38B is a chart comparing post-injection induration post-injection with ADC and ADC+rHuPH20.

FIG. 39 shows a histological evaluation of skin after SC administration of ADC and ADC+rHuPH20.

FIGS. 40A-40B show the individual animal data for the enzymatic activity of the co-mix of dupilumab+rHuPh20.

FIGS. 41A-41C show the mean and individual post-injection bleb volume, area and height values of the co-mix of dupilumab+rHuPH20, respectively.

FIG. 42 shows individual animal data for scoring by three evaluators for swelling size using the modified Draize scoring system as summarized in Table 41.

FIG. 43 shows individual animal data for scoring by three evaluators for bleb induration as summarized in Table 42.

FIG. 44 shows the concentration-time profiles of dupilumab and dupilumab+rHuPH20.

FIG. 45 shows the dose-normalized concentration versus time profiles for the two treatment groups.

FIG. 46 shows a simulation for conversion of an IV dosing regimen of Trodelvy to a SC regimen with rHuPH20, with equivalent AUC and lower Cₘₐₓ.

FIG. 47 shows a simulation for conversion of an IV dosing regimen of patritumab deruxtecan to a SC regimen with rHuPH20, with equivalent AUC and lower Cₘₐₓ.

FIG. 48 shows a simulation for conversion of an IV dosing regimen of datopotamab deruxtecan to a SC regimen with rHuPH20, with equivalent AUC and lower Cₘₐₓ.

FIG. 49 shows a simulation for conversion of an IV dosing regimen of brentuximab vedotin to a SC regimen with rHuPH20, with equivalent AUC and lower Cₘₐₓ.

FIG. 50 shows a simulation for conversion of an IV dosing regimen of trastuzumab emtansine to a SC regimen with rHuPH20, with equivalent AUC and lower Cₘₐₓ.

FIG. 51 shows a simulation for conversion of an IV dosing regimen of trastuzumab deruxtecan to a SC regimen with rHuPH20, with equivalent AUC and lower Cₘₐₓ.

FIG. 52 shows a simulation for conversion of an IV dosing regimen of tisotumab vedotin to a SC regimen with rHuPH20, with equivalent AUC and lower Cₘₐₓ.

FIG. 53 shows a simulation for conversion of an IV dosing regimen of mirvetuximab soravtansine to a SC regimen with rHuPH20, with equivalent AUC and lower Cₘₐₓ.

FIG. 54 shows a simulation for conversion of an IV dosing regimen of loncastuximab tesirine to a SC regimen with rHuPH20, with equivalent AUC and lower Cₘₐₓ.

FIG. 55 is a plot of mean total ADC (TADC) serum concentration versus time following administration of ADC-IV, ADC-SC, and ADC+rHuPH20-SC.

FIG. 56 is a plot of total antibody (TAB) serum concentration versus time following administration of ADC-IV, ADC-SC, and ADC+rHuPH20-SC.

FIG. 57 is a plot of TADC/TAB serum ratio over time for ADC-IV, ADC-SC, and ADC+rHuPH20-SC.

FIG. 58 is a plot of total free payload serum concentration versus time following administration of ADC-IV, ADC-SC and ADC+rHuPH20-SC.

FIG. 59 is a plot of the TAB concentration in tissue homogenates of the injection site (μg/mg protein±SD).

FIG. 60 is a plot of the TADC concentration in tissue homogenates of the injection site (μg/mg protein±SD).

FIG. 61 is a plot of the free payload concentration in tissue homogenates of the injection site (μg/mg protein±SD).

FIG. 62 is a plot of the total ADC/total antibody (TADC/TAB) ratios over time from skin tissue homogenates (Mean±SD).

FIG. 63 is a plot of serum concentration-time profiles for total antibody (TAB) for individual animals (ADC-IV-purple; ADC-SC-red; ADC+rHuPH20-blue).

FIG. 64 is a plot of serum concentration-time profiles for total ADC (TADC) for individual animals (ADC-IV-purple; ADC-SC-red; ADC+rHuPH20-blue).

FIG. 65 is a plot of serum concentration-time profiles for free payload (unconjugated SN-38) for individual animals (ADC-IV-purple; ADC-SC-red; ADC+rHuPH20-blue)

FIG. 66 is a plot of total ADC (TADC)/total antibody (TAB) ratios for individual animals (ADC-IV-purple; ADC-SC-red; ADC+rHuPH20-blue).

FIG. 67 is a plot of skin tissue concentration-time profiles for total antibody (TAB) for individual animals (ADC-IV-purple; ADC-SC-red; ADC+rHuPH20-blue).

FIG. 68 is a plot of skin tissue concentration-time profiles for total ADC (TADC) for individual animals (ADC-IV-purple; ADC-SC-red; ADC+rHuPH20-blue).

FIG. 69 is a plot of skin tissue concentration-time profiles for free payload (Payload) for individual animals (ADC-IV-purple; ADC-SC-red; ADC+rHuPH20-blue).

FIG. 70 is a plot of skin tissue total ADC (TADC)/total antibody (TAB) ratios for individual animals (ADC-IV-purple; ADC-SC-red; ADC+rHuPH20-blue).

FIG. 71 is a plot of total antibody (TAB) serum concentration versus time following administration of ADC-IV, ADC-SC, and ADC+rHuPH20-SC.

FIG. 72 is a plot of mean total ADC (TADC) serum concentration versus time following administration of ADC-IV, ADC-SC, and ADC+rHuPH20-SC.

FIG. 73 is a plot of total free payload serum concentration versus time following administration of ADC-IV, ADC-SC, and ADC+rHuPH20-SC.

FIG. 74 is a plot of TADC/TAB ratio over time for ADC-IV, ADC-SC, and ADC+rHuPH20-SC.

FIG. 75 is a plot of the TAB concentration in tissue homogenates of injection site (μg/mg protein±SD).

FIG. 76 is a plot of the TADC concentration in tissue homogenates of injection site (μg/mg protein±SD).

FIG. 77 is a plot of the free payload concentration in tissue homogenates of injection site (μg/mg protein±SD).

FIG. 78 is a plot of the TADC/TAB ratio in tissue homogenates of injection site (μg/mg protein±SD).

FIG. 79 is a plot of serum concentration-time profiles for total antibody (TAB) for individual animals (ADC-IV-purple; ADC-SC-red; ADC+rHuPH20-blue).

FIG. 80 is a plot of serum concentration-time profiles for total ADC (TADC) for individual animals (ADC-IV-purple; ADC-SC-red; ADC+rHuPH20-blue).

FIG. 81 is a plot of serum concentration-time profiles for free payload (unconjugated SN-38) for individual animals (ADC-IV-purple; ADC-SC-red; ADC+rHuPH20-blue).

FIG. 82 is a plot of total ADC (TADC)/total antibody (TAB) ratios for individual animals (ADC-IV-purple; ADC-SC-red; ADC+rHuPH20-blue).

FIG. 83 is a plot of skin tissue concentration-time profiles for total antibody (TAB) for individual animals (ADC-IV-purple; ADC-SC-red; ADC+rHuPH20-blue).

FIG. 84 is a plot of skin tissue concentration-time profiles for total ADC (TADC) for individual animals (ADC-IV-purple; ADC-SC-red; ADC+rHuPH20-blue).

FIG. 85 is a plot of skin tissue concentration-time profiles for free payload (Payload) for individual animals (ADC-IV-purple; ADC-SC-red; ADC+rHuPH20-blue).

FIG. 86 is a plot of skin tissue total ADC (TADC)/total antibody (TAB) ratios for individual animals (ADC-IV-purple; ADC-SC-red; ADC+rHuPH20-blue).

FIGS. 87A-87B provide plots of total payload in minipigs administered Trodelvy. FIG. 88A provides a plot for total payload in serum. FIG. 88B provides a plot for total payload in skin.

FIGS. 88A-88B provide plots of total payload in minipigs administered Trodelvy. FIG. 88A provides a plot for total payload in serum. FIG. 88B provides a plot for total payload in skin.

### DETAILED DESCRIPTION

### A. Definitions

Unless defined otherwise, all technical and scientific terms used herein have the same meaning as is commonly understood by one of skill in the art to which the invention(s) belong. All patents, patent applications, published applications and publications, GenBank® sequences, databases, websites and other published materials referred to throughout the entire disclosure herein, unless noted otherwise, are incorporated by reference in their entirety. If there are a plurality of definitions for terms herein, those in this section prevail. Where reference is made to a URL or other such identifier or address, it is understood that such identifiers can change and particular information on the internet can come and go, but equivalent information can be found by searching the internet. Reference thereto evidences the availability and public dissemination of such information.

As used herein the term ‘combination dosing regimen’ refers to at least two components administered together to a patient.

As used herein, the term ‘treatment’ or ‘treating’ refers to alleviating the specified condition, eliminating or reducing the symptoms of the condition, slowing or eliminating the progression, invasion, or spread of the condition and reducing or delaying the reoccurrence of the condition in a previously afflicted subject.

As used herein, the term ‘prevention’ or ‘preventing’ refers to precluding developing a disease, disorder, or condition or reducing the risk of developing the disease, disorder, or condition or reducing the symptoms thereof.

As used herein, “infusion related reaction” means a type of adverse event that occurs during or after the administration of a pharmacological or biological substance via infusion. These reactions can range from mild to severe and may involve various body systems. Common symptoms include itching, flushing, rash, hives, shortness of breath, wheezing, chest discomfort, changes in blood pressure, rapid heartbeat, dizziness nausea, vomiting and abdominal pain. Symptoms may appear within minutes to hours after infusion and usually within 24 hours. Infusion related reactions may also be referred to as hypersensitivity reactions or administration related reactions.

As used herein the term ‘injection site reaction’ means side effects at or near the spot where the infusion/injection was received. This includes pain or discomfort, redness, swelling, itching, bruising, lumps, infection complications (cellulitis or abscess), and irritation.

As used herein, a soluble hyaluronidase is a hyaluronidase of form thereof that is not GPI anchored, and that is soluble under physiological conditions and is secreted upon expression. Hyaluronidases, such as ovine and bovine hyaluronidases occur as soluble hyaluronidases. Human PH20 hyaluronidase does not occur as a soluble hyaluronidase. It is known in the art that removal of all or a part of the GPI anchor results in soluble forms.

As used herein the term ‘rHuPH20’ refers to the soluble hyaluronidase composition produced upon expression in a mammalian cell, such as a CHO cell, or other cell that effects glycosylation, of nucleic acid encoding residues 36-482 of SEQ ID NO:1. For expression in cells the encoding nucleic acid is linked to the native (residues 1-35 of SEQ ID NO:1) or a heterologous signal sequence for trafficking and secretion of the encoded polypeptides. The resulting secreted soluble glycoprotein is a heterogeneous mixture of polypeptides, including polypeptides that terminate at residues 479, 480, 481, and 482, and are composed of residues 36-479, 36-480, 36-481, and 36-482 with reference to SEQ ID NO:1. Shorter C-terminally truncated forms also may be included.

As used herein, “combination therapy” refers to a treatment in which a subject if given two or more therapeutic agents, such as at least two or at least three therapeutic agents, for treating a single disease.

As used herein, “hyaluronidase activity” refers to the ability to enzymatically catalyse the cleavage of hyaluronic acid. The United States Pharmacopeia (USP) XXII assay for hyaluronidase determines hyaluronidase activity indirectly by measuring the amount of higher molecular weight hyaluronic acid, or hyaluronan, (HA) substrate remaining after the enzyme is allowed to react with the HA for 30 min at 37° C. (USP XXII-NF XVII (1990) 644-645 United States Pharmacopeia Convention, Inc, Rockville, MD). A Reference Standard solution can be used in an assay to ascertain the relative activity, in units, of any hyaluronidase. In vitro assays to determine the hyaluronidase activity of hyaluronidases, such as PH20, including soluble PH20 and esPH20, are known in the art and described herein. Exemplary assays include the micro turbidity assay that measures cleavage of hyaluronic acid by hyaluronidase indirectly by detecting the insoluble precipitate formed when the uncleaved hyaluronic acid binds with serum albumin and the biotinylated-hyaluronic acid assay that measures the cleavage of hyaluronic acid indirectly by detecting the remaining biotinylated-hyaluronic acid non-covalently bound to microtiter plate wells with a streptavidin-horseradish peroxidase conjugate and a chromogenic substrate. Reference Standards can be used, for example, to generate a standard curve to determine the activity in Units of the hyaluronidase being tested.

As used herein, specific activity refers to Units of activity per mg protein. The milligrams of hyaluronidase is defined by the absorption of a solution of at 280 nm assuming a molar extinction coefficient of approximately 1.7, in units of M-1 cm-1.

As used herein, “neutral active” refers to the ability of a PH20 polypeptide to enzymatically catalyse the cleavage of hyaluronic acid at neutral pH (e.g. at or about pH 7.0).

As used herein, a “GPI-anchor attachment signal sequence” is a C-terminal sequence of amino acids that directs addition of a preformed GPI-anchor to the polypeptide within the lumen of the ER. GPI-anchor attachment signal sequences are present in the precursor polypeptides of GPI-anchored polypeptides, such as GPI-anchored PH20 polypeptides. The C-terminal GPI-anchor attachment signal sequence typically contains a predominantly hydrophobic region of 8-20 amino acids, preceded by a hydrophilic spacer region of 8-12 amino acids, immediately downstream of the @-site, or site of GPI-anchor attachment. GPI-anchor attachment signal sequences can be identified using methods well known in the art, such as but not limited to, in silico methods and algorithms (see, e.g. Udenfriend et al. (1995) Methods Enzymol. 250:571-582, Eisenhaber et al., (1999) J. Biol. Chem. 292: 741-758, Fankhauser et al., (2005) Bioinformatics 21:1846-1852, Omaetxebarria et al., (2007) Proteomics 7:1951-1960, Pierleoni et al., (2008) BMC Bioinformatics 9:392), including those that are readily available on bioinformatic websites, such as the ExPASy Proteomics tools site (e.g. the World Wide Web site expasy.ch/tools/).

As used herein, sequence identity refers to the relatedness between or among polypeptides among nucleic acid molecules. Sequence identity can be assessed by aligning two sequences and counting the number of differences between the aligned portion and the sequence to which it is compared. Whether any two molecules have nucleotide sequences or amino acid sequences that are at least 60%, 70%, 80%, 859%, 90%, 95%, 96%, 97%, 98% or 99% “identical” or “homologous” can be determined using known computer algorithms such as the “FASTA” program, using for example, the default parameters as in Pearson (1988) Proc. Natl. Acad. Sci. USA 85:2444 (other programs include the GCG program package (Devereux (1984) Nucleic Acids Research 12:387), BLASTP, BLASTN, FASTA (Altschul (1990) J. Mol. Biol. 215:403); Guide to Huge Computers, Bishop, ed., Academic Press, 1994, and Carrillo (1988) SIAM J. Applied Math 48:1073). For example, the BLAST function of the National Center for Biotechnology Information database can be used to determine identity. Other commercially or publicly available programs include, DNAStar “MegAlign” program and the University of Wisconsin Genetics Computer Group (UWG) “Gap” program. Percent homology or identity of proteins and/or nucleic acid molecules can be determined, for example, by comparing sequence information using a GAP computer program (e.g. Needleman (1970) J. Mol. Biol. 48:443, as revised by Smith and Waterman (1981) Adv. Appl. Math. 2:482. Briefly, the GAP program defines similarity as the number of aligned symbols (i.e. nucleotides or amino acids), which are similar, divided by the total number of symbols in the shorter of the two sequences. Default parameters for the GAP program can include: (1) a unary comparison matrix (containing a value of 1 for identities and 0 for non-identities) and the weighted comparison matrix of Gribskov (1986) Nucl. Acids Res. 14:6745, as described by Schwartz and Dayhoff, eds., Atlas of Protein Sequence and Structure, National Biomedical Research Foundation, pp. 353-358 (1979); (2) a penalty of 3.0 for each gap and an additional 0.10 penalty for each symbol in each gap; and (3) no penalty for end gaps.

Therefore, as used herein, the term “identity” or “homology” represents a comparison between a test and a reference polypeptide or polynucleotide.

As used herein, the term at least “90% identical to” refers to percent identities from 90 to 99.99 relative to the reference nucleic acid or amino acid sequence of the polypeptide. Identity at a level of 90% or more is indicative of the fact that, assuming for exemplification purposes a test and reference polypeptide length of 100 amino acids are compared. No more than 10% (i.e. 10 out of 100) of the amino acids in the test polypeptide differs from that of the reference polypeptide. Similar comparisons can be made between test and reference polynucleotides. Such differences can be represented as point mutations randomly distributed over the entire length of a polypeptide or they can be clustered in one or more locations of varying length up to the maximum allowable, e.g. 10/100 amino acid difference (approximately 90% identity). Differences are defined as nucleic acid or amino acid substitutions, insertions or deletions. At the level of homologies or identities above about 85-90%, the result should be independent of the program and gap parameters set; such high levels of identity can be assessed readily, often by manual alignment without relying on software.

As used herein, an aligned sequence refers to the use of homology (similarity and/or identity) to align corresponding positions in a sequence of nucleotides or amino acids. Typically, two or more sequences that are related by 50% or more identity are aligned. An aligned set of sequences refers to 2 or more sequences that are aligned at corresponding positions and can include aligning sequences derived from RNAs, such as ESTs and other cDNAs, aligned with genomic DNA sequence.

As used herein, “denaturing condition” or “denaturation condition” refers to any condition or agent that, when exposed to a protein, affects or influences the degradation or denaturation of the protein, generally as a result of a loss or partial loss of the tertiary or secondary structure of the protein. Denaturing conditions can result in effects such as loss or reduction in activity, loss or reduction of solubility, aggregation and/or crystallization.

As used herein, “resistance to a denaturation condition” refers to any amount of decreased reduction or elimination of a property or activity of the protein associated with or caused by denaturation. For example, denaturation is associated with or causes increased crystallization or aggregation, reduced solubility or decreased activity. Hence, resistance to denaturation means that the protein exhibits decreased aggregation or crystallization, increased solubility or increased or greater activity (e.g. hyaluronidase activity) when exposed to a denaturing condition compared to a reference protein (e.g. unmodified enzyme).

As used herein, stability of a modified PH20 hyaluronidase means that it exhibits resistance to denaturation caused by a denaturation condition or denaturing agent.

As used herein, a “payload” is an active substance (e.g., a therapeutic agent, gene, or diagnostic compound) that is transported by a delivery system to a target site within the body. The delivery system (which can be thought of as the “vehicle”) may include nanoparticles, viral vectors, liposomes, antibody-drug conjugates, antibody, protein, peptide, or fragment of any of these. The payload, in this case, is distinct from the structural or functional components of the delivery mechanism and in various embodiments ensures stability, targets specificity, and controls release of the therapeutic agent.

As used herein, a “linker” is a chemical or molecular segment that covalently or non-covalently joins two or more biomolecules (e.g., proteins, peptides, nucleic acids, and small molecules) into a single functional entity. This connecting component is designed to provide structural flexibility, maintain stability, and often incorporate specific cleavage sites to enable controlled interactions or the release of connected components when required. Linkers can be an essential modular component used to connect two or more biomolecules without interfering with their individual functions. Its design can be customized for flexibility, fixed orientation, or controlled release, depending on the application. Whether in fusion proteins, ADCs, or nucleic acid constructs, the incorporation of an appropriate linker preserves the biological activity, enhancing stability, and achieving targeted interactions in a range of biomedical applications.

As used herein, a “tissue” is a structurally organized group of similar cells and their associated extracellular matrix that work together to perform specific functions vital for an organism's survival. The structural and functional complexities of tissues underpin the formation of organs and the integrated systems that sustain life, making them a central subject in biomedical research and tissue engineering.

As used herein, “plasma” is the liquid matrix of blood, primarily composed of water, and containing proteins, salts, hormones, nutrients, waste products, and clotting factors (like fibrinogen) responsible for transporting blood cells and various substances throughout the body. Plasma is the liquid component of blood that serves as the medium for transporting nutrients, hormones, waste products, and other substances throughout the body. It makes up about 55% of total blood volume and consists primarily of water (about 90%), along with dissolved proteins (such as albumin, fibrinogen, and globulins), electrolytes, glucose, lipids, gases (like oxygen and carbon dioxide), and waste products. Plasma also contains clotting factors, which play a crucial role in blood coagulation. It is essential for maintaining blood pressure, regulating body temperature, and supporting the immune system by transporting antibodies and other defense molecules.

As used herein, “blood” or “whole blood” is the complete fluid circulating in the body, composed of formed elements (red blood cells, white blood cells, platelets) suspended in plasma. It is a vital bodily fluid in humans and most animals that delivers essential substances, such as oxygen, nutrients, and hormones, to cells while removing waste products like carbon dioxide and toxins. It is composed of plasma (a liquid that contains water, proteins, and other dissolved substances) and cellular components, including red blood cells, white blood cells, and platelets. Blood circulates through the body via the cardiovascular system, driven by the pumping action of the heart. It plays a critical role in maintaining homeostasis, immune defense, and regulating body temperature. In embodiments of the present disclosure, “blood” may be used to refer to serum or plasma. One of skill in the art would understand that the analytical measurements used herein, including pharmacokinetic measurements such as AUC, Tmax, and Cmax, may use the term “blood” when referring to serum or plasma.

As used herein, “serum” is like plasma but lacks clotting factors which have been removed during the process of blood clotting. Serum is a fluid component of blood that remains after blood has clotted and the clotting factors have been removed. It is essentially blood plasma without the clotting proteins such as fibrinogen. Serum contains a variety of dissolved substances, including electrolytes, hormones, antibodies, and proteins like albumin.

As used herein, “therapeutically effective dose” (TED) is the amount of a drug or therapeutic agent that produces the intended beneficial effect in a patient under defined clinical conditions. This dose is determined during clinical development and is critical for achieving the desired therapeutic outcome while minimizing adverse effects.

The term “equivalent dose” refers to the amount of a therapeutic agent that, when administered, produces a comparable level of clinical effect—be it efficacy or toxicity—to a reference treatment. This ensures consistent patient outcomes, particularly in the evaluation of generic products and the adjustment of dosing regimens for individualized therapies. An equivalent dose can be the actual equivalent to a reference treatment of a similar dose to a reference treatment. A similar dose can be 80% to 125%, 85% to 120%, 90% to 115%, 95% to 110%, 95% to 105%, or 100% of the reference treatment dose.

As used herein, “AUC” or “Area under the Curve” refers to the area under the plasma or serum concentration versus time curve, indicative of the systemic exposure to the drug over a defined period of time. The value of AUC is dependent on the duration over which plasma or serum concentrations are measured and reflects the extent of drug absorption and bioavailability within that specified interval.

As used herein, “Cmax” or “maximum concentration” refers to the peak plasma or serum concentration of the drug achieved after administration, reflecting the maximum systemic exposure at a single time point. The value of Cmax is dependent on factors including the administered dose and the route of administration (e.g., subcutaneous, intravenous, oral), which influence the rate and extent of drug absorption.

As used herein, “subject” refers to an individual, organism, or entity that is being studied, observed, or experimented upon. Subjects can range from mammals (e.g., humans and non-human primates), birds, reptiles, amphibians, and other species.

As used herein, “antibody” refers to a molecule which comprises or contains: (a) one or more immunoglobulin variable domains; or (b) fragments, variants, modifications or derivatives of such immunoglobulin variable domains irrespective of origin or source, including but not limited to antigen binding portions including Fab, Fab′, F(ab′)2, Fv, dAb and CDR fragments, single chain antibodies (scFv), chimeric antibodies, monospecific antibodies, multi-specific antibodies, diabodies and polypeptides (including humanized versions thereof) that contain at least a portion of an immunoglobulin that is sufficient to confer specific antigen binding to a polypeptide.

As used herein, “antibody-drug-conjugate” or “ADC” or “Total ADC” is a specialized molecule composed of an antibody, which is designed to target specific cell types, chemically linked to one or more payloads that can be cytotoxic or have other therapeutic effects. ADCs also include one or more linkers that connect the antibody to the payload(s), enabling precise delivery of the therapeutic agent to the target cells or tissues.

As used herein, the “drug-to-antibody ratio” or “DAR” refers to the number of payload molecules attached to each antibody, which is typically greater than or equal to one (1). ADCs are widely used in targeted therapies, particularly in cancer treatment, to minimize damage to healthy cells while enhancing the efficacy of the drug payload.

As used herein, “total antibody” refers to the combined amount of the intact antibody-drug conjugate (ADC) and the antibody component of the ADC that is no longer attached to its linker or payload due to detachment. This measurement accounts for both the fully or partially assembled ADC molecule and the free antibody that remains after the payload or linker has been released. Monitoring total antibody levels is important in pharmacokinetics and drug development to assess the stability, efficacy, and safety of ADC-based therapies.

As used herein, “free antibody” refers to the portion of the total antibody present in a sample that is not part of an intact antibody-drug conjugate (ADC). It is calculated as the difference between the total antibody (which includes both intact ADC and any detached antibody) and the intact ADC itself. Free antibody is typically monitored in pharmacokinetic studies to evaluate the stability of the ADC and to understand its behavior in vivo, including potential off-target effects or immune responses.

As used herein, “free payload” refers to the portion of the drug payload that is not conjugated to an antibody in an antibody-drug conjugate (ADC). This can include payload molecules that have been released from the ADC due to linker cleavage or degradation, as well as payloads that were not attached during the conjugation process. Monitoring free payload levels is critical in pharmacokinetics and toxicology studies to assess potential off-target effects, systemic toxicity, and therapeutic efficacy of ADC-based treatments.

As used herein, “total payload” refers to the combined amount of drug payload present in a sample, regardless of its state. This includes payload molecules that are conjugated to an antibody via a linker (as part of an intact antibody-drug conjugate, or ADC), payload molecules attached to a linker but no longer bound to the antibody, and payload molecules that exist independently without any linker or antibody. Measuring total payload is essential in pharmacokinetics and drug development to evaluate the overall distribution, efficacy, and potential toxicity of ADC-based therapies.

As used herein, “site of administration” refers to the specific anatomical location where a therapeutic agent, such as a drug or biologic, is introduced into the body for absorption or action. This can encompass various layers of tissue depending on the route of delivery, including the epidermis (outermost layer of the skin), dermis (middle layer containing connective tissue, blood vessels, and nerves), hypodermis (also known as the subcutaneous tissue, which consists of fat and connective tissue beneath the dermis), and the subcutaneous space (the broader region within the hypodermis where subcutaneous injections are typically administered). The site of administration is chosen based on factors such as the drug's formulation, the desired absorption rate, and patient-specific considerations.

### Systemic Circulation

As used herein, “systemic circulation” refers to the overall process of distributing oxygenated blood, nutrients, hormones, and other essential substances throughout the entire body, and simultaneously collecting deoxygenated blood and metabolic waste products for transport back to the heart. It is the overarching circulatory pathway that ensures all body tissues receive the necessary supplies transported by whole blood, which includes both the cellular and fluid components (plasma and serum). This vital process involves blood, plasma and serum.

As used herein, “site of action” refers to the specific anatomical location, encompassing tissues, organs, or individual cells (including, for instance, tumor tissue or specific target cells), where a drug or therapeutic agent exerts its intended biological or pharmacological effect.

For clarity of disclosure, and not by way of limitation, the detailed description is divided into the subsections that follow.

### B. Overview

Provided are combination dosing regimens, comprising administering a hyaluronidase; and administering an antibody-drug conjugate. The agents in the dosing regimens can be administered sequentially, intermittently, serially, in the same composition, and/or in other combinations of the agents. The combination dosing regimens described herein are dosing regimens suitable to be provided to a patient in order to treat or prevent a disease/disorder.

In an embodiment of the combination dosing regimens provided are dosing regimens suitable for treating a cancer. In an alternative embodiment, the combination dosing regimen provided herein is for preventing cancer. The combination dosing regimen is administered to a patient in need of treatment for a disease/disorder.

In embodiments herein, provided are combination dosing regimens in which an antibody-drug conjugate and a soluble hyaluronidase are administered.

### C. Antibody-Drug Conjugates

In embodiments herein, antibody-drug conjugates (ADC) are comprised of a monoclonal antibody (mAb), a cytotoxic payload, and a linker. Once the ADC reaches the target cells, the mAb component recognizes and binds to antigens, and the ADC-antigen complex is then internalized within the cancer cell by endocytosis to form an early endosome, which, following a maturation, forms late endosomes and finally fuses with lysosomes. The cytotoxic drug payload is then released from the mAb via either a chemical reaction or enzyme digestion in the lysosomes, and exerts its cytotoxic effect, causing cell apoptosis or death.

In addition to the cytotoxic properties from the payload, the Fc portion of the monoclonal antibody aids in immune-related cytotoxicities, such as antibody-dependent cell mediated cytotoxicity (ADCC), antibody-dependent phagocytosis (ADP), and complement-dependent cytotoxicity (CDC). Genetic engineering technologies have advanced to enhance the effector function of the antibody in the Fc region. Additionally, the binding of the antibody component of ADC with the specific antigen epitope of cancer cells can inhibit the downstream signal transduction of the antigen receptor.

ADCs have proven to be effective for focused delivery of toxic chemotherapeutics. However, dialing in an optimal therapeutic profile of ADCs remains a challenge, as common adverse reactions, such as decreased blood cell count (Nguyen, T. D. et al., “Mechanisms of ADC Toxicity and Strategies to Increase ADC Tolerability.” Cancers, 2023, 15(3):713; incorporated by reference herein), are driven by peak concentrations, or Cmax (Patel M. et al., “Dose schedule optimization and the pharmacokinetic driver of neutropenia.” PLOS One, 2014, 9(10):e109892; incorporated by reference herein), while efficacy is driven by the average concentration, Cavg or AUC (Hinrichs M. J. M., et al., “Fractionated Dosing Improves Preclinical Therapeutic Index of Pyrrolobenzodiazepine-Containing Antibody Drug Conjugates,” Clin. Cancer Res., 2017, 23(19):5858-5868; incorporated by reference herein). To date, ADCs have been delivered intravenously (IV), which inherently results in large swings between these maximum and average concentrations. Subcutaneously (SC) delivery offers the opportunity for flattening the PK curve, potentially enabling improved safety and/or efficacy of ADCs.

An appropriate selection of target antigen is central to the design of an ADC. First, the antigen should be expressed, either exclusively or predominantly, in the tumor cells to reduce the off-target toxicity. Secondly, the binding to the target antigen should ideally lead to the internalization of the antigen-antibody complex. Additionally, it should ideally be on the surface rather than intracellular for it to be recognized, and lastly, it should not be secretory since a secreted antigen in the circulation would cause the undesirable ADC to bind outside of the tumor sites. Exemplary target antigens for ADC include but are not limited to, CD19, CD22, CD30, CD33, and CD79b in hematological malignancies and HER2, trop2, nectin4, tissue factor, and folate receptor alpha (FRα) in solid cancers. Additional non-limiting examples of ADC targets are below.

5T4
actM
ADAM9
Alkaline phosphatase, placental-like 2
ASCT2
Ax1
B7-H3
B7-H4
BCMA
CA242
CAFs
Calnexin
Cancer-specific Tn-glycosylated proteins
CBLB
CCR7
CCR8
CD123
CD155
CD166
CD205
CD228
CD239
CD24
CD3
CD37
CD38
CD44v9
CD45
CD46
CD47
CD56
CD6
CD7
CD70
CD73
CD74
CDCP1
CDH17
CDH6
CEACAM5
CEACAM6
cKIT
CLDN1
CLDN18.2
CLDN6
CLDN9
c-MET
CNTN4
core 2 O-glycan
CSF-1R
CT22
CXCR5
DLK-1
DLL3
Doppel
DPP4
DR5
EGFR
Endoglin
ENPP3
EpCAM
EphA2
EphA5
FAP
FcγR
FGFR2b
FGFR3
FGFR4
Fibronectin extra-domain B
FLT3
G protein-coupled receptors
GCC
GD2
GDF-15
GIPR
Globo H
GLP-1R
Glycan
GP75
GPC3
gpNMB
GPRC5D
GUCY2C
HER-3
HERV-H LTR-associating protein 2
HLA-G
Human Cytomegalovirus
ICAM-1
IGF-1R
IL-12
IL13Rα2
IL1RAP
IL-2R Alpha
IL-6
Immunoglobulin G
Integrin beta-6
Integrin α10β1
Integrin αVβ6
ITGB4
KIF20A
L1CAM
LAIR1
LGALS3BP
LILRB2
LIV-1
LRRC15
Ly6E
LY6G6D
Meflin
MerTK
Mesothelin
MET
MHC class I chain-related protein A and B
MRC2
MTX7
MUC1
MUC-1
MUC13
MUC16
MUC18
MUC1-C
NaPi2b
Neuraminidase
NPTxR
P-Cadherin
PD-1
PD-L1
PD-L2
PSMA
PTK7
RET
RON (Recepteur d'Origine nantais)
ROR1
ROR2
RYK
SEZ6
SIGLEC2
SIGLEC3
SLITRK6
SSTR2
STEAP-1
STEAP2
TAA
Tfr1
TIM-3
TLR7
TLR7/8
TM4SF1
TRA-1-60
TRP1
TSLPR
Tumor microenvironment
Tumor-Associated Carbohydrate Antigens
Tumor-associated macrophages
Tyrosine kinases
USP7
VEGF

An ideal antibody moiety should facilitate an effective internalization, have high antigen affinity, preserve long plasma half-life, and demonstrate low immunogenicity. The mAb are large-sized and account for over 90% of the mass of any given ADC. This is favorable because it encounters reduced distribution or permeation into healthy tissue, including those normally functioning as metabolizing and eliminating organs. No such problem is encountered at the tumor site as the vasculature in the tumor is characteristically leaky and allows the distribution and permeation of the ADC to the tumor cells.

In an embodiment, there are two types of linkers in the ADC, including cleavable and non-cleavable. The cleavable linkers are either chemically labile (hydrazone bond and disulfide bond) or enzymatically labile. Hydrazone linkers are generally stable in alkaline environments and are hydrolyzed in low pH environments, such as that in the lysosome and endosome. Hence, the cleavage of ADC with hydrazone linkers occurs predominantly in the lysosome and endosome upon internalization, with occasional hydrolysis in the plasma, resulting in off-target, systemic toxicity. Similarly, a disulfide bond linker can be stable in the plasma while specifically releasing the active payloads in the cancer cells with an elevated reductive glutathione level. The enzyme sensitive linkers are sensitive to the lysosomal protease that is generally overexpressed in cancer cells, enabling an accurate drug release in the cells after internalization. ADC with non-cleavable linkers are resistant to chemical or enzymatic digestion in the plasma and will require complete degradation of the antibody within the late endosomes and lysosome to release the payload. Therefore, ADC with non-cleavable linkers may have the lowest off-target systemic toxicity due to increased plasma stability and thus they are most suitable in the treatment of tumors with homogenous antigen expression. In an embodiment, some of the ADC have been engineered to have desirable “off-target effect” for “by-stander killing” extending the cytotoxic effect to the low or negative antigen-expressing cells in the tumor proximity. For this mechanism to work, several characteristics of the ADC molecules are crucial: namely, a cleavable linker and a non-polar, freely membrane-permeable payload. Conversely, to reduce the undesirable systemic toxicity from payload molecules permeating out of the tumor cells, ionizable payloads (e.g. containing carboxylic acids) can be used. Non-limiting examples of specific types of linkers useful in the invention described here are below.

ABNO
AcBut acyl hydrazone-disulfide
Acetyl Butyrate
Alanine-Alanine
Alanine-Proline
Alco5
Disulfonyl-ethyl carbonate/carbamate
D-leucine-alanine-glutamate (DLAE)
Fleximer Polymer
Furin-Cleavable Linker
G4S
GGFG (Glycine-Glycine-Phenylalanine-
Glycine)
Glucuronide
Glu-Gly-Cit (Glutamic acid-Glycine-
Citrulline [EGCit])
Glycine-Serine
Gly-Gly-Gly
L-Ala-D-Ala-L-Ala
Lys-Gly-Asp-Glu-Val-Asp (KGDEVD)
Maleimide
Maleimide-Alanine-Alanine-Alanine (MC-
AAA)
Maleimide-PEG24
Maleimidocaproyl
Maleimidomethyl cyclohexane-1-carboxylate
MCC
MC-vc-PAB
Met-His
Ortho-Hydroxy Protected Aryl Sulfate
(OHPAS)
Oxime
PEG24
Pentaglycine
pentaglycine-EDA
Phosphine-azide
polyethylene glycol
Benzimidazole
beta-Glucuronide
CL2A
Covalently Linked
Dibenzocyclooctyne (DBCO)
dibromomaleimide (DBM)
Protease Cleavable
SIAB (N-succinimidyl [4-iodoacetyl]
aminobenzoate)
SMCC
SPDB
Sulfo-SPDB
T1000
TCO (trans-cyclooctene)
TMALIN
Valine-Alanine
Valine-Citrulline
Valine-Glucoserine(GlcA)
Valine-Lysine

In an embodiment, the cytotoxic payloads should ideally have the following properties. High potency, in vitro high cytotoxic activity (sub-nanomolar half maximal inhibitory concentration (IC₅₀) value), high stability in the systemic circulation, sufficient solubility in the aqueous environment of antibody and biochemical properties to allow easier conjugation to the antibody, low immunogenicity, small molecular weight, and a long half-life. In an embodiment, there are mainly two classes of cytotoxic drugs used as payloads, microtubule inhibitors or DNA damaging agents.

In an embodiment, auristatins and maytansines payloads are both cytotoxic agents that work as tubulin inhibitors. Auristatin is a dolastatin synthetic analog. There are two auristatin derivatives: one is monomethyl auristatin E (MMAE) and the other is monomethyl auristatin F (MMAF). These two cytotoxic agents differ structurally wherein the phenylalanine present at the C-terminus renders MMAF membrane-impermeable, whereas the MMAE can exit the cell and thus diffuse to nearby cells and kill them through the bystander effects. In an embodiment, the cytoxic agent is selected from maytansinoids which are natural cytotoxic agents isolated from the cortex of Maytenus serrata, which possesses a macrolide structure.

In an embodiment, the cytotoxic agents are selected from calicheamicins, pyrrolobenzodiazepines and topoisomerase inhibitors which are DNA-damaging agents that act through DNA double strand breaks, crosslinking, and intercalation, respectively. Both gemtuzumab ozogamicin and inotuzumab Ozogamicin have N-acetyl gamma calicheamicin as a payload. Calicheamicins belong to a class of potent anti-tumor antibiotics that cleave the DNA in a site-specific, double-stranded manner. Pyrrolobenzodiazepines are another class of antibiotics derived from Streptomyces species and is used as a cytotoxic payload in Loncastuximab Tesirine. SN-38 and Deruxtecan are topoisomerase inhibitors that are the cytotoxic components of Sacituzumab Govitecan and Trastuzumab Deruxtecan, respectively. Any of the above cytotoxic agents are embodied in the ADC. Non-limiting examples of payloads useful in the invention described herein are shown below.

7PB-100
AF-HEA (Auristatin)
(MMAF)
AMDCPT (Camptothecin)
Anthracycline
AP052
ATI020
Auristatin F-HPA (XMT-1267)
(Auristatin)
Auristatin W analog
AxcynDOT (Trabectedin)
AZ13599185
AZ14170132 (AZ′0132)
(Exatecan)
Azonafide
BCPT02
Belotecan
BLD1102
BRD4 degrader
C24
Camhexin
Camptothecin
CEN371
Ceralacertib
Clezutoclax
CLYP-71 (Lytic Peptide)
CPI (Cyclopropylpyrroloindole)
CPT-113
CPT-113 (Camptothecin)
CPT116
cross-linking PBD/IGN
Cryptophycin
Cyclopropylpyrroloindole (CPI)
Cytolysin
d HBD
D2102
D211
DDDXd (DXd/DX8951 (MAAA-
1181a))
DGN462
DGN549
DGN549 (Indolino-
benzodiazepine dimer (IGN))
dHBD (heterocycle-fused
LDX2
Lenalinomide
Lidamycin
LMP517 (dual action)
LP2
LPDUP5
Maytansine
MED-A/DNAMGBA toxin
MH30010008 (Exatecan)
MMAD
MMAU (Auristatin)
monoalklyating PBD/IGN
Mtoxin ™ (MF-6)
NMS-P528
NT1
P1003
P1021
Paclitaxel (Taxanes)
PBX-7
PBX-7016
PE-E2K
PF-06380101 (Aur 101)
(Auristatin)
PH5
PH6
PL2202 (Camptothecin)
PM050489
PNU-159682 (Anthracycline)
ProAlk (Duocarmycin)
PROTAC
PRT3789 (SMARCA2)
PY-4car2 (Camptothecin)
QLS6916
RIPK2 degrader
SC3386 (Exatecan)
SC-DR003
SG2000
SG2057 (Pyrrolobenzodiazepine
(PBD))
SG3199 (Pyrrolobenzodiazepine
(PBD))
SG3249 (Pyrrolobenzodiazepine
Amanitin
Amberstatin269 (AS269)
benzodiazepine dimer)
(Pyrrolobenzodiazepine (PBD))
DIACC2010
DM1 (Maytansine)
DM21 (Maytansine)
DM4 (Maytansine)
Docetaxel (Taxanes)
Doxorubicin
DUBA
Duocarmycin
DUocarmycin-hydroxyBenzamide
Azaindole (DUBA) (Duocarmycin)
Duostatin 5.2 (MMAF)
Duostatin5 (MMAF)
Duostatin5.2
Dxh (Exatecan)
Ed-04 (Alkaloid Camptothecin)
E-P125A
Eribulin
Exatecan (Camptothecin)
F554443 (MMAF)
FGX20-75
FGX2-62
(Pyridinobenzodiazepines (PDD))
GNE-987
GS-P-000
GSPT1 degrader
HC74
HLX91-048
HS-9265 (Exatecan)
I-BiPs
IGN-P1
indolinobenzodiazepine dimer
(IGN)
JS-1
KL610023 (Belotecan)
KLG10023
KSP inhibitor
LCB20-0187
(Pyrrolobenzodiazepine (PBD))
LD2
LD-38
(PBD))
SG3376
SG3552
SGD-1882
(Pyrrolobenzodiazepine (PBD))
SHR152852
SHR9265 (Exatecan)
SMol006
Synthis-003
T01
TAM470 (Cytolysin)
Temozolomide
Thailanstatin (PH1)
Thienoindole
Triptolide
Tub114 (Tubulysin)
Tub196 (Tubulysin)
Tub201 (Tubulysin)
Tub255 (Tubulysin)
Tubulysin
Tubulysin A
Tubutecan
Utidelone
VIP716
YL0010014 (Camptothecin)
YL0014 (Camptothecin)
ZD02044
ZD06519 (Camptothecin)

| Payload Mechanism | Example Payloads |
|---|---|
| Topoisomerase I Inhibitor | A-1743332, AMDCPT, ATI020, AZ14170132, |
| | AZ14170133, BCPT02, Belotecan, BLD1102, |
| | CAMP59, Camptothecin and |
| | derivatives/analogues, CPT-113, CPT116, |
| | CPT2, D2102, DDDXd, DXd/DX8951, Dxh, |
| | Ed-04, Exatecan and derivatives/analogues, |
| | GS-P-000, HC74, HS-9265, Irinotecan and |
| | derivatives/analogues, JS-1, KLG10023, LD2, |
| | LD-38, LDX2, LMP517, MF-6, MH30010008, |
| | NT1, P1003, P1021, PBX-7, PBX-7016, |
| | PL2202, PY-4car2, QLS6916, SC3386, |
| | SHR9265, SN-38, T01, Tavatecan, Tubutecan, |
| | VIP126, YL0010014, YL0014, ZD06519 |
| Topoisomerase II Inhibitor | Anthracyclines, Doxorubicin, |
| | Epirubicin, PNU-159682 |
| Tubulin Inhibitor | AF-HEA, Amberstatin269, AP052, Auristatin |
| | and derivatives/analogues, Auristatin F-HPA, |
| | Auristatin W analog, AZ13599185, |
| | Cryptophycin, Cytolysin, DIACC2010, DM1, |
| | DM21, DM4, Docetaxel, Duostatin5, |
| | Duostatin5.2, ER300, Eribulin, F55443, |
| | Hemiasterlin, KSP inhibitor, LP2, M24, |
| | Maytansine and derivatives/analogues, |
| | MMAD, MMAE, MMAF, MMAU, Paclitaxel, |
| | PE-E2K, PF-06380101/Aur0101, PM050489, |
| | SC209, SHR152852, TAM470, Tub114, |
| | Tub196, Tub201, Tub255, Tubulysin A, |
| | Tubulysin and derivatives/analogues, |
| | Utidelone, ZD02044 |
| DNA Damaging Agent | AST-2660, AxcynDOT, Azonafide, |
| | Calicheamicin and derivatives/analogues, |
| | Cyclopropylpyrroloindole, D211, DGN462, |
| | DGN549, dHBD, DUBA (DUocarmycin- |
| | hydroxyBenzamide Azaindol), Duocarmycin |
| | and derivatives/analogues, FGX20-75, FGX2- |
| | 62, I-BiPs, IGN-P1, LCB20-0187, Lidamycin, |
| | MED-A/DNAMGBA toxin, Melphalan, |
| | Methotrexate, NMS-P528, PH5, PH6, |
| | Pyridinobenzodiazepine and |
| | derivatives/analogues, Pyrrolobenzodiazepine |
| | and derivatives/analogues, SC-DR003, |
| | SG2000, SG2057, SG3199, SG3249, SG3376, |
| | SG3552, SGD1882, SGD-1882, |
| | Temozolomide, Thienoindole |
| Degradation | BRD4 degraders, CDG0501, dKIF976, EBET- |
| | 1055, EBET-1593, GNE-987, MZ1, |
| | PROTACs, PRT3789, RIPK2 degraders, |
| | SMol006 |
| Immunomodulator | CEN371, CpG ODNs, CRD5500, diABZI, |
| | E7766, Imidazoquinoline, IMSA172, JAB- |
| | 27670, Lenalidomide, MTT-5, Phosphonate, |
| | Resiquimod, SZU-101, Tacrolimus, TAK676, |
| | Tofacitinib |
| Protein Toxin | deglycosylated ricin A, Diphtheria Toxin and |
| | derivatives/analogues, dmDT390, DT388, |
| | DT390, PE24, PE25, PE38, Pseudomonas |
| | exotoxin A, ricin A, SEA/E-120 |
| TLR agonists, including TLR7, TLR8, | Imidazoquinoline, Resiquimod (R848), |
| and TLR 7/8 agonists | E104, MTT-5 |
| STING Agonist | diABZI, E7766, TAK-676 |
| RNA Polymerase Inhibitor, RNA | Amanitin, Thailanstatin, Triptolide |
| Polymerase II Inhibitor | |
| PARP Inhibitor | Rucaparib, Talazoparib |
| Cell Membrane Disruption | CLYP-71, Hecate |
| Mitochondrial Disruption | Coenzyme Q10, d(KLA)2 |
| NMT Inhibitor | MYX2339, Zelenirstat |
| ATR Inhibitor | Ceralacertib |
| BAK activation | 7PB-100 |
| BCL-XL Inhibitor | Clezutoclax |
| Cystine-glutamate Antiporter Inhibitor | Erastin (Quinazolinone) |
| DNA Damage Response Inhibitor | |
| GLP1 receptor agonist | Peptides |
| Glucocorticoid Receptor Modulator | Glucocorticoids (GCs) |
| G-protein coupled receptor (GPCR) | FR900359 (Peptides) |
| inhibitor | |
| Kinase inhibitor | |
| KSP Inhibitor (KSPi)/Eg5 Inhibitors | VIP716 |
| MCL-1 Inhibitor | S64315 (MIK665) |
| Na, K-ATPase Inhibitor | CEN-106 |
| NAMPT Inhibitor | |
| Phosphatase inhibitor | Microcystin |
| Phosphoinositide 3-kinase (“PI3K”) | |
| inhibitor | |
| Proteasome Inhibitor | |
| Protein Alkylation | ProAlk (Duocarmycin) |
| Tyrosine kinase inhibitor | |
| TβRI/ALK5 Inhibitor | Synthis-003 |
| Other mechanisms | 7PB-100, Bcl-xL inhibitor, Buparlisib, |
| | Camhexin, CEN-106, Ceralacertib, |
| | Clezutoclax, CLYP-71, Curcumin, des-methyl- |
| | erastin, DNA Damage Response Inhibitors, E- |
| | P125A, FR900359, Glucagon-like peptide-1 |
| | analogues, Glucocorticoids/Steroids, |
| | Granzyme B, HLX91-048, LPDUP5, |
| | Microcystins, MK-801, MYX2339, NAMPT |
| | Inhibitor, N-linked glycosylation inhbitor-1, |
| | NMT inhibitor, PI3K inhibitors, ProAlk, |
| | Proteasome Inhibitors, S64315, Synthis-003, |
| | Talazoparib, TGFβR antagonist, Thapsigargin, |
| | Tyrosine kinase inhibitor, Urease, |
| | Venadaparib, VIP716, Zelenirstat |

In addition to the choice of the antibody, the linker, and the payload, the method of conjugation is also important for the successful structure of ADC. In an embodiment, the lysine and cysteine residues on the antibody provide the accessible reaction sites for conjugation. In an embodiment, a varying number (0-8) of small-molecule toxins may be attached to an antibody, as the conventional conjugation methods are random, resulting in a wide drug-antibody ratio (DAR) distribution. In an embodiment, the ideal DAR is 2-4. A low DAR can lower the efficacy, while a high DAR may increase the drug potency. Non-limiting examples of conjugation technologies useful in various embodiments of the invention described herein are shown below

AbClick ® Pro
AbClick ® Standard
AbYlink ™
Actibody
ADCplex ™
AJICAP ® site specific conjugation
AraLinQ ™
Araris' Proprietary Linker-Conjugation
Technology
AxcynCYS ™
Bacterial Transglutaminase (BTG) Site-
specific Technology
Bb-Dar ™
BiM technology
BTG (Bacterial transglutamination)
ByonShieLD ®
CD38 tag mediated
Click-chemistry-based drug conjugation
technology
C-Lock ™
Co-Nectar
CoNectar ™
ConjuAll ™
CROSSCONJU ™
CysLink ™
CYSMAB technology
Cystine sites: full reduction of interchain
disulfides
CysTyr ® platform
DBCO tag
mediated
EuCODE ™
Flexible Antibody Conjugation Technology
(FACT)
GeminiMab ™
GL-DisacLink ™
GlyCLICK
GlycOBI ™
GlycoConnect ™
Glycosyl conjugation technology
i-Conjugation ™
IDDC ™ (Interchain-Disulfide Drug
Conjugate)
iGDC ®
iLDC ®
iLDC ® and iGDC ®
K-Lock ™
MATE ™
McSAF Inside ®
MuSC ™ platform
N terminal serine conjugation
NECOR ™ (N-Glycan Enzymatic
Customized One-pot Remodeling)
NexMab ™ ADC technology
NTERM Conjugation
N-terminal cysteine conjugation
N-terminal glutamate conjugation
OmniLink ™
P5 conjugation platform
pClick Technology
Peptide Asparaginyl Ligase (PAL) one-pot
conjugation
PermaLink
RESPECT ®
RESPECT-H
RESPECT-L
Selenomab
SMAC-Technology ™
SMARTag ®
SpyCatcher-SpyTag
Stochastic conjugation-cysteine sites,
including partial reduction of interchain
disulfide bonds
Stochastic conjugation-lysines sites
TheranoStick ™
ThioBridge ®
THIOMAB ™
TRAAC Platform
Tub-tag ® conjugation technology
Universal Antibody Connectors ™ (UAC)
WuXiDARx
XL-XDC
XpressCF+ ®
ZymeLink ™
π-clamp tag
mediated

In one embodiment, the ADC comprises a cleavable linker. Exemplary classes of cleavable linkers include, but are not limited to, an acid labile linker, an enzyme cleavable linker, a reducible disulfide linker/glutathione-sensitive linker, an Fe (II)-responsive linker, an oxidation labile/ROS (reactive oxygen species) sensitive linker, a photo-responsive linker, a bioorthogonal linker, or a combination thereof. In embodiments, the enzyme cleavable linker is a dual or multiple enzyme cleavable linker. In embodiments, the cleavable linker comprises a chemically cleavable linker that can be cleaved by any chemical means known to a person of skill in the art.

Exemplary ADC, antigen targets, linkers and cytotoxins for use in the present invention are included in the table below.

| | Antigen | | | Payload | |
|---|---|---|---|---|---|
| ADC | Target | Linker | Cytotoxin | Target | Dose |
| Gemtuzumab | CD33 | Cleavable | N-acetyl gamma | Minor | 2-3 mg/m² |
| ozogamicin | | acid-labile | Calicheamicin | groove | mono |
| (Mylotarg) | | hydrazone | dimethyl | of DNA | 3-6 mg/m² |
| | | | hydrazide | | combo |
| | | | (cytotoxic | | |
| | | | antibiotic) | | |
| Brentuximab | CD30 | Cleavable | Monomethyl | Tubulin- | 1.8-180 mg/m² |
| vedotin | | (enzymatic) | auristatin E | microtubule | mono |
| (Adcetris) | | | (microtubule- | | 1.2-120 mg/m² |
| | | | targeting) | | combo |
| Ado- | Her-2 | Non-cleavable | DM1, derivative | Tubulin- | 3.6 mg/kg |
| Trastuzumab | | (thioether) | of maytansine | microtubule | |
| emtansine | | | (emtansine) | | |
| (Kadcyla) | | | (microtubule- | | |
| | | | targeting) | | |
| Inotuzumab | CD22 | Cleavable acid- | N-acetyl gamma | Minor | 0.5-0.8 mg/m² |
| ozogamicin | | labile | Calicheamicin | groove | |
| (Besponsa) | | hydrazone | imethyl | of DNA | |
| | | linker | hydrazide | | |
| | | (chemical) | (cytotoxic | | |
| | | | antibiotic) | | |
| Polatuzumab | CD79b | Cleavable | Monomethyl | Tubulin- | 1.8 mg/kg |
| vedotin | | (Enzymatic) | auristatin E | microtubule | |
| (Polivy) | | | (microtubule- | | |
| | | | targeting) | | |
| Enfortumab | Nectin-4 | Cleavable | Monomethyl | Tubulin- | 1.25-125 mg/kg |
| vedotin- | | (Enzymatic) | auristatin E | microtubule | |
| (Padcev) | | | (microtubule- | | |
| | | | targeting) | | |
| Fam- | Her-2 | Cleavable | Topoisomerase I | DNA | 5.4-6.4 mg/kg |
| Trastuzumab | | (Enzymatic) | inhibitor | Topoisomerase I | |
| deruxtecan | | | (exatecan | | |
| (Enhertu) | | | derivative) | | |
| | | | (DNA-targeting) | | |
| Sacituzumab | Trop-2 | Cleavable acid- | SN-38 (active | DNA | 10 mg/kg |
| govitecan | | labile | metabolite) of | Topoisomerase I | |
| (Trodelvy) | | hydrazone | Irinotecan, | | |
| | | (chemical) | topoisomerase-1 | | |
| | | | inhibitor | | |
| | | | (DNA-targeting) | | |
| Loncastuximab | CD19 | Cleavable | SG3199, | DNA | 0.15-0.075 |
| Tesirine | | (Enzymatic) | alkylating agent | crosslinking | mg/kg |
| (Zynlonta) | | | (Pyrrolobenzodi | | |
| | | | azepine dimer) | | |
| | | | (DNA-targeting) | | |
| Tisotumab | Tissue factor | Cleavable | Monomethyl | Tubulin- | 2 mg/kg |
| vedotin | (TF) | (Enzymatic) | auristatin E | microtubule | |
| (Tivdak) | CF-III | | (microtubule- | | |
| | | | targeting) | | |
| Mirvetuximab | Folate factor | Cleavable | DM4 | Tubulin- | 6 mg/kg |
| soravtansine- | alpha | Disufide bond | (maytansinoid | microtubule | |
| gynx (Elahere) | | based | derivative | | |
| (FRα) | | (chemical) | ravtansine) | | |
| | | | (microtubule- | | |
| | | | targeting) | | |
| Datopotamab | Trop-2 | Protease | Deruxtecan | DNA | 6 mg/kg |
| deruxtecan | | cleavable | | Topoisomerase I | |
| (Datroway) | | | | | |
| Anvatabart | Erb-b2 | Stable | AS269 | Tubulin- | 1.3-1.5 mg/kg |
| opadotin | receptor | (non-cleavable) | | microtubule | |
| (ARX788) | tyrosine | | | | |
| | kinase-2 | | | | |
| AZD0901 | Claudin-18 | Protease | Monomethyl | Tubulin- | 1.8-2.2 mg/kg |
| | | cleavable | auristatin E | microtubule | |
| DB-1303 | Erb-b2 | Cathepsin | P1003 | DNA | 8 mg/kg |
| | receptor | cleavable | | Topoisomerase I | |
| | tyrosine | | | | |
| | kinase-2 | | | | |
| Rinatabart | Folate | Cleavable | Exatecan | DNA | 120 mg/m² |
| sesutecan | receptor alpha | | | Topoisomerase I | |
| DS-7300 | CD276 | Protease | Deruxtecan | DNA | 12 mg/kg |
| | | cleavable | | Topoisomerase I | |
| Luveltamab | Folate | Protease | 3-aminophenyl | Tubulin- | 4.3-5.2 mg/kg |
| tazevibulin | receptor alpha | cleavable | hemiasterlin | microtubulin | |
| (Luvelta) | | | | | |
| Patritumab | Erb-b2 | Protease | Exatecan | DNA | 5.6 mg/kg |
| deruxtecan | receptor | cleavable | | Topoisomerase I | |
| | tyrosine | | | | |
| | kinase-3 | | | | |
| Raludotatug | Cadherin 6 | Protease | Exatecan | DNA | 4.8, 5.6, 6.4 |
| deruxtecan | | cleavable | | Topoisomerase I | mg/kg |
| Sacituzumab | Tumor | pH sensitive | Belotecan | DNA | 4 mg/kg |
| triumotecan | associated | and enzyme | | Topoisomerase I | |
| (SKB264) | calcium signal | cleavable | | | |
| | transducer 4 | | | | |
| Sigvotatug | Integrin | Protease | Monomethyl | Tubulin- | 1.8 mg/kg |
| vedotin | subunit beta-6 | cleavable | auristatin E | microtubulin | |
| | secretory | | | | |
| | leukocyte | | | | |
| | peptidase | | | | |
| | inhibitor | | | | |
| Telisotuzumab | Met-proto- | Cleavable | Adizutecan | DNA | 2.4-3 mg/kg |
| adizutecan | oncogene | | | Topoisomerase I | |
| (ABBV-400) | receptor | | | | |
| | tyrosine | | | | |
| | kinase | | | | |
| Telisotuzumab | Met-proto- | Protease | Monomethyl | Tubulin- | 1.9 mg/kg |
| vidotin | oncogene | cleavable | auristatin E | microtubulin | |
| (Teliso-V) | receptor | | | | |
| | tyrosine | | | | |
| | kinase | | | | |
| Zilovertamab | ROR1 | Protease | Monomethyl | Tubulin- | 1.5-2.5 mg/kg |
| vedotin | | cleavable | auristatin E | microtubulin | |
| BAT8006 | Folate | Cleavable | Exatecan | DNA | 84-93 mg/m² |
| | Receptor | | (Camptothecin) | Topoisomerase I | |
| | Alpha | | | | |
| Becotatug | EGFR | Protease | MMAE | Tubulin- | 2.3 mg/kg |
| vedotin | | cleavable | (Auristatin) | microtubulin | |
| BL-M07D1 | HER-2 | Cleavable | Ed-04 (Alkaloid | DNA | 4.4 mg/kg |
| | | | Camptothecin) | Topoisomerase I | |
| Bulumtatug | Nectin-4 | Protease | MMAE | Tubulin- | 1.25 mg/kg |
| fuvedotin | | cleavable | (Auristatin) | microtubulin | |
| Caxmotabart | HER-2 | Cleavable | MMAF | Tubulin- | 2.3 mg/kg |
| entudotin | | | (Auristatin) | microtubulin | |
| CPO-301 | EGFR | Protease | JS-1 | DNA | 4.8 mg/kg |
| | | cleavable | | Topoisomerase I | |
| Disitamab | HER-2 | Protease | MMAE | Tubulin- | 2-2.5 mg/kg |
| vedotin | | cleavable | (Auristatin) | microtubulin | |
| FDA018 | TROP-2 | | SN-38 | DNA | 10 mg/kg |
| | | | (Irinotecan | Topoisomerase I | |
| | | | (CPT-11)) | | |
| GQ1005 | HER-2 | Cleavable | DXd/DX8951 | DNA | 7.2 mg/kg |
| | | | (MAAA-1181a) | Topoisomerase I | |
| | | | (Exatecan) | | |
| GSK5764227 | B7-H3 | Protease | HS-9265 | DNA | 8-12 mg/kg |
| | | cleavable | (Exatecan) | Topoisomerase I | |
| HS-20089 | B7-H4 | Protease | HS-9265 | DNA | 4.8-5.8 mg/kg |
| | | cleavable | (Exatecan) | Topoisomerase I | |
| IBI-343 | CLDN18.2 | Protease | Exatecan | DNA | 6 mg/kg |
| | | cleavable | (Camptothecin) | Topoisomerase I | |
| IBI354 | HER-2 | | Camptothecin | DNA | 12 mg/kg |
| | | | | Topoisomerase I | |
| Izalontamab | EGFR; HER-3 | Cleavable | Ed-04 (Alkaloid | DNA | 2.2-2.5 mg/kg |
| brengitecan | | | Camptothecin) | Topoisomerase I | |
| JSKN-003 | HER-2 | Protease | DXd/DX8951 | DNA | 6.3 mg/kg |
| | | cleavable | (MAAA-1181a) | Topoisomerase I | |
| | | | (Exatecan) | | |
| JSKN033 | HER-2; PD- | Protease | DXd/DX8951 | DNA | 1.1-15 mg/kg |
| | L1 | cleavable | (MAAA-1181a) | Topoisomerase I | |
| | | | (Exatecan) | | |
| MHB088C | B7-H3 | Cleavable | | DNA | 0.8-4 mg/kg |
| | | | | Topoisomerase I | |
| OQY-3258 | TROP-2 | Protease | SN-38 | DNA | 16 mg/kg |
| | | cleavable | (Irinotecan | Topoisomerase I | |
| | | | (CPT-11)) | | |
| Sacituzumab | TROP-2 | pH sensitive | KL610023 | DNA | 4-5 mg/kg |
| tirumotecan | | | (Belotecan) | Topoisomerase I | |
| SHR-A1904 | CLDN18.2 | Cleavable | | DNA | |
| | | | | Topoisomerase I | |
| SHR-A1912 | CD79b | | | DNA | 0.1-3.6 mg/kg |
| | | | | Topoisomerase I | |
| SHR-A2009 | HER-3 | Cleavable | | DNA | 1.5-9 mg/kg |
| | | | | Topoisomerase I | |
| SHR-A2102 | Nectin-4 | Cleavable | | DNA | |
| | | | | Topoisomerase I | |
| Tecotabart | CLDN18.2 | Protease | MMAE | Tubulin- | 1.8-2 mg/kg |
| vedotin | | cleavable | (Auristatin) | microtubulin | |
| Tizetatug | TROP-2 | Cleavable | SHR9265 | DNA | 1.5-12 mg/kg |
| rezetecan | | | (Exatecan) | Topoisomerase I | |
| TQB2102 | HER-2 | Cleavable | | DNA | 7.5 mg/kg |
| | | | | Topoisomerase I | |
| Trastuzumab | HER-2 | Protease | Duostatin5 | Tubulin- | 4.8 mg/kg |
| botidotin | | cleavable | (MMAF) | microtubulin | |
| Trastuzumab | HER-2 | Protease | DUocarmycin- | DNA Damaging | 0.3-2.4 mg/kg |
| duocarmazine | | cleavable | hydroxyBenzam | Agent | |
| | | | ide Azaindole | | |
| | | | (DUBA) | | |
| | | | (Duocarmycin) | | |
| Trastuzumab | HER-2 | Protease | MMAE | Tubulin- | 2-3 mg/kg |
| envedotin | | cleavable | (Auristatin) | microtubulin | |
| Trastuzumab | HER-2 | Cleavable | SHR9265 | DNA | 4.8-6.4 mg/kg |
| rezetecan | | | (Exatecan) | Topoisomerase I | |
| Trastuzumab | HER-2 | Protease | MMAE | Tubulin- | 2.2-2.6 mg/kg |
| vedotin | | cleavable | (Auristatin) | microtubulin | |
| Ujvira | HER-2 | Non-Cleavable | DM1 | Tubulin- | 3.6 mg/kg |
| | | | (Maytansine) | microtubulin | |
| YL201 | B7-H3 | Protease | YL0010014 | DNA | 2-2.4 mg/kg |
| | | cleavable | (Camptothecin) | Topoisomerase I | |
| Vobramitamab | B7-H3 | Protease | DUocarmycin- | DNA Damaging | 2-2.7 mg/kg |
| duocarmazine | | cleavable | hydroxyBenzam | Agent | |
| | | | ide Azaindole | | |
| | | | (DUBA) | | |
| | | | (Duocarmycin) | | |
| Anetumab | Mesothelin | Cleavable | DM4 | Tubulin- | 6.5 mg/kg |
| ravtansine | (MSLN) | | (Maytansine) | microtubulin | |
| BB-1701 | HER-2 | Protease | Eribulin | Tubulin- | 0.8-1.6 mg/kg |
| | | cleavable | | microtubulin | |
| Camidanlumab | IL-2R Alpha | Protease | SG3199 | DNA Damaging | 20-150 mcg/kg |
| Tesirine | | cleavable | (Pyrrolobenzodi | Agent | |
| | | | azepine (PBD)) | | |
| DB-1305 | TROP-2 | Cleavable | P1021 | DNA | 2-8 mg/kg |
| | | | | Topoisomerase I | |
| DB-1311 | B7-H3 | Cleavable | P1021 | DNA | 3-15 mg/kg |
| | | | | Topoisomerase I | |
| DX126-262 | HER-2 | Non-Cleavable | Tub114 | Tubulin- | 4.8-8 mg/kg |
| | | | (Tubulysin) | microtubulin | |
| DXC-008 | STEAP-1 | Cleavable | Tubulysin | Tubulin- | 2.8-28 mg/kg |
| | | | | microtubulin | |
| Farletuzumab | Folate | Protease | Eribulin | Tubulin- | 0.3-1.6 mg/kg |
| Ecteribulin | Receptor | cleavable | | microtubulin | |
| | Alpha | | | | |
| FDA022 | HER-2 | Protease | DXd/DX8951 | DNA | 1.6-8 mg/kg |
| | | cleavable | (MAAA-1181a) | Topoisomerase I | |
| | | | (Exatecan) | | |
| FOR46 | CD46 | Protease | MMAE | Tubulin- | 1.8-2.7 mg/kg |
| | | cleavable | (Auristatin) | microtubulin | |
| HLX43 | PD-L1 | Protease | Camptothecin | DNA | 2-3 mg/kg |
| | | cleavable | | Topoisomerase I | |
| JSKN016 | HER-3; | | | DNA | 5-6 mg/kg |
| | TROP-2 | | | Topoisomerase I | |
| Mecbotamab | Axl | Protease | MMAE | Tubulin- | 0.3-2.4 mg/kg |
| vedotin | | cleavable | (Auristatin) | microtubulin | |
| Misitatug | Mesothelin | Protease | MMAE | Tubulin- | 1-30 mg/kg |
| blivedotin | (MSLN) | cleavable | (Auristatin) | microtubulin | |
| MRG001 | CD20 | Protease | MMAE | Tubulin- | |
| | | cleavable | (Auristatin) | microtubulin | |
| Ozuriftamab | ROR2 | Protease | MMAE | Tubulin- | 0.3-3.3 mg/kg |
| vedotin | | cleavable | (Auristatin) | microtubulin | |
| PDS01ADC | IL-12 | | | | 2-16.8 mcg/kg |
| Praluzatamab | CD166 | Cleavable | DM4 | Tubulin- | 0.25-10 mg/kg |
| ravtansine | | Disufide bond | (Maytansine) | microtubulin | |
| | | based | | | |
| | | (chemical) | | | |
| RC108 | c-MET | | MMAE | Tubulin- | 1.5-2.5 mg/kg |
| | | | (Auristatin) | microtubulin | |
| SHR-1826 | c-MET | | | | 2.2-9 mg/kg |
| SHR-4602 | HER-2 | Cleavable | | DNA | 0.08-2.5 mg/kg |
| | | | | Topoisomerase I | |
| SYS6002 | Nectin-4 | Protease | MMAE | Tubulin- | 1.8-4.5 mg/kg |
| | | cleavable | (Auristatin) | microtubulin | |
| TORL-1-23 | CLDN6 | Protease | MMAE | Tubulin- | 0.2-4 mg/kg |
| | | cleavable | (Auristatin) | microtubulin | |
| TRS005 | CD20 | Protease | MMAE | Tubulin- | 0.1-2.1 mg/kg |
| | | cleavable | (Auristatin) | microtubulin | |
| YL202 | HER-3 | Protease | YL0014 | DNA | |
| | | cleavable | (Camptothecin) | Topoisomerase I | |
| 7MW3711 | B7-H3 | Cleavable | Mtoxin ™ (MF- | DNA | |
| | | | 6) | Topoisomerase I | |
| 9MW2921 | TROP-2 | Cleavable | | DNA | |
| | | | | Topoisomerase I | |
| ADCT-602 | SIGLEC2 | Protease | SG3199 | DNA Damaging | |
| | | cleavable | (Pyrrolobenzodi | Agent | |
| | | | azepine (PBD)) | | |
| ALE.P02 | CLDN1 | | | Tubulin- | |
| | | | | microtubulin | |
| ALK201 | FGFR2b | Cleavable | Exatecan | DNA | |
| | | | (Camptothecin) | Topoisomerase I | |
| AMT-116 | CD44v9 | | KL610023 | DNA | |
| | | | (Belotecan) | Topoisomerase I | |
| AMT253 | MUC18 | Cleavable | Exatecan | DNA | |
| | | Disufide bond | (Camptothecin) | Topoisomerase I | |
| | | based | | | |
| | | (chemical) | | | |
| AZD4360 | CLDN18.2 | | | | |
| AZD5335 | Folate | | AZ14170132 | DNA | |
| | Receptor | | (AZ'0132) | Topoisomerase I | |
| | Alpha | | (Exatecan) | | |
| AZD9829 | CD123 | | AZ14170132 | DNA | |
| | | | (AZ'0132) | Topoisomerase I | |
| | | | (Exatecan) | | |
| BAT8008 | TROP-2 | Cleavable | | DNA | |
| | | | | Topoisomerase I | |
| BAT8010 | HER-2 | Cleavable | | DNA | |
| | | | | Topoisomerase I | |
| Bezetabart | CD74 | Non-Cleavable | Maytansine | Tubulin- | |
| debotansine | | | | microtubulin | |
| BIO-106 | TROP-2 | | | Tubulin- | |
| | | | | microtubulin | |
| BL-M02D1 | TROP-2 | Cleavable | Ed-04 (Alkaloid | DNA | |
| | | | Camptothecin) | Topoisomerase I | |
| DB-1202 | | | | | |
| DB-1310 | HER-3 | Protease | P1021 | DNA | |
| | | cleavable | | Topoisomerase I | |
| DB-1419 | B7-H3; PD-L1 | Cleavable | P1003 | DNA | |
| | | | | Topoisomerase I | |
| Debio 1562M | CD37 | Cleavable | DM1 | Tubulin- | |
| | | | (Maytansine) | microtubulin | |
| DS-3939 | MUC-1 | Protease | DXd/DX8951 | DNA | |
| | | cleavable | (MAAA-1181a) | Topoisomerase I | |
| | | | (Exatecan) | | |
| GEN1286 | c-MET; EGFR | | Exatecan | DNA | |
| | | | (Camptothecin) | Topoisomerase I | |
| GQ1001 | HER-2 | Non-Cleavable | DM1 | Tubulin- | |
| | | | (Maytansine) | microtubulin | |
| GQ1010 | TROP-2 | | Camptothecin | DNA | |
| | | | | Topoisomerase I | |
| HB0052 | CD73 | | | DNA | |
| | | | | Topoisomerase I | |
| HMBD-501 | HER-3 | Cleavable | Exatecan | DNA | |
| | | | (Camptothecin) | Topoisomerase I | |
| HS-20105 | TROP-2 | Cleavable | | DNA | |
| | | | | Topoisomerase I | |
| IBI129 | B7-H3 | | | | |
| IBI130 | TROP-2 | | | | |
| IBI133 | HER-3 | | | | |
| IBI3014 | PD-L1; | Cleavable | NT1 | DNA | |
| | TROP-2 | | | Topoisomerase I | |
| ILB-3101 | B7-H3 | | Eribulin | Tubulin- | |
| | | | | microtubulin | |
| JK06 | 5T4 | | MMAE | Tubulin- | |
| | | | (Auristatin) | microtubulin | |
| JS212 | EGFR; HER-3 | | | | |
| KY-0301 | EGFR; MET | | MMAE | Tubulin- | |
| | | | (Auristatin) | microtubulin | |
| LCB84 | TROP-2 | Cleavable | MMAE | Tubulin- | |
| | | | (Auristatin) | microtubulin | |
| LM-102 | CLDN18.2 | Protease | MMAE | Tubulin- | |
| | | cleavable | (Auristatin) | microtubulin | |
| LM-305 | GPRC5D | Cleavable | MMAE | Tubulin- | |
| | | | (Auristatin) | microtubulin | |
| Lonigutamab | IGF-1R | Non-Cleavable | F554443 | Tubulin- | |
| Ugodotin | | | (MMAF) | microtubulin | |
| MBRC-101 | EphA5 | Protease | MMAE | Tubulin- | |
| | | cleavable | (Auristatin) | microtubulin | |
| MHB036C | TROP-2 | Cleavable | | DNA | |
| | | | | Topoisomerase I | |
| Micvotabart | Fibronectin | Protease | PF-06380101 | Tubulin- | |
| pelidotin | extra-domain | cleavable | (Aur 101) | microtubulin | |
| | B | | (Auristatin) | | |
| MRG004 | Tissue factor | Protease | MMAE | Tubulin- | |
| | | cleavable | (Auristatin) | microtubulin | |
| MRG006 | GPC3 | Cleavable | | DNA | |
| | | | | Topoisomerase I | |
| NBE-002 | ROR1 | Non-Cleavable | PNU-159682 | Topoisomerase | |
| | | | (Anthracycline) | II Inhibitor | |
| OBI-992 | TROP-2 | Protease | Exatecan | DNA | |
| | | cleavable | (Camptothecin) | Topoisomerase I | |
| OQY-6129 | CD38 | | Duostatin 5.2 | Tubulin- | |
| | | | (MMAF) | microtubulin | |
| Pamlectabart | BCMA | Protease | Amanitin | RNA | |
| tismanitin | | cleavable | | Polymerase II | |
| | | | | Inhibitor | |
| Pivekimab | CD123 | Protease | DGN549 | DNA Damaging | |
| Sunirine | | cleavable | (Indolino- | Agent | |
| | | | benzodiazepine | | |
| | | | dimer (IGN)) | | |
| Precemtabart | CEACAM5 | beta- | Exatecan | DNA | |
| tocentecan | | glucuronidase | (Camptothecin) | Topoisomerase I | |
| | | cleavable | | | |
| PRO1107 | PTK7 | Cleavable | MMAE | Tubulin- | |
| | | | (Auristatin) | microtubulin | |
| PRO1160 | CD70 | Cleavable | Exatecan | DNA | |
| | | | (Camptothecin) | Topoisomerase I | |
| Puxitatug | B7-H4 | Protease | AZ14170132 | DNA | |
| samrotecan | | cleavable | (AZ'0132) | Topoisomerase I | |
| | | | (Exatecan) | | |
| RC118 | CLDN18.2 | Cleavable | MMAE | Tubulin- | |
| | | | (Auristatin) | microtubulin | |
| SKB315 | CLDN18.2 | | KL610023 | DNA | |
| | | | (Belotecan) | Topoisomerase I | |
| SKB410 | Nectin-4 | | | | |
| SOT102 | CLDN18.2 | Non-Cleavable | PNU-159682 | Topoisomerase | |
| | | | (Anthracycline) | II Inhibitor | |
| TUB-030 | 5T4 | Cleavable | Exatecan | DNA | |
| | | | (Camptothecin) | Topoisomerase I | |
| TUB-040 | NaPi2b | Cleavable | Exatecan | DNA | |
| | | | (Camptothecin) | Topoisomerase I | |
| XNW27011 | CLDN18.2 | Protease | | DNA | |
| | | cleavable | | Topoisomerase I | |
| XNW28012 | Tissue factor | | | | |
| YL205 | NaPi2b | Protease | Camptothecin | DNA | |
| | | cleavable | | Topoisomerase I | |
| ZL-1310 | DLL3 | Protease | YL0010014 | DNA | |
| | | cleavable | (Camptothecin) | Topoisomerase I | |
| ABBV-291 | CD79b | | | DNA | |
| | | | | Topoisomerase I | |
| ABBV-706 | SEZ6 | Protease | | DNA | |
| | | cleavable | | Topoisomerase I | |
| ABBV-969 | PSMA; | | | DNA | |
| | STEAP-1 | | | Topoisomerase I | |
| ACR246 | 5T4 | Cleavable | D2102 | DNA | |
| | | | | Topoisomerase I | |
| ADC2122 | HER-2 | Protease | Duostatin5 | Tubulin- | |
| | | cleavable | (MMAF) | microtubulin | |
| ADCE-T02 | Tissue factor | Cleavable | Exatecan | DNA | |
| | | | (Camptothecin) | Topoisomerase I | |
| ADCT-701 | DLK-1 | Protease | SG3199 | DNA Damaging | |
| | | cleavable | (Pyrrolobenzodi | Agent | |
| | | | azepine (PBD)) | | |
| ADRX-0405 | STEAP-1 | Cleavable | | Topoisomerase | |
| | | | | Inhibitor | |
| ADRX-0706 | Nectin-4 | Cleavable | AP052 | Tubulin- | |
| | | | | microtubulin | |
| AGS62P1 | FLT3 | Non-Cleavable | MMAF | Tubulin- | |
| | | | (Auristatin) | microtubulin | |
| AGX101 | TM4SF1 | | Maytansine | Tubulin- | |
| | | | | microtubulin | |
| AK138D1 | HER-3 | Protease | DXd/DX8951 | DNA | |
| | | cleavable | (MAAA-1181a) | Topoisomerase I | |
| | | | (Exatecan) | | |
| AK146D1 | Nectin-4; | | | | |
| | TROP-2 | | | | |
| ALK202 | c-MET; EGFR | | | | |
| ALT-P7 | HER-2 | Protease | MMAE | Tubulin- | |
| | | cleavable | (Auristatin) | microtubulin | |
| AMT-151 | Folate | | | | |
| | Receptor | | | | |
| | Alpha | | | | |
| AMT-562 | HER-3 | Protease | Exatecan | DNA | |
| | | cleavable | (Camptothecin) | Topoisomerase I | |
| ARX305 | CD70 | Non-Cleavable | MMAF | Tubulin- | |
| | | | (Auristatin) | microtubulin | |
| ARX517 | PSMA | Non-Cleavable | Amberstatin269 | Tubulin- | |
| | | | (AS269) | microtubulin | |
| | | | (MMAF) | | |
| AT65474 | CLDN6 | Cleavable | AxcynDOT ™ | DNA Damaging | |
| | | | (Trabectedin) | Agent | |
| ATG-022 | CLDN18.2 | Protease | MMAE | Tubulin- | |
| | | cleavable | (Auristatin) | microtubulin | |
| AURIXIM | CD20 | | | | |
| BA1301 | CLDN18.2 | | Duostatin5 | Tubulin- | |
| | | | (MMAF) | microtubulin | |
| BA1302 | CD228 | Cleavable | MMAE | Tubulin- | |
| | | | (Auristatin) | microtubulin | |
| BAT8007 | Nectin-4 | Cleavable | Exatecan | DNA | |
| | | | (Camptothecin) | Topoisomerase I | |
| BB-1705 | EGFR | Protease | Eribulin | Tubulin- | |
| | | cleavable | | microtubulin | |
| BB-1709 | CD73 | | MMAE | Tubulin- | |
| | | | (Auristatin) | microtubulin | |
| BB-1710 | | | | | |
| BB-1712 | B7-H3 | | Eribulin | Tubulin- | |
| | | | | microtubulin | |
| BC3195 | P-Cadherin | Protease | MMAE | Tubulin- | |
| | | cleavable | (Auristatin) | microtubulin | |
| BCG011 | EGFR; | Cleavable | BCPT02 | DNA | |
| | TROP-2 | | | Topoisomerase I | |
| BCG012 | HER-3; | Protease | MMAE | Tubulin- | |
| | MUC1* | cleavable | (Auristatin) | microtubulin | |
| BG-C137 | FGFR2b | | | DNA | |
| | | | | Topoisomerase I | |
| BG-C477 | CEACAM5 | Cleavable | | DNA | |
| | | | | Topoisomerase I | |
| BHV-1530 | FGFR3 | | | DNA | |
| | | | | Topoisomerase I | |
| BL-B16D1 | | | | | |
| BL-M09D1 | | | | | |
| BL-M11D1 | SIGLEC3 | Cleavable | Ed-04 (Alkaloid | DNA | |
| | | | Camptothecin) | Topoisomerase I | |
| BL-M15D1 | CLDN18.2 | Cleavable | Ed-04 (Alkaloid | DNA | |
| | | | Camptothecin) | Topoisomerase I | |
| BL-M17D1 | | | | | |
| BL-M19D1 | | | | | |
| BL-M25D1 | DLL3 | | | | |
| BR111A | ROR1 | | Eribulin | Tubulin- | |
| | | | | microtubulin | |
| BRY812 | LIV-1 | | MMAE | Tubulin- | |
| | | | (Auristatin) | microtubulin | |
| BYON3521 | c-MET | Protease | DUocarmycin- | DNA Damaging | |
| | | cleavable | hydroxyBenzam | Agent | |
| | | | ide Azaindole | | |
| | | | (DUBA) | | |
| | | | (Duocarmycin) | | |
| BYON4413 | CD123 | Protease | DUocarmycin- | DNA Damaging | |
| | | cleavable | hydroxyBenzam | Agent | |
| | | | ide Azaindole | | |
| | | | (DUBA) | | |
| | | | (Duocarmycin) | | |
| CS5001 | ROR1 | Cleavable | SG2057 | DNA Damaging | |
| | | | (Pyrrolobenzodi | Agent | |
| | | | azepine (PBD)) | | |
| CUSP06 | CDH6 | Protease | Exatecan | DNA | |
| | | cleavable | (Camptothecin) | Topoisomerase I | |
| CX-2051 | EpCAM | Cleavable | Camptothecin | DNA | |
| | | | | Topoisomerase I | |
| DB-1312 | B7-H4 | | | DNA | |
| | | | | Topoisomerase I | |
| DS-9606a | CLDN6 | | Pyrrolobenzodia | DNA Damaging | |
| | | | zepine (PBD) | Agent | |
| DXC-004 | EGFR | | Tubulysin | Tubulin- | |
| | | | | microtubulin | |
| DXC-005 | MUC-1 | | Tub201 | Tubulin- | |
| | | | (Tubulysin) | microtubulin | |
| DXC-006 | CD56 | Cleavable | CPT-113 | DNA | |
| | | | (Camptothecin) | Topoisomerase I | |
| DXC-007 | SIGLEC3 | | Tub255 | Tubulin- | |
| | | | (Tubulysin) | microtubulin | |
| DXC-009 | BCMA | | | | |
| DXC1002 | TROP-2 | | CPT-113 | DNA | |
| | | | (Camptothecin) | Topoisomerase I | |
| Emiltatug | B7-H4 | Cleavable | Auristatin F- | Tubulin- | |
| ledadotin | | | HPA (XMT- | microtubulin | |
| | | | 1267) | | |
| | | | (Auristatin) | | |
| ESG406 | | Cleavable | | DNA | |
| | | | | Topoisomerase I | |
| ETx-22 | Nectin-4 | Cleavable | Exatecan | DNA | |
| | | | (Camptothecin) | Topoisomerase I | |
| F0002-ADC | CD30 | Non-Cleavable | DM1 | Tubulin- | |
| | | | (Maytansine) | microtubulin | |
| FZ-AD004 | TROP-2 | Cleavable | | DNA | |
| | | | | Topoisomerase I | |
| FZ-AD005 | DLL3 | Protease | DXd/DX8951 | DNA | |
| | | cleavable | (MAAA-1181a) | Topoisomerase I | |
| | | | (Exatecan) | | |
| GB251 | HER-2 | Protease | MMAE | Tubulin- | |
| | | cleavable | (Auristatin) | microtubulin | |
| HBM9033 | Mesothelin | Cleavable | Camptothecin | DNA | |
| | (MSLN) | | | Topoisomerase I | |
| HDM2005 | ROR1 | Protease | MMAE | Tubulin- | |
| | | cleavable | (Auristatin) | microtubulin | |
| HDP-102 | CD37 | Non-Cleavable | Amanitin | RNA | |
| | | | | Polymerase II | |
| | | | | Inhibitor | |
| HLX42 | EGFR | Protease | Camptothecin | DNA | |
| | | cleavable | | Topoisomerase I | |
| HS-20110 | | | | | |
| HS-20122 | c-MET; EGFR | | | | |
| IBI3001 | B7-H3; EGFR | | Exatecan | DNA | |
| | | | (Camptothecin) | Topoisomerase I | |
| IBI3005 | EGFR; HER-3 | | Camptothecin | DNA | |
| | | | | Topoisomerase I | |
| IBI3009 | DLL3 | | Camptothecin | DNA | |
| | | | | Topoisomerase I | |
| IBI3020 | CEACAM5 | | | | |
| IKS03 | CD19 | Cleavable | SG2057 | DNA Damaging | |
| | | | (Pyrrolobenzodi | Agent | |
| | | | azepine (PBD)) | | |
| IM-1021 | ROR1 | Cleavable | Camptothecin | DNA | |
| | | | | Topoisomerase I | |
| IPH45 | Nectin-4 | Cleavable | Exatecan | DNA | |
| | | | (Camptothecin) | Topoisomerase I | |
| Ispectamab | BCMA | Non-Cleavable | Maytansine | Tubulin- | |
| debotansine | | | | microtubulin | |
| JBH492 | CCR7 | Cleavable | DM4 | Tubulin- | |
| | | Disufide bond | (Maytansine) | microtubulin | |
| | | based | | | |
| | | (chemical) | | | |
| JS107 | CLDN18.2 | | MMAE | Tubulin- | |
| | | | (Auristatin) | microtubulin | |
| JS108 | TROP-2 | | Tub196 | Tubulin- | |
| | | | (Tubulysin) | microtubulin | |
| KH815 | TROP-2 | | | RNA | |
| | | | | Polymerase II | |
| | | | | Inhibitor; DNA | |
| | | | | Topoisomerase I | |
| KK2845 | TIM-3 | Protease | SG3249 | DNA Damaging | |
| | | cleavable | (Pyrrolobenzodi | Agent | |
| | | | azepine (PBD)) | | |
| KM-501 | HER-2 | | MMAE | Tubulin- | |
| | | | (Auristatin) | microtubulin | |
| LNCB74 | B7-H4 | Cleavable | MMAE | Tubulin- | |
| | | | (Auristatin) | microtubulin | |
| LY4052031 | Nectin-4 | Protease | Camptothecin | DNA | |
| | | cleavable | | Topoisomerase I | |
| LY4170156 | Folate | Cleavable | Exatecan | DNA | |
| | Receptor | | (Camptothecin) | Topoisomerase I | |
| | Alpha | | | | |
| M3554 | GD2 | Cleavable | Exatecan | DNA | |
| | | | (Camptothecin) | Topoisomerase I | |
| MG1901 | | | | | |
| MG2001 | | | | | |
| MG2002A | | | | | |
| MGC026 | B7-H3 | Protease | Exatecan | DNA | |
| | | cleavable | (Camptothecin) | Topoisomerase I | |
| MGC028 | ADAM9 | Protease | Exatecan | DNA | |
| | | cleavable | (Camptothecin) | Topoisomerase I | |
| MHB118C | IGF-1R | | MH30010008 | DNA | |
| | | | (Exatecan) | Topoisomerase I | |
| Mirzotamab | B7-H3 | Protease | Clezutoclax | BCL-XL | |
| clezutoclax | | cleavable | | Inhibitor | |
| MK-6204 | | | | | |
| MT-8633 | c-MET | Protease | SG3199 | DNA Damaging | |
| | | cleavable | (Pyrrolobenzodi | Agent | |
| | | | azepine (PBD)) | | |
| MTX-13 | PTK7 | Protease | Exatecan | DNA | |
| | | cleavable | (Camptothecin) | Topoisomerase I | |
| MYTX-011 | c-MET | Protease | MMAE | Tubulin- | |
| | | cleavable | (Auristatin) | microtubulin | |
| NC18 | HER-2 | | AF-HEA | Tubulin- | |
| | | | (Auristatin) | microtubulin | |
| NN3201 | cKIT | Cleavable | MMAE | Tubulin- | |
| | | | (Auristatin) | microtubulin | |
| Oba01 | DR5 | Protease | MMAE | Tubulin- | |
| | | cleavable | (Auristatin) | microtubulin | |
| Oberotatug | CD205 | Cleavable | DM4 | Tubulin- | |
| ravtansine | | Disufide bond | (Maytansine) | microtubulin | |
| | | based | | | |
| | | (chemical) | | | |
| OMTX705 | FAP | Protease | TAM470 | Tubulin- | |
| | | cleavable | (Cytolysin) | microtubulin | |
| Opugotamig | Folate | Protease | DM21 | Tubulin- | |
| olatansine | Receptor | cleavable | (Maytansine) | microtubulin | |
| | Alpha | | | | |
| PF-08046031 | CD228 | Protease | MMAE | Tubulin- | |
| | | cleavable | (Auristatin) | microtubulin | |
| PF-08046032 | IL-2R Alpha | Protease | MMAE | Tubulin- | |
| | | cleavable | (Auristatin) | microtubulin | |
| PHN-010 | | | | DNA | |
| | | | | Topoisomerase I | |
| QLS5132 | CLDN6 | Cleavable | QLS6916 | DNA | |
| | | | | Topoisomerase I | |
| Serclutamab | EGFR | Protease | SGD-1882 | DNA Damaging | |
| talirine | | cleavable | (Pyrrolobenzodi | Agent | |
| | | | azepine (PBD)) | | |
| SG2918 | | Cleavable | | Tubulin- | |
| | | | | microtubulin | |
| SGN-35C | CD30 | beta- | Camptothecin | DNA | |
| | | glucuronidase | | Topoisomerase I | |
| | | cleavable | | | |
| SGN-35T | CD30 | Protease | MMAE | Tubulin- | |
| | | cleavable | (Auristatin) | microtubulin | |
| SGN-CD228A | CD228 | beta- | MMAE | Tubulin- | |
| | | glucuronidase | (Auristatin) | microtubulin | |
| | | cleavable | | | |
| SGN- | CEACAM5 | beta- | AMDCPT | DNA | |
| CEACAM5C | | glucuronidase | (Camptothecin) | Topoisomerase I | |
| | | cleavable | | | |
| SGN-PDL1V | PD-L1 | Protease | MMAE | Tubulin- | |
| | | cleavable | (Auristatin) | microtubulin | |
| SHR-4849 | DLL3 | Cleavable | Dxh (Exatecan) | DNA | |
| | | | | Topoisomerase I | |
| SHR-7631 | | | | | |
| SIBP-A13 | HER-3 | | | | |
| SIBP-A17 | HER-2 | Cleavable | | | |
| SIBP-A18 | | | | | |
| SIBP-A19 | | | | | |
| SIM 0505 | CDH6 | | Camptothecin | DNA | |
| | | | | Topoisomerase I | |
| SIM 0686 | FGFR2b | Cleavable | Camptothecin | DNA | |
| | | | | Topoisomerase I | |
| SKB445 | | | | | |
| SKB500 | | | | | |
| SKB518 | | | | DNA | |
| | | | | Topoisomerase I | |
| SKB571 | | | | | |
| SMP-190 | CEACAM5 | | DXd/DX8951 | DNA | |
| | | | (MAAA-1181a) | Topoisomerase I | |
| | | | (Exatecan) | | |
| SMP-656 | HER-2 | | Eribulin | Tubulin- | |
| | | | | microtubulin | |
| SMP-869 | B7-H3 | | Eribulin | Tubulin- | |
| | | | | microtubulin | |
| SYS6005 | ROR1 | | MMAE | Tubulin- | |
| | | | (Auristatin) | microtubulin | |
| SYS6023 | HER-3 | | | | |
| SYS6040 | | | | | |
| SYS6041 | Folate | Cleavable | Exatecan | DNA | |
| | Receptor | | (Camptothecin) | Topoisomerase I | |
| | Alpha | | | | |
| SYS6043 | | | | | |
| T320-ADC | Tissue factor | | MMAE | Tubulin- | |
| | | | (Auristatin) | microtubulin | |
| Tilatamig | c-MET; EGFR | Cleavable | AZ14170132 | DNA | |
| samrotecan | | | (AZ'0132) | Topoisomerase I | |
| | | | (Exatecan) | | |
| TORL-2-307- | CLDN18.2 | Cleavable | MMAE | Tubulin- | |
| ADC | | | (Auristatin) | microtubulin | |
| TORL-3-600 | CDH17 | Cleavable | MMAE | Tubulin- | |
| | | | (Auristatin) | microtubulin | |
| TORL-4-500 | DLK-1 | Cleavable | MMAE | Tubulin- | |
| | | | (Auristatin) | microtubulin | |
| TQB2101 | ROR1 | | | | |
| TQB2103 | CLDN18.2 | Cleavable | | DNA Damaging | |
| | | | | Agent | |
| VIP943 ADC | CD123 | Cleavable | VIP716 | KSP Inhibitor | |
| | | | | (KSPi)/ Eg5 | |
| | | | | Inhibitors | |
| XB010 | 5T4 | | MMAE | Tubulin- | |
| | | | (Auristatin) | microtubulin | |
| YH013 | EGFR; MET | Cleavable | MMAE | Tubulin- | |
| | | | (Auristatin) | microtubulin | |
| YL211 | c-MET | Protease | Camptothecin | DNA | |
| | | cleavable | | Topoisomerase I | |
| YL217 | CDH17 | Protease | YL0010014 | DNA | |
| | | cleavable | (Camptothecin) | Topoisomerase I | |
| ZW191 | Folate | Protease | ZD06519 | DNA | |
| | Receptor | cleavable | (Camptothecin) | Topoisomerase I | |
| | Alpha | | | | |
| AMT-676 | CDH17 | | | | |
| INA03 | Tfr1 | Protease | MMAE | Tubulin- | |
| | | cleavable | (Auristatin) | microtubulin | |
| 1ST-EP1 | | | | | |
| 1ST-EP2 | | | | | |
| 20D9h3-DUBA | FLT3 | | Duocarmycin | DNA Damaging | |
| | | | | Agent | |
| 20D9-ADC | FLT3 | Cleavable | MMAF | Tubulin- | |
| | | | (Auristatin) | microtubulin | |
| 3A11-vc- | FGFR4 | Protease | MMAE | Tubulin- | |
| MMAE | | cleavable | (Auristatin) | microtubulin | |
| 7B8-MMAE | MUC1-C | Protease | MMAE | Tubulin- | |
| | | cleavable | (Auristatin) | microtubulin | |
| 7MW4811 | | | Mtoxin ™ (MF- | | |
| | | | 6) | | |
| 7MW4911 | CDH17 | Cleavable | Mtoxin ™ (MF- | DNA | |
| | | | 6) | Topoisomerase I | |
| 7PB-300 | | | 7PB-100 | BAK activation | |
| 9B02x | | | | | |
| 9B03x | | | | | |
| A0401 | | | | | |
| A1801 | gpNMB | Protease | MMAE | Tubulin- | |
| | | cleavable | (Auristatin) | microtubulin | |
| AB003 | PD-L1; PD-L2 | | | | |
| ABC-007 | HER-2; USP7 | | | Inhibitor | |
| ABC-014 | TROP-2 | | | | |
| ABC-020 | | | | | |
| ABC101-1 | HER-2 | Non-Cleavable | DM1 | Tubulin- | |
| | | | (Maytansine) | microtubulin | |
| ABC103-1 | HER-2 | | | | |
| ABC104-1 | HER-2 | | | | |
| ABC105-1 | HER-2 | | | | |
| ABL206 | | | | DNA | |
| | | | | Topoisomerase I | |
| ABL209 | | | | DNA | |
| | | | | Topoisomerase I | |
| ABL210 | | | | DNA | |
| | | | | Topoisomerase I | |
| AD-02 | | | | | |
| ADC 1779 | DPP4 | | | | |
| ADC 5311 | | | | | |
| ADC-012 | | | | | |
| ADC-018 | | | | | |
| ADC-042 | | | | | |
| ADC-051 | | | | | |
| ADC2192 | TROP-2 | | | | |
| ADC2202 | HER-2 | Cleavable | | | |
| ADC2204 | Nectin-4 | | | | |
| ADC2313 | c-MET; EGFR | | | | |
| ADC2317 | | | | | |
| ADC2336 | | | | | |
| ADC2403 | | | | | |
| ADCE-017 | MRC2 | | DXd/DX8951 | DNA | |
| | | | (MAAA-1181a) | Topoisomerase I | |
| | | | (Exatecan) | | |
| ADCE-202 | MRC2 | | DXd/DX8951 | DNA | |
| | | | (MAAA-1181a) | Topoisomerase I | |
| | | | (Exatecan) | | |
| ADCE-B05 | | | | | |
| ADCGEN-04 | | Cleavable | | | |
| ADCITMER ® | CD56 | | MMAE | Tubulin- | |
| | | | (Auristatin) | microtubulin | |
| ADCT-241 | PSMA | Protease | PL2202 | DNA | |
| | | cleavable | (Camptothecin) | Topoisomerase I | |
| ADCT-242 | CLDN6 | | Exatecan | DNA | |
| | | | (Camptothecin) | Topoisomerase I | |
| ADRX-0134 | NaPi2b | Cleavable | AP052 | Tubulin- | |
| | | | | microtubulin | |
| ADV101 | IL1RAP | Cleavable | | DNA | |
| | | | | Topoisomerase I | |
| AKTX-101 | TROP-2 | | Thailanstatin | RNA | |
| | | | (PH1) | Polymerase II | |
| | | | | Inhibitor | |
| AKTX-102 | | | PH5 | DNA Damaging | |
| | | | | Agent | |
| AKTX-103 | | | PH6 | DNA Damaging | |
| | | | | Agent | |
| ALB-02 | Calnexin | | | | |
| ALE.P03 | CLDN1 | | | DNA | |
| | | | | Topoisomerase I | |
| ALG01 | PD-L1; | | Exatecan | DNA | |
| | TROP-2 | | (Camptothecin) | Topoisomerase I | |
| ALKN203 | | | | | |
| ALKN204 | EGFR; HER-3 | | | | |
| ALM-401 | EGFR; ROR1 | Protease | MMAE | Tubulin- | |
| | | cleavable | (Auristatin) | microtubulin | |
| ALX2004 | EGFR | | | | |
| ALY-302 | | | | | |
| AM E3-SG3249 | TSLPR | | SG3249 | DNA Damaging | |
| | | | (Pyrrolobenzodi | Agent | |
| | | | azepine (PBD)) | | |
| AM1-15 | EGFR | Protease | | BCL-XL | |
| | | cleavable | | Inhibitor | |
| AMB-104 | CSF-1R | | | | |
| AMB201 | | | | | |
| AMB202 | | | | | |
| AMB302 | FGFR3 | Cleavable | | DNA | |
| | | | | Topoisomerase I | |
| AMB303 | ROR1 | | | DNA | |
| | | | | Topoisomerase I | |
| AMB304 | | | | DNA | |
| | | | | Topoisomerase I | |
| AMT-B85 | Tyrosine | | | | |
| | kinase (TK) | | | | |
| APB-ADC | | | | | |
| APH-0912 | | | Docetaxel | Tubulin- | |
| | | | (Taxanes) | microtubulin | |
| APL-2501 | CLDN6; | | | | |
| | CLDN9 | | | | |
| APN-497444 | Glycan | | | | |
| APN-685612 | | | | | |
| APN-987481 | Glycan | | | | |
| AR081 | | | | | |
| AR153 | B7-H3 | Protease | MMAE | Tubulin- | |
| | | cleavable | (Auristatin) | microtubulin | |
| ARB1002 | CDH17 | | | | |
| ARB102A | CDH17 | | | | |
| ARB1035 | CDH17 | | | | |
| ARR-002 | | | | | |
| ARR-003 | | | | | |
| ARX111 | TROP-2 | | | | |
| AT-00 | HER-2 | | AxcynDOT ™ | DNA Damaging | |
| | | | (Trabectedin) | Agent | |
| AT-001 ADC | PD-L1 | | | | |
| AT-002 ADC | TROP-2 | | | | |
| AT-401-CD20 | CD20 | | CLYP-71 (Lytic | Other | |
| ADC | | | Peptide) | | |
| AT-401-HER2 | HER-2 | | CLYP-71 (Lytic | Other | |
| ADC | | | Peptide) | | |
| AT2604 | Alkaline | Cleavable | MMAE | Tubulin- | |
| | phosphatase, | | (Auristatin) | microtubulin | |
| | placental-like | | | | |
| | 2 | | | | |
| AT86474 | ROR1 | Cleavable | AxcynDOT ™ | DNA Damaging | |
| | | | (Trabectedin) | Agent | |
| ATN301 | | | | | |
| ATRC-301 | EphA2 | | | | |
| ATX101 | CD6 | | MMAE | Tubulin- | |
| | | | (Auristatin) | microtubulin | |
| AZD0516 | STEAP2 | Cleavable | Exatecan | DNA | |
| | | | (Camptothecin) | Topoisomerase I | |
| B2C4-MMAE | EGFR; HER-2 | Protease | MMAE | Tubulin- | |
| | | cleavable | (Auristatin) | microtubulin | |
| BA3361 | Nectin-4 | Cleavable | | | |
| BB-201 | HER-2; | Protease | MMAE | Tubulin- | |
| | TROP-2 | cleavable | (Auristatin) | microtubulin | |
| BB-204 | HER-2 | | | | |
| BB-205 | c-MET; HER- | Protease | MMAE | Tubulin- | |
| | 2 | cleavable | (Auristatin) | microtubulin | |
| BB-209 | | | | | |
| BC2027 | GPC3 | | | | |
| BCG013 | 5T4; MET | | | | |
| BCG014 | P-Cadherin | | BLD1102 | DNA | |
| | | | | Topoisomerase I | |
| BCG016 | 5T4; MUC-1 | | MMAE | Tubulin- | |
| | | | (Auristatin) | microtubulin | |
| BCG017 | EGFR; PTK7 | | MMAE | Tubulin- | |
| | | | (Auristatin) | microtubulin | |
| BCG019 | EGFR; HER-3 | Cleavable | BCPT02 | DNA | |
| | | | | Topoisomerase I | |
| BCG022 | HER-3; MET | Protease | MMAE | Tubulin- | |
| | | cleavable | (Auristatin) | microtubulin | |
| BCG022- | HER-3; MET | Cleavable | BCPT02 | DNA | |
| BLD1102 | | | | Topoisomerase I | |
| BCG023 | Folate | Protease | MMAE | Tubulin- | |
| | Receptor | cleavable | (Auristatin) | microtubulin | |
| | Alpha; MUC- | | | | |
| | 1 | | | | |
| BCG027 | B7-H3; SEZ6 | Cleavable | BCPT02 | DNA | |
| | | | | Topoisomerase I | |
| BCG033 | PTK7; TROP- | Protease | MMAE | Tubulin- | |
| | 2 | cleavable | (Auristatin) | microtubulin | |
| BCG034 | B7-H3; PTK7 | | BCPT02 | DNA | |
| | | | | Topoisomerase I | |
| BFACT1 | Meflin | Cleavable | | | |
| BHV-1500 | CD30 | | MMAE | Tubulin- | |
| | | | (Auristatin) | microtubulin | |
| Bi-specific Anti- | EGFR; HER-2 | | | | |
| Her2 Zybodies | | | | | |
| ADC | | | | | |
| BIO-107 | | | | | |
| BIO-108 | | | | | |
| BIO-109 | | | | | |
| BIO-110 | | | | | |
| BIO-112 | | | | | |
| BIO-201 | HER-2; | Cleavable | | DNA | |
| | TROP-2 | | | Topoisomerase I | |
| BL-M21D1 | | | | | |
| BL-M24D1 | | | | | |
| BLB-101 | CLDN6; | | | DNA | |
| | CLDN9 | | | Topoisomerase I | |
| BLB-301 | | Cleavable | | | |
| BPI-9301A | | | | | |
| BPI-9302A | | | | | |
| BPI-9303A | | | | | |
| BPI-9304A | | | | | |
| BPI-9305A | | | | | |
| BR-2302 | EGFR; HER-3 | | | | |
| BR113 | TROP-2 | | | Immunomodulat | |
| | | | | or; DNA | |
| | | | | Topoisomerase I | |
| BR116 | CDH17 | | | DNA | |
| | | | | Topoisomerase I | |
| BRKB-20 | | | | | |
| BRKB-28 | HER-2 | | Auristatin | Tubulin- | |
| | | | | microtubulin | |
| BRKB-300 | B7-H3 | | Pyrrolobenzodia | DNA Damaging | |
| | | | zepine (PBD) | Agent | |
| BRKB-400 | | | | | |
| BRKB-44 | | | | | |
| BRKB-500 | | | | | |
| BSA01 | EGFR; MUC- | Cleavable | MMAE | Tubulin- | |
| | 1 | | (Auristatin) | microtubulin | |
| BSI-706 ADC | CLDN18.2 | Protease | DXd/DX8951 | DNA | |
| | | cleavable | (MAAA-1181a) | Topoisomerase I | |
| | | | (Exatecan) | | |
| BSI-715 | | | | | |
| BSI-721 | CDH17 | Protease | MMAE | Tubulin- | |
| | | cleavable | (Auristatin) | microtubulin | |
| BSI-729 | | | | | |
| BSI-730 | HER-2; PD- | | | | |
| | L1 | | | | |
| BVX001 | CD7; | Non-Cleavable | MMAF | Tubulin- | |
| | SIGLEC3 | | (Auristatin) | microtubulin | |
| BVX002 | | | | | |
| BVX003 | | | | | |
| BVX004 | | | | | |
| C6 CEACAM6- | CEACAM6 | | | | |
| GIT Bispecific | | | | | |
| ADC | | | | | |
| C6 CEACAM6- | CEACAM6 | | | | |
| GYN Bispecific | | | | | |
| ADC | | | | | |
| C6P | CLDN6 | Cleavable | Pyrrolobenzodia | DNA Damaging | |
| | | | zepine (PBD) | Agent | |
| Camptothecin- | CLDN6 | Protease | Camptothecin | DNA | |
| Claudin-6 ADC | | cleavable | | Topoisomerase I | |
| CATB-101 | | Cleavable | | Tubulin- | |
| | | | | microtubulin | |
| CATB-102 | TROP-2 | Cleavable | | | |
| CB-120 | TROP-2 | Cleavable | | | |
| | | (probably | | | |
| | | protease) | | | |
| CBS103 ADC | | | | | |
| CCI-02-03 | HER-2 | Protease | Ceralacertib; | ATR Inhibitor; | |
| | | cleavable | Duocarmycin | DNA Damaging | |
| | | | | Agent | |
| CIM053-ADC | CD45 | | | | |
| CM-09 | TRA-1-60 | Protease | MMAE | Tubulin- | |
| | | cleavable | (Auristatin) | microtubulin | |
| CM-09-(FGX-2- | TRA-1-60 | | FGX2-62 | DNA Damaging | |
| 62) ADC | | | (Pyridinobenzod | Agent | |
| | | | iazepines | | |
| | | | (PDD)) | | |
| CM-14 | TRA-1-60 | Protease | MMAE | Tubulin- | |
| | | cleavable | (Auristatin) | microtubulin | |
| CM518D1 | CDH17 | | | | |
| CO-1002 | | | | | |
| CO-1008 | | | | | |
| CO-1024 | CDH17; | | | | |
| | CLDN18.2 | | | | |
| CO-1025 | | | | | |
| CR-002 | | | | Topoisomerase | |
| | | | | Inhibitor | |
| CR-003 | | | | Topoisomerase | |
| | | | | Inhibitor | |
| CRB-2107 | | | | | |
| CS5005 | SSTR2 | Cleavable | DXd/DX8951 | DNA | |
| | | | (MAAA-1181a) | Topoisomerase I | |
| | | | (Exatecan) | | |
| CS5006 | ITGB4 | Protease | DXd/DX8951 | DNA | |
| | | cleavable | (MAAA-1181a) | Topoisomerase I | |
| | | | (Exatecan) | | |
| CS5007 | EGFR; HER-3 | | Exatecan | DNA | |
| | | | (Camptothecin) | Topoisomerase I | |
| CT-P71 | Nectin-4 | Cleavable | PBX-7016 | DNA | |
| | | | | Topoisomerase I | |
| CT-P73 | | Cleavable | PBX-7016 | DNA | |
| | | | | Topoisomerase I | |
| CT109 - ADC | CEACAM5; | | SN-38 | DNA | |
| | CEACAM6 | | (Irinotecan | Topoisomerase I | |
| | | | (CPT-11)) | | |
| CTPH-02 | HER-2 | | MMAE | Tubulin- | |
| | | | (Auristatin) | microtubulin | |
| CUBT906 | CD56 | | Pyrrolobenzodia | DNA Damaging | |
| | | | zepine (PBD) | Agent | |
| DB-1314 | DLL3 | Protease | P1021 | DNA | |
| | | cleavable | | Topoisomerase I | |
| DB-1316 | | | | | |
| DB-1317 | | | | DNA | |
| | | | | Topoisomerase I | |
| DB-1324 | | | | | |
| DB-1325 | BCMA | Cleavable | LPDUP5 | Inhibitor | |
| DB-1418 | EGFR; HER-3 | | | | |
| DB-1421 | | | | | |
| DCB Globo H | Globo H | Protease | MMAE | Tubulin- | |
| ADC | | cleavable | (Auristatin) | microtubulin | |
| DCB MSLN | Mesothelin | Protease | MMAE | Tubulin- | |
| ADC | (MSLN) | cleavable | (Auristatin) | microtubulin | |
| DEC002 | SEZ6 | Cleavable | CPT-113 | DNA | |
| | | | (Camptothecin) | Topoisomerase I | |
| DEC006 | PTK7 | Cleavable | CPT-113 | DNA | |
| | | | (Camptothecin) | Topoisomerase I | |
| DEC008 | MUC16 | Cleavable | CPT-113 | DNA | |
| | | | (Camptothecin) | Topoisomerase I | |
| DM005- | EGFR; MET | Cleavable | BCPT02 | DNA | |
| BLD1102 | | | | Topoisomerase I | |
| bsADC | | | | | |
| DM008 | | | | | |
| DM009 | | | | | |
| DXA023-G017 | PD-1; VEGF | | | | |
| DXC-003 | TROP-2 | | | | |
| DXC-010 | | | | | |
| DXC-011 | | | | | |
| DXC-012 | | | | | |
| DXC-013 | | | | | |
| DXC-014 | B7-H3; | | | DNA | |
| | PSMA | | | Topoisomerase I | |
| DXC-015 | | | | | |
| DXC-016 | | | | | |
| DXC-017 | | | | | |
| DXC-018 | HER-2 | | | Inhibitor; DNA | |
| | | | | Topoisomerase I | |
| DXC-019 | | | | | |
| DXC-020 | ICAM-1 | | | | |
| DXC022 | GUCY2C | | | | |
| DXC023 | PD-1; VEGF | | | | |
| DXC024 | EGFR; | | Tubulysin | Tubulin- | |
| | TROP-2 | | | microtubulin | |
| DXC025 | EGFR; MUC- | | Tubulysin | Tubulin- | |
| | 1 | | | microtubulin | |
| E-cM-Topi | c-MET; EGFR | Cleavable | | DNA | |
| | | | | Topoisomerase I | |
| E-M-TOPi | EGFR; MUC1 | Cleavable | | DNA | |
| | | | | Topoisomerase I | |
| ED2 | CAFs | | | | |
| ED4 | Tumor | | | | |
| | microenviron | | | | |
| | ment | | | | |
| EDC9 | CD20 | Non-Cleavable | CEN371 | Other | |
| EM28 | c-MET; EGFR | Cleavable | | DNA | |
| | | | | Topoisomerase I | |
| EO-1022 | HER-3 | Protease | MMAE | Tubulin- | |
| | | cleavable | (Auristatin) | microtubulin | |
| ESG407 | DLL3 | Cleavable | | DNA | |
| | | | | Topoisomerase I | |
| ESG408 | | Cleavable | | DNA | |
| | | | | Topoisomerase I | |
| ESG409 | B7-H3 | | | DNA | |
| | | | | Topoisomerase I | |
| ESG411 | | | | DNA | |
| | | | | Topoisomerase I | |
| ESG412 | | Cleavable | | DNA | |
| | | | | Topoisomerase I | |
| ESG413 | | Cleavable | | DNA | |
| | | | | Topoisomerase I | |
| ESG414 | | Cleavable | | DNA | |
| | | | | Topoisomerase I | |
| ESG415 | | Cleavable | | DNA | |
| | | | | Topoisomerase I | |
| ESG416 | | Cleavable | | DNA | |
| | | | | Topoisomerase I | |
| ETX-ATACs | Nectin-4 | | Amanitin | RNA | |
| | | | | Polymerase II | |
| | | | | Inhibitor | |
| EVX-020 | KIF20A | Cleavable | DIACC2010 | Tubulin- | |
| | | | | microtubulin | |
| EX108 | | | | | |
| FG-B310 | | | | | |
| FG-M131 | | | | | |
| FS001 | | Cleavable | | DNA | |
| | | | | Topoisomerase I | |
| GB-4362 | | | MMAE | Tubulin- | |
| | | | (Auristatin) | microtubulin | |
| GENA-104 | CNTN4 | | | | |
| ADC | | | | | |
| GENA- | CNTN4 | Non-Cleavable | Exatecan | DNA | |
| 104A16.1-Exa | | | (Camptothecin) | Topoisomerase I | |
| GENA-111 | CD239 | Cleavable | MMAF | Tubulin- | |
| | | | (Auristatin) | microtubulin | |
| GENA-120 | | | | | |
| GENA-121 | | | | | |
| GENA-122 | | | | | |
| GenSci139 | EGFR; HER-2 | Cleavable | | DNA | |
| | | | | Topoisomerase I | |
| GenSci140 | Folate | Cleavable | | DNA | |
| | Receptor | | | Topoisomerase I | |
| | Alpha | | | | |
| GenSci143 | B7-H3; | Cleavable | | DNA | |
| | PSMA | | | Topoisomerase I | |
| GFS784 | EGFR | | | | |
| GLK-10 | HER-2 | Cleavable | MMAU | Tubulin- | |
| | | | (Auristatin) | microtubulin | |
| GLK-21 | TRP1 | Cleavable | MMAU | Tubulin- | |
| | | | (Auristatin) | microtubulin | |
| GLK-33 | SIGLEC3 | Cleavable | MMAU | Tubulin- | |
| | | | (Auristatin) | microtubulin | |
| GLK-40 | | | | | |
| GLK-50 | | | | | |
| GLR1059 | Nectin-4 | Cleavable | | Tubulin- | |
| | | | | microtubulin | |
| GLR1061 | B7-H3 | Cleavable | | DNA | |
| | | | | Topoisomerase I | |
| GNX102-ADC | Glycan | | | | |
| GNX1021 | Glycan | Cleavable | MMAE | Tubulin- | |
| | | | (Auristatin) | microtubulin | |
| GO-3D1-ADC | MUC1-C | Protease | MMAE | Tubulin- | |
| | | cleavable | (Auristatin) | microtubulin | |
| GQ1009 | | | | | |
| GQ1012 | HER-2 | | | DNA | |
| | | | | Topoisomerase I | |
| GQ1030 | DLL3 | | | | |
| GQ1033 | c-MET; EGFR | | | | |
| GT-00X ADC | | Cleavable | | | |
| h16A-DXd | MUC1 | Protease | DXd/DX8951 | DNA | |
| | | cleavable | (MAAA-1181a) | Topoisomerase I | |
| | | | (Exatecan) | | |
| h16A-MF6 | MUC1 | | Mtoxin ™ (MF- | DNA | |
| | | | 6) | Topoisomerase I | |
| HB004 | | | | | |
| HB_002 | | | | | |
| HB_003 | | | | | |
| HDM2017 | CDH17 | | Camptothecin | DNA | |
| | | | | Topoisomerase I | |
| HDM2020 | FGFR2b | | | Topoisomerase | |
| | | | | Inhibitor | |
| HDP-103 | PSMA | Cleavable | Amanitin | RNA | |
| | | | | Polymerase II | |
| | | | | Inhibitor | |
| HDP-104 | GCC | | Amanitin | RNA | |
| | | | | Polymerase II | |
| | | | | Inhibitor | |
| HDP-201 | GCC | Protease | Exatecan | DNA | |
| | | cleavable | (Camptothecin) | Topoisomerase I | |
| Hetero | HER-2 | Non-Cleavable | DM1 | Tubulin- | |
| Trastuzumab | | | (Maytansine) | microtubulin | |
| Emtansine | | | | | |
| Biosimilar | | | | | |
| HLX91-048- | HER-2 | Protease | HLX91-048 | | |
| based ADC | | cleavable | | | |
| HMA800067 | CD38 | | | | |
| HMBD-802 | HER-2 | | | ATR Inhibitor; | |
| | | | | DNA | |
| | | | | Topoisomerase I | |
| HMBD-803 | | | | | |
| HMBD-804 | | | | | |
| Homocamptothe | HER-2 | Protease | Camptothecin | DNA | |
| cin ADC | | cleavable | | Topoisomerase I | |
| HRA00129- | c-MET | Cleavable | SHR9265 | DNA | |
| C004 | | | (Exatecan) | Topoisomerase I | |
| HRA00130- | DLL3 | Cleavable | Dxh (Exatecan) | DNA | |
| C004 | | | | Topoisomerase I | |
| HRA00184- | Tissue factor | Cleavable | Dxh (Exatecan) | DNA | |
| C004 | | | | Topoisomerase I | |
| HRA00242- | PSMA | Cleavable | Dxh (Exatecan) | DNA | |
| C004 | | | | Topoisomerase I | |
| HSB-0059 | HER-2; HER- | | | | |
| | 3 | | | | |
| huB 12-MMAE | FAP | Protease | MMAE | Tubulin- | |
| ADC | | cleavable | (Auristatin) | microtubulin | |
| HuB 14-VA- | ASCT2 | Protease | PL2202 | DNA | |
| PL2202 | | cleavable | (Camptothecin) | Topoisomerase I | |
| HY-0001 | | | | | |
| HY0001a | CDCP1 | Protease | MMAE | Tubulin- | |
| | | cleavable | (Auristatin) | microtubulin | |
| IAR116 | c-MET; EGFR | | | | |
| IBI3010 | Folate | | NT1 | DNA | |
| | Receptor | | | Topoisomerase I | |
| | Alpha | | | | |
| IBI3022 | B7-H4; | | | DNA | |
| | TROP-2 | | | Topoisomerase I | |
| IBX13 ADC | | | | | |
| ICAM-1 ADC | ICAM-1 | Cleavable | | DNA | |
| | | | | Topoisomerase I | |
| ICP-B794 | B7-H3 | Cleavable | | DNA | |
| | | | | Topoisomerase I | |
| Idience ADC | TROP-2 | Cleavable | | PARP Inhibitor | |
| IDP-001 | CDCP1; | Protease | MMAE | Tubulin- | |
| | EGFR | cleavable | (Auristatin) | microtubulin | |
| IDP-004 | MET | | | | |
| IDP-005 | | | | | |
| IKS04 | CA242 | Cleavable | LCB20-0187 | DNA Damaging | |
| | | | (Pyrrolobenzodi | Agent | |
| | | | azepine (PBD)) | | |
| IKS073 | B7-H3 | | ProAlk | Protein | |
| | | | (Duocarmycin) | Alkylation | |
| ILB-3102 | | | | | |
| ILB-3103 | B7-H3; DLL3 | | | DNA | |
| | | | | Topoisomerase I | |
| IM-1335 | | | HC74 | DNA | |
| | | | | Topoisomerase I | |
| IM-1340 | | | HC74 | DNA | |
| | | | | Topoisomerase I | |
| IM-1617 | | | HC74 | DNA | |
| | | | | Topoisomerase I | |
| IMB-201 ADC | HLA-G | | | | |
| IMB-202 | | | | | |
| IMD-1135 | CLDN18.2 | | | | |
| IMD-1628 | CLDN6; | | DXd/DX8951 | TLR 7/8 | |
| | TLR7/8 | | (MAAA-1181a) | Agonist; DNA | |
| | | | (Exatecan) | Topoisomerase I | |
| IMD-2101 | HER-2 | | | | |
| IMD-2109 | PD-L1 | | | | |
| IMD-2113 | EGFR; | | DXd/DX8951 | TLR 7/8 | |
| | TLR7/8; | | (MAAA-1181a) | Agonist; DNA | |
| | TROP-2 | | (Exatecan) | Topoisomerase I | |
| IMD-2128 | c-MET; EGFR | | | | |
| IMD-2206 | LY6G6D | | | | |
| IMD-2316 | DLL3 | | DXd/DX8951 | DNA | |
| | | | (MAAA-1181a) | Topoisomerase I | |
| | | | (Exatecan) | | |
| IMD-2329 | B7-H3; DLL3 | | | | |
| IMD-2358 | ROR1 | | DXd/DX8951 | DNA | |
| | | | (MAAA-1181a) | Topoisomerase I | |
| | | | (Exatecan) | | |
| IMD-2408 | CCR8 | | | | |
| IMD-2503 | SSTR2 | | | | |
| IMD-2509 | B7-H3; | | | | |
| | SSTR2 | | | | |
| IMD-3236 | B7-H3 | | | | |
| IMD2126 | PD-L1; | | | TLR 7/8 | |
| | TLR7/8 | | | Agonist; DNA | |
| | | | | Topoisomerase I | |
| IMD526 | HER-2; | | | TLR 7/8 | |
| | TLR7/8 | | | Agonist; DNA | |
| | | | | Topoisomerase I | |
| IMT-101-A01 | | | | | |
| IMT-105-A01 | | | | | |
| IMTO 4842 | | | | | |
| IN30705 | FAP | | | | |
| IN30718 | | | | | |
| IN30728 | | | | | |
| IN30738 | | | | | |
| IN30758 | | | | | |
| IO-0001 | CBLB; PD-1 | | | Inhibitor | |
| IPH43 | MHC class I | Protease | Pyrrolobenzodia | DNA Damaging | |
| | chain-related | cleavable | zepine (PBD) | Agent | |
| | protein A and | | | | |
| | B (MICA and | | | | |
| | MICB) | | | | |
| ITC-6102RO | B7-H3 | Cleavable | dHBD | DNA Damaging | |
| | | | (heterocycle- | Agent | |
| | | | fused | | |
| | | | benzodiazepine | | |
| | | | dimer) | | |
| | | | (Pyrrolobenzodi | | |
| | | | azepine (PBD)) | | |
| JSKN020 | | | | | |
| JSKN021 | EGFR; HER-3 | Cleavable | MMAE | DNA | |
| | | | (Auristatin) | Topoisomerase | |
| | | | | I; Tubulin- | |
| | | | | microtubulin | |
| JSKN022 | Integrin beta- | Cleavable | T01 | DNA | |
| | 6; PD-L1 | | | Topoisomerase I | |
| JSKN027 | | | | | |
| JSKN028 | | | | | |
| JY207 | CD47; PD-L1 | | MMAE | Tubulin- | |
| | | | (Auristatin) | microtubulin | |
| JY207b | CD47; PD-L1 | | MMAE | Tubulin- | |
| | | | (Auristatin) | microtubulin | |
| K-679 | EGFR | | DM1 | Tubulin- | |
| | | | (Maytansine) | microtubulin | |
| KA-2886-LD38 | c-MET; EGFR | Cleavable | LD-38 | DNA | |
| | | | | Topoisomerase I | |
| KA-2887-LD38 | EGFR; PD-L1 | Cleavable | | DNA | |
| | | | | Topoisomerase I | |
| KA-3123-LD38 | B7-H3; EGFR | Cleavable | LD-38 | DNA | |
| | | | | Topoisomerase I | |
| KHN922 | HER-3 | | | RNA | |
| | | | | Polymerase II | |
| | | | | Inhibitor; DNA | |
| | | | | Topoisomerase I | |
| KIVU-107 | | | | DNA | |
| | | | | Topoisomerase I | |
| KIVU-202 | | | | DNA | |
| | | | | Topoisomerase I | |
| KIVU-305 | | | | DNA | |
| | | | | Topoisomerase I | |
| KNP-701 | c-MET; EGFR | | | | |
| KY-0601 | MET | | | | |
| LA-057-MMAE | LAIR1 | | MMAE | Tubulin- | |
| | | | (Auristatin) | microtubulin | |
| LAIR-1 ADC | LAIR1 | | | | |
| LBL-013 ADC | | | | | |
| LBL-052 | | | | | |
| LBL-054 | CDH17 | Cleavable | Exatecan | DNA | |
| | | | (Camptothecin) | Topoisomerase I | |
| LBL-058 | CD3; DLL3 | | | DNA | |
| | | | | Topoisomerase I | |
| LCB02A | CLDN18.2 | Cleavable | | DNA | |
| | | | | Topoisomerase I | |
| LCB22A | Cancer- | Cleavable | MMAE | Tubulin- | |
| | specific Tn- | | (Auristatin) | microtubulin | |
| | glycosylated | | | | |
| | proteins | | | | |
| LCB28A | | Cleavable | | | |
| LCB36 | CD20; | Cleavable | Pyrrolobenzodia | DNA Damaging | |
| | SIGLEC2 | | zepine (PBD) | Agent | |
| LCB37 | | | | DNA | |
| | | | | Topoisomerase I | |
| LCB45A | Cancer- | Cleavable | MMAE | Tubulin- | |
| | specific Tn- | | (Auristatin) | microtubulin | |
| | glycosylated | | | | |
| | proteins | | | | |
| LCB97 | LICAM | Cleavable | | | |
| LM-001 | NaPi2b | | | | |
| LM-002 | CDH17 | | LDX2 | DNA | |
| | | | | Topoisomerase I | |
| LM-004 | CEACAM5 | | LDX2 | DNA | |
| | | | | Topoisomerase I | |
| LM-005 | GPC3 | | | | |
| LM-006 | | | LDX2 | DNA | |
| | | | | Topoisomerase I | |
| LM-007 | Folate | | LDX2 | DNA | |
| | Receptor | | | Topoisomerase I | |
| | Alpha | | | | |
| LM-317 | NaPi2b | Cleavable | LDX2 | DNA | |
| | | | | Topoisomerase I | |
| LM-350 | CDH17 | Cleavable | LDX2 | DNA | |
| | | | | Topoisomerase I | |
| LY4175408 | PTK7 | Protease | Exatecan | DNA | |
| | | cleavable | (Camptothecin) | Topoisomerase I | |
| M0121 | | | Exatecan | DNA | |
| | | | (Camptothecin) | Topoisomerase I | |
| M0251 | EGFR; MUC- | | Exatecan | DNA | |
| | 1 | | (Camptothecin) | Topoisomerase I | |
| M7152 | NaPi2b | | Exatecan | DNA | |
| | | | (Camptothecin) | Topoisomerase I | |
| M7437 | | | Exatecan | DNA | |
| | | | (Camptothecin) | Topoisomerase I | |
| MABS-03 | | Cleavable | DXd/DX8951 | DNA | |
| | | | (MAAA-1181a) | Topoisomerase I | |
| | | | (Exatecan) | | |
| MBK-103 | Folate | Protease | Exatecan | DNA | |
| | Receptor | cleavable | (Camptothecin) | Topoisomerase I | |
| | Alpha | | | | |
| MBRC-201 | | | | | |
| MBRC-301 | | | | | |
| MC001 | Folate | | | | |
| | Receptor | | | | |
| | Alpha | | | | |
| MC002 | CT22 | | | | |
| MC003 | Folate | | | | |
| | Receptor | | | | |
| | Alpha | | | | |
| MG2002B | | | | | |
| MG2106E | | | | | |
| MGC030 | | | | DNA | |
| | | | | Topoisomerase I | |
| MGTA-45 ADC | CD45 | | | | |
| MHA112 | | Cleavable | Paclitaxel | Tubulin- | |
| | | | (Taxanes) | microtubulin | |
| MHB009C | B7-H4 | Cleavable | | DNA | |
| | | | | Topoisomerase I | |
| MHB048C | PSMA | | | DNA | |
| | | | | Topoisomerase I | |
| MP-1959(SP-2)- | LGALS3BP | Cleavable | DM4 | Tubulin- | |
| ADC | | Disufide bond | (Maytansine) | microtubulin | |
| | | based | | | |
| | | (chemical) | | | |
| MP-HER3-ADC | HER-3 | Non-Cleavable | MMAF | Tubulin- | |
| | | | (Auristatin) | microtubulin | |
| MQI-181 | | | | | |
| MQI-191 | | | | | |
| MRG007 | CDH17 | | Exatecan | DNA | |
| | | | (Camptothecin) | Topoisomerase I | |
| MRX-23 | | | Camptothecin | DNA | |
| | | | | Topoisomerase I | |
| MT-40X | | | | | |
| MT0001-ADC | PD-L1 | | | | |
| MW-C1 | CLDN1 | Cleavable | MMAE | Tubulin- | |
| | | | (Auristatin) | microtubulin | |
| NaPi2b-PL2202 | NaPi2b | Protease | PL2202 | DNA | |
| ADC | | cleavable | (Camptothecin) | Topoisomerase I | |
| NAV-001 | Mesothelin | Protease | PNU-159682 | Topoisomerase | |
| | (MSLN); RET | cleavable | (Anthracycline) | II Inhibitor | |
| NB001 ADC | B7-H3 | | MMAE | Tubulin- | |
| | | | (Auristatin) | microtubulin | |
| NBD07 | | Cleavable | PBX-7016 | DNA | |
| | | | | Topoisomerase I | |
| NBD08 | | | PBX-7016 | DNA | |
| | | | | Topoisomerase I | |
| NBD13 | | | PBX-7016 | DNA | |
| | | | | Topoisomerase I | |
| NBE-105 | | Non-Cleavable | PNU-159682 | Topoisomerase | |
| | | | (Anthracycline) | II Inhibitor | |
| NC030-107 | HER-2 | | AF-HEA | Tubulin- | |
| | | | (Auristatin) | microtubulin | |
| NN3206 | | | | | |
| NPX125 | HERV-H | | | | |
| | LTR- | | | | |
| | associating | | | | |
| | protein 2 | | | | |
| NTX-1105 | Nectin-4 | | | | |
| NTX8090 | CD155 | | | | |
| NXV01c | c-MET; EGFR | Protease | MMAE | Tubulin- | |
| | | cleavable | (Auristatin) | microtubulin | |
| OBI-201 | HER-2; | | | DNA | |
| | TROP-2 | | | Topoisomerase I | |
| OBI-902 | TROP-2 | | Exatecan | DNA | |
| | | | (Camptothecin) | Topoisomerase I | |
| OBI-904 | Nectin-4 | | | DNA | |
| | | | | Topoisomerase I | |
| OBT035 | | | | | |
| OBT227 | | | | | |
| OBT417 | | | | | |
| OBT542 | | | | | |
| OMTX103 | Endoglin | | MMAE | Tubulin- | |
| | | | (Auristatin) | microtubulin | |
| OMTX603-3 | Endoglin | | Eribulin | Tubulin- | |
| | | | | microtubulin | |
| OMTX703 | Endoglin | Protease | Cytolysin | Tubulin- | |
| | | cleavable | | microtubulin | |
| OMTX707 | MTX7 | | | | |
| ONA-255 | | | | | |
| ONA-389 | | | | | |
| ONC-784 | CD24 | | | | |
| Oqory B7-H3 | B7-H3 | | Camhexin | | |
| ADC | | | | | |
| Oqory CD25 | IL-2R Alpha | | Camhexin; | Tubulin- | |
| ADC | | | Duostatin5 | microtubulin | |
| | | | (MMAF) | | |
| Oqory Next Gen | TROP-2 | | Camhexin | | |
| TROP2 ADC | | | | | |
| Oqory ROR1 | ROR1 | | Camhexin; | Tubulin- | |
| ADC | | | Duostatin5 | microtubulin | |
| | | | (MMAF) | | |
| OQY-8811 | BCMA | Non-Cleavable | Duostatin5 | Tubulin- | |
| | | | (MMAF) | microtubulin | |
| Padynex | HER-2 | Non-Cleavable | DM1 | Tubulin- | |
| | | | (Maytansine) | microtubulin | |
| PB-MMAE-2 | core 2 O- | Protease | MMAE | Tubulin- | |
| ADC | glycan | cleavable | (Auristatin) | microtubulin | |
| PB-MMAE-5 | core 2 O- | Protease | MMAE | Tubulin- | |
| ADC | glycan | cleavable | (Auristatin) | microtubulin | |
| PB-MMAE-6 | core 2 O- | Protease | MMAE | Tubulin- | |
| ADC | glycan | cleavable | (Auristatin) | microtubulin | |
| PBX-001 | TROP-2 | Cleavable | Camptothecin | DNA | |
| | | | | Topoisomerase I | |
| PBX-CB-01 | | Cleavable | | DNA | |
| | | | | Topoisomerase I | |
| PBX-CT01 | c-MET | Cleavable | PBX-7016 | DNA | |
| | | | | Topoisomerase I | |
| PCG-C002 | | | | | |
| PCG-C004 | | | | | |
| PCG-C006 | | | | | |
| PCMdt-MMAE | c-MET; RON | Protease | MMAE | Tubulin- | |
| | (Recepteur | cleavable | (Auristatin) | microtubulin | |
| | d'Origine | | | | |
| | nantais) | | | | |
| PF-08046876 | Integrin beta-6 | beta- | AMDCPT | DNA | |
| | | glucuronidase | (Camptothecin) | Topoisomerase I | |
| | | cleavable | | | |
| PG-217 | | | | | |
| PHN-02X | | | | | |
| PLB-001 | DLL3 | Cleavable | Exatecan | DNA | |
| | | | (Camptothecin) | Topoisomerase I | |
| PLB-002 | CLDN6 | Cleavable | Eribulin | Tubulin- | |
| | | | | microtubulin | |
| PLB-003 | | | | | |
| PLB-004 | | | | | |
| PLB-005 | | | | | |
| PLB-006 | | | | | |
| PLBC-007 | | | | | |
| PLBC-008 | | | | | |
| PLD-107 | | | | | |
| PM1300 | EGFR; HER-3 | Cleavable | | DNA | |
| | | | | Topoisomerase I | |
| POZ-ADCs | | | | | |
| PPAB002 | Tissue factor | | | | |
| PPAB003 | CD47 | | | | |
| PPMX-T004 | P-Cadherin | | | | |
| PRN 201 | | | | | |
| PRN 211 | | | | | |
| PRN 222 | | | | | |
| PRO1106 | SLITRK6 | Cleavable | Exatecan | DNA | |
| | | | (Camptothecin) | Topoisomerase I | |
| PRO1135 | | | | | |
| ProD001 | | | | | |
| ProD002 | | | | | |
| ProD003 | | | | | |
| PSM-101 | | | | | |
| PTM-001-ADC | | Non-Cleavable | DM1 | Tubulin- | |
| | | | (Maytansine) | microtubulin | |
| PYX-203 | CD123 | Cleavable | CPI | DNA Damaging | |
| | | | (Cyclopropylpyr | Agent | |
| | | | roloindole) | | |
| QBK249-G | HER-2 | | | | |
| QHL-1096 | TLR7/8 | | DXd/DX8951 | TLR 7/8 | |
| | | | (MAAA-1181a) | Agonist; DNA | |
| | | | (Exatecan) | Topoisomerase I | |
| QLi5 ADC | Immunoglobul | | | | |
| | in G | | | | |
| QLS5133 | CDH6 | | QLS6916 | DNA | |
| | | | | Topoisomerase I | |
| RB-205 | | | | | |
| RC278 | | | | | |
| RGX-019 ADC | MerTK | | MMAE | Tubulin- | |
| | | | (Auristatin) | microtubulin | |
| RI-ACT | TAA | | | | |
| RS-5 | TAMs | | | | |
| | (Tumor- | | | | |
| | associated | | | | |
| | macrophages) | | | | |
| RS-7 | TAMs | | | | |
| | (Tumor- | | | | |
| | associated | | | | |
| | macrophages) | | | | |
| RS-8 | TAMs | | | | |
| | (Tumor- | | | | |
| | associated | | | | |
| | macrophages) | | | | |
| RS7-DL 11 | TROP-2 | Protease | MMAE | Tubulin- | |
| | | cleavable | (Auristatin) | microtubulin | |
| RT022 | EGFR; HER-3 | | | | |
| SBO-154 | MUC-1 | | | | |
| SCR-A0011 | B7-H3; c- | | CPT116 | DNA | |
| | MET | | | Topoisomerase I | |
| SCR-A003 | LIV-1 | Cleavable | | DNA | |
| | | | | Topoisomerase I | |
| SCR-A006 | c-MET; EGFR | Cleavable | | DNA | |
| | | | | Topoisomerase I | |
| SCR-A008 | CDH17 | Cleavable | CPT116 | DNA | |
| | | | | Topoisomerase I | |
| SDP01873 | c-MET; HER- | Cleavable | | DNA | |
| | 3 | | | Topoisomerase I | |
| SDP03923-000- | LIV-1 | Cleavable | Exatecan | DNA | |
| 9106 | | | (Camptothecin) | Topoisomerase I | |
| SDV2102 | PSMA | | MMAE | Tubulin- | |
| | | | (Auristatin) | microtubulin | |
| SDV2103 | TROP-2 | | | | |
| SGZ007 | | | | | |
| SGZ008 | | | | | |
| SGZ009 | | | | | |
| SGZ010 | | | | | |
| SIM 0618 | c-MET; EGFR | | | | |
| SIM 0682 | | | | | |
| SIM0680 | ENPP3 | | | | |
| SKB501 | | | | | |
| SKM-104 | G protein- | Cleavable | | | |
| | coupled | | | | |
| | receptors | | | | |
| | (GPCRs) | | | | |
| SKM-201 | G protein- | Cleavable | | | |
| | coupled | | | | |
| | receptors | | | | |
| | (GPCRs) | | | | |
| SKM-301 | G protein- | Cleavable | | | |
| | coupled | | | | |
| | receptors | | | | |
| | (GPCRs) | | | | |
| SLV-404 | RYK | Cleavable | DXd/DX8951 | DNA | |
| | | | (MAAA-1181a) | Topoisomerase I | |
| | | | (Exatecan) | | |
| SMP-771 | CD38 | | Eribulin | Tubulin- | |
| | | | | microtubulin | |
| SMP-ADeC | | | | | |
| SMP-Dual | | | | | |
| SMP-xxx | 5T4 | | Eribulin | Tubulin- | |
| | | | | microtubulin | |
| SO-N107 | | | Anthracycline | Topoisomerase | |
| | | | | II Inhibitor | |
| SOT106 | LRRC15 | beta- | MMAE | Tubulin- | |
| | | glucuronidase | (Auristatin) | microtubulin | |
| | | cleavable | | | |
| SOT109 | CDH17 | Protease | Exatecan | DNA | |
| | | cleavable | (Camptothecin) | Topoisomerase I | |
| SOT110 | | | | | |
| SOT112 | | | | | |
| SOT113 | | | | | |
| SPT-101 | LILRB2 | Cleavable | | | |
| SPX-603 | PD-L1; VEGF | Cleavable | | | |
| SRB21 | HER-2; HER- | Cleavable | | | |
| | 3 | | | | |
| STRO-003 | ROR1 | beta- | SC3386 | DNA | |
| | | glucuronidase | (Exatecan) | Topoisomerase I | |
| | | cleavable | | | |
| STRO-004 | Tissue factor | beta- | Exatecan | DNA | |
| | | glucuronidase | (Camptothecin) | Topoisomerase I | |
| | | cleavable | | | |
| STRO-006 | Integrin αVβ6 | | | | |
| STRO-00X | FcγR | beta- | Exatecan | DNA | |
| Exatecan ADC | | glucuronidase | (Camptothecin) | Topoisomerase I | |
| | | cleavable | | | |
| STRO-00X | HER-2 | beta- | MMAE | DNA | |
| Topoli + anti- | | glucuronidase | (Auristatin) | Topoisomerase | |
| Tubulin Dual- | | cleavable | | I; Tubulin- | |
| payload ADC | | | | microtubulin | |
| STRO-00X | Tissue factor | beta- | Exatecan | PARP Inhibitor; | |
| Topoli + PARPi | | glucuronidase | (Camptothecin); | DNA | |
| Dual-payload | | cleavable | Talazoparib | Topoisomerase I | |
| ADC | | | | | |
| STRO-00Y | | | | | |
| STX-1 | DPP4 | Protease | Exatecan | DNA | |
| | | cleavable | (Camptothecin) | Topoisomerase I | |
| SWY2321 | c-MET; EGFR | Cleavable | | DNA | |
| | | | | Topoisomerase I | |
| SYN303 | FAP | | Synthis-003 | Inhibitor | |
| SYS6042 | TROP-2 | | | DNA | |
| | | | | Topoisomerase I | |
| SYS6045 | | | | | |
| SYS6051 | Tissue factor | Protease | Exatecan | DNA | |
| | | cleavable | (Camptothecin) | Topoisomerase I | |
| T-GS-LP | HER-2 | | GS-P-000 | DNA | |
| | | | | Topoisomerase I | |
| T340-ADC | | | | | |
| TA99 ADC | GP75; TLR7 | Protease | | | |
| | | cleavable | | | |
| TAE020 | | | | | |
| TAK-ATACs | | | Amanitin | RNA | |
| | | | | Polymerase II | |
| | | | | Inhibitor | |
| TARG9 | Integrin α10β1 | | | | |
| TAVO307 | CDH17 | Cleavable | MMAE | Tubulin- | |
| | | | (Auristatin) | microtubulin | |
| TAVO605 | CDCP1 | | MMAE | Tubulin- | |
| | | | (Auristatin) | microtubulin | |
| TB-ADC-01 | MUC13 | | | | |
| TCX-101 ADC | Tumor- | | MMAE | Tubulin- | |
| | Associated | | (Auristatin) | microtubulin | |
| | Carbohydrate | | | | |
| | Antigens | | | | |
| TGW111 | | Cleavable | | | |
| TGW121 | | Cleavable | | | |
| TH0059 | HER-2; HER- | | | | |
| | 3 | | | | |
| THEL-002 ADC | Human | | | | |
| | Cytomegalovirus | | | | |
| | (HCMV) | | | | |
| THEL-003 ADC | G protein- | | | | |
| | coupled | | | | |
| | receptors | | | | |
| | (GPCRs) | | | | |
| TJ101 | B7-H3; EGFR | Cleavable | PY-4car2 | DNA | |
| | | | (Camptothecin) | Topoisomerase I | |
| TJ102 | CDH6; Folate | Cleavable | PE-E2K; PY- | DNA | |
| | Receptor | | 4car2 | Topoisomerase | |
| | Alpha | | (Camptothecin) | I; Tubulin- | |
| | | | | microtubulin | |
| TNT ADC | Doppel | | | | |
| TQB6411 | c-MET; EGFR | Cleavable | DDDXd | DNA | |
| | | | (DXd/DX8951 | Topoisomerase I | |
| | | | (MAAA- | | |
| | | | 1181a)) | | |
| TriOar A-01 | HER-2 | | | | |
| TriOar T-01 | | | | | |
| TriOar T-02 | | | | | |
| Trop-2 TIE- | TROP-2 | | | | |
| ADC | | | | | |
| TROP2-Eribulin | TROP-2 | Protease | Eribulin | Tubulin- | |
| ADC | | cleavable | | microtubulin | |
| TSD101 | BCMA | | MMAF | Tubulin- | |
| | | | (Auristatin) | microtubulin | |
| TSRF 786C- | CD155 | | | | |
| ADC | | | | | |
| TST012 | FGFR2b | | | | |
| TST013 | LIV-1 | | | DNA | |
| | | | | Topoisomerase I | |
| TST105 | FGFR2b | | | DNA | |
| | | | | Topoisomerase I | |
| TSY0110 | HER-2 | Non-Cleavable | DM1 | Tubulin- | |
| | | | (Maytansine) | microtubulin | |
| TT-01 ADC | | | | | |
| TUB-010 | CD30 | Protease | MMAE | Tubulin- | |
| | | cleavable | (Auristatin) | microtubulin | |
| TUB-050 | | | | | |
| TUB-060 | | | | | |
| VBC101-F11 | c-MET; EGFR | | | | |
| VBC103 | Nectin-4; | | | DNA | |
| | TROP-2 | | | Topoisomerase I | |
| VBC108 | CDH17; | | | DNA | |
| | CLDN18.2 | | | Topoisomerase I | |
| VCR-018 | CD205 | | | | |
| VIP924 ADC | CXCR5 | Cleavable | VIP716 | Tubulin- | |
| | | | | microtubulin | |
| VIR-2981 ADC | Neuraminidase | | | | |
| VX-A902 | HER-2 | | | | |
| VX-A904 | | | | | |
| VX-A905 | | | | | |
| WTx-03005 | | | | | |
| WTX201 | | | | | |
| WTX202 | | | | | |
| WTX301 | | | | | |
| XB033 | IL13Rα2 | | | DNA | |
| | | | | Topoisomerase I | |
| XB371 | Tissue factor | Cleavable | | DNA | |
| | | | | Topoisomerase I | |
| XBH-102 | | | | | |
| XBH107 | | | | | |
| XBH40 | B7-H3 | | | | |
| XCN-010 | actM | Cleavable | MMAE | Tubulin- | |
| | | | (Auristatin) | microtubulin | |
| YB-800ADC1 | NPTxR | | | Tubulin- | |
| | | | | microtubulin | |
| YB-800ADC2 | NPTxR | | | Topoisomerase | |
| | | | | Inhibitor; | |
| | | | | Tubulin- | |
| | | | | microtubulin | |
| YB1-ADC- | CD47 | | | | |
| CD47 | | | | | |
| YB1-ADC-Her2 | HER-2 | | | | |
| YB1-ADC- | PD-L1 | | | | |
| PDL1 | | | | | |
| YBL-001 | DLK-1 | beta- | MMAE | Tubulin- | |
| | | glucuronidase | (Auristatin) | microtubulin | |
| | | cleavable | | | |
| YBL-015 | B7-H3 | | | | |
| YH012 | HER-2; | Cleavable | MMAE | Tubulin- | |
| | TROP-2 | | (Auristatin) | microtubulin | |
| YL212 | DLL3 | Protease | Camptothecin | DNA | |
| | | cleavable | | Topoisomerase I | |
| YL221 | EGFR | Protease | Camptothecin | DNA | |
| | | cleavable | | Topoisomerase I | |
| YL242 | VEGF | Protease | C24 | DNA | |
| | | cleavable | | Topoisomerase I | |
| ZL-6301 | ROR1 | | | | |
| ZW220 | NaPi2b | Protease | ZD06519 | DNA | |
| | | cleavable | (Camptothecin) | Topoisomerase I | |
| ZW251 | GPC3 | Protease | ZD06519 | DNA | |
| | | cleavable | (Camptothecin) | Topoisomerase I | |
| ZW327 | Ly6E | Protease | ZD06519 | DNA | |
| | | cleavable | (Camptothecin) | Topoisomerase I | |
| AEGFR-E- | EGFR | | E-P125A | Inhibitor | |
| P125A | | | | | |
| VBC104 | CD20; CD79b | | | | |
| ZL-6201 | LRRC15 | Protease | Camptothecin | DNA | |
| | | cleavable | | Topoisomerase I | |

In embodiments herein, an antibody-drug conjugate is provided in a suspension or a solution. The suspension or solution includes any suitable suspension or solution of an antibody-drug conjugate, such as those exemplified. Combinations and treatment regimens are provided herein in which an antibody-drug conjugate is administered in combination with a soluble hyaluronidase.

In various embodiments of the disclosure, when a composition described is administered to a subject in need, the systemic, subcutaneous, and/or lymphatic pharmacokinetic properties of the antibody drug conjugates fall withing the ranges shown in the table below.

| Subcutaneous (SC) + rHuPH20 | | | | | |
|---|---|---|---|---|---|
| Relative to IV Administration | | | SC + rHuPH20 Relative to SC Alone | | |
| Cmin | Cmax | AUC | Cmin | Cmax | AUC |

Systemic

| Total ADC | lower-higher | lower | lower-higher | Higher | Higher | Higher |
|---|---|---|---|---|---|---|
| concentration | (50-200%) | (25-100%) | (50-200%) | (100-200%) | (100-200%) | (100-200%) |
| Total | lower-equal (75-100%) | | | higher (100-200%) | | |
| ADC:Free | | | | | | |
| antibody¹ | | | | | | |
| Total | lower-equal (75-100%) | | | higher (100-200%) | | |
| ADC:Free | | | | | | |
| payload² | | | | | | |

Subcutaneous

| Total ADC | Lower | Lower | Lower |
|---|---|---|---|
| concentration | (50-100%) | (50-100%) | (50-100%) |
| Total | higher (100-200%) | | |
| ADC:Free | | | |
| antibody¹ | | | |
| Total | higher (100-200%) | | |
| ADC:Free | | | |
| payload² | | | |

Lymphatic

| Total ADC | Higher | Higher | Higher | Higher | Higher | Higher |
|---|---|---|---|---|---|---|
| concentration | (100-500%) | (100-500%) | (100-500%) | (100-300%) | (100-300%) | (100-300%) |
| Total | Higher | | | Higher | | |
| ADC:Free | (100-500%) | | | (100-300%) | | |
| antibody¹ | | | | | | |
| Total | Higher | | | Higher | | |
| ADC:Free | (100-500%) | | | (100-300%) | | |
| payload² | | | | | | |

| rHuPH20 Treated Subjects | | | |
|---|---|---|---|
| Day 1 | Day 7 | Day 14 | Day 21 |

Systemic

| | | | | |
|---|---|---|---|---|
| Free antibody:Total ADC | 0-45% | 0-190% | 0-300% | 0-425% |
| Free antibody:Total ADC (ADCs | 20-45% | 85-130% | 150-220% | 225-310% |
| with Ab-linker instability) | | | | |
| Total ADC:Free antibody (very | 50-70% | 0-5% | 0-5% | 0-5% |
| unstable linker, such as carbonate- | | | | |
| based) | | | | |
| Free payload: Total ADC (mass | 0-1.3% | 0-4% | 0-4% | 0-4% |
| concentrations) | | | | |
| Total ADC:Free antibody¹ | | | | |
| Total ADC:Free payload² | | | | |

NOTE:
percentages provided are for amount of rHuPH20 relative to IV or SC alone, not percent improvement. The higher the ratio of total ADC:free antibody, the better; this means the ADC is comprised of more attached payload.

Provided in this disclosure are combination dosing regimens comprising subcutaneously administering to a human subject a therapeutically effective amount of an antibody drug conjugate; and administering a soluble hyaluronidase.

In embodiments disclosed, the antibody drug conjugate comprises a monoclonal antibody. In some embodiments disclosed, the antibody drug conjugate comprises a payload. In various embodiments the payload is cytotoxic. In some embodiments the antibody is bispecific.

In various embodiments, a detectable amount of intact antibody drug conjugate reaches systemic circulation.

In some embodiments, serum levels of the antibody drug conjugate are between 10-200% of the equivalent intravenous dose of the antibody drug conjugate. In some cases, the serum levels of the monoclonal antibody are between 10-200% of the equivalent intravenous. In other embodiments, the serum levels of the free payload are between 10-200% of the equivalent intravenous dose.

In some embodiments, serum levels of the antibody drug conjugate are higher than an equivalent dose of the antibody drug conjugate without hyaluronidase. In some cases, the serum levels of the monoclonal antibody are higher than an equivalent dose of the antibody drug conjugate without hyaluronidase. In other embodiments, the serum levels of the payload are higher than an equivalent dose of the antibody drug conjugate without hyaluronidase.

In the disclosure provided, serum levels can be taken at one or more time points after administration, for example, 6, 24, 48, 72, 96, 168 and/or 336 hours after administration.

In some embodiments, subcutaneous tissue samples are taken at one or more time points after administration, for example, 6, 24, 48, 72, 96, 168 and/or 336 hours after administration.

Disclosed herein are embodiments where the subcutaneous tissue concentration is less than 100%, 90%, 80%, 70%, 60%, 50%, 40%, 30%, 20% or 10% relative to delivery of the total antibody drug conjugate without hyaluronidase.

In some embodiments disclosed, the subcutaneous tissue concentration of the monoclonal antibody is less than 100%, 90%, 80%, 70%, 60%, 50%, 40%, 30%, 20%, or 10% relative to delivery without hyaluronidase.

Disclosed herein are combination dosing regimen where the subcutaneous tissue concentration of the free payload is less than is less than 100%, 90%, 80%, 70%, 60%, 50%, 40%, 30%, 20%, or 10% relative to delivery without hyaluronidase.

In embodiments disclosed, the AUC of the combination dosing regimen is at least about 5% to about 250% of an equivalent intravenous dose of the antibody drug conjugate. For example, the AUC is about 10%, 20%, 30%, 40%, 50%, 60%, 70%, 80%, 90%, 100%, 110%, 120%, 130%, 140%, 150%, 160%, 170%, 180%, 190%, and/or 200% of an equivalent intravenous dose of the antibody drug conjugate.

In embodiments disclosed, the AUC of the combination dosing regimen is about 5% to about 250% higher than an equivalent dose of the antibody drug conjugate without hyaluronidase. For example, the AUC is at least 10%, 20%, 30%, 40%, 50%, 60%, 70%, 80%, 90%, 100%, 110%, 120%, 130%, 140%, 150%, 160%, 170%, 180%, 190%, and/or 200% higher than an equivalent dose of the antibody drug conjugate without hyaluronidase.

In embodiments disclosed, the AUC of the combination dosing regimen is greater than 5% to 250% relative to delivery of the antibody drug conjugate without hyaluronidase. For example, the AUC is greater than 10%, 20%, 30%, 40%, 50%, 60%, 70%, 80%, 90%, 100%, 110%, 120%, 130%, 140%, 150%, 160%, 170%, 180%, 190%, and/or 200% relative to delivery of the antibody drug conjugate without hyaluronidase.

In embodiments disclosed, the Cmin of the combination dosing regimen is 5% to 250% of an equivalent intravenous dose of the antibody drug conjugate. For example, the Cmin is at least 10%, 20%, 30%, 40%, 50%, 60%, 70%, 80%, 90%, 100%, 110%, 120%, 130%, 140%, 150%. 160%, 170%, 180%, 190%, and/or 200% of an equivalent intravenous dose of the antibody drug conjugate.

In embodiments disclosed, the Cmin of the combination dosing regimen is 5% to 250% higher than an equivalent dose of the antibody drug conjugate without hyaluronidase. For example, the Cmin is at least 10%, 20%, 30%, 40%, 50%, 60%, 70%, 80%, 90%, 100%, 110%, 120%, 130%, 140%, 150%, 160%, 170%, 180%, 190%, and/or 200% higher than an equivalent dose of the antibody drug conjugate without hyaluronidase.

In embodiments disclosed, the Cmin of the combination dosing regimen is greater than 5% to 250% relative to delivery of the antibody drug conjugate without hyaluronidase. For example, the Cmin is greater than 10%, 20%, 30%, 40%, 50%, 60%, 70%, 80%, 90%, 100%, 110%, 120%, 130%, 140%, 150%, 160%, 170%, 180%, 190%, and/or 200% relative to delivery of the antibody drug conjugate without hyaluronidase.

In embodiments disclosed, the Cmax of the combination dosing regimen is 5% to 100% of an equivalent intravenous dose of the antibody drug conjugate. For example, the Cmax is at least 100%, 90%, 80%, 70%, 60%, 50%, 40%, 30%, 20%, 10%, and/or 5% of an equivalent intravenous dose of the antibody drug conjugate.

In embodiments disclosed, the Cmax of the combination dosing regimen is 5% to 100% less than an equivalent dose of the antibody drug conjugate without hyaluronidase. For example, the Cmax is at least 100%, 90%, 80%, 70%, 60%, 50%, 40%, 30%, 20%, 10%, and/or 5% less than an equivalent dose of the antibody drug conjugate without hyaluronidase.

In embodiments disclosed, the Cmax of the combination dosing regimen is greater than 5% to 100% relative to delivery of the antibody drug conjugate without hyaluronidase. For example, the Cmax is at least 100%, 90%, 80%, 70%, 60%, 50%, 40%, 30%, 20%, 10%, and/or 5% greater relative to delivery of the antibody drug conjugate without hyaluronidase.

Embodiments of the dosing regimens provided herein have a time to Cmax (Tmax) for the antibody drug conjugate of greater than about 1.5 hours to 180 hours. For example, the time to Cmax (Tmax) for the antibody drug conjugate is greater than about 2 hours, 4 hours, 6 hours, 12 hours, 24 hours, 48 hours, 72 hours, 96 hours, 120 hours, 144 hours, and/or 168 hours.

Embodiments of the dosing regimens provided herein have a time to Cmax (Tmax) for the monoclonal antibody of greater than about 1.5 hours to 180 hours. For example, the time to Cmax (Tmax) for the monoclonal antibody is greater than about 2 hours, 4 hours, 6 hours, 12 hours, 24 hours, 48 hours, 72 hours, 96 hours, 120 hours, 144 hours, and/or 168 hours.

In embodiments of the combination dosing regimen disclosed, when a higher dose of the antibody drug conjugate with hyaluronidase is delivered subcutaneously than a therapeutically effective dose administered intravenously with an equal or lower Cmax. In some embodiments, the equal or lower Cmax is for antibody drug conjugate. In other embodiments, the equal or lower Cmax is for monoclonal antibody. In still other embodiments, the equal or lower Cmax is for free payload.

In various embodiments of the combination dosing regimen disclosed, there is a lower concentration of antibody drug conjugate in the subcutaneous tissue relative to administration without hyaluronidase. In some cases, there is a lower concentration of antibody drug conjugate in the subcutaneous tissue relative to administration without hyaluronidase. In some cases, there is a lower concentration is a lower concentration of monoclonal antibody, and/or the antibody drug conjugate has a payload, and the lower concentration is a lower concentration in the free payload.

Disclosed herein are combination dosing regimens wherein antibody drug conjugate is present in an amount greater than antibody drug conjugate which is not intact. In various embodiments, the antibody drug conjugate is present in an amount less than free payload.

In various embodiments disclosed, subcutaneous delivery of antibody drug conjugate with soluble hyaluronidase results in greater therapeutic efficacy of the antibody-drug candidate in the human subject relative to intravenous administration of the antibody drug conjugate without hyaluronidase. In some embodiments, subcutaneous delivery of antibody drug conjugate with soluble hyaluronidase increases overall survival in the human subject relative to intravenous administration of the antibody drug conjugate without hyaluronidase. Included in these embodiments are instances where the increased survival is disease free.

Disclosed herein are combination dosing regimens, wherein subcutaneous delivery of antibody drug conjugate with soluble hyaluronidase increases objective response rate of the antibody-drug in the human subject candidate relative to intravenous administration of the antibody drug conjugate without hyaluronidase. In embodiments disclosed, subcutaneous delivery of antibody drug conjugate with soluble hyaluronidase produces greater complete response of the antibody-drug candidate in the human subject relative to intravenous administration of the antibody drug conjugate without hyaluronidase.

Embodiments of the combination dosing regimen include subcutaneous delivery of antibody drug conjugate with soluble hyaluronidase increases progression-free survival of the antibody-drug candidate in the human subject relative to intravenous administration of the antibody drug conjugate without hyaluronidase. In various embodiments, subcutaneous delivery of antibody drug conjugate with soluble hyaluronidase increases progression-free survival of the antibody-drug candidate in the human subject relative to intravenous administration of the antibody drug conjugate without hyaluronidase. In specific embodiments, subcutaneous delivery of antibody drug conjugate with soluble hyaluronidase decreases time to treatment failure of the antibody-drug candidate in the human subject relative to intravenous administration of the antibody drug conjugate without hyaluronidase. In some embodiments, subcutaneous delivery of antibody drug conjugate with soluble hyaluronidase increases duration of response of the antibody-drug candidate in the human subject relative to intravenous administration of the antibody drug conjugate without hyaluronidase.

Provided herein are combination dosing regimens wherein subcutaneous delivery of antibody drug conjugate with soluble hyaluronidase reduces toxicity of the antibody-drug candidate in the human subject relative to intravenous administration of the antibody drug conjugate without hyaluronidase. In some embodiments, the toxicity is local toxicity at the site of injection. In other embodiments, the local toxicity is selected from the group consisting of subcutaneous cytotoxicity, necrosis, lesions, ulcers, pruritis, infection, rash and dry skin.

Provided herein are combination dosing regimens wherein subcutaneous delivery of antibody drug conjugate with soluble hyaluronidase reduces adverse events of the antibody-drug candidate in the human subject relative to intravenous administration of the antibody drug conjugate without hyaluronidase. In various embodiments, the antibody drug conjugate is administered subcutaneously at a dose equivalent or higher than a dose administered intravenously. In some embodiments, the adverse events are hypersensitivity and infusion-related reactions.

Non-limiting examples of adverse events are itching, redness, rash, hives, fever, chills, back or belly pain, muscle or joint pain, fast heartbeat, and nausea or vomiting, and severe anaphylactic reactions, that could include signs and symptoms of cardiac arrest, hypotension, wheezing, angioedema, swelling, pneumonitis, and skin reactions. In specific embodiments, the adverse events are cytopenia, neutropenia, thrombocytopenia, anemia, leukopenia and/or lymphocytopenia. In other specific embodiments, the averse events are diarrhea, constipation, abdominal pain, gastroenteritis, nausea, vomiting, decreased appetite, mucositis and stomatitis. Adverse events can also include, rash, pruritic, edema, dry skin, alopecia, back pain, arthralgia, hypersensitivity and infusion-related reactions (for example, neuropathy).

Provided herein are combination dosing regimens wherein: subcutaneous administration of the antibody drug candidate increases serum hemoglobin in the human subject relative to intravenous administration of the antibody drug candidate; subcutaneous administration of the antibody drug candidate increases serum albumin in the human subject relative to intravenous administration of the antibody drug candidate; subcutaneous administration of the antibody drug candidate increases creatinine clearance in the human subject relative to intravenous administration of the antibody drug candidate; subcutaneous administration of the antibody drug candidate decreases serum alkaline phosphatase in the human subject relative to intravenous administration of the antibody drug candidate; subcutaneous administration of the antibody drug candidate increases serum and/or plasma magnesium in the human subject relative to intravenous administration of the antibody drug candidate; subcutaneous administration of the antibody drug candidate increases serum and/or plasma sodium in the human subject relative to intravenous administration of the antibody drug candidate; and/or subcutaneous administration of the antibody drug candidate increases serum and/or plasma potassium in the human subject relative to intravenous administration of the antibody drug candidate

Provided herein are combination dosing regimens that provide a higher lymphatic concentration relative to an equivalent intravenous dose. In various embodiments the lymphatic concentration is about 90% to about 550% higher relative to an equivalent intravenous dose. For example, the lymphatic concentration is about 100%; 150%; 200%; 250%; 300%; 350%; 400%; 450%; or 500% higher relative to an equivalent intravenous dose.

Disclosed herein are combination dosing regimen wherein the ratio of total intact antibody drug conjugate to total monoclonal antibody in serum is between about 1% to about 100%. For example, the ratio of total intact antibody drug conjugate to total monoclonal antibody in serum is about 10%, 20%, 30%, 40%, 50%, 60%, 70%, 80%, 90%, or 100%.

Disclosed herein are combination dosing regimen wherein the ratio of free payload to total intact antibody drug conjugate in serum is between about 1% to about 100%. For example, the ratio of free payload to total intact antibody drug conjugate in serum is about 10%, 20%, 30%, 40%, 50%, 60%, 70%, 80%, 90%, or 100%.

Disclosed herein are combination dosing regimen wherein the ratio of total intact antibody drug conjugate to total monoclonal antibody in tissue is between about 1% to about 100%. For example the ratio of total intact antibody drug conjugate to total monoclonal antibody in tissue is about 10%, 20%, 30%, 40%, 50%, 60%, 70%, 80%, 90%, or 100%.

Disclosed herein are combination dosing regimen wherein the ratio of free payload to total intact antibody drug conjugate in tissue is between about 1% to about 100%. For example, the ratio of free payload to total intact antibody drug conjugate in tissue is about 10%, 20%, 30%, 40%, 50%, 60%, 70%, 80%, 90%, or 100%.

### D. Soluble Hyaluronidases

Soluble hyaluronidases include any that, upon expression, are secreted from a cell and exist in soluble form. Such soluble hyaluronidases include, for example, but are not limited to, bacterial soluble hyaluronidases, non-human soluble hyaluronidases, such as bovine PH20 and ovine PH20, human soluble PH20, and variants thereof. Generally soluble forms of PH20 are produced using protein expression systems that facilitate correct N-glycosylation to ensure the polypeptide retains activity, since glycosylation is important for the catalytic activity and stability of hyaluronidases. Such cells include, for example Chinese Hamster Ovary (CHO) cells (e.g. DG44 CHO cells).

Soluble PH20 hyaluronidase is available and sold, for example, under the trademark ENHANZE®. ENHANZE® technology provides to a drug delivery technology, employing the soluble hyaluronidases to facilitate the delivery of injected drugs and fluids. When co-formulated with other drugs or administered with other drugs, the ENHANZE® technology reduces treatment burden for patients. It can allow for large volume subcutaneous injections with increased dispersion and absorption of co-administered therapies.

rHuPH20 refers to the composition produced upon expression in a cell, such as CHO cell, of nucleic acid encoding residues 36-482 of SEQ ID NO: 10, generally linked to the native or a heterologous signal sequence (residues 1-35 of SEQ ID NO: 10). rHuPH20 is produced by expression of a nucleic acid molecule, such as encoding amino acids 1-482 (set forth in SEQ ID NO: 10) in a mammalian cell. Translational processing removes the 35 amino acid signal sequence. As produced in the culture medium there is heterogeneity at the C-terminus such that the product, designated rHuPH20, includes a mixture of species that can include any one or more of the polypeptides 36-480, 36-481, and 36-482 of SEQ ID NO: 10, and some shorter polypeptides, in various abundance. rHuPH20 and forms of soluble hyaluronidase are produced in cells, such as CHO cells, for example DG44 CHO cells, that facilitate N-glycosylation. PH20 is a glycoprotein, and as known in the art, requires glycosylation retain activity. See, e.g. U.S. Pat. Nos. 8,927,249 and 9,284,543 (and PCT Publication No. WO 2010/077297), which describe the effects of glycosylation and partial glycosylation and elimination of glycosylation on the activity of soluble forms of PH20. These patents and publications also describe and exemplify I soluble C-terminally truncated forms of PH20.

### 1. Forms of Soluble Human PH20

Soluble hyaluronidases include bovine and ovine PH20, and recombinant and humanized forms thereof. Human PH20 in nature includes a GPI anchor and exists linked to sperm cells; it is not soluble. C-terminally-truncated forms thereof are soluble. Soluble forms of recombinant human PH20 have been produced and can be used in the compositions, combinations and methods described herein. Descriptions of and production of such soluble forms of PH20 are described, for example, in U.S. Pat. Nos. 7,767,429; 8,202,517; 8,431,380; 8,431,124; 8,450,470; 8,765,685; 8,772,246; 7,871,607; 7,846,431; 7,829,081; 8,105,586; 8,187,855; 8,257,699; 8,580,252; 9,677,061; and 9,677,062, each incorporated by reference herein. The soluble hyaluronidases, thus include forms of human PH20, which are neutral active hyaluronidases and which require glycosylation for activity.

SEQ ID NO: 1 sets forth the sequence of the precursor polypeptides; the mature PH20 polypeptide (residues 36-509); soluble forms also include those with amino acid truncations at the N-terminal, such as deletions of the first one, two, three, or fours residues, such that the resulting polypeptides have an N-terminus, for example, at residue 36, 37, 38, 39, or 40, and a C-terminus at a residue from 465 to 500, and variants thereof, including, but not limited to, variants discussed below, variants known in the art, and allelic variants.

Hyaluronidases for use in the compositions, combinations and methods herein are soluble neutral active hyaluronidases. Exemplary thereof are the soluble C-terminally truncated forms of mature human PH20. Soluble forms that have hyaluronidase activity, include but are not limited to, those that are truncated at residues from 465 to 500 of SEQ ID NO: 1, and that are, upon expression, secreted. Exemplary thereof are polypeptides that have sequence 36-465 of SEQ ID NO: 1, 36-466 of SEQ ID NO: 1, 36-467 of SEQ ID NO: 1, 36-468 of SEQ ID NO: 1, 36-469 of SEQ ID NO: 1, 35-470 of SEQ ID NO: 1, 36-471 of SEQ ID NO: 1, 36-472 of SEQ ID NO: 1, 36-474 of SEQ ID NO: 1, 36-475 of SEQ ID NO: 1, 36-476 of SEQ ID NO: 1, 35-477 of SEQ ID NO: 1, 36-478 of SEQ ID NO: 1, 36-479 of SEQ ID NO: 1, 36-480 of SEQ ID NO: 1, 36-481 of SEQ ID NO: 1, 36-482 of SEQ ID NO: 1, 36-483 of SEQ ID NO: 1, 35-484 of SEQ ID NO: 1, 36-485 of SEQ ID NO: 1, 36-486 of SEQ ID NO: 1, 36-487 of SEQ ID NO: 1, 36-488 of SEQ ID NO: 1, 36-489 of SEQ ID NO: 1, 36-490 of SEQ ID NO: 1, 35-491 of SEQ ID NO: 1, 36-492 of SEQ ID NO: 1, 36-493 of SEQ ID NO: 1, 36-494 of SEQ ID NO: 1, 36-495 of SEQ ID NO: 1, 36-496 of SEQ ID NO: 1, 36-497 of SEQ ID NO: 1, 35-498 of SEQ ID NO: 1, 36-499 of SEQ ID NO: 1, and 36-500 of SEQ ID NO:1, as well as N-terminally truncated forms of each of the preceding that lack two to five residues at the N-terminus, such as for example 37-368 of SEQ ID NO: 1, 38-468 of SEQ ID NO: 1, and any others that exhibit hyaluronidase activity at neutral pH, such as pH in the range of 7.0-7.4.

Thus, such soluble forms include truncated forms of the mature form of human PH20 lacking all or a portion of the C-terminal GPI anchor, so long as the hyaluronidase is soluble and retains hyaluronidase activity. Soluble forms are secreted upon expression in mammalian cells, and are encoded with a signal sequence, such are residues 1-35 of SEQ ID NO: 1 or a heterologous signal sequence that is cleaved by the cell to effect secretion. Soluble forms are forms that, when expressed in a cell, lack the signal peptide. Also included among soluble hyaluronidases are variants of the soluble PH20 polypeptides that exhibit hyaluronidase activity. Variants include polypeptides having at least 60%, 70%, 80%, 85%, 90%, 91%, 92%, 93%, 94%, 95%, 96%, 97%, 98%, 99% or more sequence identity to any of the PH20 polypeptides 36-465 of SEQ ID NO: 1, 36-466 of SEQ ID NO: 1, 36-467 of SEQ ID NO: 1, 36-468 of SEQ ID NO: 1, 36-469, 35-470 of SEQ ID NO: 1, 36-471 of SEQ ID NO: 1, 36-472 of SEQ ID NO: 1, 36-474 of SEQ ID NO: 1, 36-475 of SEQ ID NO: 1, 36-476 of SEQ ID NO: 1, 35-477 of SEQ ID NO: 1, 36-478 of SEQ ID NO: 1, 36-479 of SEQ ID NO: 1, 36-480 of SEQ ID NO: 1, 36-481 of SEQ ID NO: 1, 36-482 of SEQ ID NO: 1, 36-483 of SEQ ID NO: 1, 35-484 of SEQ ID NO: 1, 36-485 of SEQ ID NO: 1, 36-486 of SEQ ID NO: 1, 36-487 of SEQ ID NO: 1, 36-488 of SEQ ID NO: 1, 36-489 of SEQ ID NO: 1, 36-490 of SEQ ID NO: 1, 35-491 of SEQ ID NO: 1, 36-492 of SEQ ID NO: 1, 36-493 of SEQ ID NO: 1, 36-494 of SEQ ID NO: 1, 36-495 of SEQ ID NO: 1, 36-496 of SEQ ID NO: 1, 36-497 of SEQ ID NO: 1, 35-498 of SEQ ID NO: 1, 36-499 of SEQ ID NO: 1, and 36-500 of SEQ ID NO:1. Amino acid variants include conservative and non-conservative insertions, or deletions, or replacements, and include the modifications, singly or combinations of the modifications detailed, for example, in U.S. Pat. No. 11,041,149 and International PCT publication No. WO 2013/102144. U.S. Pat. No. 11,041,149 and International PCT publication No. WO 2013/102144 describe a systematic analysis and results identifying the effects of amino acid modifications at each residue in PH20 to thereby provide a structure/function map of PH20; a skilled person can identify replacement residues and consequent alterations in properties and activities, such as for effecting increases in enzymatic activity, stability in denaturing conditions, and also residues whose replacement or deletion decreases or eliminates enzymatic activity.

It is understood that residues that are important or otherwise required for the activity of a hyaluronidase, such as any described above or known to those of skill in the art, are generally invariant and, except for possible conservative amino acid substitutions, cannot be changed. These include, for example, active site residues. For example, amino acid residues 111, 113 and 176 (corresponding to residues in the mature PH20 polypeptide) of a human PH20 polypeptide, or soluble form thereof, are generally invariant and are not altered. Other residues that confer glycosylation and formation of disulfide bonds required for proper folding also can be invariant.

The soluble human PH20 hyaluronidase is GPI-anchored and is rendered soluble by truncation at the C-terminus by removal of all or a part of the GPI anchor. Such truncation can remove all of the GPI anchor attachment sequence or can remove only some of the GPI anchor attachment sequence. The resulting polypeptide, however, is soluble. In instances where the soluble hyaluronidase retains a portion of the GPI anchor attachment signal sequence, 1, 2, 3, 4, 5, 6, 7 or more amino acid residues in the GPI anchor attachment signal sequence can be retained, provided the polypeptide is soluble. Polypeptides containing one or more amino acids of the GPI anchor are termed extended soluble hyaluronidases. One of skill in the art can determine whether a polypeptide is GPI-anchored using methods well known in the art. Such methods include, but are not limited to, using known algorithms to predict the presence and location of the GPI anchor attachment signal sequence and @-site, and performing solubility analyses before and after digestion with phosphatidylinositol-specific phospholipase C (PI-PLC) or D (PI-PLD).

Extended soluble hyaluronidases, which terminate for example, at residues 495, 496, 497, 498, 499, and 500, with reference to SEQ ID NO:1, can be produced by making C-terminal truncations to any naturally GPI-anchored hyaluronidase such that the resulting polypeptide is soluble and contains one or more amino acid residues from the GPI anchor attachment signal sequence (see, e.g. U.S. Pat. No. 8,927,249). These include hyaluronidases that are neutral active, soluble, contain amino acid substitutions, and have at least 60%, 70%, 80%, 85%, 90%, 91%, 92%, 93%, 94%, 95%.

Typically, for use in the compositions, combinations and methods herein, a soluble human hyaluronidase, such as a soluble human PH20, is used, such as a PH20 and variants having, for example, at least 91% or 95% or 98% sequence identity thereto, including those with 1 to 5 N-terminal residues deleted. Hyaluronidases used in the regimens, combinations, compositions, and methods herein can be recombinantly produced or can be purified or partially purified from natural sources, such as, for example, from testes extracts. Methods for production of recombinant proteins, including recombinant hyaluronidases, are well known in the art.

Recombinant soluble forms of human PH20 have been generated and can be used in the compositions, combinations and methods provided herein. For example, with reference to SEQ ID NO: 1, which sets forth the sequence of full length precursor PH20, which includes a signal sequence (residues 1-35), soluble forms include, but are not limited to, C-terminal truncated polypeptides of human PH20 set forth in SEQ ID NO: 1 having a C-terminal amino acid residue 467 of the sequence of amino acids set forth in SEQ ID NO: 1, 468 of the sequence of amino acids set forth in SEQ ID NO: 1, 469 of the sequence of amino acids set forth in SEQ ID NO: 1, 470 of the sequence of amino acids set forth in SEQ ID NO: 1, 471 of the sequence of amino acids set forth in SEQ ID NO: 1, 472 of the sequence of amino acids set forth in SEQ ID NO: 1, 473 of the sequence of amino acids set forth in SEQ ID NO: 1, 474 of the sequence of amino acids set forth in SEQ ID NO: 1, 475 of the sequence of amino acids set forth in SEQ ID NO: 1, 476 of the sequence of amino acids set forth in SEQ ID NO: 1, 477 of the sequence of amino acids set forth in SEQ ID NO: 1 (i.e., SEQ ID NO: 11), 478 of the sequence of amino acids set forth in SEQ ID NO: 1 (i.e., SEQ ID NO: 12), 479 of the sequence of amino acids set forth in SEQ ID NO: 1 (i.e., SEQ ID NO: 13), 480 of the sequence of amino acids set forth in SEQ ID NO: 1 (i.e., SEQ ID NO: 14), 481 of the sequence of amino acids set forth in SEQ ID NO: 1 (i.e., SEQ ID NO: 15), 482 of the sequence of amino acids set forth in SEQ ID NO: 1 (i.e., SEQ ID NO: 3), 483 of the sequence of amino acids set forth in SEQ ID NO: 1 (i.e., SEQ ID NO: 16), 484 of the sequence of amino acids set forth in SEQ ID NO: 1, 485 of the sequence of amino acids set forth in SEQ ID NO: 1, 486 of the sequence of amino acids set forth in SEQ ID NO: 1, 487 of the sequence of amino acids set forth in SEQ ID NO: 1, 488 of the sequence of amino acids set forth in SEQ ID NO: 1, 489 of the sequence of amino acids set forth in SEQ ID NO: 1, 490 of the sequence of amino acids set forth in SEQ ID NO: 1, 491 of the sequence of amino acids set forth in SEQ ID NO: 1, 492 of the sequence of amino acids set forth in SEQ ID NO: 1, 493 of the sequence of amino acids set forth in SEQ ID NO: 1, 494 of the sequence of amino acids set forth in SEQ ID NO: 1, 495 of the sequence of amino acids set forth in SEQ ID NO: 1, 496 of the sequence of amino acids set forth in SEQ ID NO: 1, 497 of the sequence of amino acids set forth in SEQ ID NO: 1, 498 of the sequence of amino acids set forth in SEQ ID NO: 1, 499 of the sequence of amino acids set forth in SEQ ID NO: 1 or 500 of the sequence of amino acids set forth in SEQ ID NO: 1, or polypeptides that exhibit at least 85%, 90%, 91%, 92%, 93%, 94%, 95%, 96%, 97%, 98%, 99%, or more sequence identity thereto, when aligned with the unmodified sequence of the soluble PH20, have activity at neutral pH, and are soluble (secreted into the medium when expressed in a mammalian cell). Soluble forms of human PH20 generally include those that contain amino acids 36-464 set forth in SEQ ID NO: 1 and terminate at any of residues, 465-500 and optionally include a 1-3 amino acid deletion at the N-terminus (i.e. lack residues 36, 36-37, or 36-38 of SEQ ID NO:1). For example, when expressed in mammalian cells, the 35 amino acid N-terminal signal sequence (residues 1-35 of SEQ ID NO:1) is cleaved during processing, and a soluble form of the protein is secreted. Thus, the mature soluble polypeptides include those that contain amino acids 36 to 467 of SEQ ID NO: 1, 468 of SEQ ID NO: 1, 469 of SEQ ID NO: 1, 470 of SEQ ID NO: 1, 471 of SEQ ID NO: 1, 472 of SEQ ID NO: 1, 473 of SEQ ID NO: 1, 474 of SEQ ID NO: 1, 475 of SEQ ID NO: 1, 476 of SEQ ID NO: 1, 477 of SEQ ID NO: 1 (i.e., SEQ ID NO: 9), 478 of SEQ ID NO: 1 (i.e., SEQ ID NO: 8), 479 of SEQ ID NO: 1 (i.e., SEQ ID NO: 7), 480 of SEQ ID NO: 1 (i.e., SEQ ID NO: 6), 481 of SEQ ID NO: 1 (i.e., SEQ ID NO: 5), 482 of SEQ ID NO: 1 (i.e., SEQ ID NO: 4), 483 of SEQ ID NO: 1 (i.e., SEQ ID NO: 17), and up to and including 500 of SEQ ID NO: 1. Exemplary of soluble hyaluronidases are soluble human PH20 polypeptides that are 442 (i.e., SEQ ID NO: 9), 443 (i.e., SEQ ID NO: 8), 444 (i.e., SEQ ID NO: 7), 445 (i.e., SEQ ID NO: 6), 446 (i.e., SEQ ID NO: 5) or 447 (i.e., SEQ ID NO: 4) amino acids in length, such as set forth those set forth above, and variants thereof that have, for example, at least 85%, 86%, 87%, 88%, 89%, 90%, 91%, 92%, 93%, 94%, 95%, 96%, 97%, 98% or 99% sequence identity thereto and retains hyaluronidase activity. The generation of such soluble forms of recombinant human PH20 are described, for example, in U.S. Pat. Nos. 7,767,429; 8,202,517; 8,431,380; 8,431,124; 8,450,470; 8,765,685; 8,772,246; 7,871,607; 7,846,431; 7,829,081; 8,105,586; 8,187,855; 8,257,699; 8,580,252; 9,677,061; and 9,677,062.

Generally soluble forms of PH20 are produced using protein expression systems that facilitate correct N-glycosylation to ensure the polypeptide retains activity, since glycosylation is important for the catalytic activity and stability of hyaluronidases. Such cells include, for example Chinese Hamster Ovary (CHO) cells (e.g. DG44 CHO cells).

The composition that recombinantly produced from mammalian cells, such as CHO cells, has been referred to rHuPH20. It refers to the composition produced upon expression in a cell, such as CHO cell, of nucleic acid encoding residues 36-482 of SEQ ID NO: 1 (i.e., SEQ ID NO: 4), generally linked to the native (residues 1-35 of SEQ ID NO: 1; residues 1-482 of SEQ ID NO: 1 are set forth in SEQ ID NO: 3) or a heterologous signal sequence. rHuPH20 is produced by expression of a nucleic acid molecule, such as encoding amino acids 1-482 (set forth in SEQ ID NO: 1; residues 1-482 of SEQ ID NO: 1 are set forth in SEQ ID NO: 3) or 36 to 482 (residues 36-482 of SEQ ID NO: 1 are set forth in SEQ ID NO: 4) with a heterologous signal sequence. Post translational processing removes the 35 amino acid signal sequence, resulting in polypeptide or a mixture of polypeptides, including those set forth in SEQ ID NO:4-8. As produced in the culture medium there is heterogeneity at the C-terminus such that the product, designated rHuPH20, includes a mixture of species that can include any one or more of SEQ ID NO: 4-8 in various abundance. Generally, the soluble hyaluronidases, rHuPH20 is produced in cells that facilitate correct N-glycosylation to retain activity, such as CHO cells (e.g. DG44 CHO cells). Human soluble PH20 hyaluronidase requires glycosylation for activity. When produced recombinantly from a vector encoding residues 36-582, the most abundant species is the 446 amino acid polypeptides corresponding to residues 36-481 of SEQ ID NO: 1 (i.e., SEQ ID NO: 5). The particular distribution of resulting polypeptides can depend upon the particular method of production. An exemplary method for production of high levels of PH20 is detailed, for example in U.S. Pat. Nos. 8,187,855 and 8,343,487.

### 2. Glycosylation of Hyaluronidases

Glycosylation, including N- and O-linked glycosylation, of some hyaluronidases, including the soluble PH20 hyaluronidases, can be important for their catalytic activity and stability. For some hyaluronidases, removal of N-linked glycosylation can result in near complete inactivation of the hyaluronidase activity. For such hyaluronidases, the presence of N-linked glycans can be important for generating an active enzyme.

N-linked oligosaccharides fall into several primary types (oligomannose, complex, hybrid, sulfated), all of which have (Man) 3-GlcNAc-GlcNAc-cores attached via the amide nitrogen of Asn residues that fall within-Asn-Xaa-Thr/Ser-sequences (where Xaa is not Pro). Glycosylation at an -Asn-Xaa-Cys-site has been reported for coagulation protein C. In some instances, a hyaluronidase, such as a PH20 hyaluronidase, can contain N-glycosidic and O-glycosidic linkages. For example, PH20 has O-linked oligosaccharides as well as N-linked oligosaccharides. There are six potential N-linked glycosylation sites at N82, N166, N235, N254, N368, N393 of human PH20 exemplified in SEQ ID NO: 1.

### 3. Variants

As discussed above, variants of PH20 are known to those of skill in the art, or readily can be prepared in view of the skill and knowledge in the art. Variants include those with amino acid replacements, insertions, and deletions. Variants of the soluble PH20 polypeptides that have altered properties, such as increased stability and/or activity, have been produced. U.S. Pat. No. 9,447,401 and family members U.S. Pat. Nos. 10,865,400, 11,041,149 and 11,066,656 describe and provide a structure/function map of human PH20 detailing the effects of amino acid replacements at every residue in the catalytic domain of PH20. These patents provide about 7000 examples in which the effects of replacing each amino acid with 15 other amino acids on activity and stability were identified and described. By virtue of those patents, and earlier publications/patents, describing virtually all variants of soluble PH20 polypeptides are known in the art. A skilled person readily can prepare soluble hyaluronidases and variants thereof and know the properties of the resulting hyaluronidase.

Other variants also are known to those of skill in the art, and can be used in the combinations, regimens, and methods described herein. For example, see, International PCT Publication No. WO2020/022791 and WO2020197230A which are incorporated by reference, and which describe modified PH20 polypeptides. These polypeptides, which include variants of the PH20 polypeptides that generally span residues 38-468, and include replacements, insertions, and deletions. The variants include for example one or more amino acid residues changes S343E, I344N, M345T, M348K, K349E, L353A, L354I, N356E, and I361T (with reference to SEQ ID NO: 1), and others, including about 15 amino acid variations, and truncations at the N-terminus and C-terminus. Variants that contain such modifications and others are set forth in SEQ ID NO: 60-115 of International PCT publication No. WO2020/022791. Exemplary of these polypeptides is the polypeptide of SEQ ID NO:99, therein. International PCT Publication No. WO2021/150079 provides variant PH20 polypeptides described as having increased stability relative to unmodified PH20, such as those in rHuPH20. These variant polypeptides have been shown to have PH20 activity and are described as having use for subcutaneous co-administration with other agents.

### E. Methods of Administration, Regimens, and Combinations

### 1. Methods of Administration

In an embodiment, each of the hyaluronidase and antibody-drug conjugate can be administered to a patient via injection. In an embodiment the hyaluronidase and antibody-drug conjugate is administered subcutaneously. For example, the hyaluronidase and antibody-drug conjugate can be administered to a patient subcutaneously in the abdominal tissue, leg or arm. The hyaluronidase and antibody-drug conjugate can be administered separately or in the same composition.

The compositions for administration to a patient via an injection (e.g. subcutaneously) also may comprise suitable inert additives, stabilizers, carriers, or excipients. In an embodiment, the injectable composition comprises an excipient. In an embodiment, the excipient is selected from one or more of 2-(N-morpholino) ethane sulfonic acid (MES), citric acid monohydrate, dextran, d-mannitol, glacial acetic acid, histidine, histidine hydrochloride monohydrate, L-histidine, L-histidine hydrochloride monohydrate, L-histidine monohydrochloride, polysorbate, sodium acetate, sodium chloride, sodium citrate dihydrate, sodium hydroxide, sodium phosphate dibasic anhydrous, sodium phosphate monobasic monohydrate, sodium succinate, succinic acid, sucrose, trehalose, trehalose dihydrate, and tromethamine. In an embodiment, the injectable composition comprises histidine. In an embodiment, the injectable composition comprises sodium chloride. In an embodiment, the injectable composition comprises polysorbate. In an embodiment, the polysorbate comprises polysorbate 80. In an embodiment, the injectable composition comprises an antioxidant. In an embodiment, the antioxidant comprises methionine.

It is shown and described herein that when an antibody-drug conjugate is administered in combination with the hyaluronidase, dispersion of the co-injected drugs or co-delivered is enhanced. By depolymerizing hyaluronan, hyaluronidase temporarily facilitates dispersion by reducing the viscosity of interstices. The permeability barrier in these tissues is restored to pre-injection levels within 24 to 48 hours after injection of hyaluronidase. This allows for higher volumes in a single injection of the antibody-drug conjugate to be administered to the patient.

When administered in separate compositions, the hyaluronidase and antibody-drug conjugate are injected as close to the same site as possible. For example, in an embodiment, hyaluronidase is first injected to a patient at a first injection site and subsequently the antibody-drug conjugate is injected at the same injection site or at an injection site as close to the first injection site as possible.

In an embodiment, the antibody-drug conjugate is administered at a concentration of about 0.1 mg/kg to about 5 mg/kg, about 5 mg/kg to about 10 mg/kg, about 10 mg/kg to about 20 mg/kg, about 20 mg/kg to about 30 mg/kg, about 30 mg/kg to about 40 mg/kg, about 40 mg/kg to about 50 mg/kg, about 50 mg/kg to about 60 mg/kg, about 60 mg/kg to about 70 mg/kg, about 70 mg/kg to about 80 mg/kg, about 80 mg/kg to about 90 mg/kg, or about 90 mg/kg to about 100 mg/kg.

In an embodiment, the antibody-drug conjugate is administered at a concentration of about 0.1 mg/kg, about 0.5 mg/kg, about 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 15 mg/kg, 20 mg/kg, 25 mg/kg, 30 mg/kg, 35 mg/kg, 40 mg/kg, 45 mg/kg, 50 mg/kg. 55 mg/kg, 60 mg/kg, 65 mg/kg, 70 mg/kg, 75 mg/kg, 80 mg/kg, 85 mg/kg, 90 mg/kg, 95 mg/kg and 100 mg/kg. In an embodiment, the antibody-drug conjugate is administered a concentration of about 100 mg/kg.

### 2. Regimens

In one aspect, the present disclosure provides a combination dosing regimen, comprising:
(i) subcutaneously administering to a patient in need thereof a composition comprising a soluble hyaluronidase comprising a sequence of amino acids that has at least 85%, 86%, 87%, 88%, 89%, 90%, 91%, 92%, 93%, 94%, 95%, 96%, 97%, 98% or 99% sequence identity to a sequence of amino acids that contains at least amino acids 36-464 of SEQ ID NO:1 and retains hyaluronidase activity; and an antibody-drug conjugate; or
(ii) subcutaneously administering to a patient in need thereof a first composition comprising a soluble hyaluronidase comprising a sequence of amino acids that has at least 85%, 86%, 87%, 88%, 89%, 90%, 91%, 92%, 93%, 94%, 95%, 96%, 97%, 98% or 99% sequence identity to a sequence of amino acids that contains at least amino acids 36-464 of SEQ ID NO:1 and retains hyaluronidase activity; and
subcutaneously administering to the patient in need thereof a second composition comprising an antibody-drug conjugate;
   wherein the soluble hyaluronidase is subcutaneously administered to the patient in an amount sufficient to obtain at least 50% bioavailability of the antibody-drug conjugate compared to the bioavailability obtained when the antibody-drug conjugate is administered intravenously.

In one embodiment, the antibody-drug conjugate administered intravenously does not comprise soluble hyaluronidase.

In one embodiment, the soluble hyaluronidase is subcutaneously administered to the patient in an amount sufficient to obtain from at least 50% to 225%, 75% to 225%, 75% to 200%, 100% to 200%, 100% to 175%, 125% to 175%, or 125% to 150% of the bioavailability of the antibody-drug conjugate compared to the bioavailability obtained when the antibody-drug conjugate is administered intravenously.

In another aspect, the present disclosure provides a combination dosing regimen comprising subcutaneously administering to a subject an antibody-drug conjugate (ADC); and a soluble hyaluronidase (PH20); wherein subcutaneous administration of the ADC with PH20 provides:
a maximum blood concentration (Cmax) of ADC that is about 20% to about 60% of the Cmax obtained via intravenous (IV) administration of an equivalent dose of the ADC; and
an area under the concentration-time curve (AUC) in blood of ADC that is about 50% to about 90% of the AUC obtained via IV administration of an equivalent dose of the ADC. In one embodiment, the equivalent dose of the ADC does not comprise the soluble hyaluronidase.

In one embodiment, subcutaneous administration of the ADC with PH20 achieves a Cmax of free antibody that is about 20% to 60% of the Cmax achieved by IV administration of an equivalent dose of the ADC. In one embodiment, subcutaneous administration of the ADC with PH20 achieves a Cmax of the ADC that is about 20% to 55% of the Cmax achieved by IV administration of an equivalent dose of the ADC. In one embodiment, subcutaneous administration of the ADC with PH20 achieves a Cmax of the free payload that is about 30% to about 75% of the Cmax achieved by IV administration of an equivalent dose of the ADC.

In one embodiment, subcutaneous administration of the ADC with PH20 provides a lower systemic exposure (as measured by AUC) of the ADC compared to IV administration of an equivalent dose of the ADC. In one embodiment, subcutaneous administration of the ADC with PH20 provides an AUC in blood of the total antibody that is about 50% to 90% of the AUC achieved by IV administration of an equivalent dose of the ADC. In one embodiment, subcutaneous administration of the ADC with PH20 provides an AUC in blood of the ADC that is about 50% to 90% of the AUC achieved by IV administration of an equivalent dose of the ADC. In one embodiment, subcutaneous administration of the ADC with PH20 provides an AUC in blood of the free payload that is about 50% to 90% of the AUC achieved by IV administration of an equivalent dose of the ADC.

In an embodiment, subcutaneous administration of the ADC with the soluble hyaluronidase achieves a weekly average AUC in blood of the ADC that is about 100 μg/mL*day to 230 μg/mL*day. In an embodiment, subcutaneous administration of the ADC with the soluble hyaluronidase achieves a Cmax of the ADC that is about 20 μg/mL to 150 μg/mL. In an embodiment, subcutaneous administration of the ADC with the soluble hyaluronidase achieves a Cmax of the ADC that is about 25 μg/mL to 100 μg/mL.

In yet another aspect, the present disclosure provides a combination dosing regimen comprising subcutaneously administering to a subject an antibody-drug conjugate (ADC) and a soluble hyaluronidase;
wherein the dosing regimen is characterized by:
   a higher dose of the ADC being delivered subcutaneously than an a therapeutically effective dose of the ADC when administered intravenously;
   the subcutaneous administration of the ADC yields a blood area under the concentration-time curve (AUC) that is equal to or higher than the AUC obtained from an intravenously administered therapeutically effective dose of the ADC; and
   the subcutaneous administration of the ADC yields a maximum blood concentration (Cmax) that is equal to or lower than the Cmax obtained from an intravenously administered therapeutically effective dose of the ADC. In one embodiment, the dose of the ADC administered intravenously does not comprise the soluble hyaluronidase.

In one embodiment, the AUC and Cmax are for the total antibody. In one embodiment, the AUC and Cmax are for the free antibody. In one embodiment, the AUC and Cmax are for the free payload. In one embodiment, the intravenously administered equivalent dose and/or a therapeutically effective dose of the ADC does not comprise soluble hyaluronidase. In one embodiment, subcutaneous administration of the ADC with the soluble hyaluronidase achieves a weekly average AUC in blood of the ADC that is about 120 μg/mL*day to 680 μg/mL*day. In one embodiment, subcutaneous administration of the ADC with the soluble hyaluronidase achieves a Cmax of the ADC that is about 25 μg/mL to 410 μg/mL. In one embodiment, subcutaneous administration of the ADC with the soluble hyaluronidase achieves a Cmax of the ADC that is about 35 μg/mL to 330 μg/mL. In one embodiment, the dose delivered subcutaneously is delivered more frequently than the therapeutically effective dose delivered intravenously.

In yet another aspect, the present disclosure provides a combination dosing regimen comprising subcutaneously administering to a subject an antibody-drug conjugate (ADC) in combination with a soluble hyaluronidase; and wherein the subcutaneous administration of the ADC in combination with the soluble hyaluronidase results in:
greater therapeutic efficacy of the ADC in the subject relative to intravenous administration of the ADC; and/or
increased overall survival in the subject relative to intravenous administration of the antibody drug conjugate; and/or
increased objective response rate of the ADC in the subject relative to intravenous administration of the antibody drug conjugate; and/or
produces greater complete response of the ADC in the subject relative to intravenous administration of the ADC; and/or
increased progression-free survival of the ADC in the subject relative to intravenous administration of the ADC; and/or
decreased time to treatment failure of the ADC in the subject relative to intravenous administration of the ADC; and/or
increased duration of response of the ADC in the subject relative to intravenous administration of the ADC.

In one embodiment, the ADC for intravenous administration does not comprise the soluble hyaluronidase. In one embodiment, the increased survival is disease free.

In yet another aspect, the present disclosure provides a combination dosing regimen, wherein the subcutaneous administration of the ADC in combination with the soluble hyaluronidase results in:
reduced toxicity of the ADC in the subject relative to intravenous administration of the ADC; and/or reduced adverse events of the ADC in the subject relative to intravenous administration of the ADC.

In one embodiment, the ADC administered intravenously does not comprise soluble hyaluronidase.

In one embodiment, the adverse events are selected from hypersensitivity and infusion-related reactions. In one embodiment, the adverse events are selected from itching, redness, rash, hives, fever, chills, back or belly pain, muscle or joint pain, fast heartbeat, and nausea or vomiting, and severe anaphylactic reactions, that could include signs and symptoms of cardiac arrest, hypotension, wheezing, angioedema, swelling, pneumonitis, and skin reactions. In one embodiment, the adverse events are selected from cytopenia, neutropenia, thrombocytopenia, anemia, leukopenia and lymphocytopenia. In one embodiment, the adverse events are selected from diarrhea, constipation, abdominal pain, gastroenteritis, nausea, vomiting, decreased appetite, mucositis and stomatitis. In one embodiment, the adverse events are selected from rash, pruritic, edema, dry skin and alopecia. In one embodiment, the adverse events are selected from back pain and arthralgia. In one embodiment, the adverse event is neuropathy. In one embodiment, the adverse event is interstitial lung disease.

In yet another aspect, the present disclosure provides a combination dosing regimen comprising subcutaneously administering to a subject an antibody-drug conjugate (ADC) in combination with a soluble hyaluronidase;
wherein the subcutaneous administration of the ADC and soluble hyaluronidase combination results in:
   an increased blood haemoglobin in the subject relative to intravenous administration of the ADC; and/or
   an increased blood albumin in the subject relative to intravenous administration of the ADC; and/or
   an increased creatinine clearance in the subject relative to intravenous administration of the ADC; and/or
   decreased blood alkaline phosphatase in the subject relative to intravenous administration of the ADC; and/or
   increased blood magnesium in the subject relative to intravenous administration of the ADC; and/or
   increased blood potassium in the subject relative to intravenous administration of the ADC; and/or
   increased blood sodium in the subject relative to intravenous administration of the ADC.

In one embodiment, the ADC administered intravenously does not comprise soluble hyaluronidase.

In yet another aspect, the present disclosure provides a combination dosing regimen comprising:
subcutaneously administering to a subject an antibody-drug conjugate (ADC) comprising an antibody that specifically targets Trop 2; and a soluble hyaluronidase,
   wherein subcutaneous administration of the ADC with the soluble hyaluronidase provides a lower maximum blood concentration (Cmax) of ADC compared to intravenous (IV) administration of an equivalent dose of the ADC. In embodiments, the intravenous administration of an equivalent dose of the ADC is without the soluble hyaluronidase.

In one embodiment, the ADC comprises a topoisomerase I inhibitor payload conjugated to the antibody that specifically targets Trop 2 via a cleavable linker. In one embodiment, the topoisomerase I inhibitor payload is a chemotherapy drug. In one embodiment, the chemotherapy drug is SN-38. In one embodiment, the ADC is sacituzumab govitecan.

In one embodiment, subcutaneous administration of the combination of the ADC comprising the antibody that specifically targets Trop 2 with the soluble hyaluronidase provides a maximum blood concentration (Cmax) of ADC that is 20% to 50% of the Cmax achieved by intravenous (IV) administration of an equivalent dose of the ADC. In one embodiment, subcutaneous administration of the combination of the ADC with the soluble hyaluronidase provides an area under the concentration-time curve (AUC) of ADC that is about 40% to about 60% of the AUC obtained via intravenous (IV) administration of an equivalent dose of the ADC.

In one embodiment, the free payload weekly average AUC of the ADC comprising the antibody that specifically targets Trop 2 administered subcutaneously with the soluble hyaluronidase is 80% to 140% of the equivalent dose of free payload weekly average AUC of the ADC administered subcutaneously without soluble hyaluronidase; and/or the weekly average AUC of the ADC comprising the antibody that specifically targets Trop 2 administered subcutaneously with the soluble hyaluronidase is 110% to 160% of the equivalent dose total ADC weekly average AUC of the ADC administered subcutaneously without the soluble hyaluronidase. In one embodiment, the free payload weekly average AUC of the ADC administered subcutaneously with the soluble hyaluronidase is 80% to 140% of the equivalent dose of free payload weekly average AUC of the ADC administered subcutaneously without soluble hyaluronidase. In one embodiment, the weekly average AUC of the ADC administered subcutaneously with the soluble hyaluronidase is 110% to 160% of the equivalent dose total ADC weekly average AUC of the ADC administered subcutaneously without the soluble hyaluronidase.

In yet another aspect, the present disclosure provides a combination dosing regimen comprising:
subcutaneously administering to a subject an antibody-drug conjugate (ADC) comprising an antibody that specifically targets HER2 and a soluble hyaluronidase;
   wherein subcutaneous administration of the ADC with the soluble hyaluronidase provides a lower maximum blood concentration (Cmax) of ADC compared to intravenous (IV) administration of an equivalent dose of the ADC. In embodiments, the intravenous administration of an equivalent dose of the ADC is without the soluble hyaluronidase.

In one embodiment, subcutaneous administration of the combination of the ADC comprising the antibody that specifically targets HER2 with soluble hyaluronidase provides a maximum blood concentration (Cmax) of ADC that is about 15% to about 45% of the Cmax achieved by intravenous (IV) administration of an equivalent dose of the ADC. In one embodiment, subcutaneous administration of the combination of the ADC with the soluble hyaluronidase provides an area under the concentration-time curve (AUC) of ADC that is about 60% to about 90% of the AUC obtained via intravenous (IV) administration of an equivalent dose of the ADC.

In one embodiment, the free payload weekly average AUC of the ADC comprising the antibody that specifically targets HER2 administered subcutaneously with the soluble hyaluronidase is 80% to 140% of the equivalent dose of free payload weekly average AUC of the ADC administered subcutaneously without soluble hyaluronidase; and/or the weekly average AUC of the ADC comprising the antibody that specifically targets HER2 administered subcutaneously with the soluble hyaluronidase is 110% to 160% of the equivalent dose total ADC weekly average AUC of the ADC administered subcutaneously without the soluble hyaluronidase. In one embodiment, the free payload weekly average AUC of the ADC administered subcutaneously with the soluble hyaluronidase is 80% to 140% of the equivalent dose of free payload weekly average AUC of the ADC administered subcutaneously without soluble hyaluronidase. In one embodiment, the weekly average AUC of the ADC administered subcutaneously with the soluble hyaluronidase is 110% to 160% of the equivalent dose of ADC weekly average AUC of the ADC administered subcutaneously without the soluble hyaluronidase.

In one embodiment, the combination dosing regimens described herein comprise a soluble human hyaluronidase. In one embodiment, the combination dosing regimens described herein comprise a recombinant soluble human hyaluronidase. In one embodiment, the soluble hyaluronidase comprises a sequence of amino acids that has at least 85%, 86%, 87%, 88%, 89%, 90%, 91%, 92%, 93%, 94%, 95%, 96%, 97%, 98% or 99% sequence identity to a sequence of amino acids that contains at least amino acids 36-464 of SEQ ID NO: 1 and retains hyaluronidase activity.

In one embodiment, in any of the combination dosing regimens described herein, the antibody-drug conjugate comprises an antibody and a drug payload conjugated to the antibody via a cleavable linker.

In one embodiment, the ADC in any of the combination dosing regimens described herein comprises a topoisomerase inhibitor payload conjugated to the antibody via a cleavable linker. In one embodiment, the ADC in any of the combination dosing regimens described herein comprises a topoisomerase I inhibitor payload conjugated to the antibody via a cleavable linker. In one embodiment, the topoisomerase I inhibitor drug payload is a chemotherapy drug. In one embodiment, the chemotherapy drug is SN-38. In one embodiment, the ADC is sacituzumab govitecan. In one embodiment, the ADC is Trodelvy. In one embodiment, the chemotherapy drug is an exatecan derivative called DXd. In one embodiment, the ADC is trastuzumab deruxtecan. In one embodiment, the ADC is Enhertu.

In one embodiment, in any of the combination dosing regimens described herein, subcutaneous administration of the combination of the soluble hyaluronidase and ADC provides a reduced Tmax for the ADC compared to subcutaneous administration of an equivalent dose of the ADC without the soluble hyaluronidase. In one embodiment, in any of the combination dosing regimens described herein, subcutaneous administration of the combination of the soluble hyaluronidase and ADC provides a reduced Tmax for the free antibody compared to subcutaneous administration of an equivalent dose of the ADC without the soluble hyaluronidase. In one embodiment, in any of the combination dosing regimens described herein, subcutaneous administration of the combination of the soluble hyaluronidase and ADC provides a reduced Tmax for the free payload compared to subcutaneous administration of an equivalent dose of the ADC without the soluble hyaluronidase. In one embodiment, in any of the combination dosing regimens described herein, subcutaneous administration of the combination of the soluble hyaluronidase and ADC achieves systemic exposure faster than subcutaneous administration of an equivalent dose of the ADC without the soluble hyaluronidase. In one embodiment, faster systemic exposure with subcutaneous administration of the combination of the soluble hyaluronidase and ADC provides earlier therapeutic blood levels compared to subcutaneous administration of an equivalent dose of the ADC without the soluble hyaluronidase.

In one embodiment, in any of the combination dosing regimens described herein, the dose delivered subcutaneously is 25% to 400% of the dose administered intravenously. In one embodiment, the dose delivered subcutaneously is 100 to 350% of the dose administered intravenously. In one embodiment, the dose delivered subcutaneously is 150% to 300% of the dose administered intravenously. In one embodiment, the dose delivered subcutaneously is 200% to 250% of the dose administered intravenously. In one embodiment, the dose delivered subcutaneously is 150% to 200% of the dose administered intravenously. In one embodiment, the dose delivered subcutaneously is 200% of the dose administered intravenously. In one embodiment, the dose delivered subcutaneously is 100% to 150% of the dose administered intravenously.

In one embodiment, in any of the combination dosing regimens described herein, subcutaneous administration of the combination of the ADC with the soluble hyaluronidase provides a Tmax that is reduced by about 1 to 4 days compared to subcutaneous administration of an equivalent dose of the ADC without the soluble hyaluronidase. In one embodiment, subcutaneous administration of the combination of the ADC with the soluble hyaluronidase provides a Tmax that is reduced by 1 to 2 days compared to subcutaneous administration of an equivalent dose of the ADC without the soluble hyaluronidase. In one embodiment, subcutaneous administration of the combination of the ADC with the soluble hyaluronidase provides a reduced Tmax when compared to subcutaneous administration of an equivalent dose of the ADC without the soluble hyaluronidase.

In one embodiment, in any of the combination dosing regimens described herein, subcutaneous administration of the combination of the ADC with the soluble hyaluronidase provides a Tmax of total antibody that is reduced by about 1 to 4 days compared to subcutaneous administration of an equivalent dose of the ADC without the soluble hyaluronidase. In one embodiment, subcutaneous administration of the combination of the ADC with the soluble hyaluronidase provides a Tmax of total antibody that is reduced compared to subcutaneous administration of an equivalent dose of the ADC without the soluble hyaluronidase.

In one embodiment, in any of the combination dosing regimens described herein, subcutaneous administration of the combination of the ADC with soluble hyaluronidase provides a Tmax of free payload that is reduced by about 1 to 4 days compared to subcutaneous administration of an equivalent dose of the ADC without the soluble hyaluronidase. In one embodiment, in any of the combination dosing regimens described herein, subcutaneous administration of the combination of the ADC with the soluble hyaluronidase provides a Tmax of free payload that is reduced as compared to subcutaneous administration of an equivalent dose of the ADC without the soluble hyaluronidase.

In one embodiment, in any of the combination dosing regimens described herein, subcutaneous administration of the combination of the ADC with soluble hyaluronidase provides a Tmax that is increased by about 1 to 4 days compared to intravenous administration of an equivalent dose of the ADC. In one embodiment, subcutaneous administration of the combination of the ADC with the soluble hyaluronidase provides a Tmax that is increased by 1 to 2 days compared to intravenous administration of an equivalent dose of the ADC. In one embodiment, subcutaneous administration of the combination of the ADC with the soluble hyaluronidase provides an increased Tmax when compared to intravenous administration of an equivalent dose of the ADC. In one embodiment, the ADC administered intravenously does not comprise soluble hyaluronidase.

In one embodiment, in any of the combination dosing regimens described herein, subcutaneous administration of the combination of the ADC with soluble hyaluronidase provides a Tmax of total antibody that is increased by about 1 to 4 days compared to intravenous administration of an equivalent dose of the ADC. In one embodiment, subcutaneous administration of the combination of the ADC with the soluble hyaluronidase provides a Tmax of total antibody that is increased compared to intravenous administration of an equivalent dose of the ADC. In one embodiment, the ADC administered intravenously does not comprise soluble hyaluronidase.

In one embodiment, in any of the combination dosing regimens described herein, subcutaneous administration of the combination of the ADC with soluble hyaluronidase provides a Tmax of free payload that is increased by about 1 to 4 days compared to intravenous administration of an equivalent dose of the ADC. In one embodiment, in any of the combination dosing regimens described herein, subcutaneous administration of the combination of the ADC with the soluble hyaluronidase provides a Tmax of free payload that is increased as compared to intravenous administration of an equivalent dose of the ADC. In one embodiment, the ADC administered intravenously does not comprise soluble hyaluronidase.

In one embodiment, in any of the combination dosing regimens described herein, the free payload weekly average AUC of the ADC administered subcutaneously with the soluble hyaluronidase is 80% to 140% of the equivalent dose of free payload weekly average AUC of the ADC administered subcutaneously without soluble hyaluronidase; and/or
the weekly average AUC of the ADC administered subcutaneously with the soluble hyaluronidase is 110% to 160% of the equivalent dose total ADC weekly average AUC of the ADC administered subcutaneously without the soluble hyaluronidase. In one embodiment, the free payload weekly average AUC of the ADC administered subcutaneously with the soluble hyaluronidase is 80% to 140% of the equivalent dose of free payload weekly average AUC of the ADC administered subcutaneously without soluble hyaluronidase. In one embodiment, the weekly average AUC of the ADC administered subcutaneously with the soluble hyaluronidase is 110% to 160% of the equivalent dose total ADC weekly average AUC of the ADC administered subcutaneously without the soluble hyaluronidase.

In one embodiment, in any of the combination dosing regimens described herein, subcutaneous administration the combination of the ADC with the soluble hyaluronidase provides improved local tolerability in the subject relative to subcutaneous administration of an equivalent dose of the ADC without the soluble hyaluronidase, evidenced at the injection site by reduced erythema, swelling, pain, subcutaneous cytotoxicity, necrosis, lesions, ulcers, pruritis, infection, rash, or dry skin.

In one embodiment, in any of the combination dosing regimens described herein, subcutaneous administration of the combination of the ADC with the soluble hyaluronidase provides a reduced incidence of systemic adverse events in the subject compared to intravenous administration of the ADC. In one embodiment, the ADC administered intravenously does not comprise soluble hyaluronidase.

In one embodiment, in any of the combination dosing regimens described herein, the soluble hyaluronidase is administered concurrently with the ADC. In one embodiment, the soluble hyaluronidase is co-formulated with the ADC. In one embodiment, the soluble hyaluronidase and the ADC are lyophilized together and reconstituted prior to administration to the subject. In one embodiment, the soluble hyaluronidase and the ADC are lyophilized separately and reconstituted prior to administration to the subject. In one embodiment, the ADC is lyophilized and reconstituted in a solution comprising the soluble hyaluronidase prior to administration to the subject. In one embodiment, co-administration of the ADC with the soluble hyaluronidase reduces injection time by about 95%, about 90%, about 85%, about 80%, about 75%, about 70%, about 65%, about 60%, about 55%, about 50%, about 45%, about 40%, about 35%, about 30%, about 25%, about 20%, about 15%, about 10%, or about 5% compared to ADC alone. In one embodiment, co-administration of the ADC with the soluble hyaluronidase reduces injection time by about 10% compared to ADC alone.

In one embodiment, PH20 is administered within about 60 minutes prior to administration of the ADC. In one embodiment, the soluble hyaluronidase is administered within about 30 minutes prior to administration of the ADC. In one embodiment, the soluble hyaluronidase is administered within about 15 minutes prior to administration of the ADC.

In one embodiment, in any of the combination dosing regimens described herein, the combination dosing regimen is administered on a dosing schedule of once or twice weekly. In one embodiment, the combination dosing regimen is administered on a dosing schedule of once every 2-4 weeks. In one embodiment, the combination dosing regimen is administered on a dosing schedule of once every 2-6 weeks. In one embodiment, the combination dosing regimen is administered on a dosing schedule of once every 2-8 weeks. In one embodiment, the combination dosing regimen is administered on a dosing schedule of once every two (2) to twelve (12) weeks. In one embodiment, the dosing schedule is maintained for at least two cycles. In one embodiment, the dosing schedule is maintained for at least four cycles. In one embodiment, the dosing schedule is maintained for at least six cycles. In one embodiment, the dosing schedule is maintained for at least eight cycles.

In one embodiment, in any of the combination dosing regimens described herein, the ADC is administered at a dose of 0.1 mg/kg to 50 mg/kg. In one embodiment, the ADC is administered at a dose of 0.1 mg/kg to 10 mg/kg. In one embodiment, the ADC is administered at a dose of 0.1 mg/kg to 5 mg/kg. In one embodiment, the ADC is administered at a dose of 1 mg/kg to 6 mg/kg.

In one embodiment, in any of the combination dosing regimens described herein, the soluble hyaluronidase is administered at a dose of 2,000 to 100,000 U, 2,000 to 90,000 U, 2,000 to 80,000 U, 2,000 to 70,000 U, 2,000 to 60,000 U, 2,000 to 50,000 U, 2,000 to 40,000 U, 2,000 to 30,000 U, 2,000 to 25,000 U, 2,000 to 20,000 U, 2,000 to 15,000 U, 2,000 to 10,000 U, or 2,000 to 8,000 U. In one embodiment the soluble hyaluronidase is administered at a dose of about 2,000 U. In one embodiment, the soluble hyaluronidase is administered at a dose of about 4,000 U. In one embodiment, the soluble hyaluronidase is administered at a dose of about 6,000 U.

Provided are regimens for administration of an antibody-drug conjugate in combination with a soluble hyaluronidase. The antibody-drug conjugate generally is formulated as a solution for subcutaneous injection at effective concentrations, and the hyaluronidase is provided as a composition containing an effective concentration of soluble hyaluronidase for delivery of an effective amount of hyaluronidase in about 3 mL to 5 mL, 3 mL to 10 mL, 3 mL to 15 mL, 3 mL to 20 mL, 3 mL to 25 mL, 3 ml to 30 mL, 3 mL to 35 mL, 3 mL to 40 mL, 3 mL to 45 mL, 3 mL to 50 mL, 5 mL to 10 mL, 5 mL to 15 mL, 5 mL to 20 mL, 5 mL to 25 mL, 5 mL to 30 mL, 5 mL to 35 mL, 5 mL to 40 mL; 5 mL to 45 mL, 5 mL to 50 mL, 10 mL to 15 mL; 10 mL to 20 mL; 10 mL to 25 mL; 10 mL to 30 mL; 10 mL to 35 mL; 10 mL to 40 mL, 10 mL to 50 mL, 10 mL to 60 mL, 10 mL to 70 mL, 10 mL to 80 mL, 10 mL to 90 mL, 10 mL to 100 mL, 10 mL to 110 mL, 10 mL to 120 mL, 10 mL to 130 mL, 10 mL to 140 mL, 10 mL to 150 mL, 10 mL to 160 mL, 10 mL to 170 mL, 10 mL to 180 mL, 10 mL to 190 mL, 10 mL to 200 mL; optionally 0.5 mL to 10 mL, such as 1 mL to 5 mL, or 1 mL to 3 mL. The antibody-drug conjugate and hyaluronidase can be administered separately or co-formulated for administration in a single composition. When administered separately, they can be administered in any order, but generally the hyaluronidase is administered first. The antibody-drug conjugate and/or hyaluronidase can be provided as separate compositions, such as suspension and solutions, or can be provided as a co-formulation.

As described herein, the hyaluronidase and antibody-drug conjugate can be administered together or sequentially or any other defined regimen. For example, in some embodiments, the hyaluronidase is administered to a patient before the antibody-drug conjugate is administered i.e. in a first step hyaluronidase is administered to a patient; and in a second step the antibody-drug conjugate is administered to a patient. In an embodiment an antibody-drug conjugate is administered to the patient as soon as possible after hyaluronidase has been administered to the patient i.e. immediately after hyaluronidase has been administered to the patient.

In accord with regimens and compositions provided herein, the hyaluronidase is administered in an amount suitable to allow a dose of from about 100 mg of an antibody-drug conjugate to be administered to the patient. Exemplary ranges include, but are not limited to, an amount suitable to allow a dose from 1 mg to 10 mg, 10 mg to 2000 mg, 50 mg to 1950 mg, 100 mg to 1900 mg, 150 mg to 1850 mg, 200 mg to 1800 mg, 250 mg to 1750 mg, 300 mg to 1700 mg, 350 mg to 1650 mg, 400 mg to 1600 mg, 450 mg to 1550 mg, 500 mg to 1500 mg, 550 mg to 1450 mg, 600 mg to 1400 mg, 650 mg to 1350 mg, 700 mg to 1300 mg. 750 mg to 1250 mg, 800 mg to 1200 mg, 850 mg to 1150 mg, 900 mg to 1100 mg, or 950 mg to 1050 mg to be administered to the patient. In embodiments, exemplary ranges include, but are not limited to, an amount suitable to allow a dose from 1 mg to 300 mg, 10 mg to 300 mg, from 15 mg to 250 mg, from 20 mg to 200 mg, from 25 mg to 150 mg, from 30 mg to 150 mg, from 40 mg to 150 mg, from 50 mg to 150 mg, from 60 mg to 150 mg, from 70 mg to 150 mg, from 80 mg to 150 mg, from 90 mg to 150, or from 100 mg to 150 mg to be administered to the patient. In embodiments, exemplary ranges include, but are not limited to, an amount suitable to allow a dose of at least 1 mg, 10 mg, 20 mg, 30 mg, 40 mg, 50 mg, 60 mg, 70 mg, 80 mg, 90 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1100 mg, 1200 mg, 1300 mg, 1400 mg, 1500 mg. 1600 mg, 1700 mg, 1800 mg, 1900 mg or 2000 mg or more to be administered to a patient. In embodiments, exemplary ranges include, but are not limited to, an amount suitable to allow a dose of about 1 mg, about 5 mg, about 10 mg, about 20 mg, about 30 mg, about 40 mg, about 50 mg, about 60 mg, about 70 mg, about 80 mg, about 90 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1100 mg, about 1200 mg, about 1300 mg, about 1400 mg, about 1500 mg, about 1600 mg, about 1700 mg, about 1800 mg, about 1900 mg or about 2000 mg to be administered to a patient.

In other embodiments, the hyaluronidase is administered in an amount suitable to allow a dose of at least or at about a 100 mg to be administered. It is understood that a skilled practitioner can determine a particular dose, which can depend upon various parameters include the mass of the patient, the age of the patient, and other conditions of the patient.

In an exemplary embodiment, an antibody-drug conjugate is administered at a dose of from 100 mg to 2000 mg, such as, but not limited to, a dose of from 900 mg to 2000 mg, such as for example, a dose of at least or at 10 mg to 1000 mg, such as for example, a dose of at least or at 1 mg, 10 mg, 20 mg, 30 mg, 40 mg, 50 mg, 60 mg, 70 mg, 80 mg, 90 mg, 100 mg, 150 mg, 200 mg, 250 mg, 300 mg, 350 mg, 400 mg, 450 mg, 500 mg, 550 mg, 600 mg, 650 mg. 700 mg, 750 mg, 800 mg, 850 mg, 900 mg, 950 mg, 1000 mg, 1100 mg, 1200 mg, 1300 mg, 1400 mg, 1500 mg, 1600 mg, 1700 mg, 1800 mg, 1900 mg or 2000 mg. In embodiments, the antibody-drug conjugate is administered at a dose of 1000 mg to 2000 mg or in amounts in between such doses. For example, an antibody-drug conjugate is administered at a dose of about 1500 mg, or at a dose of about 1750 mg, or at a dose of about 1900 mg, or at a dose of about 1950 mg.

In an embodiment, the amount of the disclosed formulation administered to the subject is dependent on the subject being treated, the severity of the disorder or condition, the rate of administration, the disposition of the compounds, and/or the discretion of the prescribing physician. In an embodiment, an effective dosage of the antibody-drug conjugate in the disclosed formulation is in the range of about 0.001 to about 100 mg per kg body weight per day, such as about 1 to about 35 mg/kg/day, in single or divided doses. For a 70 kg human, this would amount to about 0.05 to 7 g/day, such as about 0.05 to about 2.5 g/day. In some instances, dosage levels below the lower limit of the aforesaid range may be more than adequate, while in other cases still larger doses may be employed without causing any harmful side effect, for example by dividing such larger doses into several small doses for administration throughout the day. Low dose administration of an antibody-drug conjugate in combination with hyaluronidase is possible due to decreased residence time at the site of administration.

In an embodiment, the disclosed formulation is administered to the subject in multiple doses. Dosing may be about once, twice, three times, four times, five times, six times, or more than six times per day. Dosing may be about once a month, once every three weeks (Q3W) every two weeks (Q2W), once a week (QW), or once every other day. In some embodiments, dosing may be once every five weeks (Q5W), once every six weeks (Q6W), once every seven weeks (Q7W), once every eight weeks (Q8W), once every nine weeks (Q9W), once every ten weeks (Q10W), once every eleven weeks (Q11W), once every twelve weeks (Q12W), once every thirteen weeks (Q13W), once every fourteen weeks (Q14W) or once every fifteen weeks (Q15W). In one embodiment, the disclosed formulation is administered about once per day to about 6 times per day. In one embodiment, the administration of the disclosed formulation continues for less than about 7 days. In yet another embodiment the administration continues for more than about 6, 10, 14, 28 days, two months, six months, or one year. In some cases, continuous dosing is achieved and maintained as long as necessary.

Hyaluronidases have been used clinically since the 1950s. For example, rHuPH20, approved by the FDA in 2004, has been shown to be well tolerated in clinical evaluation of subcutaneous doses of up to 96,000 U, wherein U is USP units. For example, for purposes herein, hyaluronidase is administered at a dose of from 2000 to 15,000 U, such as, but not limited to from 5,000 to 15,000 U, such as 6,000 to 12,000 U, 8,000 to 12,000 U, such as at or about a dose of about 10,000 U, for example 10,000 U. The hyaluronidase is administered in injection volumes that range from at or about 0.5 mL to 10 mL, such as 1 mL to 5 mL, or at or about 1 mL to 3 mL, such as a 1 mL injection; the volume is a function of the specific activity of a particular formulation of the hyaluronidase. The particular amount depends upon parameters understood by those of skill in the art. Co-administration of an antibody-drug conjugate with hyaluronidase, as noted, allows for administration of a higher doses and larger volumes of the antibody-drug conjugate potentially affording a longer interval between injections. This can increase the convenience of long-acting regimens and can result in better adherence to therapy and positively impact treatment outcomes and acceptability.

As discussed above, the combination of an antibody-drug conjugate and hyaluronidase can allow less frequent dosing compared to administration of the antibody-drug conjugate alone, in dosing regimens that do not include a hyaluronidase. For example, in accord with the instant disclosure, hyaluronidase and antibody-drug conjugate are administered once every 3 months to once every year, such as once every 3 months, once every 4 months, once every 5 months or once every 6 months or other intervals that are longer than 3 months and less than 6 months, 9 months, or one year. In an exemplary regimen, hyaluronidase and antibody-drug conjugate are administered once every 3 months.

In an embodiment, combination dosing regimen is provided that comprises administering hyaluronidase; and an antibody-drug conjugate, wherein hyaluronidase is administered at a dose of from 4,000 to 15,000 U; and the antibody-drug conjugate is administered at a dose of from 10 to 100 mg/kg wherein the combination dosing regimen is administered once every 3 months to once every 6 months.

In another embodiment, provided is a combination dosing regimen comprising administering hyaluronidase; and administering an antibody-drug conjugate, wherein hyaluronidase is administered at a dose of from 6,000 to 12,000 U; and the antibody-drug conjugate is administered at a dose of from 10 to 100 mg/kg. Wherein the combination dosing regimen is administered once every 3 months to once every 6 months.

In another embodiment, provided a combination dosing regimen comprising administering hyaluronidase; and administering an antibody-drug conjugate, wherein hyaluronidase is administered at a dose of 10,000 U; and the antibody-drug conjugate is administered at a dose of from 10 mg/kg to 100 mg/kg. In this regime the antibody-drug conjugate has a concentration of 10 to 100 mg/kg, and the combination dosing regimen is administered once every 3 months.

The regimens provided herein can be used to treat or prevent a disease or disorder in a patient or subject in need thereof. In embodiments, the present application provides a method of treating or preventing a disease or disorder in a patient or subject in need thereof, the method comprising administering a combination dosing regimen described herein to the patient or subject in need thereof. Although not wishing to be limited by theory, prevention, as described herein, includes reducing the risk of infection. In embodiments, the administration comprises subcutaneous administration.

In embodiments, the regimens provided herein can be used to treat or prevent cancer in a patient or subject in need thereof. In embodiments, the present application provides a method of treating or preventing a cancer in a patient or subject in need thereof, the method comprising administering a combination dosing regimen described herein to the patient or subject in need thereof. Hence in embodiments herein, provided are methods of treating a cancer, the method comprising administering to a patient a combination dosing regimen described herein. In an alternative embodiment, provided is a method of preventing cancer, the method comprising administering to a human the combination dosing regimen described herein. In the first method, the patient has been diagnosed with cancer; in the latter, the subject has not been diagnosed with a cancer, but, generally is a subject at risk of cancer or suspected to have cancer.

In a further aspect, the combination dosing regimens as described herein are for use in the treatment or prevention of cancer. In an embodiment, the combination dosing regimens described herein are for use in the treatment of cancer. In an alternative embodiment, the present invention provides the combination dosing regimen as described herein for use in the prevention of cancer.

In an embodiment, cancers for treatment or prevention as described herein include but are not limited to, anaplastic large cell lymphoma (ALCL), peripheral T-cell lymphoma (PTCL), adult T-cell leukemia/lymphoma, cutaneous T-cell lymphoma (CTCL), extra-nodal NK-T-cell lymphoma, non-Hodgkin's lymphoma, diffuse large B-cell lymphoma, particularly EBV-positive diffuse large B-cell lymphoma, B cell acute lymphoblastic leukemia, breast cancer, lung cancer, gastric cancer, ovarian cancer, colon cancer, gastric cancer, endometrial cancer, cervical cancer, colorectal cancer, esophageal cancer, squamous cell carcinoma, pancreatic cancer, prostate cancer, stomach cancer, thyroid cancer, glioma, melanoma, urinary bladder cancer, urogenital cancer and uterine cancer.

In embodiments, the regimens provided herein can be used to treat or prevent a cardiometabolic disease or disorder in a patient or subject in need thereof. In embodiments, the present application provides a method of treating or preventing a cardiometabolic disease or disorder in a patient or subject in need thereof, the method comprising administering a combination dosing regimen described herein to the patient or subject in need thereof. Hence in embodiments herein, provided are methods of treating a cardiometabolic disease or disorder, the method comprising administering to a patient a combination dosing regimen described herein. In an alternative embodiment, provided is a method of preventing a cardiometabolic disease or disorder, the method comprising administering to a human the combination dosing regimen described herein. In the first method, the patient has been diagnosed with a cardiometabolic disease or disorder; in the latter, the subject has not been diagnosed with a cardiometabolic disease or disorder, but, generally is a subject at risk of a cardiometabolic disease or disorder or suspected to have a cardiometabolic disease or disorder.

In a further aspect, the combination dosing regimens as described herein are for use in the treatment or prevention of a cardiometabolic disease or disorder. In an embodiment, the combination dosing regimens described herein are for use in the treatment of a cardiometabolic disease or disorder. In an alternative embodiment, the present invention provides the combination dosing regimen as described herein for use in the prevention of a cardiometabolic disease or disorder.

In an embodiment, cardiometabolic diseases or disorders for treatment or prevention as described herein include but are not limited to, linked conditions affecting both the heart and metabolic systems of the patient or subject, including, but not limited to, type 2 diabetes, obesity, hypertension, high cholesterol, and heart disease. Exemplary cardiometabolic diseases or disorders include, but are not limited to, insulin resistance, type 2 diabetes, impaired glucose tolerance, obesity, hypertension (high blood pressure), dyslipidemia (high cholesterol and triglycerides), cardiovascular conditions (such as coronary heart disease), chronic kidney disease (CKD), metabolic associated steatohepatitis (MASH), and metabolic dysfunction-associated steatotic liver disease (MASLD).

In embodiments, the regimens provided herein can be used to treat or prevent an inflammatory and/or autoimmune disease or disorder in a patient or subject in need thereof. In embodiments, the present application provides a method of treating or preventing an inflammatory and/or autoimmune disease or disorder in a patient or subject in need thereof, the method comprising administering a combination dosing regimen described herein to the patient or subject in need thereof. Hence in embodiments herein, provided are methods of treating an inflammatory and/or autoimmune disease or disorder, the method comprising administering to a patient a combination dosing regimen described herein. In an alternative embodiment, provided is a method of preventing an inflammatory and/or autoimmune disease or disorder, the method comprising administering to a human the combination dosing regimen described herein. In the first method, the patient has been diagnosed with an inflammatory and/or autoimmune disease or disorder; in the latter, the subject has not been diagnosed with an inflammatory and/or autoimmune disease or disorder, but, generally is a subject at risk of an inflammatory and/or autoimmune disease or disorder or suspected to have an inflammatory and/or autoimmune disease or disorder.

In a further aspect, the combination dosing regimens as described herein are for use in the treatment or prevention of an inflammatory and/or autoimmune disease or disorder. In an embodiment, the combination dosing regimens described herein are for use in the treatment of an inflammatory and/or autoimmune disease or disorder. In an alternative embodiment, the present invention provides the combination dosing regimen as described herein for use in the prevention of an inflammatory and/or autoimmune disease or disorder.

In an embodiment, inflammatory and/or autoimmune diseases or disorders for treatment or prevention as described herein include but are not limited to, ankylosing spondylitis, antiphospholipid antibody syndrome, autoimmune encephalitis, chronic recurrent multifocal osteomyelitis, gout, Henoch Schönlein purpura, dermatomyositis, scleroderma, vasculitis, Kawasaki disease, lupus (systemic lupus erythematosus), mixed connective tissue disease, myositis, poststreptococcal inflammatory syndromes, arthritis (e.g., idiopathic arthritis, psoriatic arthritis, reactive arthritis, rheumatoid arthritis), Sjogren's syndrome, spondyloarthritis, spondyloarthropathy, undifferentiated connective tissue disease, uveitis, psoriasis, multiple sclerosis, Hashimoto's thyroiditis, inflammatory bowel disease, Crohn's disease, ulcerative colitis, celiac disease, asthma, chronic obstructive pulmonary disease (COPD), eczema, atopic dermatitis, hidradenitis suppurativa, vitiligo, type 1 diabetes, Addison's disease, Graves' disease, myasthenia gravis (MG), Guillain-Barré syndrome, and chronic inflammatory demyelinating polyneuropathy (CIPD).

### 3. Combinations, Compositions and Kits

Provided herein are compositions, combinations, and kits. The combinations comprise a composition containing the hyaluronidase; and a composition that is suspension comprising an antibody-drug conjugate. The compositions comprising the antibody-drug conjugate are formulated as a suspension in amounts for administering a dose of the antibody-drug conjugate, such as in an amount that is 10 mg to 2000 mg, such as such as, but not limited to, a dose of from 10 mg to 1000 mg, such as for example, a dose of at least or at 10 mg, 20 mg, 30 mg, 40 mg, 50 mg, 60 mg, 70 mg, 80 mg, 90 mg, 100 mg, 150 mg, 200 mg, 250 mg, 300 mg, 350 mg, 400 mg, 450 mg, 500 mg, 550 mg, 600 mg, 650 mg, 700 mg. 750 mg, 800 mg, 850 mg, 900 mg, 950 mg, 1000 mg, 1100 mg, 1200 mg, 1300 mg, 1400 mg, 1500 mg, 1600 mg, 1700 mg, 1800 mg, 1900 mg or 2000 mg as detailed above. Each of the compositions can be formulated for single dosage or multiple dosage administration or for dilution as appropriate.

The hyaluronidase is formulated for administration of a dose from 2000 to 15,000 U, such as, but not limited to from 5,000 to 15,000 U, such as 6,000 to 12,000 U, 8,000 to 12,000 U, such as at or about a dose of about 10,000 U, for example 10,000 U. Compositions containing hyaluronidase for administration are well known, and generally are formulated at a pH of about 7 to about 7.4, in appropriate buffers, salts, stabilizers and surfactant as needed. See e.g. U.S. Pat. No. 7,767,429. Variants, as described herein, that are more stable in denaturing conditions, such as those described in U.S. Pat. No. 9,447,401 and family members and variants designed for increased activity and/or stability can be formulated. The hyaluronidase and antibody-drug conjugate can be co-formulated as suspensions or mixed prior to use for administration in a single composition. The compositions containing both are formulated to deliver an appropriate dose of each.

The combinations can contain the two compositions or the single co-formulations, and optionally instructions for use. The combinations can be packaged as kits. Exemplary combinations and kits can include a syringe or other container containing the hyaluronidase, and a syringe or other container containing the antibody-drug conjugate. Alternatively, the antibody-drug conjugate and hyaluronidase can be provided in a dual compartment container, such as a dual compartment where the compositions are separated, such as by a membrane that can be punctured prior to administration. In these aspects, the hyaluronidase and antibody-drug conjugate are as described herein.

The syringe or other container may be sized and shaped to hold a volume of the hyaluronidase corresponding to a volume selected from:
(a) 3 mL to 5 mL, 3 mL to 10 mL. 3 mL to 15 mL, 3 mL to 20 mL, 3 mL to 25 mL, 3 ml to 30 mL, 3 mL to 35 mL, 3 mL to 40 mL, 3 mL to 45 mL, 3 mL to 50 mL, 5 mL to 10 mL, 5 mL to 15 mL, 5 mL to 20 mL, 5 mL to 25 mL, 5 mL to 30 mL, 5 mL to 35 mL, 5 mL to 40 mL; 5 mL to 45 mL, 5 mL to 50 mL, 10 mL to 15 mL; 10 mL to 20 mL: 10 mL to 25 mL; 10 mL to 30 mL; 10 mL to 35 mL; 10 mL to 40 mL, 10 mL to 50 mL, 10 mL to 60 mL, 10 mL to 70 mL, 10 mL to 80 mL, 10 mL to 90 mL, 10 mL to 100 mL, 10 mL to 110 mL, 10 mL to 120 mL, 10 mL to 130 mL, 10 mL to 140 mL, 10 mL to 150 mL, 10 mL to 160 mL, 10 mL to 170 mL, 10 mL to 180 mL, 10 mL to 190 mL, 10 mL to 200 mL;
(b) about 3 mL to about 5 mL, about 3 mL to about 10 mL, about 3 mL to about 15 mL, about 3 mL to about 20 mL, about 3 mL to about 25 mL, about 3 ml to about 30 mL, about 3 mL to about 35 mL, about 3 mL to about 40 mL, about 3 mL to about 45 mL, about 3 mL to about 50 mL, about 5 mL to about 10 mL, about 5 mL to about 15 mL, about 5 mL to about 20 mL, about 5 mL to about 25 mL, about 5 mL to about 30 mL, about 5 mL to about 35 mL, about 5 mL to about 40 mL; about 5 mL to about 45 mL, about 5 mL to about 50 mL, about 10 mL to about 15 mL; about 10 mL to about 20 mL; about 10 mL to about 25 mL; about 10 mL to about 30 mL; about 10 mL to about 35 mL; about 10 mL to about 40 mL, about 10 mL to about 50 mL, about 10 mL to about 60 mL, about 10 mL to about 70 mL, about 10 mL to about 80 mL, about 10 mL to about 90 mL, about 10 mL to about 100 mL, about 10 mL to about 110 mL, about 10 mL to about 120 mL, about 10 mL to about 130 mL, about 10 mL to about 140 mL, about 10 mL to about 150 mL, about 10 mL to about 160 mL, about 10 mL to about 170 mL, about 10 mL to about 180 mL, about 10 mL to about 190 mL, about 10 mL to about 200 mL;
(c) at least about 3 mL, at least about 3.5 mL, at least about 4 mL, at least about 4.5 mL, at least about 5.5 mL, at least about 6 mL, at least about 6.5 mL, at least about 7 mL, at least about 7.5 mL, at least about 8 mL, at least about 8.5 mL, at least about 9 mL, at least about 9.5 mL, at least about 10 mL, at least about 10.5 mL, at least about 11 mL, at least about 11.5 mL, at least about 12 mL, at least about 12.5 mL, at least about 13 mL, at least about 13.5 mL, at least about 14 mL, at least about 14.5 mL, at least about 15 mL, at least about 15.5 mL, at least about 16 mL, at least about 16.5 mL, at least about 17 mL, at least about 17.5 mL, at least about 18 mL, at least about 18.5 mL, at least about 19 mL, at least about 19.5 mL, at least about 20 mL, at least about 25 mL, at least about 30 mL, at least about 35 mL, at least about 40 mL, at least about 45 mL, at least about 50 mL, at least about 60 mL, at least about 70 mL, at least about 80 mL, at least about 90 mL, at least about 100 mL, at least about 110 mL, at least about 120 mL, at least about 130 mL, at least about 140 mL, at least about 150 mL, at least about 160 mL, at least about 170 mL, at least about 180 mL, at least about 190 mL, at least about 200 mL; and
(d) at least 3 mL, at least 3.5 mL, at least 4 mL, at least 4.5 mL, at least 5.5 mL, at least 6 mL, at least 6.5 mL, at least 7 mL, at least 7.5 mL, at least 8 mL, at least 8.5 mL, at least 9 mL, at least 9.5 mL, at least 10 mL, at least 10.5 mL, at least 11 mL, at least 11.5 mL, at least 12 mL, at least 12.5 mL, at least 13 mL, at least 13.5 mL, at least 14 mL, at least 14.5 mL, at least 15 mL, at least 15.5 mL, at least 16 mL, at least 16.5 mL, at least 17 mL, at least 17.5 mL, at least 18 mL, at least 18.5 mL, at least 19 mL, at least 19.5 mL, at least 20 mL, at least 25 mL, at least 30 mL, at least 35 mL, at least 40 mL, at least 45 mL, at least 50 mL, at least 60 mL, at least 70 mL, at least 80 mL, at least 90 mL, at least 100 mL, at least 110 mL, at least 120 mL, at least 130 mL, at least 140 mL, at least 150 mL, at least 160 mL, at least 170 mL, at least 180 mL, at least 190 mL, at least 200 mL.

The hyaluronidase may be delivered at a rate of approximately 0.08-0.75 mL/sec. For example, this would provide target delivery time ranges of 13-120 seconds for a 10 mL dose volume. 10 mL of the hyaluronidase may be delivered at a rate of 0.33 mL/sec. In one embodiment, the hyaluronidase is delivered at a rate of:
(a) 0.5 mL/10 sec., 0.75 mL/10 sec., 1 mL/10 sec., 1.25 mL/10 sec., 1.5 mL/10 sec., 1.75 mL/10 sec, 2 mL/10 sec., 2.25 mL/10 sec, 2.5 mL/10 sec., 2.75 mL/10 sec, 3 mL/10 sec., 3.25 mL/10 sec, 3.5 mL/10 sec., 3.75 mL/10 sec, 4 mL/10 sec., 4.25 mL/10 sec, 4.5 mL/10 sec., 4.75 mL/10 sec, 5 mL/10 sec;
(b) 2 mL/30 sec., 2.5 mL/30 sec., 3 mL/30 sec., 3.5 mL/30 sec., 4 mL/30 sec., 4.5 mL/30 sec., 5 mL/30 sec., 5.5 mL/30 sec., 6 mL/30 sec., 6.5 mL/30 sec., 7 mL/30 sec., 7.5 mL/30 sec., 8 mL/30 sec., 8.5 mL/30 sec., 9 mL/30 sec., 9.5 mL/30 sec., 10 mL/30 sec., 10.5 mL/30 sec.; and
(c) 4 mL/min, 5 mL/min, 6 mL/min, 7 mL/min, 8 mL/min, 9 mL/min, 10 mL/min, 11 mL/min, 12 mL/min, 13 mL/min, 14 mL/min, 15 mL/min, 16 mL/min, 17 mL/min, 18 mL/min, 19 mL/min, 20 mL/min, 21 mL/min.

The hyaluronidase may be delivered at a delivery time of approximately of 13-120 seconds. In one embodiment, the hyaluronidase is delivered at a delivery time of:
(a) about 10 seconds, about 12 seconds, about 16 seconds, about 18 seconds, about 20 seconds, about 22 seconds, about 24 seconds, about 26 seconds, about 28 seconds, about 30 seconds, about 32 seconds, about 34 seconds, about 36 seconds, about 38 seconds, about 40 seconds, about 42 seconds, about 44 seconds, about 46 seconds, about 48 seconds, about 50 seconds, about 52 seconds, about 54 seconds, about 56 seconds, about 58 seconds, about 60 seconds, about 65 seconds, about 70 seconds, about 75 seconds, about 80 seconds, about 85 seconds, about 90 seconds, about 95 seconds, about 100 seconds, about 105 seconds, about 110 seconds, about 115 seconds, or about 120 seconds.
(b) about 10 seconds to about 120 seconds, about 12 seconds to about 115 seconds, about 16 seconds to about 110 seconds, about 18 seconds to about 105 seconds, about 20 seconds to about 100 seconds, about 22 seconds to about 95 seconds, about 24 seconds to about 90 seconds, about 26 seconds to about 85 seconds, about 28 seconds to about 80 seconds, about 30 seconds to about 75 seconds, about 32 seconds to about 70 seconds, about 34 seconds to about 65 seconds, about 36 seconds to about 60 seconds, about 38 seconds to about 58 seconds, about 40 seconds to about 56 seconds, about 42 seconds to about 54 seconds, about 44 seconds to about 52 seconds or about 46 seconds to about 50 seconds.

In one aspect, the present disclosure provides a pharmaceutical composition for subcutaneous administration, the pharmaceutical composition comprising an antibody-drug conjugate (ADC) and a soluble hyaluronidase. In one embodiment, the ADC comprises an antibody and a payload conjugated to the antibody via a cleavable linker. In one embodiment, the ADC comprises an antibody and a payload conjugated to the antibody via a non-cleavable linker. In an embodiment, the pharmaceutical composition comprises one or more excipients, carriers, solubilizing agents, buffers, surfactants, tonicity-adjusting agents, antioxidants, or preservatives. In an embodiment, the pharmaceutical composition comprises an excipient. In an embodiment, the excipient is selected from one or more of 2-(N-morpholino) ethane sulfonic acid (MES), citric acid monohydrate, dextran, d-mannitol, glacial acetic acid, histidine, histidine hydrochloride monohydrate, L-histidine, L-histidine hydrochloride monohydrate, L-histidine monohydrochloride, polysorbate, sodium acetate, sodium chloride, sodium citrate dihydrate, sodium hydroxide, sodium phosphate dibasic anhydrous, sodium phosphate monobasic monohydrate, sodium succinate, succinic acid, sucrose, trehalose, trehalose dihydrate, and tromethamine.

In one embodiment, the antibody of the ADC binds to one or more of Trop-2, HER-2, B7-H3, EGFR, DLL3, HER-3, CDH17, folate receptor alpha, Nectin-4, CLDN18.2, c-MET, NaPI2b, CEACAM5, PSMA, CLDN6, FGFR2b, ROR1, CD33, CD30, CD22, CD79b, CD19, integrin beta-6, or Tissue Factor. In one embodiment, the antibody of the ADC is specific for Trop-2, HER-2, B7-H3, EGFR, DLL3, HER-3, CDH17, folate receptor alpha, Nectin-4, CLDN18.2, c-MET, NaPI2b, CEACAM5, PSMA, CLDN6, FGFR2b, ROR1, CD33, CD30, CD22, CD79b, CD19, integrin beta-6, or Tissue Factor. In one embodiment, the antibody is monospecific for Trop-2, HER-2, B7-H3, EGFR, DLL3, HER-3, CDH17, folate receptor alpha, Nectin-4, CLDN18.2, c-MET, NaPI2b, CEACAM5, PSMA, CLDN6, FGFR2b, ROR1, CD33, CD30, CD22, CD79b, CD19, integrin beta-6, or Tissue Factor. In one embodiment, the antibody binds to more than one antigen. In one embodiment, the antibody is multispecific for more than one antigen. In one embodiment, the antibody binds to a tumor-associated antigen. In one embodiment, the antibody is specific for a tumor-associated antigen. In one embodiment, the tumor-associated antigen is selected from CD33, CD30, CD22, HER2, CD79b, Nectin-4, CD19, Tissue Factor, folate receptor alpha, ROR1, MET, and integrin beta-6.

In one embodiment, the payload of the ADC is a topoisomerase inhibitor. In one embodiment, the payload is a topoisomerase I inhibitor. In one embodiment, the topoisomerase inhibitor is selected from SN-38, deruxtecan, exatecan, topotecan, camptothecin, or a derivative of any one thereof. In one embodiment, the topoisomerase I inhibitor is SN-38. In one embodiment, topoisomerase I inhibitor is deruxtecan. In one embodiment, the cleavable linker is selected from an acid labile linker, an enzyme cleavable linker, and a reducible disulfide linker. In one embodiment, the acid labile linker is a hydrazone linker or a CL2A linker. In one embodiment, the cleavable linker is a hydrazone linker. In one embodiment, the cleavable linker is cleaved in the acidic environment of endosomes or lysosomes. In one embodiment, the ADC has a drug-antibody ratio of 2-8. In one embodiment, the ADC has a drug-antibody ratio of 2-4. In one embodiment, the ADC is sacituzumab govitecan or trastuzumab deruxtecan.

In one embodiment, the pharmaceutical composition further comprises one or more buffers selected from histidine, MES, citrate, acetate, phosphate, or TRIS. In one embodiment, the pharmaceutical composition further comprises one or more stabilizers selected from trehalose, sucrose, mannitol, sorbitol, glycine, or arginine. In one embodiment, the pharmaceutical composition further comprises one or more surfactants selected from polysorbate 20, polysorbate 80, poloxamer 188, or sodium deoxycholate. In one embodiment, the pharmaceutical composition further comprises one or more tonicity-adjusting agents selected from sodium chloride, potassium chloride, calcium chloride, or glycerol. In one embodiment, the pharmaceutical composition further comprises one or more antioxidants selected from methionine, cysteine, ascorbic acid, a tocopherol, or BHT. In one embodiment, the pharmaceutical composition further comprises one or more preservatives selected from benzyl alcohol, phenol, m-cresol, or a paraben. In one embodiment, the pharmaceutical composition further comprises a combination of a buffer, a stabilizer, a surfactant, and a tonicity-adjusting agent.

In one embodiment, the pharmaceutical composition a soluble human hyaluronidase. In one embodiment, the soluble hyaluronidase comprises a recombinant soluble human hyaluronidase. In one embodiment, the soluble hyaluronidase comprises a sequence of amino acids that has at least 85%, 86%, 87%, 88%, 89%, 90%, 91%, 92%, 93%, 94%, 95%, 96%, 97%, 98% or 99% sequence identity to a sequence of amino acids that contains at least amino acids 36-464 of SEQ ID NO:1 and retains hyaluronidase activity.

In an embodiment, the compositions and combinations described herein can comprise one or more of inactive ingredients, including but not limited to, a divalent cation, a buffer, a pH adjusting agent, an anti-oxidation agent, a tonicity modifier, a surfactant, and other inactive ingredients/agents described below. Provided below is a description of the inactive ingredients that can be included in the hyaluronidase compositions and combinations described herein. The inactive ingredients are exemplary only and provide a platform from which minor adjustments can be made. It is understood that very small changes in the concentrations of the various excipients and other components (e.g. +15% of the stated concentrations), or small changes in pH, can be made while retaining some if not all of the hyaluronan degrading enzyme stability. Further changes also can be made by adding or removing excipients. For example, the type of stabilizing surfactant can be changed.

### Divalent Cation

In some embodiments, the hyaluronidase compositions and combinations provided herein comprise an amount of a divalent cation to achieve at least 50%, and generally at least 70%, of the initial enzymatic activity of the hyaluronidase at temperatures of between or approximately between 37° C. to 42° C., such as at least or about or approximately 37° C. or 40° C., for at least three (3) days and generally at least one month (e.g. 4 weeks) as described herein. For example, the amount of divalent cation is an amount to achieve at least 75%, 80%, 85%, 86%, 87%, 88%, 89%, 90%, 91%, 92%, 93%, 94%, 95%, 96%, 97%, 98%, 99% or more of the initial enzymatic activity of the hyaluronidase for at least three (3) days, and generally for at least 4 weeks at temperatures between or approximately between 37° C. to 42°, such as at least or about or approximately 40° C.

For example, hyaluronidase compositions and combinations provided herein can contain an amount of Lys-Lys, salt, derivative, analogue or mimetic thereof, to achieve at least 50%, and generally at least 70%, of the initial enzymatic activity of the hyaluronan-degrading enzyme at temperatures between or approximately between 37° C. to 42° C., such as at least or about or approximately 40° C., for at least three (3) days and generally for at least 4 weeks. Such a hyaluronidase composition or combination provided herein may contain between or about between 5 mM to 300 mM Lys-Lys, such as 10 mM to 200 mM, 50 mM to 150 mM or 10 mM to 50 mM. For example, a hyaluronidase composition or combination provided herein may contain at least or about at least or 5 mM, 10 mM, 20 mM, 30 mM, 40 mM, 50 mM, 60 mM, 70 mM, 80 mM, 90 mM, 100 mM, 125 mM, 150 mM, 200 mM, 300 mM or more Lys-Lys.

In another example, the hyaluronidase compositions or combinations provided herein can contain an amount of MgCl₂, a derivative, an analogue or a mimetic thereof, to achieve at least 50%, and generally at least 70%, of the initial enzymatic activity of the hyaluronidase at temperatures between or approximately between 37° C. to 42° C., such as at least or about or approximately 40° C., for at least three (3) days and generally for at least 4 weeks. The hyaluronidase compositions and combinations provided herein may contain between or about between 5 mM to 300 mM MgCl₂, such as 10 mM to 200 mM, 50 mM to 150 mM or 10 mM to 50 mM. For example, the hyaluronidase compositions and combinations provided herein may contain at least or about at least or 5 mM, 10 mM, 20 mM, 30 mM, 40 mM, 50 mM, 60 mM, 70 mM, 80 mM, 90 mM, 100 mM, 125 mM, 150 mM, 200 mM, 300 mM or more MgCl₂.

As discussed below, formulations containing a divalent cation (e.g. Lys-Lys), if necessary, also can contain a tonicity modifier (e.g. NaCl).

If necessary, the pH of the hyaluronidase compositions and combinations described herein can be adjusted using acidifying agents to lower the pH or alkalizing agents to increase the pH. Exemplary acidifying agents include, but are not limited to, acetic acid, citric acid, sulfuric acid, hydrochloric acid, monobasic sodium phosphate solution, and phosphoric acid. Exemplary alkalizing agents include, but are not limited to, dibasic sodium phosphate solution, sodium carbonate, or sodium hydroxide.

Any buffer can be used in the compositions and combinations provided herein so long as it does not adversely affect the stability of the composition/combination and supports the requisite pH range required. Examples of particularly suitable buffers include Tris, succinate, acetate, phosphate buffers, histidine, citrate, aconitate, malate and carbonate. Those of skill in the art, however, will recognize that the compositions and combinations provided herein are not limited to a particular buffer, so long as the buffer provides an acceptable degree of pH stability, or “buffer capacity” in the range indicated. Generally, a buffer has an adequate buffer capacity within about 1 pH unit of its pK. Buffer suitability can be estimated based on published pK tabulations or can be determined empirically by methods well known in the art. The pH of the solution can be adjusted to the desired endpoint within the range as described above, for example, using any acceptable acid or base.

Buffers that can be included in the compositions and combinations provided herein include, but are not limited to, Tris (Tromethamine), histidine, phosphate buffers, such as dibasic sodium phosphate, and citrate buffers. For example, the buffer can be a histidine hydrochloride (histidine/HCl) buffer. Generally, the buffering agent is present in an amount herein to maintain the pH range of the composition or combination between or about between 6.5 to 7.8, for example between or about between 6.8 to 7.8 such as between or about between 7.0 to 7.6. Such buffering agents can be present in the compositions and combinations at concentrations between or about between 1 mM to 100 mM, such as 10 mM to 50 mM or 20 mM to 40 mM, such as at or about 30 mM. For example, such buffering agents can be present in the compositions and combinations in a concentration of or about or at least 1 mM, 2 mM, 3 mM, 4 mM, 5 mM, 6 mM. 7 mM, 8 mM, 9 mM, 10 mM, 11 mM, 12 mM, 13 mM, 14 mM, 15 mM, 16 mM, 17 mM, 18 mM, 19 mM, 20 mM, 25 mM, 30 mM, 35 mM, 40 mM, 45 mM, 50 mM, 55 mM, 60 mM, 65 mM, 70 mM, 75 mM, or more.

In some examples, a buffering agent is not required. In an embodiment, the hyaluronidase compositions and combinations described herein comprise a surfactant. The surfactants generally are non-ionic surfactants. Surfactants that can be included in the compositions and combinations herein include, but are not limited to, partial and fatty acid esters and ethers of polyhydric alcohols such as of glycerol, or sorbitol, poloxamers and polysorbates. For example, exemplary surfactants in the compositions and combinations herein include any one or more of poloxamer 188 (PLURONICS® such as PLURONIC® F68), TETRONICS®, polysorbate 20, polysorbate 80, PEG 400, PEG 3000, Tween® (e.g. Tween® 20 or Tween® 80), Triton® X-100, SPAN®, MYRJ®, BRIJ®, CREMOPHOR®, polypropylene glycols or polyethylene glycols. In some examples, the compositions and combinations herein contain poloxamer 188, polysorbate 20, polysorbate 80, generally poloxamer 188 (pluronic F68).

In the compositions and combinations provided herein, the total amount of the one or more surfactants as a percentage (%) of mass concentration (w/v) in the compositions and combinations herein can be, for example, between from or between about from 0.0% to 1.0%, such as between or about between 0.0% to 0.0005%, 0.0005% to 0.005%, 0.001% to 0.01%, 0.01% to 0.5%, 0.01% to 0.1% or 0.01% to 0.02%. For example, the compositions and combinations provided herein can contain at or about 0.001%, 0.005%, 0.01%, 0.015%, 0.02%, 0.025%, 0.03%, 0.035%, 0.04%, 0.045%, 0.05%, 0.055%, 0.06%, 0.065%, 0.07%, 0.08%, or 0.09% surfactant.

### Anti-Oxidation Agent

The compositions and combinations provided herein also can contain antioxidants to reduce or prevent oxidation, in particular oxidation of the hyaluronidase. Exemplary antioxidants include, but are not limited to, cysteine, tryptophan and methionine. In particular examples, the anti-oxidant is methionine. The compositions and combinations provided herein can include an antioxidant at a concentration from between or from about between 5 mM to or to about 50 mM, such as 5 mM to 40 mM, 5 mM to 20 mM or 10 mM to 20 mM. For example, methionine can be provided in the compositions and combinations herein at a concentration from between or from about between 5 mM to or to about 50 mM, such as 5 mM to 40 mM, 5 mM to 20 mM or 10 mM to 20 mM. For example, an antioxidant, for example methionine, can be included at a concentration that is or is about or is at least 5 mM, 10 mM, 11 mM, 12 mM, 13 mM, 14 mM, 15 mM, 16 mM, 17 mM, 18 mM, 19 mM, 20 mM, 21 mM, 22 mM, 23 mM, 24 mM, 25 mM, 26 mM, 27 mM, 28 mM, 29 mM, 30 mM, 35 mM, 40 mM, 45 mM or 50 mM. In some examples, compositions and combinations described herein contain 10 mM to 20 mM methionine, such as or about or at least 10 mM or 20 mM methionine.

### Tonicity Modifier

Optionally, the stable hyaluronidase compositions and combinations provided herein can contain a tonicity modifier.

For example, in some embodiments, a tonicity modifier is included in the compositions and combinations herein to produce a solution with the desired osmolality. The compositions and combinations provided herein have an osmolality of between or about between 245 mOsm/kg to 500 mOsm/kg. For example, the osmolality is or is about or at least 245 mOsm/kg, 250 mOsm/kg, 255 mOsm/kg, 260 mOsm/kg, 265 mOsm/kg, 270 mOsm/kg, 275 mOsm/kg, 280 mOsm/kg. 285 mOsm/kg, 290 mOsm/kg, 295 mOsm/kg, 300 mOsm/kg, 350 mOsm/kg, 400 mOsm/kg, 450 mOsm/kg or 500 mOsm/kg. Typically, a tonicity modified is included in the compositions and combinations herein that contain a divalent cation, such as Lys-Lys, in a concentration that is less than 100 mM, such as less than 80 mM, 70 mM, 60 mM, 50 mM, 40 mM, 30 mM, 20 mM, 10 mM or less. For example, a tonicity modified is included in the compositions and combinations herein that contain a divalent cation, such as Lys-Lys, at a concentration of between or about between 10 mM to 50 mM, such as about or approximately 10 mM, 15 mM, 20 mM, 25 mM, 30 mM, 35 mM, 40 mM, 45 mM or 50 mM.

Tonicity modifiers include, but are not limited to, glycerin, NaCl, amino acids, polyalcohols, trehalose, and other salts and/or sugars. For example, the compositions and combinations provided herein can optionally include NaCl as a tonicity modifier. The NaCl can be included at a concentration of between or about between 0 mM to 200 mM, such as generally 30 mM to 100 mM, 50 mM to 160 mM, for example 50 mM to 120 mM or 80 mM to 140 mM. Generally, the NaCl is less than 150 mM, and generally less than 140 mM, 130 mM, 120 mM, 110 mM, 100 mM, 90 mM, 80 mM, 70 mM, 60 mM, 50 mM, 40 mM, 30 mM, 20 mM, 10 mM or less. The particular amount can be empirically determined in order to retain enzyme activity and/or tonicity.

In another example, glycerin (glycerol) is optionally included in the compositions and combinations described herein. For example, the compositions and combinations provided herein typically contain less than 60 mM glycerin, such as less than 55 mM, less than 50 mM, less than 45 mM, less than 40 mM, less than 35 mM, less than 30 mM, less than 25 mM, less than 20 mM, less than 15 mM, 10 mM or less.

### Other Agents or Excipients

The stable compositions and combinations provided herein can optionally contain one or more other agents, carriers, excipients or preservatives. For example, exemplary stabilizers that optionally can be included in the hyaluronidase compositions and combinations provided herein include, but are not limited to, amino acids, amino acid derivatives, amines, sugars, polyols, salts and buffers, surfactants, and other agents. For example, included among the types of stabilizers that optionally can be contained in the formulations herein is an amino acid stabilizer or a hyaluronidase inhibitor (e.g. a hyaluronidase substrate, such as hyaluronan). Exemplary amino acid stabilizers, amino acid derivatives or amines include, but are not limited to, L-Arginine, Glutamine, glycine, Lysine, Methionine, Proline, Lys-Lys, Gly-Gly, Trimethylamine oxide (TMAO) or betaine. Exemplary of sugars and polyols include, but are not limited to, glycerol, sorbitol, mannitol, inositol, sucrose or trehalose. Exemplary of salts and buffers include, but are not limited to, magnesium chloride, sodium sulfate, Tris such as Tris (100 mM), or sodium Benzoate. Exemplary surfactants include, but are not limited to, poloxamer 188 (e.g. Pluronic® F68), polysorbate 80 (PS80), polysorbate 20 (PS20). Other stabilizers include, but are not limited to, hyaluronic acid (HA), human serum albumin (HSA), phenyl butyric acid, taurocholic acid, polyvinylpyrolidone (PVP) or zinc.

In an embodiment, the hyaluronidase compositions and combinations also can optionally contain an amount of preservative(s) that, when combined with the components set forth above, result in a stable composition or combination. When included, the preservatives are present in a sufficient concentration to provide the anti-microbial requirements of, for example, the United States Pharmacopoeia (USP) and the European Pharmacopoeia (EP). Typically, formulations that meet EP (EPA or EPB) anti-microbial requirements contain more preservative than those formulated only to meet USP anti-microbial requirements. Generally, when included, the compositions and combinations provided herein contain preservative(s) in an amount that exhibits anti-microbial activity by killing or inhibiting the propagation of microbial organisms in a sample of the composition as assessed in an antimicrobial preservative effectiveness test (APET). Non-limiting examples of preservatives that can be included in the compositions and combinations provided herein include, but are not limited to, phenol, meta-cresol (m-cresol), methylparaben, benzyl alcohol, thimerosal, benzalkonium chloride, 4-chloro-1-butanol, chlorhexidine dihydrochloride, chlorhexidine digluconate, L-phenylalanine, EDTA, bronopol (2-bromo-2-nitropropane-1,3-diol), phenylmercuric acetate, glycerol (glycerin), imidurea, chlorhexidine, sodium dehydroacetate, ortho-cresol (o-cresol), para-cresol (p-cresol), chlorocresol, cetrimide, benzethonium chloride, ethylparaben, propylparaben or butylparaben and any combination thereof. In one example, the compositions and combinations contain at least one phenolic preservative. For example, the composition or combination contains phenol, m-cresol or phenol and m-cresol. When included in the compositions and combinations provided herein, the total amount of the one or more preservative agents as a percentage (%) of mass concentration (w/v) in the composition and combination can be, for example, between from or between about from 0.1% to 0.4%, such as 0.1% to 0.3%, 0.15% to 0.325%, 0.15% to 0.25%. 0.1% to 0.2%, 0.2% to 0.3%, or 0.3% to 0.4%, and generally less than 0.4% (w/v) preservative, for example, at least or about at least 0.1%, 0.12%, 0.125%, 0.13%, 0.14%, 0.15%, 0.16%, 0.17%, 0.175%, 0.18%, 0.19%, 0.2%, 0.25%, 0.3%, 0.325%, 0.35% but less than 0.4% total preservative.

Optionally, the compositions and combinations can include carriers such as a diluent, adjuvant, excipient, or vehicle with which the formulation is administered. Examples of suitable pharmaceutical carriers are described in “Remington's Pharmaceutical Sciences” by E. W. Martin. Such compositions will contain a therapeutically effective amount of the compound, generally in purified form or partially purified form, together with a suitable amount of carrier so as to provide the form for proper administration to the patient. Such pharmaceutical carriers can be sterile liquids, such as water and oils, including those of petroleum, animal, vegetable or synthetic origin, such as peanut oil, soybean oil, mineral oil, and sesame oil. Water is a typical carrier when the pharmaceutical composition is administered intravenously. Saline solutions and aqueous dextrose and glycerol solutions also can be employed as liquid carriers, particularly for injectable solutions.

For example, pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances. Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations can be added to parenteral preparations packaged in multiple-dose containers, which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcellulose, hydroxypropyl methylcellulose and polyvinylpyrrolidone. Emulsifying agents include Polysorbate 80 (TWEEN 80). A sequestering or chelating agent of metal ions include EDTA. Pharmaceutical carriers also include ethyl alcohol, polyethylene glycol and propylene glycol for water miscible vehicles and sodium hydroxide, hydrochloric acid, citric acid or lactic acid for pH adjustment.

Compositions can contain along with an active ingredient: a diluent such as lactose, sucrose, dicalcium phosphate, or carboxymethylcellulose; a lubricant, such as magnesium stearate, calcium stearate and talc; and a binder such as starch, natural gums, such as gum acacia, gelatin, glucose, molasses, polyvinylpyrrolidone, celluloses and derivatives thereof, povidone, crospovidones and other such binders known to those of skill in the art.

For example, an excipient protein can be added to the composition or combination that can be any of a number of pharmaceutically acceptable proteins or peptides. Generally, the excipient protein is selected for its ability to be administered to a mammalian subject without provoking an immune response. For example, human serum albumin is generally well-suited for use in pharmaceutical formulations. Other known pharmaceutical protein excipients include, but are not limited to, starch, glucose, lactose, sucrose, gelatin, malt, rice, flour, chalk, silica gel, sodium stearate, glycerol monostearate, talc, sodium chloride, dried skim milk, glycerol, propylene, glycol, water, and ethanol. The excipient is included in the composition or combination at a sufficient concentration to prevent adsorption of the protein to the holding vessel or vial. The concentration of the excipient will vary according to the nature of the excipient and the concentration of the protein in the composition or combination.

A composition or combination, if desired, also can contain minor amounts of wetting or emulsifying agents, or pH buffering agents, for example, acetate, sodium citrate, cyclodextrin derivatives, sorbitan monolaurate, triethanolamine sodium acetate, triethanolamine oleate, and other such agents.

In an embodiment, the injectable combination comprises a hyaluronidase and Gemtuzumab ozogamicin. For example, Gemtuzumab ozogamicin is mixed with an inert additive, carrier, or excipient selected from dextran (e.g. dextran 40), sodium chloride, sodium phosphate dibasic anhydrous, sodium phosphate monobasic monohydrate, and sucrose. For example, Gemtuzumab ozogamicin is mixed with each dextran (e.g. dextran 40), sodium chloride, sodium phosphate dibasic anhydrous, sodium phosphate monobasic monohydrate, and sucrose. In an embodiment, the injectable combination comprises a hyaluronidase and Brentuximab vedotin. For Example, Brentuximab vedotin is mixed with an inert additive, carrier, or excipient selected from trehalose dihydrate, sodium citrate dihydrate, citric acid monohydrate, and polysorbate (e.g., polysorbate 80). For example, Brentuximab vedotin is mixed with each of trehalose dihydrate, sodium citrate dihydrate, citric acid monohydrate, and polysorbate (e.g., polysorbate 80). In an embodiment, the injectable combination comprises a hyaluronidase and Ado-Trastuzumab emtansine. For example, Ado-Trastuzumab emtansine is mixed with an inert additive, carrier, or excipient selected from polysorbate (e.g. polysorbate 20), sodium succinate, and sucrose. For example, Ado-Trastuzumab emtansine is mixed with each of polysorbate (e.g. polysorbate 20), sodium succinate, and sucrose. In an embodiment, the injectable combination comprises a hyaluronidase and Inotuzumab ozogamicin. For example, Inotuzumab ozogamicin emtansine is mixed with an inert additive, carrier, or excipient selected from polysorbate (e.g. polysorbate 80), sodium chloride, sucrose, and tromethamine. For example, Inotuzumab ozogamicin emtansine is mixed with each of polysorbate (e.g. polysorbate 80), sodium chloride, sucrose, and tromethamine. In an embodiment, the injectable combination comprises a hyaluronidase and Polatuzumab vedotin. For example, Polatuzumab vedotin is mixed with an inert additive, carrier, or excipient selected from polysorbate (e.g. polysorbate 20), sodium hydroxide, succinic acid, and sucrose. For example, Polatuzumab vedotin is mixed with each of polysorbate (e.g. polysorbate 20), sodium hydroxide, succinic acid, and sucrose. In an embodiment, the injectable combination comprises a hyaluronidase and Enfortumab vedotin. For example, Enfortumab vedotin is mixed with an inert additive, carrier, or excipient selected from histidine, histidine hydrochloride monohydrate, polysorbate (e.g. polysorbate 20), and trehalose dihydrate. For example, Enfortumab vedotin is mixed with each of histidine, histidine hydrochloride monohydrate, polysorbate (e.g. polysorbate 20), and trehalose dihydrate. In an embodiment, the injectable combination comprises a hyaluronidase and Fam-Trastuzumab deruxtecan. For example, Fam-Trastuzumab deruxtecan is mixed with an inert additive, carrier, or excipient selected from histidine (e.g. L-histidine), histidine hydrochloride monohydrate (e.g. L-histidine hydrochloride monohydrate), polysorbate (e.g. polysorbate 80), and sucrose. For example, Fam-Trastuzumab deruxtecan is mixed with each of histidine (e.g. L-histidine), histidine hydrochloride monohydrate (e.g. L-histidine hydrochloride monohydrate), polysorbate (e.g. polysorbate 80), and sucrose. In an embodiment, the injectable combination comprises a hyaluronidase and Sacituzumab govitecan. For example, Sacituzumab govitecan is mixed with an inert additive, carrier, or excipient selected from 2-(N-morpholino) ethane sulfonic acid (MES), polysorbate (e.g. polysorbate 80), trehalose dihydrate, and sodium chloride. In an embodiment, Sacituzumab govitecan is mixed with each of 2-(N-morpholino) ethane sulfonic acid (MES), polysorbate (e.g. polysorbate 80), trehalose dihydrate, and sodium chloride. In an embodiment, the injectable combination comprises a hyaluronidase and Loncastuximab Tesirine. For example, Loncastuximab Tesirine is mixed with an inert additive, carrier, or excipient selected from histidine (e.g. L-histidine), histidine monohydrochloride (e.g. L-histidine monohydrochloride), polysorbate (e.g. polysorbate 20), and sucrose. For example, Loncastuximab Tesirine is mixed with each of histidine (e.g. L-histidine), histidine monohydrochloride (e.g. L-histidine monohydrochloride), polysorbate (e.g. polysorbate 20), and sucrose. In an embodiment, the injectable combination comprises a hyaluronidase and Tisotumab vedotin. For example, Tisotumab vedotin is mixed with an inert additive, carrier, or excipient selected from mannitol (e.g. d-mannitol), histidine (e.g. L-histidine), histidine monohydrochloride (e.g. L-histidine monohydrochloride), and sucrose. For example, Tisotumab vedotin is mixed with an each of mannitol (e.g. d-mannitol), histidine (e.g. L-histidine), histidine monohydrochloride (e.g. L-histidine monohydrochloride), and sucrose. In an embodiment, the injectable combination comprises a hyaluronidase and Mirvetuximab soravtansine-gynx. For example, Mirvetuximab soravtansine-gynx is mixed with an inert additive, carrier, or excipient selected from acetic acid (e.g. glacial acetic acid), polysorbate (e.g. polysorbate 20), sodium acetate, and sucrose. For example, Mirvetuximab soravtansine-gynx is mixed with each of acetic acid (e.g. glacial acetic acid), polysorbate (e.g. polysorbate 20), sodium acetate, and sucrose.

### 3. Methods of Administration

In an embodiment, each of the hyaluronidase and antibody-drug conjugate can be administered to a patient via injection. In an embodiment the hyaluronidase and antibody-drug conjugate are administered subcutaneously. For example, the hyaluronidase and antibody-drug conjugate can be administered to a patient subcutaneously in the abdominal tissue. The hyaluronidase and antibody-drug conjugate can be administered separately or in the same composition.

In an embodiment, the hyaluronidase and antibody-drug conjugate are administered topically by mucosal delivery. In an embodiment, the mucosal delivery is selected from the group consisting of buccal delivery, pulmonary delivery, ocular delivery, nasal delivery, intranasal delivery, vaginal delivery, and oral delivery. In an embodiment, the hyaluronidase is administered directly to a mucosal tissue of the human subject, including at the affected site. In an embodiment, the mucosal tissue is selected from the group consisting of anterior nostril, nasal sinus, vaginal, esophagus, urethral, sublingual and buccal.

In an embodiment, various delivery systems are known and can be used to administer the hyaluronidase in combination with an antibody-drug conjugate. For example, the hyaluronidase can be encapsulated in liposomes, microparticles, microcapsules for topical delivery. In addition, pulmonary administration can also be used, such as inhalers or nebulizers, and aerosol formulations.

If the hyaluronidase and antibody-drug conjugate comprise pulmonary or intranasal administration, the composition can be formulated in the form of an aerosol, spray, mist or drip. In particular, the hyaluronidase and antibody-drug conjugate can be provided by the use of suitable propellants (such as dichlorodifluoromethane, trichlorofluoromethane, dichlorotetrafluoroethane, carbon dioxide, or other suitable gases). Aerosol sprays are delivered from pressurized packaging or sprayers. In the case of pressurized aerosols, the dosage unit can be determined by providing a valve that can deliver a metered amount. Capsules and cartridges (made of, for example, gelatin) containing powder mixtures of compounds and suitable powder bases such as lactose or starch can be formulated and used in inhalers.

If the hyaluronidase and antibody-drug conjugate is administered topically, including directly to the affected site, the compositions can be in the form of ointment, cream, transdermal patch, lotion, gel, shampoo, spray, aerosol, solution, emulsion. With regard to non-sprayable topical dosage forms, it is generally employed to include a carrier or one or more excipients compatible with topical application, and the dynamic viscosity is preferably greater than the viscosity of water to a semi-solid or solid form. Suitable formulations include (but are not limited to) solutions, suspensions, emulsions, creams, ointments, powders, wipes, ointments, which are sterilized or used to affect various properties such as osmotic pressure, if necessary, adjuvants (such as preservatives, stabilizers, wetting agents, buffers or salts) are present in the composition.

Other suitable topical dosage forms include sprayable aerosol formulations, where the active ingredient, optionally combined with a solid or liquid inert carrier, is mixed and encapsulated with a pressurized volatile substance (such as a gaseous propellant such as freon) or encapsulated in a squeeze bottle. If necessary, a moisturizing agent or humectant may also be added to the composition.

In an embodiment, it may be necessary to locally administer the hyaluronidase to the affected site in need of treatment; this may be achieved by, for example, but not limited to, topical administration, local infusion, injection, or by means of an implant. The implant may be a porous or non-porous material, including membranes and matrices, such as silicone membranes, polymers, fibrous matrices or collagen matrices.

In an embodiment, the invention provides a composition for transdermal delivery containing the hyaluronidase, antibody-drug conjugate and a pharmaceutical excipient suitable for transdermal delivery. Compositions of the present invention can be formulated into preparations in solid, semi-solid, or liquid forms suitable for local or topical administration, such as gels, water soluble jellies, creams, lotions, suspensions, foams, powders, slurries, ointments, solutions, oils, pastes, suppositories, sprays, emulsions, saline solutions, dimethylsulfoxide (DMSO)-based solutions. In general, carriers with higher densities are capable of providing an area with a prolonged exposure to the active ingredients. In contrast, a solution formulation may provide more immediate exposure of the active ingredient to the chosen area.

The transdermal compositions also may comprise suitable solid or gel phase carriers or excipients, which are compounds that allow increased penetration of, or assist in the delivery of, the hyaluronidase and antibody-drug conjugate across the stratum corneum permeability barrier of the skin. There are many of these penetration-enhancing molecules known to those skilled in the field of topical formulation. Examples of such carriers and excipients include, but are not limited to, humectants (e.g. urea), glycols (e.g. ethylene glycol, propylene glycol), alcohols (e.g. methanol, ethanol, propanol, including isopropanol and n-propanol; butanol, including n-butanol, isobutanol, tert-butanol, and sec-butanol; pentanol, including 1-pentanol, 2-pentanol, 3-pentanol, 2-methyl-1-butanol, 3-methyl-1-butanol, 2-methyl-2-butanol, 2-methyl-3-butanol, and 2,2-dimethylpropanol; and hexanol, including hexan-1-ol, hexan-2-ol, hexan-3-ol, 2-methylpentan-1-ol, 3-methylpentan-1-ol, 4-methylpentan-1-ol, 2-methylpentan-2-ol, 3-methylpentan-2-ol, 4-methylpentan-2-ol, 2-methylpentan-3-ol, 3-methylpentan-3-ol, 2,2-dimethylbutan-1-ol, 2,3-dimethylbutan-1-ol, 3,3-dimethylbutan-1-ol, 2,3-dimethylbutan-2-ol, 3,3-dimethylbutan-2-ol, and 2-ethylbutan-1-ol), fatty acids (e.g. oleic acid, α-linolenic acid, linoleic acid, γ-linolenic acid, palmitoleic acid), surfactants (e.g. isopropyl myristate and sodium lauryl sulfate), pyrrolidones (e.g. N-methyl-2-pyrrolidone, 2-pyrrolidone), glycerol monolaurate, sulfoxides (e.g. dimethyl sulfoxide, decylmethylsulfoxide), terpenes (e.g. menthol, 1,8-cineole, limonene, menthone, nerolidol), amines, amides, alkanes, alkanols, water, calcium carbonate, calcium phosphate, various sugars, starches, cellulose derivatives, gelatin, and polymers such as polyethylene glycols and polypropylene glycols.

Another exemplary formulation for delivery hyaluronidase and antibody-drug conjugate employs transdermal delivery devices (“patches”). Such transdermal patches may be used to provide continuous or discontinuous infusion of the hyaluronidase in controlled amounts, either with or without another active pharmaceutical ingredient. The construction and use of transdermal patches for the delivery of pharmaceutical agents is known. See, e.g. U.S. Pat. Nos. 5,023,252; 4,992,445 and 5,001,139 incorporated by reference herein. Such patches may be constructed for continuous, pulsatile, or on demand delivery of hyaluronidase and antibody-drug conjugate.

A diverse range of delivery methods may be utilized to cater to a wide range of medical needs. These methods offer patients and healthcare providers options based on the nature of the medication, dosage requirements, and the specific condition being treated. In some embodiments, an injector, or other handheld devices for injections that allow patients to self-administer preset doses, is used to administer the hyaluronidase and antibody-drug conjugate to the affected site. In some embodiments, the autoinjector is a prefilled syringe, a high volume autoinjector or a large volume autoinjector. The injector may be button actuated or needle guard actuated and may be configured to deliver a single dose or a plurality of doses.

A wearable administration apparatus may be utilized to administer the hyaluronidase and antibody-drug conjugate to the affected site in cases that require prolonged delivery. In some embodiments, an on-body injector, or other wearable device that deliver a larger volume of medication subcutaneously over an extended period, is used to administer the hyaluronidase and antibody-drug conjugate to the affected site. In some embodiments, a patch pump, or other wearable device adhering to the skin that delivers medication subcutaneously through an injection site, is used to administer the hyaluronidase and antibody-drug conjugate to the affected site. In some embodiments, a wearable infusion pump, or other device worn on the body for continuous subcutaneous infusion of medication, is used to administer the hyaluronidase and antibody-drug conjugate to the affected site.

An implantable administration apparatus may be utilized to administer the hyaluronidase and antibody-drug conjugate. The implantable apparatus may be made of porous or non-porous materials (such as silicone membranes, polymers, fibrous matrices, or collagen matrices) that release medication gradually over time. In some embodiments, a subcutaneous impact, or other implant specifically designed to be placed beneath the skin for controlled and sustained release of medication, is used to administer the hyaluronidase and antibody-drug conjugate to the affected site. In some embodiments, an intramuscular implant, or other implant designed for insertion into muscle tissue, providing a localized and sustained release of medication, is used to administer the hyaluronidase to the affected site. In some embodiments, an intradermal implant, or other implant placed within the dermal layer of the skin for targeted and controlled delivery of medication, is used to administer the hyaluronidase and antibody-drug conjugate to the affected site. In some embodiments, topical administration, or other application directly onto the skin, including creams, gels, ointments, and transdermal patches, is used to administer the hyaluronidase and antibody-drug conjugate to the affected site.

The compositions for administration to a patient via an injection (e.g. subcutaneously) also may comprise suitable inert additives, carriers, or excipients. In an embodiment, the injectable composition or combination comprises histidine. In an embodiment, the injectable composition comprises sodium chloride. In an embodiment, the injectable composition comprises polysorbate. In an embodiment, the polysorbate comprises polysorbate 80. In an embodiment, the injectable composition comprises an antioxidant. In an embodiment, the antioxidant comprises methionine.

In an embodiment, the injectable combination comprises a hyaluronidase and Gemtuzumab ozogamicin. In an embodiment, Gemtuzumab ozogamicin is mixed with an inert additive, carrier, or excipient selected from dextran (e.g. dextran 40), sodium chloride, sodium phosphate dibasic anhydrous, sodium phosphate monobasic monohydrate, and sucrose. In an embodiment, Gemtuzumab ozogamicin is mixed with each dextran (e.g. dextran 40), sodium chloride, sodium phosphate dibasic anhydrous, sodium phosphate monobasic monohydrate, and sucrose. In an embodiment, the injectable combination comprises a hyaluronidase and Brentuximab vedotin. In an embodiment, Brentuximab vedotin is mixed with an inert additive, carrier, or excipient selected from trehalose dihydrate, sodium citrate dihydrate, citric acid monohydrate, and polysorbate (e.g., polysorbate 80). In an embodiment, Brentuximab vedotin is mixed with each of trehalose dihydrate, sodium citrate dihydrate, citric acid monohydrate, and polysorbate (e.g., polysorbate 80). In an embodiment, the injectable combination comprises a hyaluronidase and Ado-Trastuzumab emtansine. In an embodiment, Ado-Trastuzumab emtansine is mixed with an inert additive, carrier, or excipient selected from polysorbate (e.g. polysorbate 20), sodium succinate, and sucrose. In an embodiment, Ado-Trastuzumab emtansine is mixed with each of polysorbate (e.g. polysorbate 20), sodium succinate, and sucrose. In an embodiment, the injectable combination comprises a hyaluronidase and Inotuzumab ozogamicin. In an embodiment, Inotuzumab ozogamicin emtansine is mixed with an inert additive, carrier, or excipient selected from polysorbate (e.g. polysorbate 80), sodium chloride, sucrose, and tromethamine. In an embodiment, Inotuzumab ozogamicin emtansine is mixed with each of polysorbate (e.g. polysorbate 80), sodium chloride, sucrose, and tromethamine. In an embodiment, the injectable combination comprises a hyaluronidase and Polatuzumab vedotin. In an embodiment, Polatuzumab vedotin is mixed with an inert additive, carrier, or excipient selected from polysorbate (e.g. polysorbate 20), sodium hydroxide, succinic acid, and sucrose. In an embodiment, Polatuzumab vedotin is mixed with each of polysorbate (e.g. polysorbate 20), sodium hydroxide, succinic acid, and sucrose. In an embodiment, the injectable combination comprises a hyaluronidase and Enfortumab vedotin. In an embodiment, Enfortumab vedotin is mixed with an inert additive, carrier, or excipient selected from histidine, histidine hydrochloride monohydrate, polysorbate (e.g. polysorbate 20), and trehalose dihydrate. In an embodiment, Enfortumab vedotin is mixed with each of histidine, histidine hydrochloride monohydrate, polysorbate (e.g. polysorbate 20), and trehalose dihydrate. In an embodiment, the injectable combination comprises a hyaluronidase and Fam-Trastuzumab deruxtecan. In an embodiment, Fam-Trastuzumab deruxtecan is mixed with an inert additive, carrier, or excipient selected from histidine (e.g. L-histidine), histidine hydrochloride monohydrate (e.g. L-histidine hydrochloride monohydrate), polysorbate (e.g. polysorbate 80), and sucrose. In an embodiment, Fam-Trastuzumab deruxtecan is mixed with each of histidine (e.g. L-histidine), histidine hydrochloride monohydrate (e.g. L-histidine hydrochloride monohydrate), polysorbate (e.g. polysorbate 80), and sucrose. In an embodiment, the injectable combination comprises a hyaluronidase and Sacituzumab govitecan. In an embodiment, Sacituzumab govitecan is mixed with an inert additive, carrier, or excipient selected from 2-(N-morpholino) ethane sulfonic acid (MES), polysorbate (e.g. polysorbate 80), trehalose dihydrate, and sodium chloride. In an embodiment, Sacituzumab govitecan is mixed with each of 2-(N-morpholino) ethane sulfonic acid (MES), polysorbate (e.g. polysorbate 80), trehalose dihydrate, and sodium chloride. In an embodiment, the injectable combination comprises a hyaluronidase and Loncastuximab Tesirine. In an embodiment, Loncastuximab Tesirine is mixed with an inert additive, carrier, or excipient selected from histidine (e.g. L-histidine), histidine monohydrochloride (e.g. L-histidine monohydrochloride), polysorbate (e.g. polysorbate 20), and sucrose. In an embodiment, Loncastuximab Tesirine is mixed with each of histidine (e.g. L-histidine), histidine monohydrochloride (e.g. L-histidine monohydrochloride), polysorbate (e.g. polysorbate 20), and sucrose. In an embodiment, the injectable combination comprises a hyaluronidase and Tisotumab vedotin. In an embodiment, Tisotumab vedotin is mixed with an inert additive, carrier, or excipient selected from mannitol (e.g. d-mannitol), histidine (e.g. L-histidine), histidine monohydrochloride (e.g. L-histidine monohydrochloride), and sucrose. In an embodiment, Tisotumab vedotin is mixed with an each of mannitol (e.g. d-mannitol), histidine (e.g. L-histidine), histidine monohydrochloride (e.g. L-histidine monohydrochloride), and sucrose. In an embodiment, the injectable combination comprises a hyaluronidase and Mirvetuximab soravtansine-gynx. In an embodiment, Mirvetuximab soravtansine-gynx is mixed with an inert additive, carrier, or excipient selected from acetic acid (e.g. glacial acetic acid), polysorbate (e.g. polysorbate 20), sodium acetate, and sucrose. In an embodiment, Mirvetuximab soravtansine-gynx is mixed with each of acetic acid (e.g. glacial acetic acid), polysorbate (e.g. polysorbate 20), sodium acetate, and sucrose.

In an embodiment, the injection of a high volume of the disclosed formulation in a subject leads to fewer side effects in the subject compared to an identical subject administered the same volume of a comparable formulation that does not comprise the hyaluronidase. In an embodiment, the injection of a high volume disclosed elsewhere herein with the disclosed formulation has reduced back leakage compared similar formulation that does not comprise the hyaluronidase. In an embodiment, the back leakage is reduced about 54%, about 56%, about 58%, about 60%, about 62%, about 64%, about 66%, about 68%, about 70%, about 72%, about 74%, about 76%, or about 78% when a high volume of the disclosed formulation is administered to a subject using a HVAI fitted with a 23 gauge needle compared to a similar formulation that does not comprise the hyaluronidase. In an embodiment, the back leakage is reduced about 62%, about 64%, about 68%, about 70%, about 72%, about 74%, about 76%, about 78%, about 80%, about 82%, about 84%, or about 86% when a high volume of the disclosed formulation is administered to a subject using a HVAI fitted with a 25 gauge needle compared to a similar formulation that does not comprise the hyaluronidase.

In an embodiment, the swelling (bleb) volume is reduced following the injection of the disclosed formulation into a subject when compared to a similar formulation that does not comprise the hyaluronidase. In an embodiment, the swelling height is reduced following the injection of the disclosed formulation when compared to a similar formulation that does not comprise the hyaluronidase. In an embodiment, the swelling size is reduced following the injection of the disclosed formulation when compared to a similar formulation that does not comprise the hyaluronidase. In an embodiment, the swelling area is reduced following the injection of the disclosed formulation when compared to a similar formulation that does not comprise the hyaluronidase. In an embodiment, the swelling induration following the initial injection of the disclosed formulation is minimized compared to a similar formulation that does not comprise the hyaluronidase. In an embodiment, the swelling resolves quicker when the disclosed formulation is injected compared to a similar formulation that does not comprise the hyaluronidase. In an embodiment, the disclosed formulation permits for more consistent delivery (i.e., time to delivery, reduction in bleb swelling volume, height and induration) from injection to injection, compared to a similar formulation that does not comprise the hyaluronidase. In an embodiment, the disclosed formulation permits for faster delivery of the full volume from a HVAI than a comparable formulation that does not comprise the hyaluronidase which results in less pain and discomfort for the subject.

In an embodiment, the systemic toxicity of the disclosed formulation is lower when compared to a similar formulation that does not comprise the hyaluronidase. In an embodiment, the systemic toxicity following the subcutaneous injection of the disclosed formulation is lower when compared to the subcutaneous injection of a similar formulation that does not comprise the hyaluronidase. In an embodiment, the systemic toxicity following the subcutaneous injection of the disclosed formulation is lower when compared to an intravenous injection of a similar formulation that does not comprise the hyaluronidase.

In an embodiment, the disclosed formulation produced less of a wound response in the subject compared to a similar formulation that does not comprise the hyaluronidase. In an embodiment, wound response in the subject following subcutaneous injection of the disclosed formulation is lower when compared to the subcutaneous injection of a similar formulation that does not comprise the hyaluronidase. In an embodiment, wound response in the subject following subcutaneous injection of the disclosed formulation is lower when compared to an intravenous injection of a similar formulation that does not comprise the hyaluronidase. In an embodiment, no focal keratosis is observed as a wound response in the subject following administration of the disclosed formulation whereas mild focal keratosis is observed as a wound response in a subject who is administered a similar formulation that does not comprise the hyaluronidase. In an embodiment, no focal keratosis is observed as a wound response in the subject following subcutaneous administration of the disclosed formulation whereas mild focal keratosis is observed as a wound response in a subject who is subcutaneously administered a similar formulation that does not comprise the hyaluronidase. In an embodiment, no focal keratosis is observed as a wound response in the subject following subcutaneous administration of the disclosed formulation whereas mild focal keratosis is observed as a wound response in a subject who is intravenously administered a similar formulation that does not comprise the hyaluronidase. In an embodiment, no immune cell infiltrates are observed as a wound response in the subject following administration of the disclosed formulation whereas immune cell infiltrates are observed as a wound response in a subject who is administered a similar formulation that does not comprise the hyaluronidase. In an embodiment, no immune cell infiltrates are observed as a wound response in the subject following subcutaneous administration of the disclosed formulation whereas immune cell infiltrates are observed as a wound response in a subject who is subcutaneously administered a similar formulation that does not comprise the hyaluronidase. In an embodiment, no immune cell infiltrates are observed as a wound response in the subject following subcutaneous administration of the disclosed formulation whereas immune cell infiltrates are observed as a wound response in a subject who is intravenously administered a similar formulation that does not comprise the hyaluronidase.

In an embodiment, the area under the curve (AUC) of the disclosed formulation is substantially equivalent to the AUC of a similar formulation comprising the same ADC at the same concentration that does not comprise hyaluronidase. In an embodiment, the area under the curve (AUC) following subcutaneous administration of the disclosed formulation is substantially equivalent to the AUC following intravenous administration of a similar formulation comprising the same ADC at the same concentration that does not comprise hyaluronidase. In an embodiment, the peak plasma concentration (Cₘₐₓ) of the disclosed formulation is lower than the Cₘₐₓ of a similar formulation comprising the same ADC at the same concentration that does not comprise hyaluronidase. In an embodiment, the Cₘₐₓ of the disclosed formulation about 10%, about 15%, about 20%, about 25%, about 30%, about 35%, about 40%, about 50%, about 55%, about 60%, about 65%, about 70%, about 75%, about 80%, or about 85% lower than the Cₘₐₓ of a similar formulation comprising the same ADC at the same concentration that does not comprise hyaluronidase. In an embodiment, the Cₘₐₓ following subcutaneous administration of the disclosed formulation is lower than the Cₘₐₓ following intravenous administration of a similar formulation comprising the same ADC at the same concentration that does not comprise hyaluronidase. In an embodiment, the Cₘₐₓ following subcutaneous administration of the disclosed formulation about 10%, about 15%, about 20%, about 25%, about 30%, about 35%, about 40%, about 50%, about 55%, about 60%, about 65%, about 70%, about 75%, about 80%, or about 85% lower than the Cₘₐₓ following intravenous administration of a similar formulation comprising the same ADC at the same concentration that does not comprise hyaluronidase.

The following clauses describe particular embodiments disclosed in the specification, examples and figures.

### CLAUSES OF THE DISCLOSURE

Clause 1. A combination dosing regimen, comprising: administering a soluble hyaluronidase; and administering an antibody-drug conjugate.

Clause 2. The combination dosing regimen of clause 1, wherein the hyaluronidase is a soluble PH20 hyaluronidase.

Clause 3. The combination dosing regimen of clause 2, wherein the soluble hyaluronidase is the composition designated rHuPH20.

Clause 4. The combination dosing regimen of clause 1, wherein the soluble hyaluronidase has the sequence set forth as amino acids 36-482 set forth in SEQ ID NO: 1 (i.e., SEQ ID NO: 4) or 36-483 set forth in SEQ ID NO: 1 (i.e., SEQ ID NO: 46) or has at least 98% sequence identity to the sequence set forth in SEQ ID NO: 4 or SEQ ID NO: 46.

Clause 5. The combination dosing regimen of clause 1, wherein:
the soluble hyaluronidase comprises amino acids 36-464 of SEQ ID NO: 1, or comprises a sequence of amino acids that has at least 85% sequence identity to a sequence of amino acids that contains at least amino acids 36-464 of SEQ ID NO:1, and retains hyaluronidase activity.

Clause 6. The combination dosing regimen of clause 1, wherein the soluble hyaluronidase comprises a sequence of amino acids that has at least 86%, 87%, 88%, 89%, 90%, 91%, 92%, 93%, 94%, 95%, 96%, 97%, 98% or 99% sequence identity to a sequence of amino acids that contains at least amino acids 36-464 of SEQ ID NO:1 and retains hyaluronidase activity.

Clause 7. The combination dosing regimen of clause 1, wherein:
a) the soluble hyaluronidase has an amino acid sequence selected from amino acids 36-465 of SEQ ID NO: 1, 36-466 of SEQ ID NO: 1, 36-467 of SEQ ID NO: 1, 36-468 of SEQ ID NO: 1, 36-469 of SEQ ID NO: 1, 35-470 of SEQ ID NO: 1, 36-471 of SEQ ID NO: 1, 36-472 of SEQ ID NO: 1, 36-474 of SEQ ID NO: 1, 36-475 of SEQ ID NO: 1, 36-476 of SEQ ID NO: 1, 35-477 of SEQ ID NO: 1, 36-478 of SEQ ID NO: 1 (i.e., SEQ ID NO: 8), 36-479 of SEQ ID NO: 1 (i.e., SEQ ID NO: 7), 36-480 of SEQ ID NO: 1 (i.e., SEQ ID NO: 6), 36-481 of SEQ ID NO: 1 (i.e., SEQ ID NO: 5), 36-482 of SEQ ID NO: 1 (i.e., SEQ ID NO: 4), 36-483 of SEQ ID NO: 1 (i.e., SEQ ID NO: 46), 35-484 of SEQ ID NO: 1, 36-485 of SEQ ID NO: 1, 36-486 of SEQ ID NO: 1, 36-487 of SEQ ID NO: 1, 36-488 of SEQ ID NO: 1, 36-489 of SEQ ID NO: 1, 36-490 of SEQ ID NO: 1, 35-491 of SEQ ID NO: 1, 36-492 of SEQ ID NO: 1, 36-493 of SEQ ID NO: 1, 36-494 of SEQ ID NO: 1, 36-495 of SEQ ID NO: 1, 36-496 of SEQ ID NO: 1, 36-497 of SEQ ID NO: 1, 35-498 of SEQ ID NO: 1, 36-499 of SEQ ID NO: 1, and 36-500 of SEQ ID NO: 1, or an N-terminally truncated variant thereof lacking residues 36, 36-37, 36-38, 36-39, or 36-40; or
b) the soluble hyaluronidase is a variant soluble hyaluronidase that has at least 91% sequence identity to a soluble hyaluronidase of a).

Clause 8. The combination dosing regimen of clause 7, wherein the soluble hyaluronidase has at least 95% sequence identity to a soluble hyaluronidase of clause 7.

Clause 9. The combination dosing regimen of clause 7, wherein:
the soluble hyaluronidase comprises the replacement F204P, and has increased stability relative the unmodified PH20 that does not comprise F204P; and optionally has one or more substitutions selected from:
   (a) T341S, L342W, S343E, I344N, M345T, S347T, M348K, K349E, L352Q, L353A, L3541, D355K, N356E, E359D and I361T;
   (b) L342W, S343E, I344N, M345T, S347T, M348K, K349E, L352Q, L353A, L3541, D355K, N356E, E359D and I361T;
   (c) M345T, S347T, M348K, K349E, L352Q, L353A, L354I, D355K, N356E, E359D, I361T and N363G;
   (d) T341G, L342W, S343E, I344N, M345T, S347T, M348K, K349E, L352Q, L353A, L354I, D355K, N356E, E359D and I361T;
   (e) T341A, L342W, S343E, I344N, M345T, S347T, M348K, K349E, L352Q, L353A, L354I, D355K, N356E, E359D and I361T;
   (f) T341C, L342W, S343E, I344N, M345T, S347T, M348K, K349E, L352Q, L353A, L354I, D355K, N356E, E359D and I361T;
   (g) T341D, L342W, S343E, I344N, M345T, S347T, M348K, K349E, L352Q, L353A, L354I, D355K, N356E, E359D and I361T;
   (h) I344N, M345T, S347T, M348K, K349E, L352Q, L353A, L354I, D355K, N356E, E359D and I361T, and
   (i) S343E, I344N, M345T, S347T, M348K, K349E, L352Q, L353A, L354I, D355K, N356E, E359D and I361T,
wherein increased stability is measured as increased stability in a denaturing condition or at elevated temperatures.

Clause 10. The combination dosing regimen of clause 7, wherein:
the soluble hyaluronidase is a variant modified polypeptide or catalytically active portion thereof that comprises one or more amino acid residue substitutions selected from among T341A, T341C, T341D, T341G, T341S, L342W, S343E, I344N, M348K, and N363G; wherein
   amino acid numbering is with reference to SEQ ID NO:1;
   the polypeptides have an N-terminus, at amino acids 36, 37, 38, 39, or 40, and a C-terminus at an amino acid corresponding to amino acids 465 to 500.

Clause 11. The combination dosing regimen of clause 7, wherein the modified PH20 comprises amino acid substitutions selected from among: T341S, L342W, S343E, I344N, M345T, S347T, M348K, K349E, L352Q, L353A, L354I, D355K, N356E, E359D and I361T.

Clause 12. The combination dosing regimen of clause 7, wherein the soluble hyaluronidase comprises amino acid modifications selected from one or more up to all of the following T341S, L342W, S343E, I344N, M345T, S347T, M348K, K349E, L352Q, L353A, L354I, D355K, N356E, E359D and I361T.

Clause 13. The combination dosing regimen of clause 7, wherein the C-terminus of variant PH20 polypeptide is at an amino acid corresponding to amino acid 467, 468, 469, 470, or 471 with reference to SEQ ID NO:1.

Clause 14. The combination dosing regimen of clause 1, wherein the soluble hyaluronidase comprises the sequence of amino acids set forth in SEQ ID NO:2 or is a catalytically active fragment thereof.

Clause 15. The combination dosing regimen of clause 1, wherein the hyaluronidase is administered at a dose of 2000 to 15,000 U.

Clause 16. The combination dosing regimen of clause 15, wherein the hyaluronidase is administered at a dose of 10,000 U.

Clause 17. The combination dosing regimen of clause 1, wherein the antibody-drug conjugate is administered at a dose of 10 mg/kg.

Clause 18. The combination dosing regimen of clause 1, wherein the antibody-drug conjugate is administered at a dose of at least or at 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 15 mg/kg, 20 mg/kg, 30 mg/kg, 40 mg/kg, 50 mg/kg, 60 mg/kg, 70 mg/kg, 80 mg/kg, 90 mg/kg, and 100 mg/kg.

Clause 19. The combination dosing regimen of clause 1, wherein the hyaluronidase and antibody-drug conjugate are administered subcutaneously.

Clause 20. The combination dosing regimen of clause 1, wherein: in a first step the soluble hyaluronidase is administered to a patient; and in a second step the antibody-drug conjugate is administered to the patient.

Clause 21. The combination dosing regimen of clause 1, wherein the soluble hyaluronidase and antibody-drug conjugate are administered in the same composition.

Clause 22. The combination dosing regimen of clause 1, wherein the soluble hyaluronidase and antibody-drug conjugate are administered once a day.

Clause 23. The combination dosing regimen of clause 22, wherein the soluble hyaluronidase and antibody-drug conjugate are administered on day one and day eight of a twenty-one-day treatment cycle for three cycles.

Clause 24. A method of treating (i) locally advanced or metastatic breast cancer, or (ii) locally advanced or metastatic urothelial cancer comprising administering to a patient in need of treatment the combination dosing regimen of clause 1.

Clause 25. The method of clause 24, wherein (i) the locally advanced or metastatic breast cancer is unresectable locally advanced or metastatic triple-negative breast cancer (mTNBC) in adult subjects who have received two or more prior systemic therapies, at least one of them for metastatic disease, or is unresectable locally advanced or metastatic hormone receptor (HR)-positive, human epidermal growth factor receptor 2 (HER2)-negative breast cancer in adult subjects who have received endocrine-based therapy and at least two additional systemic therapies in the metastatic setting, or (ii) the locally advanced or metastatic urothelial cancer is in adult subjects who have previously received a platinum-containing chemotherapy and either programmed death receptor-1 (PD-1) or programmed death-ligand 1 (PD-L1) inhibitor.

Clause 26. A combination, comprising a soluble hyaluronidase and an antibody-drug conjugate.

Clause 27. A kit, comprising the combination of clause 26.

Clause 28. The combination of clause 26, wherein the soluble hyaluronidase and the antibody-drug conjugate are in separate compositions.

Clause 29. The combination of clause 26, wherein the soluble hyaluronidase and the antibody-drug conjugate are co-formulated.

Clause 30. The combination of clause 26, wherein the soluble hyaluronidase and antibody-drug conjugate are in separate compositions in a container with at least two compartments.

Clause 31. A composition, comprising a soluble hyaluronidase and antibody-drug conjugate.

Clause 32. A multi-compartment container, comprising a suspension comprising antibody-drug conjugate in one compartment, and a soluble hyaluronidase in a second compartment.

Clause 33. The multi-compartment container of clause 32 that is a syringe, comprising two compartments.

Clause 34. The combination dosing regimen of clause 1, wherein the soluble hyaluronidase is administered at a rate of about 0.05 mL/sec to about 1.0 mL/sec.

Clause 35. The combination dosing regimen of clause 1, wherein the soluble hyaluronidase is administered at a rate of about 0.05 mL/sec to about 0.10 mL/sec.

Clause 36. The combination dosing regimen of clause 1, wherein the soluble hyaluronidase is administered at a rate of about 0.10 mL/sec to about 0.20 mL/sec.

Clause 37. The combination dosing regimen of clause 1, wherein the soluble hyaluronidase is administered at a rate of about 0.20 mL/sec to about 0.30 mL/sec.

Clause 38. The combination dosing regimen of clause 1, wherein the soluble hyaluronidase is administered at a rate of about 0.30 mL/sec to about 0.40 mL/sec.

Clause 39. The combination dosing regimen of clause 1, wherein the soluble hyaluronidase is administered at a rate of about 0.40 mL/sec to about 0.50 mL/sec.

Clause 40. The combination dosing regimen of clause 1, wherein the soluble hyaluronidase is administered at a rate of about 0.50 mL/sec to about 0.60 mL/sec.

Clause 41. The combination dosing regimen of clause 1, wherein the soluble hyaluronidase is administered at a rate of about 0.60 mL/sec to about 0.70 mL/sec.

Clause 42. The combination dosing regimen of clause 1, wherein the soluble hyaluronidase is administered at a rate of about 0.70 mL/sec to about 0.80 mL/sec.

Clause 43. The combination dosing regimen of clause 1, wherein the soluble hyaluronidase is administered at a rate of about 0.80 mL/sec to about 0.90 mL/sec.

Clause 44. The combination dosing regimen of clause 1, wherein the soluble hyaluronidase is administered at a rate of about 0.90 mL/sec to about 1.00 mL/sec.

Clause 45. The combination dosing regimen of clause 1, wherein the soluble hyaluronidase is administered at a rate of about 0.10 mL/sec to about 0.90 mL/sec.

Clause 46. The combination dosing regimen of clause 1, wherein the soluble hyaluronidase is administered at a rate of about 0.20 mL/sec to about 0.80 mL/sec.

Clause 47. The combination dosing regimen of clause 1, wherein the soluble hyaluronidase is administered at a rate of about 0.30 mL/sec to about 0.70 mL/sec.

Clause 48. The combination dosing regimen of clause 1, wherein the soluble hyaluronidase is administered at a rate of about 0.40 mL/sec to about 0.60 mL/sec.

Clause 49. The combination dosing regimen of clause 1, wherein the soluble hyaluronidase is administered at a rate of about 0.45 mL/sec to about 0.55 mL/sec.

Clause 50. The combination dosing regimen of clause 1, wherein the administration takes about 10 seconds to about 60 seconds.

Clause 51. The combination dosing regimen of clause 1, wherein the administration takes about 10 seconds to about 20 seconds.

Clause 52. The combination dosing regimen of clause 1, wherein the administration takes about 20 seconds to about 30 seconds.

Clause 53. The combination dosing regimen of clause 1, wherein the administration takes about 30 seconds to about 40 seconds.

Clause 54. The combination dosing regimen of clause 1, wherein the administration takes about 40 seconds to about 50 seconds.

Clause 55. The combination dosing regimen of clause 1, wherein the administration takes about 50 seconds to about 60 seconds.

Clause 56. The combination dosing regimen of clause 1, wherein the administration takes about 20 seconds to about 50 seconds.

Clause 57. The combination dosing regimen of clause 1, wherein the administration takes about 30 seconds to about 40 seconds.

Clause 58. The combination dosing regimen of clause 1, wherein the administration takes at least or less than about 10 seconds to about 60 seconds.

Clause 59. The combination dosing regimen of clause 1, wherein the administration takes at least or less than about 10 seconds to about 20 seconds.

Clause 60. The combination dosing regimen of clause 1, wherein the administration takes at least or less than about 20 seconds to about 30 seconds.

Clause 61. The combination dosing regimen of clause 1, wherein the administration takes at least or less than about 30 seconds to about 40 seconds.

Clause 62. The combination dosing regimen of clause 1, wherein the administration takes at least or less than about 40 seconds to about 50 seconds.

Clause 63. The combination dosing regimen of clause 1, wherein the administration takes at least or less than about 50 seconds to about 60 seconds.

Clause 64. The combination dosing regimen of clause 1, wherein the administration takes at least or less than about 20 seconds to about 50 seconds.

Clause 65. The combination dosing regimen of clause 1, wherein the administration takes at least or less than about 30 seconds to about 40 seconds.

Clause 66. The combination dosing regimen of clause 1, wherein swelling (bleb) volume is reduced following the administration into a subject when compared to a formulation that does not comprise the soluble hyaluronidase.

Clause 67. The combination dosing regimen of clause 1, wherein administration of a high volume has reduced back leakage compared to a formulation that does not comprise the soluble hyaluronidase.

Clause 68. The combination dosing regimen of clause 1, wherein the soluble hyaluronidase is administered at a dose of 2,000 to 60,000 U.

Clause 69. The combination dosing regimen of clause 1, wherein the antibody-drug conjugate is administered at a dose of at least 0.05 mg/kg.

Clause 70. The combination dosing regimen of clause 1, wherein the soluble hyaluronidase and antibody-drug conjugate are administered once a day.

Clause 71. The combination dosing regimen of clause 70, wherein the soluble hyaluronidase and antibody-drug conjugate are administered on day one, day 15 of a twenty-one-day treatment cycle.

Clause 72. The combination dosing regimen of clause 70, wherein the soluble hyaluronidase and antibody-drug conjugate are administered on day one and day 15 and day 29 of a forty-two-day treatment cycle.

Clause 73. The combination dosing regimen of clause 70, wherein the soluble hyaluronidase and antibody-drug conjugate are administered on day one and day 8 and day 15 of a twenty-eight-day treatment cycle.

Clause 74. The combination dosing regimen of clause 70, wherein the soluble hyaluronidase and antibody-drug conjugate are administered on day one and day 4 and day 7 of an induction cycle.

Clause 75. The combination dosing regimen of clause 1, wherein the soluble hyaluronidase and antibody-drug conjugate are administered Q1, Q2, Q3, or Q4.

Clause 76. The combination dosing regimen of clause 1, wherein safety profile is improved and/or the adverse events or side effects are reduced in comparison to administration of the antibody-drug conjugate without the soluble hyaluronidase.

Clause 77. The combination dosing regimen of clause 1, wherein safety profile is improved and/or the adverse events or side effects are reduced in comparison to intravenous administration of the antibody-drug conjugate without hyaluronidase.

Clause 101. A combination dosing regimen, comprising: administering a soluble hyaluronidase; and administering an antibody-drug conjugate.

Clause 102. The combination dosing regimen of clause 101, wherein the hyaluronidase is a soluble PH20 hyaluronidase.

Clause 103. The combination dosing regimen of clause 102, wherein the soluble hyaluronidase is the composition designated rHuPH20.

Clause 104. The combination dosing regimen of clause 101, wherein the hyaluronidase has the sequence set forth as residues 36-482 set forth in SEQ ID NO: 1 (i.e., SEQ ID NO: 4) or 36-483 set forth in SEQ ID NO: 1 (i.e., SEQ ID NO: 46) or has at least 98% sequence identity to the sequence set forth in SEQ ID NO: 4 or SEQ ID NO: 46.

Clause 105. The combination dosing regimen of clause 101, wherein: the soluble hyaluronidase comprises amino acids 36-464 of SEQ ID NO: 1, or comprises a sequence of amino acids that has at least 85% sequence identity to a sequence of amino acids that contains at least amino acids 36-464 of SEQ ID NO:1, and retains hyaluronidase activity.

Clause 106. The combination dosing regimen of clause 101, wherein the soluble hyaluronidase comprises a sequence of amino acids that has at least 86%, 87%, 88%, 89%, 90%, 91%, 92%, 93%, 94%, 95%, 96%, 97%, 98% or 99% sequence identity to a sequence of amino acids that contains at least amino acids 36-464 of SEQ ID NO:1 and retains hyaluronidase activity.

Clause 107. The combination dosing regimen of clause 101, wherein:
a) the soluble hyaluronidase has of amino acids set forth as residues 36-465 of SEQ ID NO: 1, 36-466 of SEQ ID NO: 1, 36-467 of SEQ ID NO: 1, 36-468 of SEQ ID NO: 1, 36-469 of SEQ ID NO: 1, 35-470 of SEQ ID NO: 1, 36-471 of SEQ ID NO: 1, 36-472 of SEQ ID NO: 1, 36-474 of SEQ ID NO: 1, 36-475 of SEQ ID NO: 1, 36-476 of SEQ ID NO: 1, 35-477 of SEQ ID NO: 1, 36-478 of SEQ ID NO: 1 (i.e., SEQ ID NO: 8), 36-479 of SEQ ID NO: 1 (i.e., SEQ ID NO: 7), 36-480 of SEQ ID NO: 1 (i.e., SEQ ID NO: 6), 36-481 of SEQ ID NO: 1 (i.e., SEQ ID NO: 5), 36-482 of SEQ ID NO: 1 (i.e., SEQ ID NO: 4), 36-483 of SEQ ID NO: 1 (i.e., SEQ ID NO: 46), 35-484 of SEQ ID NO: 1, 36-485 of SEQ ID NO: 1, 36-486 of SEQ ID NO: 1, 36-487 of SEQ ID NO: 1, 36-488 of SEQ ID NO: 1, 36-489 of SEQ ID NO: 1, 36-490 of SEQ ID NO: 1, 35-491 of SEQ ID NO: 1, 36-492 of SEQ ID NO: 1, 36-493 of SEQ ID NO: 1, 36-494 of SEQ ID NO: 1, 36-495 of SEQ ID NO: 1, 36-496 of SEQ ID NO: 1, 36-497 of SEQ ID NO: 1, 35-498 of SEQ ID NO: 1, 36-499 of SEQ ID NO: 1, and 36-500 of SEQ ID NO: 1, or an N-terminally truncated variant thereof lacking residues 36, 36-37, 36-38, 36-39, or 36-40; or
b) a variant soluble hyaluronidase that has at least 91% sequence identity to a soluble hyaluronidase of a).

Clause 108. The combination dosing regimen of clause 107, wherein the soluble hyaluronidase has at least 95% sequence identity to a soluble hyaluronidase of clause 107.

Clause 109. The combination dosing regimen of clause 107, wherein:
the soluble hyaluronidase comprises the replacement F204P, and has increased stability relative the unmodified PH20 that does not comprise F204P; and
   (a) T341S, L342W, S343E, I344N, M345T. S347T, M348K, K349E, L352Q, L353A, L3541, D355K, N356E, E359D and I361T;
   (b) L342W, S343E, I344N, M345T, S347T, M348K, K349E, L352Q, L353A, L3541, D355K, N356E, E359D and I361T;
   (c) M345T, S347T, M348K, K349E, L352Q, L353A, L354I, D355K, N356E, E359D, I361T and N363G;
   (d) T341G, L342W, S343E, I344N, M345T, S347T, M348K, K349E, L352Q, L353A, L354I, D355K, N356E, E359D and I361T;
   (e) T341A, L342W, S343E, I344N, M345T, S347T, M348K, K349E, L352Q, L353A, L354I, D355K, N356E, E359D and I361T;
   (f) T341C, L342W, S343E, I344N, M345T, S347T, M348K, K349E, L352Q, L353A, L354I, D355K, N356E, E359D and I361T;
   (g) T341D, L342W, S343E, I344N, M345T, S347T, M348K, K349E, L352Q, L353A, L354I, D355K, N356E, E359D and I361T;
   (h) I344N, M345T, S347T, M348K, K349E, L352Q, L353A, L354I, D355K, N356E, E359D and I361T, and
   (i) S343E, I344N, M345T, S347T, M348K, K349E, L352Q, L353A, L354I, D355K, N356E, E359D and I361T.
increased stability is reflected as increased stability in denaturing condition or at elevated temperatures.

Clause 110. The combination dosing regimen of clause 107, wherein:
the soluble hyaluronidase is a variant modified polypeptide or catalytically active portion thereof that comprises one or more amino acid residue substitutions selected from among T341A, T341C, T341D, T341G, T341S, L342W, S343E, I344N, M348K, and N363G;
   numbering is with reference to SEQ ID NO:1;
   modifications comprise insertions, deletions, and replacements of amino acids;
   the polypeptides have an N-terminus, at residue 36, 37, 38, 39, or 40, and a C-terminus at a residue corresponding to residues 465 to 500.

Clause 111. The combination dosing regimen of clause 107, wherein the modified PH20 comprises amino acid residue substitutions selected from among: T341S, L342W, S343E, I344N, M345T, S347T, M348K, K349E, L352Q, L353A, L354I, D355K, N356E, E359D and I361T.

Clause 112. The combination dosing regimen of clause 107, wherein the soluble hyaluronidase comprises amino acid modifications selected from one or more up to all of the following T341S, L342W, S343E, I344N, M345T, S347T, M348K, K349E, L352Q, L353A, L354I, D355K, N356E, E359D and I361T.

Clause 113. The combination dosing regimen of clause 107, wherein the C-terminus of variant PH20 polypeptide is at a residue corresponding to amino acid 467, 468, 469, 470, or 471 with reference to SEQ ID NO:1.

Clause 114. The combination dosing regimen of clause 107, wherein the soluble hyaluronidase comprises the sequence of amino acids set forth in SEQ ID NO:2 or is a catalytically active fragment thereof.

Clause 115. The combination dosing regimen of clause 101, wherein the hyaluronidase is administered at a dose of 2000 to 15,000 U.

Clause 116. The combination dosing regimen of clause 115, wherein the hyaluronidase is administered at a dose of 10,000 U.

Clause 117. The combination dosing regimen of clause 101, wherein the antibody-drug conjugate is administered at a dose of 10 mg/kg.

Clause 118. The combination dosing regimen of clause 101, wherein the antibody-drug conjugate is administered at a dose of at least or at 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 15 mg/kg, 20 mg/kg, 30 mg/kg, 40 mg/kg, 50 mg/kg, 60 mg/kg, 70 mg/kg, 80 mg/kg, 90 mg/kg, and 100 mg/kg.

Clause 119. The combination dosing regimen of clause 101, wherein the hyaluronidase and antibody-drug conjugate are administered subcutaneously.

Clause 120. The combination dosing regimen of clause 101, wherein:
in a first step the soluble hyaluronidase is administered to a patient; and
in a second step the antibody-drug conjugate is administered to the patient.

Clause 121. The combination dosing regimen of clause 101, wherein the soluble hyaluronidase and antibody-drug conjugate are administered in the same composition.

Clause 122. The combination dosing regimen of clause 101, wherein the soluble hyaluronidase and antibody-drug conjugate are administered once a day.

Clause 123. The combination dosing regimen of clause 122, wherein the soluble hyaluronidase and antibody-drug conjugate are administered on day one and day eight of a twenty-one-day treatment cycle for three cycles.

Clause 124. A method of treating (i) locally advanced or metastatic breast cancer, or (ii) locally advanced or metastatic urothelial cancer comprising administering to a patient in need of treatment the combination dosing regimen of clause 101.

Clause 125. The method of clause 124, wherein (i) the locally advanced or metastatic breast cancer is unresectable locally advanced or metastatic triple-negative breast cancer (mTNBC) in adult subjects who have received two or more prior systemic therapies, at least one of them for metastatic disease, or is unresectable locally advanced or metastatic hormone receptor (HR)-positive, human epidermal growth factor receptor 2 (HER2)-negative breast cancer in adult subjects who have received endocrine-based therapy and at least two additional systemic therapies in the metastatic setting, or (ii) the locally advanced or metastatic urothelial cancer is in adult subjects who have previously received a platinum-containing chemotherapy and either programmed death receptor-1 (PD-1) or programmed death-ligand 1 (PD-L1) inhibitor.

Clause 126. A combination, comprising a soluble hyaluronidase and an antibody-drug conjugate.

Clause 127. A kit, comprising the combination of clause 126.

Clause 128. The combination of clause 128, wherein the soluble hyaluronidase and the antibody-drug conjugate are in separate compositions.

Clause 129. The combination of clause 128, wherein the soluble hyaluronidase and the antibody-drug conjugate are co-formulated.

Clause 130. The combination of clause 128, wherein the soluble hyaluronidase and antibody-drug conjugate are in separate compositions in a container with at least two compartments.

Clause 131. A composition, comprising a soluble hyaluronidase and antibody-drug conjugate.

Clause 132. A multi-compartment container, comprising a suspension comprising antibody-drug conjugate in one compartment, and a soluble hyaluronidase in a second compartment.

Clause 133. The multi-compartment container of clause 132 that is a syringe, comprising two compartments.

Clause 134. The combination dosing regimen of clause 101, wherein the soluble hyaluronidase is administered at a rate of about 0.05 mL/sec to about 1.0 mL/sec.

Clause 135. The combination dosing regimen of clause 101, wherein the soluble hyaluronidase is administered at a rate of about 0.05 mL/sec to about 0.10 mL/sec.

Clause 136. The combination dosing regimen of clause 101, wherein the soluble hyaluronidase is administered at a rate of about 0.10 mL/sec to about 0.20 mL/sec.

Clause 137. The combination dosing regimen of clause 101, wherein the soluble hyaluronidase is administered at a rate of about 0.20 mL/sec to about 0.30 mL/sec.

Clause 138. The combination dosing regimen of clause 101, wherein the soluble hyaluronidase is administered at a rate of about 0.30 mL/sec to about 0.40 mL/sec.

Clause 139. The combination dosing regimen of clause 101, wherein the soluble hyaluronidase is administered at a rate of about 0.40 mL/sec to about 0.50 mL/sec.

Clause 140. The combination dosing regimen of clause 101, wherein the soluble hyaluronidase is administered at a rate of about 0.50 mL/sec to about 0.60 mL/sec.

Clause 141. The combination dosing regimen of clause 101, wherein the soluble hyaluronidase is administered at a rate of about 0.60 mL/sec to about 0.70 mL/sec.

Clause 142. The combination dosing regimen of clause 101, wherein the soluble hyaluronidase is administered at a rate of about 0.70 mL/sec to about 0.80 mL/sec.

Clause 143. The combination dosing regimen of clause 101, wherein the soluble hyaluronidase is administered at a rate of about 0.80 mL/sec to about 0.90 mL/sec.

Clause 144. The combination dosing regimen of clause 101, wherein the soluble hyaluronidase is administered at a rate of about 0.90 mL/sec to about 1.00 mL/sec.

Clause 145. The combination dosing regimen of clause 101, wherein the soluble hyaluronidase is administered at a rate of about 0.10 mL/sec to about 0.90 mL/sec.

Clause 146. The combination dosing regimen of clause 101, wherein the soluble hyaluronidase is administered at a rate of about 0.20 mL/sec to about 0.80 mL/sec.

Clause 147. The combination dosing regimen of clause 101, wherein the soluble hyaluronidase is administered at a rate of about 0.30 mL/sec to about 0.70 mL/sec.

Clause 148. The combination dosing regimen of clause 101, wherein the soluble hyaluronidase is administered at a rate of about 0.40 mL/sec to about 0.60 mL/sec.

Clause 149. The combination dosing regimen of clause 101, wherein the soluble hyaluronidase is administered at a rate of about 0.45 mL/sec to about 0.55 mL/sec.

Clause 150. The combination dosing regimen of clause 101, wherein the administration takes about 10 seconds to about 60 seconds.

Clause 151. The combination dosing regimen of clause 101, wherein the administration takes about 10 seconds to about 20 seconds.

Clause 152. The combination dosing regimen of clause 101, wherein the administration takes about 20 seconds to about 30 seconds.

Clause 153. The combination dosing regimen of clause 101, wherein the administration takes about 30 seconds to about 40 seconds.

Clause 154. The combination dosing regimen of clause 101, wherein the administration takes about 40 seconds to about 50 seconds.

Clause 155. The combination dosing regimen of clause 101, wherein the administration takes about 50 seconds to about 60 seconds.

Clause 156. The combination dosing regimen of clause 101, wherein the administration takes about 20 seconds to about 50 seconds.

Clause 157. The combination dosing regimen of clause 101, wherein the administration takes about 30 seconds to about 40 seconds.

Clause 158. The combination dosing regimen of clause 101, wherein the administration takes at least or less than about 10 seconds to about 60 seconds.

Clause 159. The combination dosing regimen of clause 101, wherein the administration takes at least or less than about 10 seconds to about 20 seconds.

Clause 160. The combination dosing regimen of clause 101, wherein the administration takes at least or less than about 20 seconds to about 30 seconds.

Clause 161. The combination dosing regimen of clause 101, wherein the administration takes at least or less than about 30 seconds to about 40 seconds.

Clause 162. The combination dosing regimen of clause 101, wherein the administration takes at least or less than about 40 seconds to about 50 seconds.

Clause 163. The combination dosing regimen of clause 101, wherein the administration takes at least or less than about 50 seconds to about 60 seconds.

Clause 164. The combination dosing regimen of clause 101, wherein the administration takes at least or less than about 20 seconds to about 50 seconds.

Clause 165. The combination dosing regimen of clause 101, wherein the administration takes at least or less than about 30 seconds to about 40 seconds.

Clause 166. The combination dosing regimen of clause 101, wherein swelling (bleb) volume is reduced following the administration into a subject when compared to a formulation that does not comprise the soluble hyaluronidase.

Clause 167. The combination dosing regimen of clause 101, wherein administration of a high volume has reduced back leakage compared to a formulation that does not comprise the soluble hyaluronidase.

Clause 168. The combination dosing regimen of clause 101, wherein the soluble hyaluronidase is administered at a dose of 2,000 to 60,000 U.

Clause 169. The combination dosing regimen of clause 101, wherein the antibody-drug conjugate is administered at a dose of at least 0.05 mg/kg.

Clause 170. The combination dosing regimen of clause 101, wherein the soluble hyaluronidase and antibody-drug conjugate are administered once a day.

Clause 171. The combination dosing regimen of clause 170, wherein the soluble hyaluronidase and antibody-drug conjugate are administered on day one, day 15 of a twenty-one-day treatment cycle.

Clause 172. The combination dosing regimen of clause 170, wherein the soluble hyaluronidase and antibody-drug conjugate are administered on day one and day 15 and day 29 of a forty-two-day treatment cycle.

Clause 173. The combination dosing regimen of clause 170, wherein the soluble hyaluronidase and antibody-drug conjugate are administered on day one and day 8 and day 15 of a twenty-eight-day treatment cycle.

Clause 174. The combination dosing regimen of clause 170, wherein the soluble hyaluronidase and antibody-drug conjugate are administered on day one and day 4 and day 7 of an induction cycle.

Clause 175. The combination dosing regimen of clause 101, wherein the soluble hyaluronidase and antibody-drug conjugate are administered Q1, Q2, Q3, or Q4.

Clause 176. The combination dosing regimen of clause 101, wherein safety profile is improved and/or the adverse events or side effects are reduced in comparison to administration of the antibody-drug conjugate without the soluble hyaluronidase.

Clause 177. The combination dosing regimen of clause 101, wherein safety profile is improved and/or the adverse events or side effects are reduced in comparison to intravenous administration of the antibody-drug conjugate without hyaluronidase.

Clause 178. A combination dosing regimen comprising subcutaneously administering to a human subject a therapeutically effective amount of an antibody drug conjugate; and administering a soluble hyaluronidase.

Clause 179. The combination dosing regimen of Clause 178, wherein a detectable amount of intact antibody drug conjugate reaches systemic circulation.

Clause 180. The combination dosing regimen of Clause 179, wherein serum levels of the antibody drug conjugate are between 10-200% of the equivalent intravenous dose of the antibody drug conjugate.

Clause 181. The combination dosing regimen of Clause 178, 179 or 181, wherein the antibody drug conjugate comprises a monoclonal antibody.

Clause 182. The combination dosing regimen of Clause 181, wherein serum levels of the monoclonal antibody are between 10-200% of the equivalent intravenous dose.

Clause 183. The combination dosing regimen of any preceding Clause, wherein the antibody drug conjugate comprises a payload.

Clause 184. The combination dosing regimen of Clause 183, wherein serum levels of the free payload are between 10-200% of the equivalent intravenous dose.

Clause 185. The combination dosing regimen of Clause 180, wherein the serum levels are taken 6 hours after administration.

Clause 186. The combination dosing regimen of Clause 180, wherein the serum levels are taken 24 hours after administration.

Clause 187. The combination dosing regimen of Clause 180, wherein the serum levels are taken 48 hours after administration.

Clause 188. The combination dosing regimen of Clause 180, wherein the serum levels are taken 72 hours after administration.

Clause 189. The combination dosing regimen of Clause 180, wherein the serum levels are taken 96 hours after administration.

Clause 190. The combination dosing regimen of Clause 180, wherein the serum levels are taken 168 hours after administration.

Clause 191. The combination dosing regimen of Clause 180, wherein the serum levels are taken 336 hours after administration.

Clause 192. The combination dosing regimen of Clause 180, wherein AUC is at least 10% of an equivalent intravenous dose of the antibody drug conjugate.

Clause 193. The combination dosing regimen of Clause 180, wherein AUC is at least 20% of an equivalent intravenous dose of the antibody drug conjugate.

Clause 194. The combination dosing regimen of Clause 180, wherein AUC is at least 30% of an equivalent intravenous dose of the antibody drug conjugate.

Clause 195. The combination dosing regimen of Clause 180, wherein AUC is at least 40% of an equivalent intravenous dose of the antibody drug conjugate.

Clause 196. The combination dosing regimen of Clause 180, wherein AUC is at least 50% of an equivalent intravenous dose of the antibody drug conjugate.

Clause 197. The combination dosing regimen of Clause 180, wherein AUC is at least 60% of an equivalent intravenous dose of the antibody drug conjugate.

Clause 198. The combination dosing regimen of Clause 180, wherein AUC is at least 70% of an equivalent intravenous dose of the antibody drug conjugate.

Clause 199. The combination dosing regimen of Clause 180, wherein AUC is at least 80% of an equivalent intravenous dose of the antibody drug conjugate.

Clause 200. The combination dosing regimen of Clause 180, wherein AUC is at least 90% of an equivalent intravenous dose of the antibody drug conjugate.

Clause 201. The combination dosing regimen of Clause 180, wherein AUC is at least 100% of an equivalent intravenous dose of the antibody drug conjugate.

Clause 202. The combination dosing regimen of Clause 180, wherein AUC is at least 110% of an equivalent intravenous dose of the antibody drug conjugate.

Clause 203. The combination dosing regimen of Clause 180, wherein AUC is at least 120% of an equivalent intravenous dose of the antibody drug conjugate.

Clause 204. The combination dosing regimen of Clause 180, wherein AUC is at least 130% of an equivalent intravenous dose of the antibody drug conjugate.

Clause 205. The combination dosing regimen of Clause 180, wherein AUC is at least 140% of an equivalent intravenous dose of the antibody drug conjugate.

Clause 206. The combination dosing regimen of Clause 180, wherein AUC is at least 150% of an equivalent intravenous dose of the antibody drug conjugate.

Clause 207. The combination dosing regimen of Clause 180, wherein AUC is at least 160% of an equivalent intravenous dose of the antibody drug conjugate.

Clause 208. The combination dosing regimen of Clause 180, wherein AUC is at least 170% of an equivalent intravenous dose of the antibody drug conjugate.

Clause 209. The combination dosing regimen of Clause 180, wherein AUC is at least 180% of an equivalent intravenous dose of the antibody drug conjugate.

Clause 210. The combination dosing regimen of Clause 180, wherein AUC is at least 190% of an equivalent intravenous dose of the antibody drug conjugate.

Clause 211. The combination dosing regimen of Clause 180, wherein AUC is at least 200% of an equivalent intravenous dose of the antibody drug conjugate.

Clause 212. The combination dosing regimen of Clause 180, wherein Cmin is at least 10% of an equivalent intravenous dose of the antibody drug conjugate.

Clause 213. The combination dosing regimen of Clause 180, wherein Cmin is at least 20% of an equivalent intravenous dose of the antibody drug conjugate.

Clause 214. The combination dosing regimen of Clause 180, wherein Cmin is at least 30% of an equivalent intravenous dose of the antibody drug conjugate.

Clause 215. The combination dosing regimen of Clause 180, wherein Cmin is at least 40% of an equivalent intravenous dose of the antibody drug conjugate.

Clause 216. The combination dosing regimen of Clause 180, wherein Cmin is at least 50% of an equivalent intravenous dose of the antibody drug conjugate.

Clause 217. The combination dosing regimen of Clause 180, wherein Cmin is at least 60% of an equivalent intravenous dose of the antibody drug conjugate.

Clause 218. The combination dosing regimen of Clause 180, wherein Cmin is at least 70% of an equivalent intravenous dose of the antibody drug conjugate.

Clause 219. The combination dosing regimen of Clause 180, wherein Cmin is at least 80% of an equivalent intravenous dose of the antibody drug conjugate.

Clause 220. The combination dosing regimen of Clause 180, wherein Cmin is at least 90% of an equivalent intravenous dose of the antibody drug conjugate.

Clause 221. The combination dosing regimen of Clause 180, wherein Cmin is at least 100% of an equivalent intravenous dose of the antibody drug conjugate.

Clause 222. The combination dosing regimen of Clause 180, wherein Cmin is at least 110% of an equivalent intravenous dose of the antibody drug conjugate.

Clause 223. The combination dosing regimen of Clause 180, wherein Cmin is at least 120% of an equivalent intravenous dose of the antibody drug conjugate.

Clause 224. The combination dosing regimen of Clause 180, wherein Cmin is at least 130% of an equivalent intravenous dose of the antibody drug conjugate.

Clause 225. The combination dosing regimen of Clause 180, wherein Cmin is at least 140% of an equivalent intravenous dose of the antibody drug conjugate.

Clause 226. The combination dosing regimen of Clause 180, wherein Cmin is at least 150% of an equivalent intravenous dose of the antibody drug conjugate.

Clause 227. The combination dosing regimen of Clause 180, wherein Cmin is at least 160% of an equivalent intravenous dose of the antibody drug conjugate.

Clause 228. The combination dosing regimen of Clause 180, wherein Cmin is at least 170% of an equivalent intravenous dose of the antibody drug conjugate.

Clause 229. The combination dosing regimen of Clause 180, wherein Cmin is at least 180% of an equivalent intravenous dose of the antibody drug conjugate.

Clause 230. The combination dosing regimen of Clause 180, wherein Cmin is at least 190% of an equivalent intravenous dose of the antibody drug conjugate.

Clause 231. The combination dosing regimen of Clause 180, wherein Cmin is at least 200% of an equivalent intravenous dose of the antibody drug conjugate.

Clause 232. The combination dosing regimen of Clause 180, wherein Cmax is less than 100% of an equivalent intravenous dose of the antibody drug conjugate.

Clause 233. The combination dosing regimen of Clause 180, wherein Cmax is less than 90% of an equivalent intravenous dose of the antibody drug conjugate.

Clause 234. The combination dosing regimen of Clause 180, wherein Cmax is less than 80% of an equivalent intravenous dose of the antibody drug conjugate.

Clause 235. The combination dosing regimen of Clause 180, wherein Cmax is less than 70% of an equivalent intravenous dose of the antibody drug conjugate.

Clause 236. The combination dosing regimen of Clause 180, wherein Cmax is less than 60% of an equivalent intravenous dose of the antibody drug conjugate.

Clause 237. The combination dosing regimen of Clause 180, wherein Cmax is less than 50% of an equivalent intravenous dose of the antibody drug conjugate.

Clause 238. The combination dosing regimen of Clause 180, wherein Cmax is less than 40% of an equivalent intravenous dose of the antibody drug conjugate.

Clause 239. The combination dosing regimen of Clause 180, wherein Cmax is less than 30% of an equivalent intravenous dose of the antibody drug conjugate.

Clause 240. The combination dosing regimen of Clause 180, wherein Cmax is less than 20% of an equivalent intravenous dose of the antibody drug conjugate.

Clause 241. The combination dosing regimen of Clause 180, wherein Cmax is less than 10% of an equivalent intravenous dose of the antibody drug conjugate.

Clause 242. The combination dosing regimen of Clause 180, wherein the time to Cmax (Tmax) for the antibody drug conjugate is greater than 2 hours.

Clause 243. The combination dosing regimen of Clause 180, wherein the time to Cmax (Tmax) for the antibody drug conjugate is greater than 4 hours.

Clause 244. The combination dosing regimen of Clause 180, wherein the time to Cmax (Tmax) for the antibody drug conjugate is greater than 6 hours.

Clause 245. The combination dosing regimen of Clause 180, wherein the time to Cmax (Tmax) for the antibody drug conjugate is greater than 12 hours.

Clause 246. The combination dosing regimen of Clause 180, wherein the time to Cmax (Tmax) for the antibody drug conjugate is greater than 24 hours.

Clause 247. The combination dosing regimen of Clause 180, wherein the time to Cmax (Tmax) for the antibody drug conjugate is greater than 48 hours.

Clause 248. The combination dosing regimen of Clause 180, wherein the time to Cmax (Tmax) for the antibody drug conjugate is greater than 72 hours.

Clause 249. The combination dosing regimen of Clause 180, wherein the time to Cmax (Tmax) for the antibody drug conjugate is greater than 96 hours.

Clause 250. The combination dosing regimen of Clause 180, wherein the time to Cmax (Tmax) for the antibody drug conjugate is greater than 120 hours.

Clause 251. The combination dosing regimen of Clause 180, wherein the time to Cmax (Tmax) for the antibody drug conjugate is greater than 144 hours.

Clause 252. The combination dosing regimen of Clause 180, wherein the time to Cmax (Tmax) for the antibody drug conjugate is greater than 168 hours.

Clause 253. The combination dosing regimen of Clause 178, wherein the antibody drug conjugate comprises a monoclonal antibody.

Clause 254. The combination dosing regimen of Clause 253, wherein the time to Cmax (Tmax) for the monoclonal antibody is greater than 2 hours.

Clause 255. The combination dosing regimen of Clause 253, wherein the time to Cmax (Tmax) for the monoclonal antibody is greater than 4 hours.

Clause 256. The combination dosing regimen of Clause 253, wherein the time to Cmax (Tmax) for the monoclonal antibody is greater than 6 hours.

Clause 257. The combination dosing regimen of Clause 253, wherein the time to Cmax (Tmax) for the monoclonal antibody is greater than 12 hours.

Clause 258. The combination dosing regimen of Clause 253, wherein the time to Cmax (Tmax) for the monoclonal antibody is greater than 24 hours.

Clause 259. The combination dosing regimen of Clause 253, wherein the time to Cmax (Tmax) for the monoclonal antibody is greater than 48 hours.

Clause 260. The combination dosing regimen of Clause 253, wherein the time to Cmax (Tmax) for the monoclonal antibody is greater than 72 hours.

Clause 261. The combination dosing regimen of Clause 253, wherein the time to Cmax (Tmax) for the monoclonal antibody is greater than 96 hours.

Clause 262. The combination dosing regimen of Clause 253, wherein the time to Cmax (Tmax) for the monoclonal antibody is greater than 120 hours.

Clause 263. The combination dosing regimen of Clause 253, wherein the time to Cmax (Tmax) for the monoclonal antibody is greater than 144 hours.

Clause 264. The combination dosing regimen of Clause 253, wherein the time to Cmax (Tmax) for the monoclonal antibody is greater than 168 hours.

Clause 265. The combination dosing regimen of Clause 178, wherein a higher dose of the antibody drug conjugate is delivered subcutaneously than a therapeutically effective dose administered intravenously with an equal or lower Cmax.

Clause 266. The combination dosing regimen of Clause 265, wherein the equal or lower Cmax is for the antibody drug conjugate.

Clause 267. The combination dosing regimen of Clause 266, wherein the antibody drug conjugate comprises a monoclonal antibody.

Clause 268. The combination dosing regimen of Clause 267, wherein the equal or lower Cmax is for the monoclonal antibody.

Clause 269. The combination dosing regimen of Clause 267, wherein the antibody drug conjugate comprises a payload.

Clause 270. The combination dosing regimen of Clause 267, wherein the equal or lower Cmax is for the free payload.

Clause 271. The combination dosing regimen of Clause 267, wherein the amount of dose delivered subcutaneously is at least 10% higher than a therapeutically effective intravenous dose.

Clause 272. The combination dosing regimen of Clause 267, wherein the amount of dose delivered subcutaneously is at least 50% higher than a therapeutically effective intravenous dose.

Clause 273. The combination dosing regimen of Clause 267, wherein the amount of dose delivered subcutaneously is at least 100% higher than a therapeutically effective intravenous dose.

Clause 274. The combination dosing regimen of Clause 267, wherein the amount of dose delivered subcutaneously is at least 200% higher than a therapeutically effective intravenous dose.

Clause 275. The combination dosing regimen of Clause 267, wherein the amount of dose delivered subcutaneously is at least 300% higher than a therapeutically effective intravenous dose.

Clause 276. The combination dosing regimen of Clause 267, wherein the amount of dose delivered subcutaneously is at least 400% higher than a therapeutically effective intravenous dose.

Clause 277. The combination dosing regimen of Clause 267, wherein the amount of dose delivered subcutaneously is at least 500% higher than a therapeutically effective intravenous dose.

Clause 278. The combination dosing regimen of Clause 267, wherein there is a higher lymphatic concentration relative to an equivalent intravenous dose.

Clause 279. The combination dosing regimen of Clause 278, wherein the lymphatic concentration is at least 100% of an equivalent intravenous dose.

Clause 280. The combination dosing regimen of Clause 278, wherein the lymphatic concentration is at least 150% of an equivalent intravenous dose.

Clause 281. The combination dosing regimen of Clause 278, wherein the lymphatic concentration is at least 200% of an equivalent intravenous dose.

Clause 282. The combination dosing regimen of Clause 278, wherein the lymphatic concentration is at least 250% of an equivalent intravenous dose.

Clause 283. The combination dosing regimen of Clause 278, wherein the lymphatic concentration is at least 300% of an equivalent intravenous dose.

Clause 284. The combination dosing regimen of Clause 278, wherein the lymphatic concentration is at least 350% of an equivalent intravenous dose.

Clause 285. The combination dosing regimen of Clause 278, wherein the lymphatic concentration is at least 400% of an equivalent intravenous dose.

Clause 286. The combination dosing regimen of Clause 278, wherein the lymphatic concentration is at least 450% of an equivalent intravenous dose.

Clause 287. The combination dosing regimen of Clause 278, wherein the lymphatic concentration is at least 500% of an equivalent intravenous dose.

Clause 288. The combination dosing regimen of Clause 178, wherein there is an increase in the serum level of antibody drug conjugate relative to administration without hyaluronidase.

Clause 289. The combination dosing regimen of Clause 288, wherein the increase in an increase in antibody drug conjugate.

Clause 290. The combination dosing regimen of Clause 288, wherein the antibody drug conjugate comprises a monoclonal antibody.

Clause 291. The combination dosing regimen of Clause 290, wherein the increase is an increase in monoclonal antibody.

Clause 292. The combination dosing regimen of Clause 288, wherein the antibody drug conjugate has a payload, and the increase is an increase in the free payload.

Clause 293. The combination dosing regimen of any of Clauses 288 to 291, wherein the serum level is taken 6 hours after administration.

Clause 294. The combination dosing regimen of any of Clauses 288 to 291, wherein the serum level is taken 24 hours after administration.

Clause 295. The combination dosing regimen of any of Clauses 288 to 291, wherein the serum level is taken 48 hours after administration.

Clause 296. The combination dosing regimen of any of Clauses 288 to 291, wherein the serum level is taken 72 hours after administration.

Clause 297. The combination dosing regimen of any of Clauses 288 to 291, wherein the serum level is taken 96 hours after administration.

Clause 298. The combination dosing regimen of any of Clauses 288 to 291, wherein the serum level is taken 168 hours after administration.

Clause 299. The combination dosing regimen of any of Clauses 288 to 291, wherein the serum level is taken 336 hours after administration.

Clause 300. The combination dosing regimen of Clause 288 or Clauses 293-299, wherein AUC is greater than 100% relative to delivery of the antibody drug conjugate without hyaluronidase.

Clause 301. The combination dosing regimen of Clause 288 or Clauses 293-299, wherein AUC is greater than 110% relative to delivery of the antibody drug conjugate without hyaluronidase.

Clause 302. The combination dosing regimen of Clause 288 or Clauses 293-299, wherein AUC is greater than 120% relative to delivery of the antibody drug conjugate without hyaluronidase.

Clause 303. The combination dosing regimen of Clause 288 or Clauses 293-299, wherein AUC is greater than 130% relative to delivery of the antibody drug conjugate without hyaluronidase.

Clause 304. The combination dosing regimen of Clause 288 or Clauses 293-299, wherein AUC is greater than 140% relative to delivery of the antibody drug conjugate without hyaluronidase.

Clause 305. The combination dosing regimen of Clause 288 or Clauses 293-299, wherein AUC is greater than 150% relative to delivery of the antibody drug conjugate without hyaluronidase.

Clause 306. The combination dosing regimen of Clause 288 or Clauses 293-299, wherein AUC is greater than 160% relative to delivery of the antibody drug conjugate without hyaluronidase.

Clause 307. The combination dosing regimen of Clause 288 or Clauses 293-299, wherein AUC is greater than 170% relative to delivery of the antibody drug conjugate without hyaluronidase.

Clause 308. The combination dosing regimen of Clause 288 or Clauses 293-299, wherein AUC is greater than 180% relative to delivery of the antibody drug conjugate without hyaluronidase.

Clause 309. The combination dosing regimen of Clause 288 or Clauses 293-299, wherein AUC is greater than 190% relative to delivery of the antibody drug conjugate without hyaluronidase.

Clause 310. The combination dosing regimen of Clause 288 or Clauses 293-299, wherein AUC is greater than 200% relative to delivery of the antibody drug conjugate without hyaluronidase.

Clause 311. The combination dosing regimen of Clause 288 or Clauses 293-299, wherein Cmin is greater than 100% relative to delivery of the antibody drug conjugate without hyaluronidase.

Clause 312. The combination dosing regimen of Clause 288 or Clauses 293-299, wherein Cmin is greater than 110% relative to delivery of the antibody drug conjugate without hyaluronidase.

Clause 313. The combination dosing regimen of Clause 288 or Clauses 293-299, wherein Cmin is greater than 120% relative to delivery of the antibody drug conjugate without hyaluronidase.

Clause 314. The combination dosing regimen of Clause 288 or Clauses 293-299, wherein Cmin is greater than 130% relative to delivery of the antibody drug conjugate without hyaluronidase.

Clause 315. The combination dosing regimen of Clause 288 or Clauses 293-299, wherein Cmin is greater than 140% relative to delivery of the antibody drug conjugate without hyaluronidase.

Clause 316. The combination dosing regimen of Clause 288 or Clauses 293-299, wherein Cmin is greater than 150% relative to delivery of the antibody drug conjugate without hyaluronidase.

Clause 317. The combination dosing regimen of Clause 288 or Clauses 293-299, wherein Cmin is greater than 160% relative to delivery of the antibody drug conjugate without hyaluronidase.

Clause 318. The combination dosing regimen of Clause 288 or Clauses 293-299, wherein Cmin is greater than 170% relative to delivery of the antibody drug conjugate without hyaluronidase.

Clause 319. The combination dosing regimen of Clause 288 or Clauses 293-299, wherein Cmin is greater than 180% relative to delivery of the antibody drug conjugate without hyaluronidase.

Clause 320. The combination dosing regimen of Clause 288 or Clauses 293-299, wherein Cmin is greater than 190% relative to delivery of the antibody drug conjugate without hyaluronidase.

Clause 321. The combination dosing regimen of Clause 288 or Clauses 293-299, wherein Cmin is greater than 200% relative to delivery of the antibody drug conjugate without hyaluronidase.

Clause 322. The combination dosing regimen of Clause 288 or Clauses 293-299, wherein Cmax is greater than 100% relative to delivery of the antibody drug conjugate without hyaluronidase.

Clause 323. The combination dosing regimen of Clause 288 or Clauses 293-299, wherein Cmax is greater than 110% relative to delivery of the antibody drug conjugate without hyaluronidase.

Clause 324. The combination dosing regimen of Clause 288 or Clauses 293-299, wherein Cmax is greater than 120% relative to delivery of the antibody drug conjugate without hyaluronidase.

Clause 325. The combination dosing regimen of Clause 288 or Clauses 293-299, wherein Cmax is greater than 130% relative to delivery of the antibody drug conjugate without hyaluronidase.

Clause 326. The combination dosing regimen of Clause 288 or Clauses 293-299, wherein Cmax is greater than 140% relative to delivery of the antibody drug conjugate without hyaluronidase.

Clause 327. The combination dosing regimen of Clause 288 or Clauses 293-299, wherein Cmax is greater than 150% relative to delivery of the antibody drug conjugate without hyaluronidase.

Clause 328. The combination dosing regimen of Clause 288 or Clauses 293-299, wherein Cmax is greater than 160% relative to delivery of the antibody drug conjugate without hyaluronidase.

Clause 329. The combination dosing regimen of Clause 288 or Clauses 293-299, wherein Cmax is greater than 170% relative to delivery of the antibody drug conjugate without hyaluronidase.

Clause 330. The combination dosing regimen of Clause 288 or Clauses 293-299, wherein Cmax is greater than 180% relative to delivery of the antibody drug conjugate without hyaluronidase.

Clause 331. The combination dosing regimen of Clause 288 or Clauses 293-299, wherein Cmax is greater than 190% relative to delivery of the antibody drug conjugate without hyaluronidase.

Clause 332. The combination dosing regimen of Clause 288 or Clauses 293-299, wherein Cmax is greater than 200% relative to delivery of the antibody drug conjugate without hyaluronidase.

Clause 333. The combination dosing regimen of Clause 178, wherein there is a lower concentration of antibody drug conjugate in the subcutaneous tissue relative to administration without hyaluronidase.

Clause 334. The combination dosing regimen of Clause 265, wherein there is a lower concentration of antibody drug conjugate in the subcutaneous tissue relative to administration without hyaluronidase.

Clause 335. The combination dosing regimen of Clause 288, wherein the antibody drug conjugate comprises a monoclonal antibody.

Clause 336. The combination dosing regimen of Clause 335, wherein the lower concentration is a lower concentration of monoclonal antibody.

Clause 337. The combination dosing regimen of Clause 335, wherein the antibody drug conjugate has a payload, and the lower concentration is a lower concentration in the free payload.

Clause 338. The combination dosing regimen of any of Clauses 335 to 337, wherein the subcutaneous tissue level is taken 2 hours after administration.

Clause 339. The combination dosing regimen of any of Clauses 335 to 337, wherein the subcutaneous tissue level is taken 6 hours after administration.

Clause 340. The combination dosing regimen of any of Clauses 335 to 337, wherein the subcutaneous tissue level is taken 12 hours after administration.

Clause 341. The combination dosing regimen of any of Clauses 335 to 337, wherein the subcutaneous tissue level is taken 24 hours after administration.

Clause 342. The combination dosing regimen of any of Clauses 258 to 260, wherein the subcutaneous tissue level is taken 48 hours after administration.

Clause 343. The combination dosing regimen of any of Clauses 335 to 337, wherein the subcutaneous tissue level is taken 168 hours after administration.

Clause 344. The combination dosing regimen of any of Clauses 335 to 337, wherein the subcutaneous tissue level is taken 336 hours after administration.

Clause 345. The combination dosing regimen of Clause 334, wherein the subcutaneous tissue concentration is less than 100% relative to delivery of the total antibody drug conjugate without hyaluronidase.

Clause 346. The combination dosing regimen of Clause 334, wherein the subcutaneous tissue concentration is less than 90% relative to delivery of the total antibody drug conjugate without hyaluronidase.

Clause 347. The combination dosing regimen of Clause 334, wherein the subcutaneous tissue concentration is less than 80% relative to delivery of the total antibody drug conjugate without hyaluronidase.

Clause 348. The combination dosing regimen of Clause 334, wherein the subcutaneous tissue concentration is less than 70% relative to delivery of the total antibody drug conjugate without hyaluronidase.

Clause 349. The combination dosing regimen of Clause 334, wherein the subcutaneous tissue concentration is less than 60% relative to delivery of the total antibody drug conjugate without hyaluronidase.

Clause 350. The combination dosing regimen of Clause 334, wherein the subcutaneous tissue concentration is less than 50% relative to delivery of the total antibody drug conjugate without hyaluronidase.

Clause 351. The combination dosing regimen of Clause 334, wherein the subcutaneous tissue concentration is less than 60% relative to delivery of the total antibody drug conjugate without hyaluronidase.

Clause 352. The combination dosing regimen of Clause 334, wherein the subcutaneous tissue concentration is less than 50% relative to delivery of the total antibody drug conjugate without hyaluronidase.

Clause 353. The combination dosing regimen of Clause 334, wherein the subcutaneous tissue concentration is less than 40% relative to delivery of the total antibody drug conjugate without hyaluronidase.

Clause 354. The combination dosing regimen of Clause 334, wherein the subcutaneous tissue concentration is less than 30% relative to delivery of the total antibody drug conjugate without hyaluronidase.

Clause 355. The combination dosing regimen of Clause 334, wherein the subcutaneous tissue concentration is less than 20% relative to delivery of the total antibody drug conjugate without hyaluronidase.

Clause 356. The combination dosing regimen of Clause 336, wherein the subcutaneous tissue concentration of the monoclonal antibody is less than 100% relative to delivery without hyaluronidase.

Clause 357. The combination dosing regimen of Clause 236, wherein the subcutaneous tissue concentration of the monoclonal antibody is less than is less than 90% relative to delivery without hyaluronidase.

Clause 358. The combination dosing regimen of Clause 336, wherein the subcutaneous tissue concentration of the monoclonal antibody is less than is less than 80% relative to delivery without hyaluronidase.

Clause 359. The combination dosing regimen of Clause 336, wherein the subcutaneous tissue concentration of the monoclonal antibody is less than is less than 70% relative to delivery without hyaluronidase

Clause 360. The combination dosing regimen of Clause 336, wherein the subcutaneous tissue concentration of the monoclonal antibody is less than is less than 60% relative to delivery without hyaluronidase

Clause 361. The combination dosing regimen of Clause 336, wherein the subcutaneous tissue concentration of the monoclonal antibody is less than is less than 50% relative to delivery without hyaluronidase

Clause 362. The combination dosing regimen of Clause 336, wherein the subcutaneous tissue concentration of the monoclonal antibody is less than is less than 40% relative to delivery without hyaluronidase

Clause 363. The combination dosing regimen of Clause 336, wherein the subcutaneous tissue concentration of the monoclonal antibody is less than is less than 30% relative to delivery without hyaluronidase

Clause 364. The combination dosing regimen of Clause 336, wherein the subcutaneous tissue concentration of the monoclonal antibody is less than is less than 20% relative to delivery without hyaluronidase

Clause 365. The combination dosing regimen of Clause 336, wherein the subcutaneous tissue concentration of the monoclonal antibody is less than is less than 10% relative to delivery without hyaluronidase

Clause 366. The combination dosing regimen of Clause 337, wherein the subcutaneous tissue concentration of the free payload is less than is less than 100% relative to delivery without hyaluronidase.

Clause 367. The combination dosing regimen of Clause 337, wherein the subcutaneous tissue concentration of the free payload is less than is less than 90% relative to delivery without hyaluronidase.

Clause 368. The combination dosing regimen of Clause 337, wherein the subcutaneous tissue concentration of the free payload is less than is less than 80% relative to delivery without hyaluronidase.

Clause 369. The combination dosing regimen of Clause 337, wherein the subcutaneous tissue concentration of the free payload is less than is less than 70% relative to delivery without hyaluronidase.

Clause 370. The combination dosing regimen of Clause 337, wherein the subcutaneous tissue concentration of the free payload is less than is less than 60% relative to delivery without hyaluronidase.

Clause 371. The combination dosing regimen of Clause 337, wherein the subcutaneous tissue concentration of the free payload is less than is less than 50% relative to delivery without hyaluronidase.

Clause 372. The combination dosing regimen of Clause 337, wherein the subcutaneous tissue concentration of the free payload is less than is less than 40% relative to delivery without hyaluronidase.

Clause 373. The combination dosing regimen of Clause 337, wherein the subcutaneous tissue concentration of the free payload is less than is less than 30% relative to delivery without hyaluronidase.

Clause 374. The combination dosing regimen of Clause 337, wherein the subcutaneous tissue concentration of the free payload is less than is less than 20% relative to delivery without hyaluronidase.

Clause 375. The combination dosing regimen of Clause 337, wherein the subcutaneous tissue concentration of the free payload is less than is less than 10% relative to delivery without hyaluronidase.

Clause 376. The combination dosing regimen of Clause 334, wherein Cmax is greater than 190% relative to delivery of the total antibody drug conjugate without hyaluronidase.

Clause 377. The combination dosing regimen of Clause 334, wherein Cmax is greater than 200% relative to delivery of the total antibody drug conjugate without hyaluronidase.

Clause 378. The combination dosing regimen of any of Clauses 345 to 377, wherein total antibody drug conjugate is present in an amount greater than antibody drug conjugate which is not intact.

Clause 379. The combination dosing regimen of Clause 335 or 337, wherein the total antibody drug conjugate is present in an amount less than free payload.

Clause 380. The combination dosing regimen of Clause 178, wherein subcutaneous delivery of antibody drug conjugate with soluble hyaluronidase results in greater therapeutic efficacy of the antibody-drug candidate in the human subject relative to intravenous administration of the antibody drug conjugate without hyaluronidase.

Clause 381. The combination dosing regimen of Clause 178, wherein subcutaneous delivery of antibody drug conjugate with soluble hyaluronidase increases overall survival in the human subject relative to intravenous administration of the antibody drug conjugate without hyaluronidase.

Clause 382. The combination dosing regimen of Clause 381, wherein the increased survival is disease free.

Clause 383. The combination dosing regimen of Clause 178, wherein subcutaneous delivery of antibody drug conjugate with soluble hyaluronidase increases objective response rate of the antibody-drug in the human subject candidate relative to intravenous administration of the antibody drug conjugate without hyaluronidase.

Clause 384. The combination dosing regimen of Clause 178, wherein subcutaneous delivery of antibody drug conjugate with soluble hyaluronidase produces greater complete response of the antibody-drug candidate in the human subject relative to intravenous administration of the antibody drug conjugate without hyaluronidase.

Clause 385. The combination dosing regimen of Clause 178, wherein subcutaneous delivery of antibody drug conjugate with soluble hyaluronidase increases progression-free survival of the antibody-drug candidate in the human subject relative to intravenous administration of the antibody drug conjugate without hyaluronidase.

Clause 386. The combination dosing regimen of Clause 178, wherein subcutaneous delivery of antibody drug conjugate with soluble hyaluronidase decreases time to treatment failure of the antibody-drug candidate in the human subject relative to intravenous administration of the antibody drug conjugate without hyaluronidase.

Clause 387. The combination dosing regimen of Clause 178, wherein subcutaneous delivery of antibody drug conjugate with soluble hyaluronidase increases duration of response of the antibody-drug candidate in the human subject relative to intravenous administration of the antibody drug conjugate without hyaluronidase.

Clause 388. The combination dosing regimen of Clause 178, wherein subcutaneous delivery of antibody drug conjugate with soluble hyaluronidase reduces toxicity of the antibody-drug candidate in the human subject relative to intravenous administration of the antibody drug conjugate without hyaluronidase.

Clause 389. The combination dosing regimen of Clause 388, wherein the toxicity is local toxicity at the site of injection.

Clause 390. The combination dosing regimen of Clause 389, wherein the local toxicity is selected from the group consisting of subcutaneous cytotoxicity, necrosis, lesions, ulcers, pruritis, infection, rash and dry skin.

Clause 391. The combination dosing regimen of Clause 178, wherein subcutaneous delivery of antibody drug conjugate with soluble hyaluronidase reduces adverse events of the antibody-drug candidate in the human subject relative to intravenous administration of the antibody drug conjugate without hyaluronidase.

Clause 392. The combination dosing regimen of Clause 391, wherein the antibody drug conjugate is administered subcutaneously at a dose equivalent or higher than a dose administered intravenously.

Clause 393. The combination dosing regimen of Clause 391, wherein the adverse events are selected from the group consisting of hypersensitivity and infusion-related reactions.

Clause 394. The combination dosing regimen of Clause 391, wherein the adverse events are selected from the group consisting of itching, redness, rash, hives, fever, chills, back or belly pain, muscle or joint pain, fast heartbeat, and nausea or vomiting, and severe anaphylactic reactions, that could include signs and symptoms of cardiac arrest, hypotension, wheezing, angioedema, swelling, pneumonitis, and skin reactions.

Clause 395. The combination dosing regimen of Clause 391, wherein the adverse events are selected from the group consisting of cytopenia, neutropenia, thrombocytopenia, anemia, leukopenia and lymphocytopenia.

Clause 396. The combination dosing regimen of Clause 391, wherein the adverse events are selected from the group consisting of diarrhea, constipation, abdominal pain, gastroenteritis, nausea, vomiting, decreased appetite, mucositis and stomatitis.

Clause 397. The combination dosing regimen of Clause 391, wherein the adverse events are selected from the group consisting of rash, pruritic, edema, dry skin and alopecia.

Clause 398. The combination dosing regimen of Clause 391, wherein the adverse events are selected from the group consisting of back pain and arthralgia.

Clause 399. The combination dosing regimen of Clause 391, wherein the adverse events is neuropathy.

Clause 400. The combination dosing regimen of any preceding Clause, wherein subcutaneous administration of the antibody drug candidate increases serum hemoglobin in the human subject relative to intravenous administration of the antibody drug candidate.

Clause 401. The combination dosing regimen of any preceding Clause, wherein subcutaneous administration of the antibody drug candidate increases serum albumin in the human subject relative to intravenous administration of the antibody drug candidate.

Clause 402. The combination dosing regimen of any preceding Clause, wherein subcutaneous administration of the antibody drug candidate increases creatinine clearance in the human subject relative to intravenous administration of the antibody drug candidate.

Clause 403. The combination dosing regimen of any preceding Clause, wherein subcutaneous administration of the antibody drug candidate decreases serum alkaline phosphatase in the human subject relative to intravenous administration of the antibody drug candidate.

Clause 404. The combination dosing regimen of any preceding Clause, wherein subcutaneous administration of the antibody drug candidate increases serum and/or plasma magnesium in the human subject relative to intravenous administration of the antibody drug candidate.

Clause 405. The combination dosing regimen of any preceding Clause, wherein subcutaneous administration of the antibody drug candidate increases serum and/or plasma potassium in the human subject relative to intravenous administration of the antibody drug candidate.

Clause 406. The combination dosing regimen of any preceding Clause, wherein subcutaneous administration of the antibody drug candidate increases serum and/or plasma sodium in the human subject relative to intravenous administration of the antibody drug candidate.

Clause 407. The combination dosing regimen of Clause 183, wherein the payload is cytotoxic.

Clause 408. The combination dosing regimen of any preceding Clause, wherein the antibody is bispecific.

Clause 409. The combination dosing regimen of any preceding Clause, wherein the ratio of total intact antibody drug conjugate to total monoclonal antibody in serum is between about 1% to about 100%.

Clause 410. The combination dosing regimen of Clause 409, wherein the ratio of total intact antibody drug conjugate to total monoclonal antibody in serum is about 10%.

Clause 411. The combination dosing regimen of Clause 409, wherein the ratio of total intact antibody drug conjugate to total monoclonal antibody in serum is about 20%.

Clause 412. The combination dosing regimen of Clause 409, wherein the ratio of total intact antibody drug conjugate to total monoclonal antibody in serum is about 30%.

Clause 413. The combination dosing regimen of Clause 409, wherein the ratio of total intact antibody drug conjugate to total monoclonal antibody in serum is about 40%.

Clause 414. The combination dosing regimen of Clause 409, wherein the ratio of total intact antibody drug conjugate to total monoclonal antibody in serum is about 50%.

Clause 415. The combination dosing regimen of Clause 409, wherein the ratio of total intact antibody drug conjugate to total monoclonal antibody in serum is about 60%.

Clause 416. The combination dosing regimen of Clause 409, wherein the ratio of total intact antibody drug conjugate to total monoclonal antibody in serum is about 70%.

Clause 417. The combination dosing regimen of Clause 409, wherein the ratio of total intact antibody drug conjugate to total monoclonal antibody in serum is about 80%.

Clause 418. The combination dosing regimen of Clause 409, wherein the ratio of total intact antibody drug conjugate to total monoclonal antibody in serum is about 90%.

Clause 419. The combination dosing regimen of Clause 409, wherein the ratio of total intact antibody drug conjugate to total monoclonal antibody in serum is about 100%.

Clause 420. The combination dosing regimen of any preceding Clause, wherein the ratio of free payload to total intact antibody drug conjugate in serum is between about 1% to about 100%.

Clause 421. The combination dosing regimen of Clause 420, wherein the ratio of free payload to total intact antibody drug conjugate in serum is about 10%.

Clause 422. The combination dosing regimen of Clause 420, wherein the ratio of free payload to total intact antibody drug conjugate in serum is about 20%.

Clause 423. The combination dosing regimen of Clause 420, wherein the ratio of free payload to total intact antibody drug conjugate in serum is about 30%.

Clause 424. The combination dosing regimen of Clause 420, wherein the ratio of free payload to total intact antibody drug conjugate in serum is about 40%.

Clause 425. The combination dosing regimen of Clause 420, wherein the ratio of free payload to total intact antibody drug conjugate in serum is about 50%.

Clause 426. The combination dosing regimen of Clause 420, wherein the ratio of free payload to total intact antibody drug conjugate in serum is about 60%.

Clause 427. The combination dosing regimen of Clause 420, wherein the ratio of free payload to total intact antibody drug conjugate in serum is about 70%.

Clause 428. The combination dosing regimen of Clause 420, wherein the ratio of free payload to total intact antibody drug conjugate in serum is about 80%.

Clause 429. The combination dosing regimen of Clause 420, wherein the ratio of free payload to total intact antibody drug conjugate in serum is about 90%.

Clause 430. The combination dosing regimen of Clause 420, wherein the ratio of free payload to total intact antibody drug conjugate in serum is about 100%.

Clause 431. The combination dosing regimen of any preceding Clause, wherein the ratio of total intact antibody drug conjugate to total monoclonal antibody in tissue is between about 1% to about 100%.

Clause 432. The combination dosing regimen of Clause 431, wherein the ratio of total intact antibody drug conjugate to total monoclonal antibody in tissue is about 10%.

Clause 433. The combination dosing regimen of Clause 431, wherein the ratio of total intact antibody drug conjugate to total monoclonal antibody in tissue is about 20%.

Clause 434. The combination dosing regimen of Clause 431, wherein the ratio of total intact antibody drug conjugate to total monoclonal antibody in tissue is about 30%.

Clause 435. The combination dosing regimen of Clause 431, wherein the ratio of total intact antibody drug conjugate to total monoclonal antibody in tissue is about 40%.

Clause 436. The combination dosing regimen of Clause 431, wherein the ratio of total intact antibody drug conjugate to total monoclonal antibody in tissue is about 50%.

Clause 437. The combination dosing regimen of Clause 431, wherein the ratio of total intact antibody drug conjugate to total monoclonal antibody in tissue is about 60%.

Clause 438. The combination dosing regimen of Clause 431, wherein the ratio of total intact antibody drug conjugate to total monoclonal antibody in tissue is about 70%.

Clause 439. The combination dosing regimen of Clause 431, wherein the ratio of total intact antibody drug conjugate to total monoclonal antibody in tissue is about 80%.

Clause 440. The combination dosing regimen of Clause 431, wherein the ratio of total intact antibody drug conjugate to total monoclonal antibody in tissue is about 90%.

Clause 441. The combination dosing regimen of Clause 431, wherein the ratio of total intact antibody drug conjugate to total monoclonal antibody in tissue is about 100%.

Clause 442. The combination dosing regimen of any preceding Clause, wherein the ratio of free payload to total intact antibody drug conjugate in tissue is between about 1% to about 100%.

Clause 443. The combination dosing regimen of Clause 442, wherein the ratio of free payload to total intact antibody drug conjugate in tissue is about 10%.

Clause 444. The combination dosing regimen of Clause 442, wherein the ratio of free payload to total intact antibody drug conjugate in tissue is about 20%.

Clause 445. The combination dosing regimen of Clause 442, wherein the ratio of free payload to total intact antibody drug conjugate in tissue is about 30%.

Clause 446. The combination dosing regimen of Clause 442, wherein the ratio of free payload to total intact antibody drug conjugate in tissue is about 40%.

Clause 447. The combination dosing regimen of Clause 442, wherein the ratio of free payload to total intact antibody drug conjugate in tissue is about 50%.

Clause 448. The combination dosing regimen of Clause 442, wherein the ratio of free payload to total intact antibody drug conjugate in tissue is about 60%.

Clause 449. The combination dosing regimen of Clause 442, wherein the ratio of free payload to total intact antibody drug conjugate in tissue is about 70%.

Clause 450. The combination dosing regimen of Clause 442, wherein the ratio of free payload to total intact antibody drug conjugate in tissue is about 80%.

Clause 451. The combination dosing regimen of Clause 442, wherein the ratio of free payload to total intact antibody drug conjugate in tissue is about 90%.

Clause 452. The combination dosing regimen of Clause 442, wherein the ratio of free payload to total intact antibody drug conjugate in tissue is about 100%.

Clause 453. A combination dosing regimen, comprising:
(i) subcutaneously administering to a patient in need thereof a composition comprising a soluble hyaluronidase comprising a sequence of amino acids that has at least 85%, 86%, 87%, 88%, 89%, 90%, 91%, 92%, 93%, 94%, 95%, 96%, 97%, 98% or 99% sequence identity to a sequence of amino acids that contains at least amino acids 36-464 of SEQ ID NO: 1 and retains hyaluronidase activity; and an antibody-drug conjugate; or
(ii) subcutaneously administering to a patient in need thereof a first composition comprising a soluble hyaluronidase comprising a sequence of amino acids that has at least 85%, 86%, 87%, 88%, 89%, 90%, 91%, 92%, 93%, 94%, 95%, 96%, 97%, 98% or 99% sequence identity to a sequence of amino acids that contains at least amino acids 36-464 of SEQ ID NO: 1 and retains hyaluronidase activity; and
subcutaneously administering to the patient in need thereof a second composition comprising an antibody-drug conjugate;
   wherein the soluble hyaluronidase is subcutaneously administered to the patient in an amount sufficient to obtain at least 50% bioavailability of the antibody-drug conjugate compared to the bioavailability obtained when the antibody-drug conjugate is administered intravenously.

Clause 454. The combination dosing regimen of clause 453, wherein the soluble hyaluronidase and the antibody-drug conjugate are subcutaneously administered to the patient in an amount sufficient to obtain from at least 50% to 225%, 75% to 225%, 75% to 200%, 100% to 200%, 100% to 175%, 125% to 175%, 125% to 150%, or 90% to 110% of the bioavailability of the antibody-drug conjugate compared to the bioavailability obtained when the antibody-drug conjugate is administered intravenously at a therapeutically effective dose.

Clause 455. A combination dosing regimen comprising subcutaneously administering to a subject an antibody-drug conjugate (ADC) and a soluble hyaluronidase; wherein subcutaneous administration of the ADC with the soluble hyaluronidase provides:
a maximum blood concentration (Cmax) of ADC that is about 20% to about 60%, optionally about 20% to about 40%, of the Cmax obtained via intravenous (IV) administration of an equivalent dose of the ADC; and
an area under the concentration-time curve (AUC) in blood of ADC that is about 50% to about 90%, optionally about 50% to about 80%, of the AUC obtained via IV administration of an equivalent dose of the ADC.

Clause 456. The combination dosing regimen of clause 455, wherein the ADC comprises an antibody and a payload conjugated to the antibody via a cleavable linker, wherein upon cleavage of the cleavable linker, free antibody and payload are released.

Clause 457. The combination dosing regimen of clause 455 or 456, wherein subcutaneous administration of the ADC with the soluble hyaluronidase provides a Cmax of free antibody that is about 20% to 60% of the Cmax achieved by IV administration of an equivalent dose of the ADC.

Clause 458. The combination dosing regimen of any one of clauses 455-457, wherein subcutaneous administration of the ADC with the soluble hyaluronidase provides a Cmax of the ADC that is about 20% to 55% of the Cmax achieved by IV administration of an equivalent dose of the ADC.

Clause 459. The combination dosing regimen of any one of clauses 455-458, wherein subcutaneous administration of the ADC with the soluble hyaluronidase provides a Cmax of free payload that is about 30% to about 80% of the Cmax achieved by IV administration of an equivalent dose of the ADC.

Clause 460. The combination dosing regimen of any one of clauses 455-458, wherein subcutaneous administration of the ADC with the soluble hyaluronidase provides a Cmax of total payload that is about 30% to about 80% of the Cmax achieved by IV administration of an equivalent dose of the ADC.

Clause 461. The combination dosing regimen of any one of clauses 455-460, wherein subcutaneous administration of the ADC with the soluble hyaluronidase provides an AUC in blood of the ADC that is about 50% to 90% of the blood AUC achieved by IV administration of an equivalent dose of the ADC.

Clause 462. The combination dosing regimen of any one of clauses 455-461, wherein subcutaneous administration of the ADC with the soluble hyaluronidase provides an AUC in blood of total antibody that is about 50% to 90% of the blood AUC achieved by IV administration of an equivalent dose of the ADC.

Clause 463. The combination dosing regimen of any one of clauses 455-462, wherein subcutaneous administration of the ADC with the soluble hyaluronidase provides an AUC in blood of free payload that is about 50% to 90% of the blood AUC achieved by IV administration of an equivalent dose of the ADC.

Clause 464. The combination dosing regimen of any one of clauses 455-463, wherein the IV administration of the equivalent dose of the ADC does not comprise soluble hyaluronidase.

Clause 465. The combination dosing regimen of any one of clauses 455-464, wherein subcutaneous administration of the ADC with the soluble hyaluronidase achieves a weekly average AUC in blood of the ADC that is about 100 μg/mL*day to 230 μg/mL*day.

Clause 466. The combination dosing regimen of any one of clauses 455-465, wherein subcutaneous administration of the ADC with the soluble hyaluronidase achieves a Cmax of the ADC that is about 20 μg/mL to 150 μg/mL.

Clause 467. The combination dosing regimen of any one of clauses 455-466, wherein subcutaneous administration of the ADC with the soluble hyaluronidase achieves a Cmax of the ADC that is about 25 μg/mL to 100 μg/mL.

Clause 468. A combination dosing regimen comprising subcutaneously administering to a subject an antibody-drug conjugate (ADC) and a soluble hyaluronidase;
wherein the dosing regimen is characterized by:
   a higher dose of the ADC being delivered subcutaneously than an a therapeutically effective dose of the ADC when administered intravenously;
   the subcutaneous administration of the ADC yields a blood area under the concentration-time curve (AUC) that is equal to or higher than the AUC obtained from an intravenously administered therapeutically effective dose of the ADC; and
   the subcutaneous administration of the ADC yields a maximum blood concentration (Cmax) that is equal to or lower than the Cmax obtained from an intravenously administered therapeutically effective dose of the ADC.

Clause 469. The combination dosing regimen of clause 468, wherein the ADC comprises an antibody and a payload conjugated to the antibody via a cleavable linker, wherein upon cleavage of the cleavable linker, free antibody and payload are released.

Clause 470. The combination dosing regimen of clause 468 or 469, wherein the AUC and Cmax are for the total antibody.

Clause 471. The combination dosing regimen of clause 468 or 469, wherein the AUC and Cmax are for the free antibody.

Clause 472. The combination dosing regimen of clause 468 or 469, wherein the AUC and Cmax are for the free payload.

Clause 473. The combination dosing regimen of clause 468 or 469, wherein the AUC and Cmax are for the total payload.

Clause 474. The combination dosing regimen of clause 468 or 469, wherein the AUC is for the ADC and Cmax is for the total payload.

Clause 475. The combination dosing regimen of any one of clauses 453-474, wherein the dose delivered subcutaneously is 25% to 400% of the dose administered intravenously.

Clause 476. The combination dosing regimen of any one of clauses 453-475, wherein the dose delivered subcutaneously is 100 to 350% of the dose administered intravenously.

Clause 477. The combination dosing regimen of any one of clauses 453-476, wherein the dose delivered subcutaneously is 150% to 300% of the dose administered intravenously.

Clause 478. The combination dosing regimen of any one of clauses 453-477, wherein the dose delivered subcutaneously is 200% to 250% of the dose administered intravenously.

Clause 479. The combination dosing regimen of any one of clauses 453-478, wherein the dose delivered subcutaneously is 150% to 200% of the dose administered intravenously.

Clause 480. The combination dosing regimen of any one of clauses 453-478, wherein the dose delivered subcutaneously is 200% of the dose administered intravenously.

Clause 481. The combination dosing regimen of any one of clauses 453-476, wherein the dose delivered subcutaneously is 100% to 150% of the dose administered intravenously.

Clause 482. The combination dosing regimen of any one of clauses 468-481, wherein the intravenously administered therapeutically effective dose of the ADC does not comprise soluble hyaluronidase.

Clause 483. The combination dosing regimen of any one of clauses 468-482, wherein subcutaneous administration of the ADC with the soluble hyaluronidase achieves a weekly average AUC in blood of the ADC that is about 120 μg/mL*day to 680 μg/mL*day.

Clause 484. The combination dosing regimen of any one of clauses 468-483, wherein subcutaneous administration of the ADC with the soluble hyaluronidase achieves a Cmax of the ADC that is about 25 μg/mL to 410 μg/mL.

Clause 485. The combination dosing regimen of any one of clauses 468-484, wherein subcutaneous administration of the ADC with the soluble hyaluronidase achieves a Cmax of the ADC that is about 35 μg/mL to 330 μg/mL.

Clause 486. The combination dosing regimen of any one of clauses 468-485, wherein the dose delivered subcutaneously is delivered more frequently than the therapeutically effective dose delivered intravenously.

Clause 487. A combination dosing regimen of any one of clauses 453-486, wherein the subcutaneous administration of the ADC in combination with the soluble hyaluronidase results in:
greater therapeutic efficacy of the ADC in the subject relative to intravenous administration of the ADC; and/or
increased overall survival in the subject relative to intravenous administration of the antibody drug conjugate; and/or
increased objective response rate of the ADC in the subject relative to intravenous administration of the antibody drug conjugate; and/or
produces greater complete response of the ADC in the subject relative to intravenous administration of the ADC; and/or
increased progression-free survival of the ADC in the subject relative to intravenous administration of the ADC; and/or
decreased time to treatment failure of the ADC in the subject relative to intravenous administration of the ADC; and/or
increased duration of response of the ADC in the subject relative to intravenous administration of the ADC.

Clause 488. The combination dosing regimen of clause 487, wherein the increased survival is disease free.

Clause 489. The combination dosing regimen of clause 487 or 488, wherein the intravenous administration of the ADC is without the soluble hyaluronidase.

Clause 490. A combination dosing regimen of any one of clauses 453-489, wherein the subcutaneous administration of the ADC in combination with the soluble hyaluronidase results in:
reduced toxicity of the ADC in the subject relative to intravenous administration of the ADC; and/or
reduced adverse events of the ADC in the subject relative to intravenous administration of the ADC.

Clause 491. The combination dosing regimen of clause 490, wherein the intravenous administration of the ADC is without the soluble hyaluronidase.

Clause 492. The combination dosing regimen of clause 490 or 491, wherein the adverse events are selected from hypersensitivity and infusion-related reactions.

Clause 493. The combination dosing regimen of clause 490 or 491, wherein the adverse events are selected from itching, redness, rash, hives, fever, chills, back or belly pain, muscle or joint pain, fast heartbeat, and nausea or vomiting, and severe anaphylactic reactions, that could include signs and symptoms of cardiac arrest, hypotension, wheezing, angioedema, swelling, pneumonitis, and skin reactions.

Clause 494. The combination dosing regimen of clause 490 or 491, wherein the adverse events are selected from cytopenia, neutropenia, thrombocytopenia, anemia, leukopenia and lymphocytopenia.

Clause 495. The combination dosing regimen of clause 490 or 491, wherein the adverse events are selected from diarrhea, constipation, abdominal pain, gastroenteritis, nausea, vomiting, decreased appetite, mucositis and stomatitis.

Clause 496. The combination dosing regimen of clause 490 or 491, wherein the adverse events are selected from rash, pruritic, edema, dry skin and alopecia.

Clause 497. The combination dosing regimen of clause 490 or 491, wherein the adverse events are selected from back pain and arthralgia.

Clause 498. The combination dosing regimen of clause 490 or 491, wherein the adverse event is neuropathy.

Clause 499. The combination dosing regimen of clause 490 or 491, wherein the adverse event is interstitial lung disease.

Clause 500. A combination dosing regimen of any one of clauses 453-489, wherein the subcutaneous administration of the ADC in combination with the soluble hyaluronidase results in:
an increased blood haemoglobin in the subject relative to intravenous administration of the ADC; and/or
an increased blood albumin in the subject relative to intravenous administration of the ADC; and/or
an increased creatinine clearance in the subject relative to intravenous administration of the ADC; and/or
decreased blood alkaline phosphatase in the subject relative to intravenous administration of the ADC; and/or
increased blood magnesium in the subject relative to intravenous administration of the ADC; and/or
increased blood potassium in the subject relative to intravenous administration of the ADC; and/or
increased blood sodium in the subject relative to intravenous administration of the ADC.

Clause 501. The combination dosing regimen of clause 500, wherein the ADC comprises an antibody and a payload conjugated to the antibody via a cleavable linker, wherein upon cleavage of the cleavable linker, free antibody and payload are released.

Clause 502. The combination dosing regimen of clause 500 or 501, wherein the intravenous administration of the ADC does not comprise the soluble hyaluronidase.

Clause 503. A combination dosing regimen comprising:
subcutaneously administering to a subject an antibody-drug conjugate (ADC) comprising an antibody that specifically targets Trop 2; and a soluble hyaluronidase,
   wherein subcutaneous administration of the ADC with the soluble hyaluronidase provides a lower maximum blood concentration (Cmax) of ADC compared to intravenous (IV) administration of an equivalent dose of the ADC.

Clause 504. The combination dosing regimen of clause 503, wherein the ADC comprises a topoisomerase I inhibitor payload conjugated to the antibody via a cleavable linker.

Clause 505. The combination dosing regimen of clause 504, wherein the topoisomerase I inhibitor payload is a chemotherapy drug.

Clause 506. The combination dosing regimen of clause 505, wherein the chemotherapy drug is SN-38.

Clause 507. The combination dosing regimen of any one of clauses 503-506, wherein the ADC is sacituzumab govitecan.

Clause 508. The combination dosing regimen of any one of clauses 503-507, wherein subcutaneous administration of the combination of the ADC with the soluble hyaluronidase provides a maximum blood concentration (Cmax) of ADC that is 20% to 50% of the Cmax achieved by intravenous (IV) administration of an equivalent dose of the ADC.

Clause 509. The combination dosing regimen of any one of clauses 503-508, wherein subcutaneous administration of the combination of the ADC with the soluble hyaluronidase provides an area under the concentration-time curve (AUC) of ADC that is about 40% to about 60% of the AUC obtained via intravenous (IV) administration of an equivalent dose of the ADC.

Clause 510. The combination dosing regimen of any one of clauses 503-509, wherein the intravenous administration of an equivalent dose of the ADC is without the soluble hyaluronidase.

Clause 511. A combination dosing regimen comprising:
subcutaneously administering to a subject an antibody-drug conjugate (ADC) comprising an antibody that specifically targets HER2 and a soluble hyaluronidase;
   wherein subcutaneous administration of the ADC with the soluble hyaluronidase provides a lower maximum blood concentration (Cmax) of ADC compared to intravenous (IV) administration of an equivalent dose of the ADC.

Clause 512. The combination dosing regimen of clause 509, wherein the ADC comprises a topoisomerase I inhibitor payload conjugated to the antibody via a cleavable linker.

Clause 513. The combination dosing regimen of clause 512, wherein the topoisomerase I inhibitor payload is a chemotherapy drug.

Clause 514. The combination dosing regimen of clause 513, wherein the chemotherapy drug is exatecan derivative DXd.

Clause 515. The combination dosing regimen of any one of clauses 511-514, wherein the ADC is trastuzumab deruxtecan.

Clause 516. The combination dosing regimen of any one of clauses 511-515, wherein subcutaneous administration of the combination of the ADC with the soluble hyaluronidase provides a maximum blood concentration (Cmax) of ADC that is about 15% to about 45% of the Cmax achieved by intravenous (IV) administration of an equivalent dose of the ADC.

Clause 517. The combination dosing regimen of any one of clauses 511-516, wherein subcutaneous administration of the combination of the ADC with the soluble hyaluronidase provides an area under the concentration-time curve (AUC) of ADC that is about 60% to about 90% of the AUC obtained via intravenous (IV) administration of an equivalent dose of the ADC.

Clause 518. The combination dosing regimen of any one of clauses 511-517, wherein the intravenous administration of an equivalent dose of the ADC is without the soluble hyaluronidase.

Clause 519. The combination dosing regimen of any one of clauses 455-518, wherein:
the free payload weekly average AUC of the ADC administered subcutaneously with the soluble hyaluronidase is 80% to 140%, optionally 100% to 125%, of the equivalent dose of free payload weekly average AUC of the ADC administered subcutaneously without soluble hyaluronidase; and/or
the weekly average AUC of the ADC administered subcutaneously with the soluble hyaluronidase is 110% to 170%, optionally 125% to 160%, of the equivalent dose total ADC weekly average AUC of the ADC administered subcutaneously without the soluble hyaluronidase.

Clause 520. The combination dosing regimen of clause 519, wherein the free payload weekly average AUC of the ADC administered subcutaneously with the soluble hyaluronidase is 80% to 140%, optionally 100% to 125%, of the equivalent dose of free payload weekly average AUC of the ADC administered subcutaneously without soluble hyaluronidase.

Clause 521. The combination dosing regimen of clause 519 or 520, wherein the weekly average AUC of the ADC administered subcutaneously with the soluble hyaluronidase is 110% to 170%, optionally 125% to 160%, of the equivalent dose total ADC weekly average AUC of the ADC administered subcutaneously without the soluble hyaluronidase.

Clause 522. The combination dosing regimen of any one of clauses 453-521, wherein subcutaneous administration the combination of the ADC with the soluble hyaluronidase provides improved local tolerability in the subject relative to subcutaneous administration of an equivalent dose of the ADC without the soluble hyaluronidase, evidenced at the injection site by reduced erythema, swelling, pain, subcutaneous cytotoxicity, necrosis, lesions, ulcers, pruritis, infection, rash, or dry skin.

Clause 523. The combination dosing regimen of any one of clauses 453-522, wherein subcutaneous administration of the combination of the ADC with the soluble hyaluronidase provides a reduced incidence of adverse events in the subject compared to intravenous administration of the ADC.

Clause 524. The combination dosing regimen of any one of clauses 453-523, wherein the soluble hyaluronidase is administered concurrently with the ADC.

Clause 525. The combination dosing regimen of any one of clauses 453-524, wherein the soluble hyaluronidase is co-formulated with the ADC.

Clause 526. The combination dosing regimen of any one of clauses 453-525, wherein the soluble hyaluronidase and the ADC are lyophilized together and reconstituted prior to administration to the subject.

Clause 527. The combination dosing regimen of any one of clauses 453-525, wherein the soluble hyaluronidase and the ADC are lyophilized separately and reconstituted prior to administration to the subject.

Clause 528. The combination dosing regimen of any one of clauses 453-525, wherein the ADC is lyophilized and reconstituted in a solution comprising the soluble hyaluronidase prior to administration to the subject.

Clause 529. The combination dosing regimen of any one of clauses 453-528, wherein co-administration of the ADC with the soluble hyaluronidase subcutaneously reduces injection time by at least 10% compared to ADC administered alone either subcutaneously or intravenously.

Clause 530. The combination dosing regimen of any one of clauses 453-523, wherein the soluble hyaluronidase is administered within about 60 minutes prior to administration of the ADC.

Clause 531. The combination dosing regimen of any one of clauses 453-523, wherein the soluble hyaluronidase is administered within about 30 minutes prior to administration of the ADC.

Clause 532. The combination dosing regimen of any one of clauses 453-531, wherein the soluble hyaluronidase is administered within about 15 minutes prior to administration of the ADC.

Clause 533. The combination dosing regimen of any one of clauses 453-532, wherein the combination dosing regimen is administered on a dosing schedule of once or twice weekly.

Clause 534. The combination dosing regimen of any one of clauses 453-532, wherein the combination dosing regimen is administered on a dosing schedule of once every 2-4 weeks.

Clause 535. The combination dosing regimen of any one of clauses 453-532, wherein the combination dosing regimen is administered on a dosing schedule of once every 2-6 weeks.

Clause 536. The combination dosing regimen of any one of clauses 453-532, wherein the combination dosing regimen is administered on a dosing schedule of once every 2-8 weeks.

Clause 537. The combination dosing regimen of any one of clauses 453-532, wherein the combination dosing regimen is administered on a dosing schedule of once every two (2) to twelve (12) weeks.

Clause 538. The combination dosing regimen of any one of clauses 533-537, wherein the dosing schedule is maintained for at least two cycles.

Clause 539. The combination dosing regimen of any one of clauses 533-538, wherein the dosing schedule is maintained for at least four cycles.

Clause 540. The combination dosing regimen of any one of clauses 533-539, wherein the dosing schedule is maintained for at least six cycles.

Clause 541. The combination dosing regimen of any one of clauses 533-540, wherein the dosing schedule is maintained for at least eight cycles.

Clause 542. The combination dosing regimen of any one of clauses 453-541, wherein the ADC is administered at a dose of 0.1 mg/kg to 50 mg/kg.

Clause 543. The combination dosing regimen of any one of clauses 453-542, wherein the ADC is administered at a dose of 0.1 mg/kg to 10 mg/kg.

Clause 544. The combination dosing regimen of any one of clauses 453-543, wherein the ADC is administered at a dose of 0.1 mg/kg to 5 mg/kg.

Clause 545. The combination dosing regimen of any one of clauses 453-544, wherein the ADC is administered at a dose of 1 mg/kg to 6 mg/kg.

Clause 546. The combination dosing regimen of any one of clauses 453-545, wherein the soluble hyaluronidase is administered at a dose of 2,000 to 100,000 U, optionally at a dose of 2,000 to 25,000 U.

Clause 547. The combination dosing regimen of any one of clauses 453-546, wherein the soluble hyaluronidase is administered at a dose of about 2,000 U.

Clause 548. The combination dosing regimen of any one of clauses 453-546, wherein the soluble hyaluronidase is administered at a dose of about 4,000 U.

Clause 549. The combination dosing regimen of any one of clauses 453-546, wherein the soluble hyaluronidase is administered at a dose of about 6,000 U.

Clause 550. The combination dosing regimen of any one of clauses 455-549, wherein the soluble hyaluronidase comprises a soluble human hyaluronidase.

Clause 551. The combination dosing regimen of any one of clauses 455-550, wherein the soluble hyaluronidase comprises a recombinant soluble human hyaluronidase.

Clause 552. The combination dosing regimen of any one of clauses 455-551, wherein the soluble hyaluronidase comprises a sequence of amino acids that has at least 85%, 86%, 87%, 88%, 89%, 90%, 91%, 92%, 93%, 94%, 95%, 96%, 97%, 98% or 99% sequence identity to a sequence of amino acids that contains at least amino acids 36-464 of SEQ ID NO:1 and retains hyaluronidase activity.

Clause 553. A pharmaceutical composition for subcutaneous administration, the pharmaceutical composition comprising: an antibody-drug conjugate (ADC) comprising an antibody and a payload conjugated to the antibody via a cleavable linker; and a soluble hyaluronidase.

Clause 554. The pharmaceutical composition of clause 553, wherein the antibody binds to Trop-2, HER-2, B7-H3, EGFR, DLL3, HER-3, CDH17, folate receptor alpha, Nectin-4, CLDN18.2, c-MET, NaPI2b, CEACAM5, PSMA, CLDN6, FGFR2b, ROR1, CD33, CD30, CD22, CD79b, CD19, integrin beta-6, or Tissue Factor.

Clause 555. The pharmaceutical composition of clause 553 or 554, wherein the antibody binds to more than one antigen.

Clause 556. The pharmaceutical composition of any one of clauses 553-555, wherein the payload is a topoisomerase inhibitor, optionally a topoisomerase I inhibitor.

Clause 557. The pharmaceutical composition of any one of clauses 553-556, wherein the payload is a topoisomerase I inhibitor selected from A-1743332 (Adizutecan), AMDCPT, ATI020, AZ14170132 (AZ′0132) (Samrotecan), AZ14170133, BCPT02, Belotecan and Analogues, BLD1102, Bultecan, C24, Camptothecin, CPT-113, CPT116, CPT2, D2102, Deruxtecan, DDDXd, DXd/DX8951 (MAAA-1181a), Dxh, Ed-04, Exatecan, FL-118, GS-P-000, HC74, HS-9265/SHR9265/Rezetecan, Irinotecan (CPT-11), JS-1, KL610023, LD-38, LDX2, Masetecan, MH30010008, Mtoxin (MF-6), NT1, P1003, P1021 (Drozuntecan), PBX-7, PBX-7016, PY-4car2, PY-4car2, QLS6916, SC3386, SN-38, T01, Tavatecan, Topotecan, VIP126, YL0010014, YL0014, ZD06519, or a derivative or analogue of any one thereof; optionally wherein the payload is selected from SN-38, deruxtecan, exatecan, topotecan, camptothecin, or a derivative of any one thereof.

Clause 558. The pharmaceutical composition of clause 556 or 557, wherein the topoisomerase I inhibitor is SN-38.

Clause 559. The pharmaceutical composition of clause 556 or 557, wherein the topoisomerase I inhibitor is deruxtecan.

Clause 560. The pharmaceutical composition of any one of clauses 553-559, wherein the cleavable linker is a chemically cleavable linker.

Clause 561. The pharmaceutical composition of any one of clauses 553-560, wherein the cleavable linker is selected from an acid labile linker, an enzyme cleavable linker, a reducible disulfide linker, a glutathione-sensitive linker, an Fe (II)-responsive linker, an oxidation labile/ROS (reactive oxygen species) sensitive linker, a photo-responsive linker, a bioorthogonal linker, or a combination thereof.

Clause 562. The pharmaceutical composition of clause 561, wherein the acid labile linker is a hydrazone linker or a CL2A linker.

Clause 563. The pharmaceutical composition of any one of clauses 553-562, wherein the cleavable linker is a hydrazone linker.

Clause 564. The pharmaceutical composition of any one of clauses 553-563, wherein the cleavable linker is cleaved in the acidic environment of endosomes or lysosomes.

Clause 565. The pharmaceutical composition of any one of clauses 553-564, wherein the ADC has a drug antibody ratio of 2-16.

Clause 566. The pharmaceutical composition of any one of clauses 553-565, wherein the ADC has a drug-antibody ratio of 2-8.

Clause 567. The pharmaceutical composition of any one of clauses 553-566, wherein the ADC has a drug-antibody ratio of 2-4.

Clause 568. The pharmaceutical composition of any one of clauses 553-567, wherein the ADC is sacituzumab govitecan.

Clause 569. The pharmaceutical composition of any one of clauses 553-567, wherein the ADC is trastuzumab deruxtecan.

Clause 570. The pharmaceutical composition of any one of clauses 553-569, further comprising one or more excipients selected from 2-(N-morpholino) ethane sulfonic acid (MES), citric acid monohydrate, dextran, d-mannitol, glacial acetic acid, histidine, histidine hydrochloride monohydrate, L-histidine, L-histidine hydrochloride monohydrate, L-histidine monohydrochloride, polysorbate, sodium acetate, sodium chloride, sodium citrate dihydrate, sodium hydroxide, sodium phosphate dibasic anhydrous, sodium phosphate monobasic monohydrate, sodium succinate, succinic acid, sucrose, trehalose, trehalose dihydrate, or tromethamine.

Clause 571. The pharmaceutical composition of any one of clauses 553-570, further comprising one or more buffers selected from histidine, MES, citrate, acetate, phosphate, or TRIS.

Clause 572. The pharmaceutical composition of any one of clauses 553-571, further comprising one or more stabilizers selected from trehalose, sucrose, mannitol, sorbitol, glycine, or arginine.

Clause 573. The pharmaceutical composition of any one of clauses 553-572, further comprising one or more surfactants selected from polysorbate 20, polysorbate 80, poloxamer 188, or sodium deoxycholate.

Clause 574. The pharmaceutical composition of any one of clauses 553-573, further comprising one or more tonicity-adjusting agents selected from sodium chloride, potassium chloride, calcium chloride, or glycerol.

Clause 575. The pharmaceutical composition of any one of clauses 553-574, further comprising one or more antioxidants selected from methionine, cysteine, ascorbic acid, a tocopherol, or BHT.

Clause 576. The pharmaceutical composition of any one of clauses 553-575, further comprising one or more preservatives selected from benzyl alcohol, phenol, m-cresol, or a paraben.

Clause 577. The pharmaceutical composition of any one of clauses 553-569, comprising a combination of a buffer, a stabilizer, a surfactant, and a tonicity-adjusting agent.

Clause 578. The pharmaceutical composition of any one of clauses 553-577, wherein the soluble hyaluronidase comprises a soluble human hyaluronidase.

Clause 579. The pharmaceutical composition of any one of clauses 553-578, wherein the soluble hyaluronidase comprises a recombinant soluble human hyaluronidase.

Clause 580. The pharmaceutical composition of any one of clauses 553-579, wherein the soluble hyaluronidase comprises a sequence of amino acids that has at least 85%, 86%, 87%, 88%, 89%, 90%, 91%, 92%, 93%, 94%, 95%, 96%, 97%, 98% or 99% sequence identity to a sequence of amino acids that contains at least amino acids 36-464 of SEQ ID NO:1 and retains hyaluronidase activity.

### EXAMPLES

The following examples are included for illustrative purposes only and are not intended to limit the scope of the invention.

### Example 1: Tolerability of Sacituzumab Govitecan Following Subcutaneous Administration with and without Recombinant Human Hyaluronidase PH20 (rHuPH20)

High volume auto-injectors (HVAIs) are in development for subcutaneous (SC) delivery of biotherapeutics. However, traditional volumetric limitations have kept the development of these devices to around 2.5 to 5 milliliters (mL). Recombinant human hyaluronidase PH20 (rHuPH20) has been shown to facilitate the SC delivery of large volumes (from 5-600 mL) in clinical applications and may facilitate the delivery of large volumes of biotherapeutics using an HVAI. Both pre-clinical and clinical testing of a prototype HVAI device has demonstrated its capacity to deliver a 10 mL volume of test solution in approximately a 15-45 second timeframe.

This study investigated the local tolerability of subcutaneous (SC) administration of an antibody drug conjugate (ADC) both with and without recombinant human hyaluronidase PH20 (rHuPH20). An HVAI was used to deliver the test solutions. The HVAI had an exposed needle length of 10 mm and was vertically inserted through the dermis. Minipigs were used in this study due to the similarity of the SC skin architecture to humans. Each animal received two 10 mL SC injections into the lower abdominal region using the prototype HVAI. The first injection contained the ADC alone followed by the second injection on the contralateral side of the animal which contained the ADC+rHuPH20 at 2000 U/mL.

The duration of each injection was measured and recorded. Additional endpoints included measurement of post-injection back-leakage, local injection site measurements (bleb area and volume), qualitative scoring assessment of the local tissue for erythema, swelling size and induration, and standard imaging to quantitate post-injection changes of the skin. Qualitative assessments were taken at approximately one, two- and three-days post-injection. After the final imaging timepoint the animal was humanely euthanized and full skin thickness punch biopsies were collected of the injection sites.

Subcutaneous administration of large volumes of antibodies has been shown to be feasible when the antibody solution is co-formulated with recombinant human hyaluronidase PH20 (rHuPH20). rHuPH20 has been shown to facilitate SC administration of fluids and drugs by transiently and locally depolymerizing hyaluronan (HA) in the extracellular matrix (ECM) thereby reducing tissue backpressure in the SC space permitting rapid, large volume administration of fluid. Using this technology, subcutaneous administration of large volumes of antibody has become possible and is replacing intravenous administration as a treatment paradigm. Currently there are numerous approved antibody therapies that utilize rHuPH20 to enable subcutaneous administration including Herceptin® SC, Darzalex® SC, Phesgo™ and Hyqvia®.

A novel class of antibody-based therapeutics has been developed and recently approved by the FDA for the management or treatment of cancer. These therapeutics combine monoclonal antibodies specific to surface antigens present on particular tumor cells with highly potent anti-cancer agents linked via a chemical linker to form an antibody drug conjugate (ADC). Because these novel therapeutics are now being considered for SC administration it was important to assess their local tolerability using an appropriate animal model. Non-clinical local tolerance testing is intended to support human exposure to a compound at contact sites of the body and should reflect the proposed clinical administration. The swine model was chosen for this assessment due to the high similarity of the skin and subcutaneous space between humans and swine. Previous studies using a minipig model have demonstrated the translatability of the model for use in pre-clinical (Kang et al., 2013) and auto-injector studies.

This study utilized the FDA-approved drug Sacituzumab govitecan (Trodelvy®) as a representative ADC. This antibody was used in a prior study that demonstrated the local tolerability both alone and in combination with rHuPH20 after SC administration. However, this study was limited to a four-hour exposure and this follow-on study evaluated the local tolerability of larger dose volumes and longer exposure times.

In this study three animals were treated to assess the local tolerability of SC administration of either the antibody solution co-mixed with rHuPH20 or the antibody solution alone. Two SC injections were administered to the abdomen of each animal with one injection located on the lower left abdomen of the animal and the other injection located on the lower right of the abdomen of the animal. The injection volume was 10 mL. The test solution was delivered using a prototype high volume auto-injector (HVAI) that had been successfully used in previous preclinical and clinical studies to reproducibly deliver a 10 mL injection volume in an approximate 30 second timeframe.

After test solution administration, the injection site was monitored over a period of 72h with daily observations, photographing and qualitative assessments taken of the injection site. After the last timepoint, each animal was humanely euthanized and full thickness punch biopsies of each injection site were obtained post-mortem and preserved in 10% formalin for histological analyses using hematoxylin and eosin (H&E) staining.

### Test Articles

### Antibody Drug Conjugate (ADC)—Sacituzumab Govitecan (Trodelvy®)

Description: Clear colorless liquid
Lot number: S23C008A
Concentration: 10 mg/mL
Formulation: Reconstituted in 20 mL of 0.9% NaCl for Injection, USP
Storage Conditions: 2-8° C.
Handling Conditions: Standard laboratory precautions
Supplier: Pharmaceutical Buyers, Inc.
   Recombinant Human Hyaluronidase (rHuPH20)
Description: Clear colorless liquid
Lot number: 1-FIN-3928
Concentration: 1,039,763 U/mL; 10 mg/mL
Formulation: 10 mM Histidine, 130 mM sodium chloride, pH 6.5
Storage Conditions: ≤70° C.
Handling Conditions: Standard laboratory precautions
Supplier: Halozyme, Inc.

### 0.9% Sodium Chloride

Description: Clear colorless liquid
Lot number: GX9803
Expiration date: 1 Jan. 2025
Storage Conditions: Room temperature
Handling Conditions: Standard laboratory precautions
Supplier: Hospira

Preparation of Test Solutions. The two test solutions administered in this study were Trodelvy alone and Trodelvy+rHuPH20. Three vials of Trodelvy were reconstituted according to manufacturer's instructions using 20.2 mL of 0.9% Sodium Chloride to the lyophilized product in each vial to produce 60.6 mL of Trodelvy that was used to prepare (3) syringes of Trodelvy and (3) syringes of Trodelvy+rHuPH20.

### Preparation of Syringes Containing Trodelvy Alone

After reconstitution, three syringes were prepared that contained 10.1 mL each of Trodelvy. After filling each syringe, it was capped and stored at 2-8° C.

### Preparation of Syringes Containing Trodelvy+rHuPH20

To prepare Trodelvy+rHuPH20, 0.062 mL of rHuPH20 was added to the remaining 30.3 mL of Trodelvy. This was used to fill three syringes with 10.1 mL each of Trodelvy+rHuPH20. After filling each syringe, it was capped and stored at 2-8° C.

### Device Preparation

Assembly of all devices occurred in a sterile fill and finish hood under aseptic conditions. COC syringes were used to contain test solutions in the HVAI device. Springs used in this study had the same k spring constant as the devices used in a previous study using the HVAI (Studies 22148 and 23027). Prior to final assembly COC syringes were brought to room temperature for at least 30 minutes. The syringe cap was removed and replaced with a 25G×1-inch Becton Dickinson needle and loaded into a jig where the HVAI was assembled by Halozyme engineering staff. After final assembly of the HVAI a needle guard was placed over the needle to bring the exposed needle depth to 10 mm+1 mm. The length of each needle was recorded.

### Animal Description

Species: Pig (Sus scrofa domestica)
Strain: Yucatan miniature
Sex: Female
Age: ˜4-6 months
Body weight: ˜18-22 kg
Quantity: 3
Source: Premier BioSource (Ramona, CA)

Husbandry: The animals were housed in steel pens with automatic water provided ad libitum. The animals were fed twice daily (AM and PM) but kept NPO after midnight on the day of the study to prevent anesthesia complications. The room environment was set to maintain a temperature of ˜19-23° C. and a relative humidity of 40-70%, with a 12-hour light/12-hour dark time cycle. The animals were acclimated to the vivarium for a minimum of 3 days prior to study start.

### Test Materials

TABLE 1
Summary of test materials

| Test Material | Supplier | Catalog # |
|---|---|---|
| High Volume Auto-Injector (HVAI) | Halozyme | N/A |
| COC syringes | SCHOTT, Inc. | N/A |
| Sterile rubber plunger for COC | SCHOTT, Inc. | N/A |
| syringes | | |
| Rubber plunger positioning platform | Halozyme | N/A |
| Syringe caps | Becton Dickinson | 305819 |
| 25G × 1 inch Precision Glide needle | Becton Dickinson | 305125 |
| Surgical Eye Spears | BVI Merocel ® | 400101 |
| Standard Digital Camera | Canon | S120 |
| Digital Caliper | Fisher | 06-664-16 |
| 12 mm biopsy punch | Accuderm | NC9253254 |
| 10% neutral buffered formalin | Fisher Scientific | 22-026-435 |

This study assessed the local tolerability of an approved antibody-drug conjugate Sacituzumab govitecan (Trodelvy®) with and without rHuPH20 over time after subcutaneous administration. In this study three animals were used. Each animal received a SC injection to their abdomen of Trodelvy alone followed by a SC injection of Trodelvy+rHuPH20 on the contralateral side of the abdomen. The dose volume was 10 mL and was delivered using a HVAI. The treatment groups are shown in Table 2.

TABLE 2
Description of treatment groups.

| | | Dose | | |
|---|---|---|---|---|
| Cohort | | Volume | rHuPH20 | Evaluation |
| [N/group] | Test Solution | (mL) | (U/mL) | Times (h/d) |
| 1 | Sacituzumab govitecan | 10 | 0 | T0, 1 d, 2 d, |
| [3] | (Trodelvy) | | | 3 d |
| 2 | Sacituzumab govitecan + | 10 | 2000 | T0, 1 d, 2 d, |
| [3] | rHuPH20 (Trodelvy + | | | 3 d |
| | rHuPH20) | | | |

The duration of the injection was measured using a hand-held stopwatch (Fisher). Following administration any back-leakage was collected for a period of 30 seconds and weighed using an analytical balance with a sensitivity of 0.1 mg. Qualitative assessments and photographic images were taken immediately post-injection, and at intervals of 1d, 2d and 3d. After the final imaging timepoint, each animal was humanely euthanized and punch biopsies (12 mm) of each injection site were obtained and fixed in 10% neutral buffered formalin for histological processing and subsequent pathological analyses.

Prior to the start of study, the animals were assessed for general health, and body weights collected. All anesthesia was administered and monitored. One day prior to the study, syringes were filled with appropriate test solutions and assembled into HVAIs then stored at 2-8° C. Devices were allowed to acclimate to room temperature for at least 30 minutes prior to use and used within 2 hours. After anesthetization the animal was placed in dorsal recumbence on a heated surgical table and was maintained under isoflurane gas for the entire duration of the procedure. Following anesthetization, the abdominal region was cleaned with Nolvasan followed by wiping the injection site with gauze containing 70% isopropanol and wiped dry with sterile gauze. Injection sites were located on the left and right abdominal regions, ˜5 cm cranially from the inguinal fold towards the midline and ˜3 cm towards the midline of the animal. Each of the injection sites were marked with a permanent marker and then photographed with the standard and 3D cameras prior to needle insertion. Once injections were completed, two additional devices were brought to room temperature for use with the next animal.

### HVAI Needle Lengths

The needle length of each HVAI was measured prior to use and is shown in Table 3.

TABLE 3
Summary of needle lengths

| Animal | | Needle Length | | Needle Length |
|---|---|---|---|---|
| ID# | Left Side | (mm) | Right Side | (mm) |
| 4493 | Trodelvy | 10.0 | Trodelvy + | 10.0 |
| | | | rHuPH20 | |
| 4498 | Trodelvy + | 10.0 | Trodelvy | 9.5 |
| | rHuPH20 | | | |
| 4593 | Trodelvy | 9.5 | Trodelvy + | 10.0 |
| | | | rHuPH20 | |

The needle was inserted vertically into the marked injection site and the HVAI held in place by hand. Target needle insertion depth was ˜10 mm. Injections were timed using a stopwatch. Upon completion of the injection the needle was removed and the HVAI device discarded. Test solution back-leakage was then absorbed to a tared eye-spear for 30 seconds on the injection site and the weight of the eye spear was measured using an analytical balance. The margins of the injection site bleb were marked with a permanent marker and measured for length, width, and height using a digital caliper and recorded then photographed with the standard and 3D cameras. Caliper measurements and photographs were taken immediately post-injection. Local injection sites were qualitatively assessed and graded daily for erythema severity, swelling size appearance, and firmness, using a scoring system described in Table 4 Table 5, and Table 6, respectively.

TABLE 4
Grading scale for erythema formation

| Scale | Description |
|---|---|
| 0 | No erythema |
| 1 | Very slight erythema (barely perceptible) |
| 2 | Well defined erythema |
| 3 | Moderate to severe erythema |
| 4 | Severe erythema (beet redness) to slight eschar formation |

TABLE 5
Grading scale for swelling formation

| Scale | Description |
|---|---|
| 0 | No swelling |
| 1 | Very slight swelling |
| 2 | Slight swelling |
| 3 | Moderate swelling |
| 4 | Severe swelling |

TABLE 6
Grading scale for swelling firmness (induration)

| Scale | Description |
|---|---|
| 0 | No perceptible difference in firmness after injection |
| 1 | Very slightly firm (barely perceptible) |
| 2 | Mildly firm |
| 3 | Moderately firm |
| 4 | Very firm |

Approximately, 24 hours post-dosing of the first injection, each animal was re-anesthetized, and injection sites assessed for erythema, induration and swelling, then photographed and returned to its cage. The scoring and evaluations continued daily up to approximately three days (72h).

Following the assessment after day three, the animal was humanely euthanized using an injectable euthanasia drug provided by the vivarium staff. Following euthanasia full thickness punch biopsies of the injection site were taken (12 mm) and placed in 10% formalin. After obtaining the punch biopsies the animal carcass was removed and disposed of as biohazardous waste. Sections were made from tissue samples and assessed for histopathology using hematoxylin and eosin staining methods. Analyses were performed on each injection site (2 biopsies) and one untreated site. Three tissue depths were examined from each tissue section following level sectioning technique, representing the beginning, middle and end of each section.

Assessment of Injection Time. The duration of the injection was measured using a digital stopwatch with resolution of 0.1 seconds.

Assessment of Local Swelling Volume and Area Using Caliper Measurement and 3D Imaging. Volume and area of post-injection swelling were measured using both caliper measurement and 3D camera image analysis. For caliper measurements a digital caliper was utilized to measure length, width and height of the bleb that formed post-injection. The length and width are defined as the edge to edge measurements of the bleb (i.e., diameter) along their longest axes. These values were manually recorded, and the volume determined using the formula for half of an ellipsoid Vol=(⅔)*π*A*B*C where A=Length/2, B=Width/2 and C=Height.

3D imaging was applied as a longitudinal methodology to measure post-injection swelling. By obtaining high definition pre- and post-injection 3D images the distances between two registered surfaces can be determined. The camera captures images using a factory calibrated bifocal imaging system to measure distance between surfaces. Surface registration was performed using a multipoint method that utilized common landmarks between the pre-injection image and the post-injection image. Using the proprietary software, the volume, area and height of the post-injection swelling was calculated for each injection (Canfield Biosciences, Inc.).

Caliper measurement and 3D imaging measurement yield different values for volume, area, and bleb height. The differences are a result of the difference in the bleb size measurement. The 3D measurement calculates bleb height based on the top of the bleb to the original skin position, while the bleb height from caliper measurements measure from the top of the bleb to the height at the edge of the bleb. Due to skin curvature, this may yield an overall increase in bleb height for the caliper measurements compared to the 3D measurements, resulting in greater bleb volume and height. However, the measurements are consistent with each other and therefore differ only due to the methodology.

Statistical comparisons for Trodelvy and Trodelvy+rHuPH20 were performed using an unpaired parametric t-test (Prism v10.1.1, GraphPad Software, Inc., San Diego, California). All hypotheses' tests were performed at a 5% significance level, thus considered significant if p<0.05. Pre- and Post-Injection Quantitative Measurements

Quantitative measurements were taken immediately post-injection (T0) and daily afterwards up to ˜72 hours.

### Duration of Injection

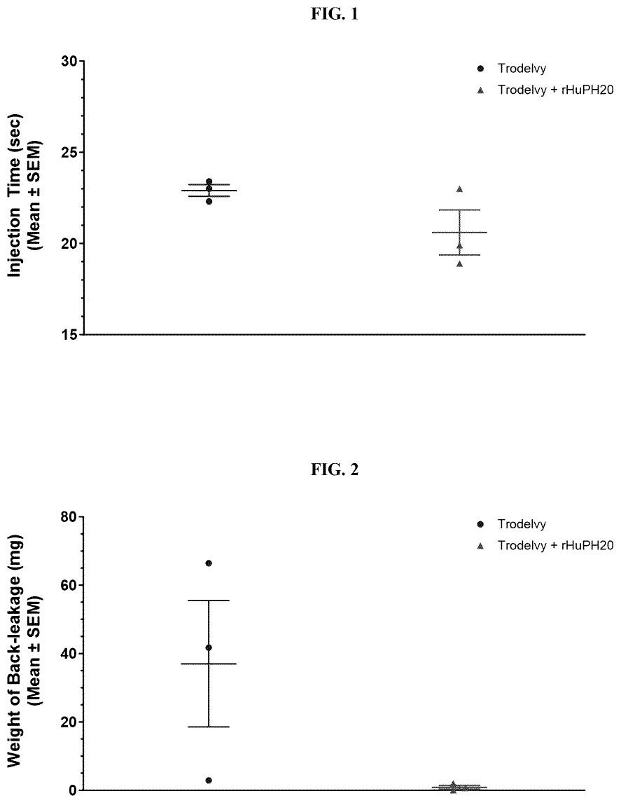

The duration of each injection was measured using a hand-held stopwatch with a precision of 0.1 seconds. The duration of the injections for each test solution is shown in Table 7 and individual animal data shown in FIG. 1.

TABLE 7
Measurement of injection time

| Test Solution | Injection Time (sec) | % Decrease |
|---|---|---|
| Trodelvy | 22.9 ± 0.6 | — |
| Trodelvy + rHuPH20 | 20.6 ± 2.1 | −10% |

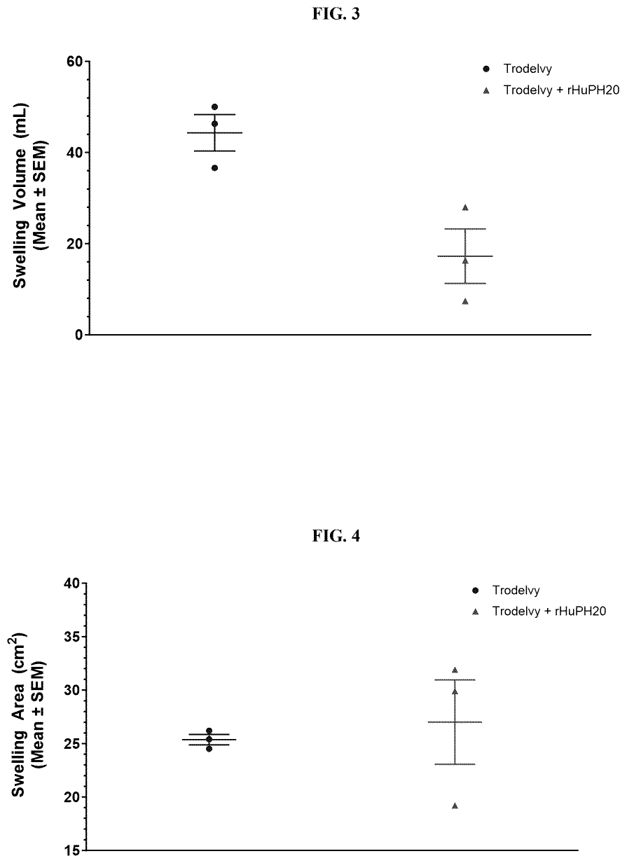

Assessment of Post-Injection Back-Leakage. The back-leakage was collected using a pre-weighed eye spear for 30 seconds post-injection. The eye spear was then re-weighed, and the weight of the back-leakage recorded. The amount of back-leakage for each test solution is shown in Table 8 and individual animal data shown in FIG. 2.

TABLE 8
Measurement of back-leakage

| Test Solution | Back-Leakage (mg ± SEM) | % Decrease |
|---|---|---|
| Trodelvy | 37.0 ± 32.0 | — |
| Trodelvy + rHuPH20 | 0.9 ± 1.0 | −98% |

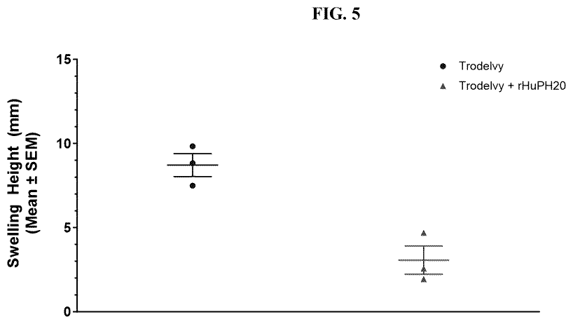

Assessment of Post-Injection Bleb Volume, Area and Height (Caliper Measurements). The local injection site swelling (bleb) was marked and measured using a digital caliper. Bleb volume dispersion area and swelling height of each bleb are summarized in Table 9. Mean and individual post-injection bleb volume, area and height values are shown in FIG. 3, FIG. 4, and FIG. 5.

TABLE 9
Post-Injection Bleb Volume, Area and Height -
Caliper Measurements (Mean ± SEM)

| | | Mean ± SEM | | |
|---|---|---|---|---|
| Cohort # | Test Solution | Volume | Area | Height |
| 1 | Trodelvy | 44.3 ± 4.0 | 25.4 ± 0.5 | 8.7 ± 0.7 |
| 2 | Trodelvy + rHuPH20 | 17.3 ± 6.0 | 27.0 ± 3.9 | 3.1 ± 0.8 |
| | % Decrease + rHuPH20 | −61% | +6% | −64% |

Assessment of Post-Injection Bleb Shape, Volume, Area and Height (3D Imaging). Pre- and post-injection photographs were taken using a 3D imaging system (Canfield Scientific). This technology permits point-to-point alignment of these two images through multipoint surface registration. The distance between any two points is then represented using a colorimetric surface contour map. Regions where there is no difference between the two images are displayed in gray. Where the post-injection image is higher than the pre-injection image, the region is displayed in shades of blue. Where the post-injection image is lower than the pre-injection image the distance is displayed in shades of orange. The color intensity is proportional to the amount of distance measured between images with darker blue color indicating greater distance from the pre-injection image. Out of range measurements (distances greater than 6 mm) are depicted in white. Bleb measurements of volume and height include regions out of range.

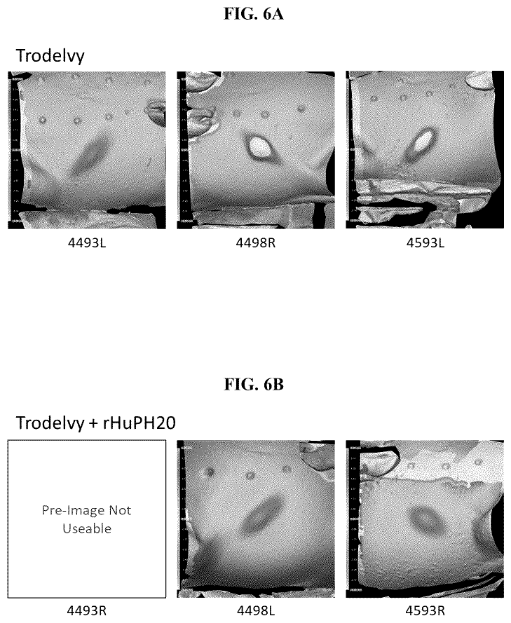

Each animal had a pre-injection 3D image taken of the injection site followed by a second image taken immediately post-injection and these images were mapped to each other using multipoint registration. These registered pre-/post-injection images were then used to calculate the bleb volume, height, circumference, length, and width for each bleb using proprietary software (Vectra H1 software; Canfield Sciences). Colorimetric surface contour maps of each post-injection bleb for Trodelvy and Trodelvy+rHuPH20 are shown in FIGS. 6A and 6B, respectively.

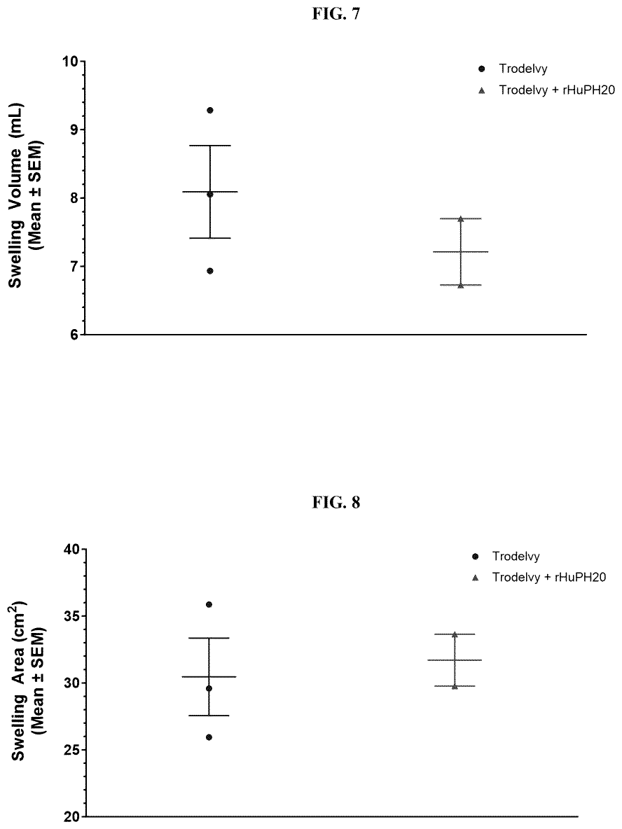

Post-injection bleb volume, area, and height for Trodelvy and Trodelvy+rHuPH20 calculated from the 3D images are summarized in Table 10. Individual post-injection bleb volume, area, and height are shown graphically in FIG. 7, FIG. 8, and FIG. 9. Because the pre-injection photo for AID #4493R was not able to be processed by the Vectra 3D software, the values for the Trodelvy+rHuPH20 are shown as the mean of the two measured values.

TABLE 10
Swelling volume after injection of Ig-120 and Ig-120 + rHuPH20
over time using caliper measurement (Mean ± SEM)

| | Mean ± SEM | | |
|---|---|---|---|
| Test Solution | Volume (mL) | Area (cm²) | Height (mm) |
| Trodelvy | 8.1 ± 0.7 | 30.5 ± 2.9 | 7.6 ± 1.2 |
| Trodelvy + rHuPH20^{a} | 7.2 ± 0.5 | 31.7 ± 1.9 | 5.7 ± 0.1 |
| % Decrease + rHuPH20 | −11% | +4% | −25% |

^{a}N = 2

### Qualitative Assessment of Local Injection Sites

Qualitative assessments were taken immediately post-injection (T0) and daily thereafter. No erythema, swelling or induration was detected on D1, D2 or D3 post-injection.

### Post-Injection Erythema

No erythema was observed for either test solution following injection at any timepoint.

### Post-Injection Swelling Size

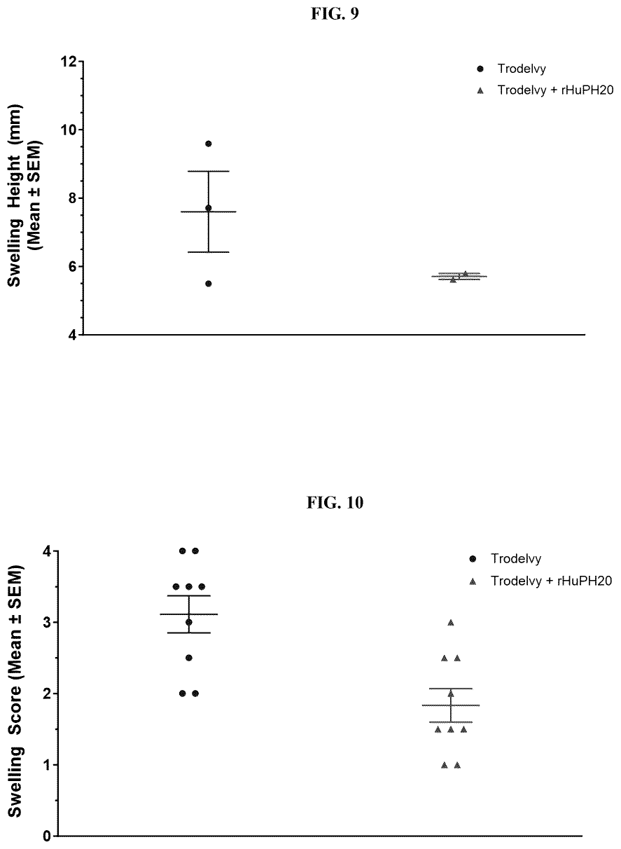

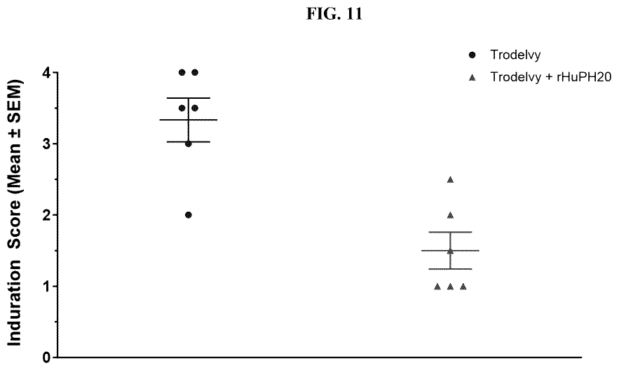

Post-injection swelling size ranged from slight (2) to severe (4) for Trodelvy alone and from very slight (1) to moderate (3) for injections of Trodelvy+rHuPH20. Scoring by three evaluators for swelling size (Mean±SEM) are summarized in Table 11 and shown in FIG. 10. No swelling was detected at the 24, 48 or 72 hour timepoints.

TABLE 11
Swelling Scored Post-Injection for Trodelvy and
Trodelvy + rHuPH20 (Mean ± SEM)

| Test Solution | Swelling Score (Mean ± SEM) | % Decrease |
|---|---|---|
| Trodelvy | 3.1 ± 0.3 | — |
| Trodelvy + rHuPH20 | 1.8 ± 0.2 | −42% |

### Post-Injection Firmness (Induration)

The hardness (induration) of the post-injection blebs for Trodelvy alone were found to be significantly firmer than the blebs that resulted from injection of Trodelvy+rHuPH20 (p<0.05). No induration was detected at the 24, 48 or 72 hour timepoints. Only two evaluators were available to score for induration and the values (Mean±SEM) are summarized in Table 12 and individual values are shown in FIG. 11. No induration was detected at the 24, 48 or 72 hour timepoints.

TABLE 12
Induration Scores Post-Injection for Trodelvy and
Trodelvy + rHuPH20 (Mean ± SEM)

| Test Solution | Induration Score (Mean ± SEM) | % Decrease |
|---|---|---|
| Trodelvy | 3.3 ± 0.3 | — |
| Trodelvy + rHuPH20 | 1.5 ± 0.3 | −55% |

### Histological Analysis of Post-Injection Tissue Samples

After fixation the samples were processed and embedded into paraffin to make formalin fixed paraffin embedded (FFPE) tissues sections Each tissue section was evaluated at three different tissue depths (1-2-3; low-medium-high). In addition to tissue samples from the injection site, a sample was taken from naïve untreated tissue for comparison. A summary of the histological findings is shown in Table 13.

TABLE 13
Summary of histological findings

| | | | Cell | |
|---|---|---|---|---|
| Sample ID | Level | Necrosis | Infiltrates | Other/Comments |
| 4493L | 1 | 0 | 0 | 0 |
| Trodelvy | 2 | 0 | 0 | 0 |
| | 3 | 0 | 0 | 0 |
| 4493 | 1 | 0 | 0 | 0 |
| Naïve | 2 | 0 | 0 | 0 |
| | 3 | 0 | 0 | 0 |
| 4493R | 1 | 0 | 0 | 0 |
| Trodelvy + | 2 | 0 | 0 | 0 |
| rHuPH20 | 3 | 0 | 0 | 0 |
| 4498R | 1 | 0 | 2 focal SC | Multiple foci in SC; mild |
| Trodelvy | | | | focal epithelial keratosis |
| | 2 | 0 | 2 focal SC | Multiple foci in SC; mild |
| | | | | focal epithelial keratosis |
| | 3 | 0 | 2 focal SC | Multiple foci in SC; mild |
| | | | | focal epithelial keratosis |
| 4498 | 1 | 0 | 0 | 0 |
| Naïve | 2 | 0 | 0 | 0 |
| | 3 | 0 | 0 | 0 |
| 4498L | 1 | 0 | 0 | 0 |
| Trodelvy + | 2 | 0 | 0 | 0 |
| rHuPH20 | 3 | 0 | 0 | 0 |
| 4593L | 1 | 0 | 0 | 0 |
| Trodelvy | 2 | 0 | 0 | 0 |
| | 3 | 0 | 0 | 0 |
| 4593 | 1 | 0 | 0 | 0 |
| Naïve | 2 | 0 | 0 | 0 |
| | 3 | 0 | 0 | 0 |
| 4583R | 1 | 0 | 0 | 0 |
| Trodelvy + | 2 | 0 | 0 | 0 |
| rHuPH20 | 3 | 0 | 0 | 0 |

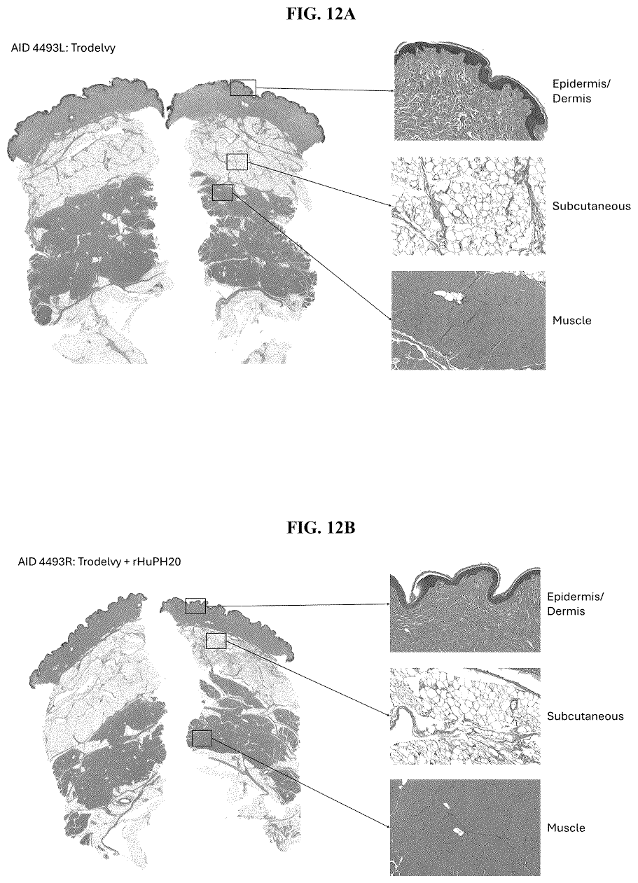

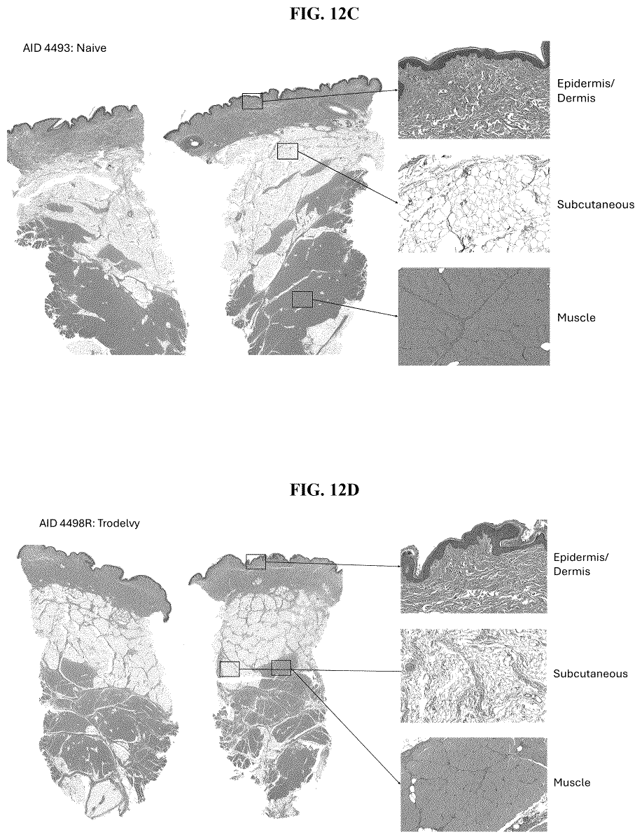

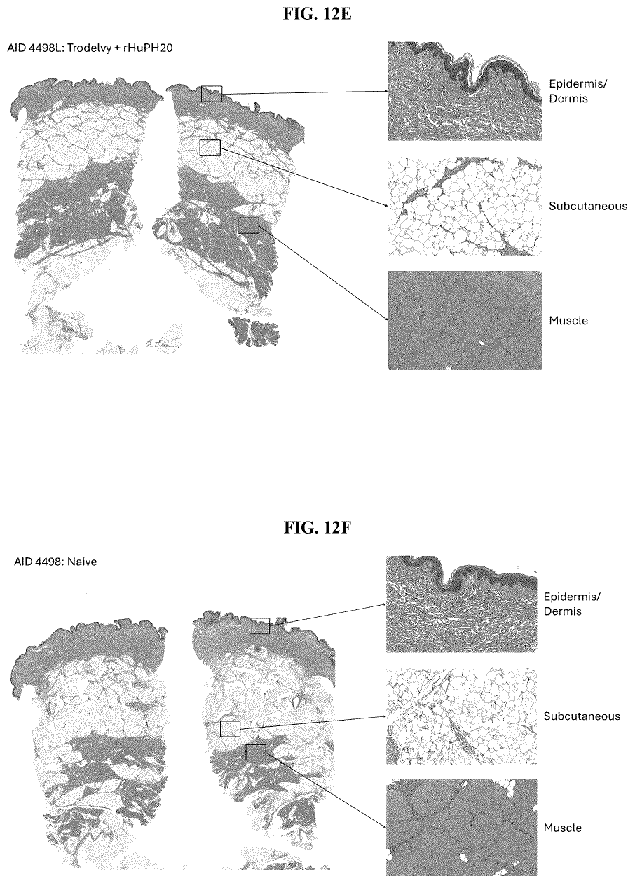

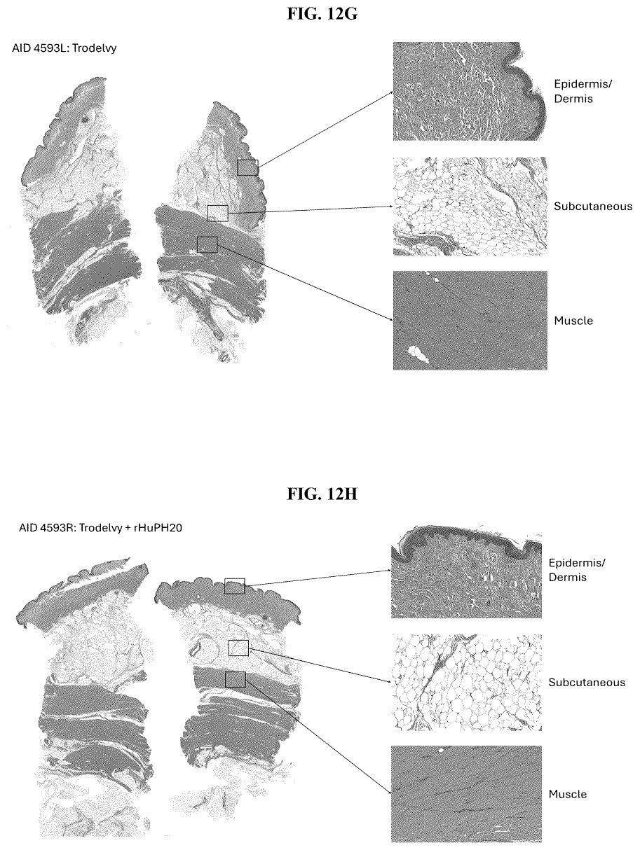

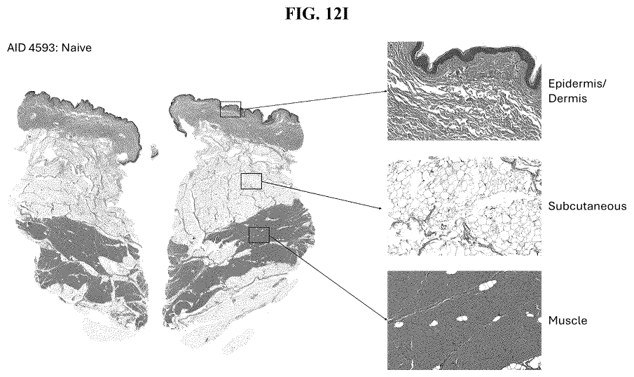

Notably no findings occurred with the injections of Trodelvy+rHuPH20 whereas 1/3 of the injections of Trodelvy alone showed mild focal epithelial keratosis in all levels of the tissue sample. In addition, there were 2 focal instances of [immune] cell infiltrates in the SC space. Histological images of tissue sections stained with hematoxylin and eosin (H&E) together with magnified images of the dermis, subcutaneous and muscle tissues (5×) of biopsies taken from the Trodelvy±rHuPH20 injection sites and naïve skin are shown in FIGS. 12A-12I.

The addition of rHuPH20 to the 10 mL dose of Trodelvy was well tolerated when delivered subcutaneously. In addition to the tolerability this study showed:

HVAI devices with Trodelvy+rHuPH20 had a reduced mean time of delivery (−10%) compared to injection times for Trodelvy alone.

Back-leakage was extremely low and showed significantly less variability for HVAI injections with Trodelvy+rHuPH20, whereas injections of Trodelvy alone had substantial and highly variable amounts of back-leakage.

Post-injection bleb volume and height using caliper measurements were found to be significantly less for injections of Trodelvy+rHuPH20 compared to Trodelvy alone.

Post-injection bleb height showed significantly less variability between injection sites that contained rHuPH20 compared to those without.

Qualitative scoring of post-injection swelling size using a 5-point modified Draize scale showed that Trodelvy+rHuPH20 blebs were −42% compared to the size of blebs of Trodelvy alone. Qualitative scoring of post-injection induration showed that the Trodelvy+rHuPH20 blebs were consistently softer (−55%) than the blebs that were assessed after injection of Trodelvy alone.

Histopathological analysis showed that one of the injection sites with Trodelvy alone resulted in subcutaneous injection site findings that included mild focal keratosis and immune cell infiltrates which may be indicative of a wound response to an acute buildup of injection pressure in the extracellular matrix.

No signs of tissue necrosis were observed suggesting that the payload of the ADC was intact and dispersed from the injection site.

### Example 2: Evaluation of Subcutaneous Administration of an Antibody Drug Conjugate (Trodelvy®) with and without Recombinant Human Hyaluronidase Ph20 (Rhuph20)

This study utilized all the same protocols and parameters as set forth in Example 1 unless disclosed otherwise. As in the previous Example, prior to study start, the enzyme activity of the test solution was measured using an in vitro activity assay (VV-QWUAL-006751 formerly TM010) to confirm the concentration of rHuPH20 in the co-mix drug solution.

Pre-study Enzymatic Activity Testing of rHuPH20 in Test Solutions. A sample of the ADC+rHuPH20 was tested for hyaluronidase activity and found to be acceptable for use. The pre-study enzyme activity is shown in Table 14.

TABLE 14
Pre-study enzymatic activity testing of rHuPH20 in test solution.

| Test Solution | Pre-study Enzyme Activity (U/mL ± SD) |
|---|---|
| ADC + rHuPH20 | 2270 ± 19 |

### Pre- and Post-Injection Quantitative Measurements

Quantitative measurements were taken immediately post-injection (T0) and daily afterwards up to ˜72 hours.

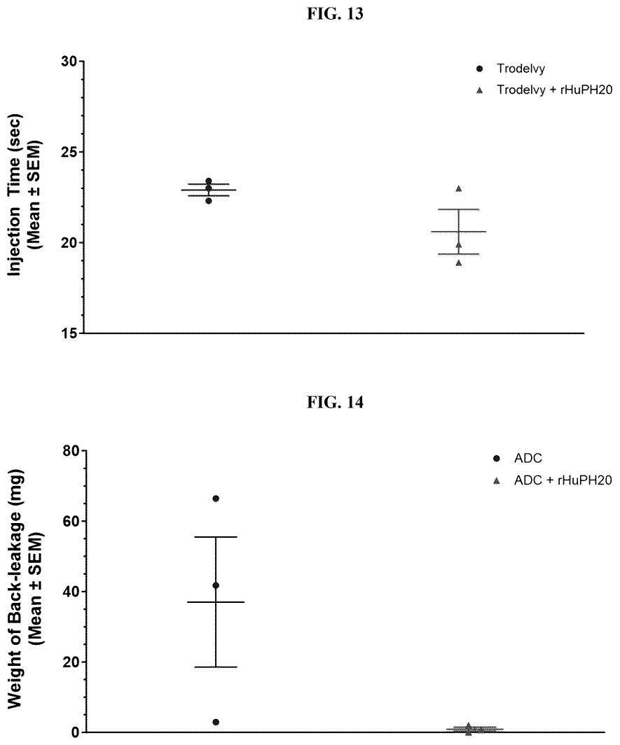

Duration of Injection. The duration of each injection was measured using a hand-held stopwatch with a precision of 0.1 seconds. The duration of the injections for each test solution is shown in Table 15 and individual animal data shown in FIG. 13.

TABLE 15
Measurement of injection time

| Test Solution | Injection Time (sec) | % Decrease |
|---|---|---|
| ADC | 22.9 ± 0.6 | — |
| ADC + rHuPH20 | 20.6 ± 2.1 | −10% |

Assessment of Post-Injection Back-Leakage. The back-leakage was collected using a pre-weighed eye spear for 30 seconds post-injection. The eye spear was then re-weighed, and the weight of the back-leakage recorded. The amount of back-leakage for each test solution is shown in Table 16 and individual animal data shown in FIG. 14.

TABLE 16
Measurement of back-leakage

| Test Solution | Back-Leakage (mg ± SEM) | % Decrease |
|---|---|---|
| ADC | 37.0 ± 32.0 | — |
| ADC + rHuPH20 | 0.9 ± 1.0 | −98% |

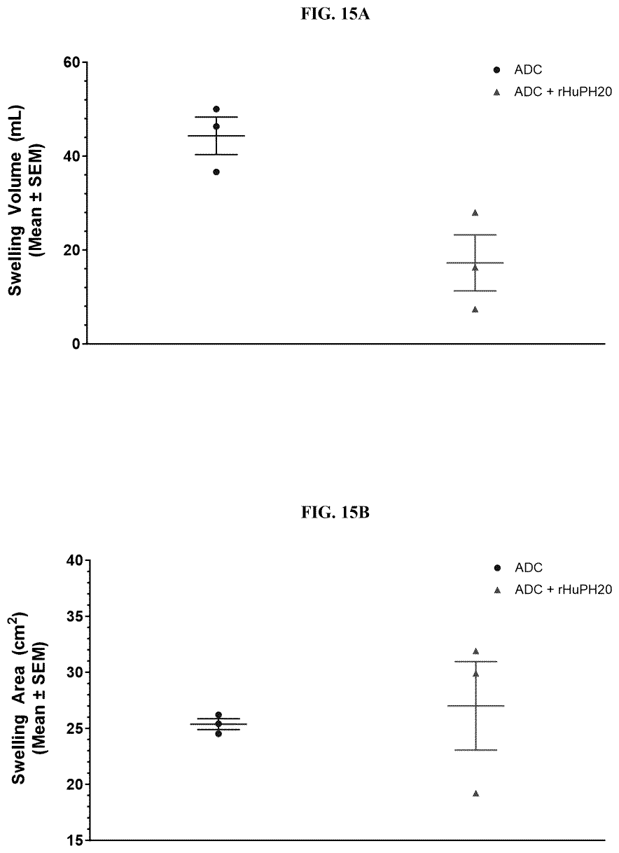

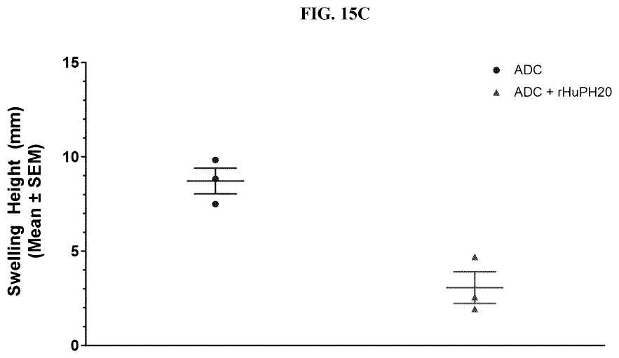

Assessment of Post-Injection Bleb Volume, Area and Height (Caliper Measurements). The local injection site swelling (bleb) was marked and measured using a digital caliper. Bleb volume dispersion area and swelling height of each bleb was determined as described in Section 6.2 and are summarized in Table 17. Mean and individual post-injection bleb volume, area and height values are shown in FIG. 15A, FIG. 15B, and FIG. 15C, respectively.

TABLE 17
Post-injection bleb volume, area, and height -
caliper measurements (mean ± SEM)

| | | Mean ± SEM | | |
|---|---|---|---|---|
| Cohort # | Test Solution | Volume | Area | Height |
| 1 | ADC | 44.3 ± 4.0 | 25.4 ± 0.5 | 8.7 ± 0.7 |
| 2 | ADC + rHuPH20 | 17.3 ± 6.0 | 27.0 ± 3.9 | 3.1 ± 0.8 |

| | | | |
|---|---|---|---|
| % Increase/Decrease | −61% | +6% | −64% |

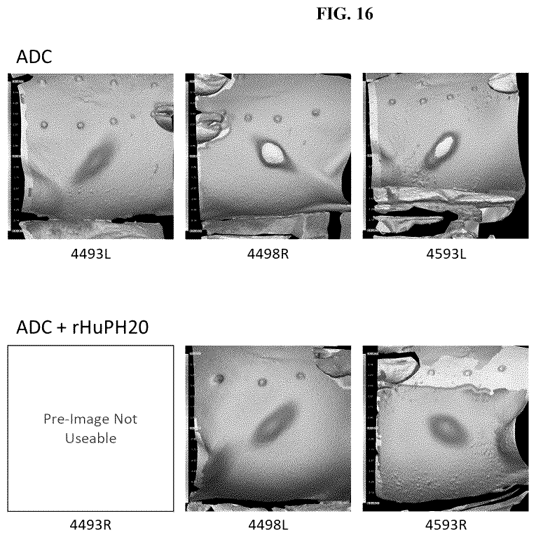

Each animal had a pre-injection 3D image taken of the injection site followed by a second image taken immediately post-injection and these images were mapped to each other using multipoint registration. These registered pre-/post-injection images were then used to calculate the bleb volume, height, circumference, length, and width for each bleb using proprietary software (Vectra H1 software; Canfield Sciences). Colorimetric surface contour maps of each post-injection bleb for ADC and ADC+rHuPH20 are shown in FIG. 16.

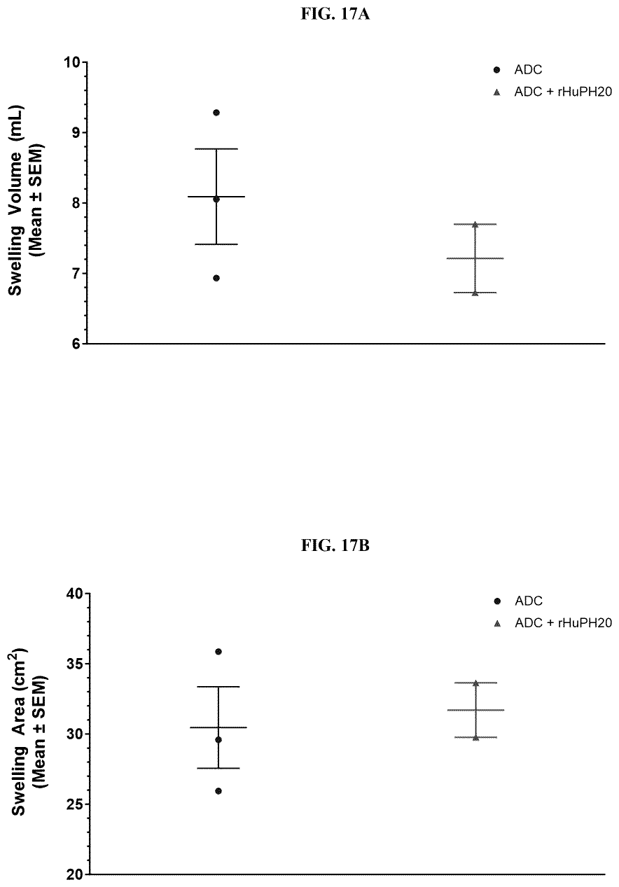

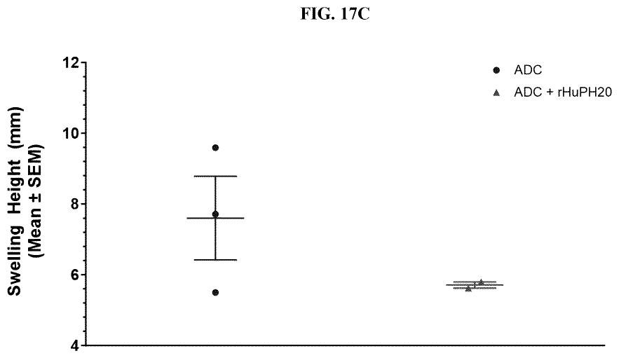

Post-injection bleb volume, area, and height for ADC and ADC+rHuPH20 calculated from the 3D images are summarized in Table 18. Individual post-injection bleb volume and height are shown graphically in FIG. 17A, FIG. 17B, and FIG. 17C. Because the pre-injection photo for AID #4493R was not able to be processed by the Vectra 3D software, the values for the ADC+rHuPH20 are shown as the mean of the two measured values.

TABLE 18
Bleb volume, area and height after injection of ADC and ADC +
rHuPH20 assessed using 3D imaging (mean ± SEM)

| | Mean ± SEM | | |
|---|---|---|---|
| Test Solution | Volume (mL) | Area (cm²) | Height (mm) |
| ADC | 8.1 ± 0.7 | 30.5 ± 2.9 | 7.6 ± 1.2 |
| ADC + rHuPH20^{a} | 7.2 ± 0.5 | 31.7 ± 1.9 | 5.7 ± 0.1 |
| % Increase/Decrease | −11% | +4% | −25% |

^{a}n = 2

Qualitative assessments were taken immediately post-injection (T0) and daily thereafter. No erythema, swelling or induration was detected on D1, D2 or D3 post-injection (data not shown).

Post-Injection Erythema. No erythema was observed for either test solution following injection at any timepoint (data not shown).

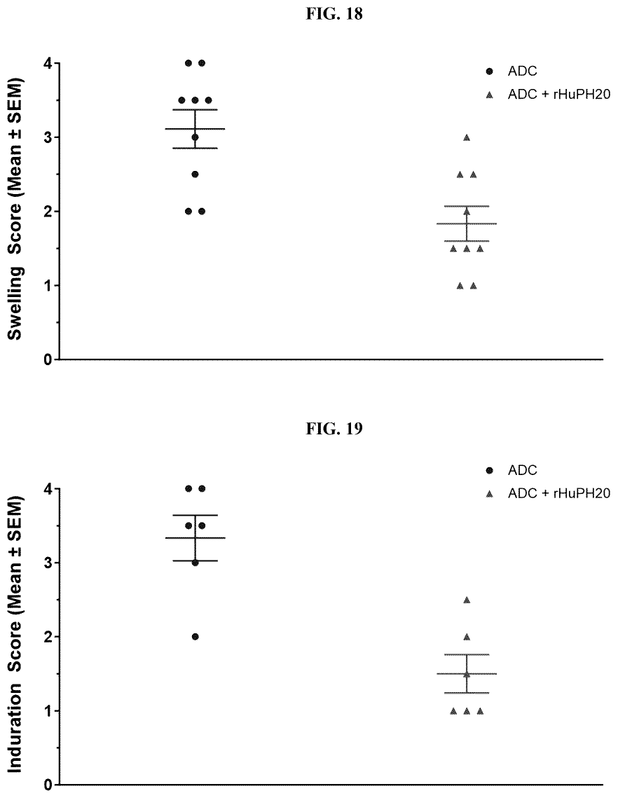

Post-Injection Swelling Size. Post-injection swelling size ranged from slight (2) to severe (4) for ADC alone and from very slight (1) to moderate (3) for injections of ADC+rHuPH20. Scoring by three evaluators for swelling size (Mean±SEM) are summarized in Table 19 and shown in FIG. 18. No swelling was detected at the 24 h, 48h or 72h timepoints (data not shown).

TABLE 19
Swelling scored post-injection for ADC and
ADC + rHuPH20 (mean ± SEM)

| Test Solution | Swelling Score (Mean ± SEM) | % Decrease |
|---|---|---|
| ADC | 3.1 ± 0.3 | — |
| ADC + rHuPH20 | 1.8 ± 0.2 | −42% |

Post-Injection Firmness (Induration). The hardness (induration) of the post-injection blebs for ADC alone were found to be significantly firmer than the blebs that resulted from injection of ADC+rHuPH20 (p<0.05). No induration was detected at the 24 h, 48h or 72h timepoints (data not shown). Only two evaluators were available to score for induration and the values (Mean±SEM) are summarized in Table 20 and individual values are shown in FIG. 19. No induration was detected at the 24 h, 48h or 72h timepoints (data not shown).

TABLE 20
Induration scores post-injection for ADC and
ADC + rHuPH20 (mean ± SEM).

| | Induration Score | |
|---|---|---|
| Test Solution | (Mean ± SEM) | % Decrease |
| ADC | 3.3 ± 0.3 | — |
| ADC + rHuPH20 | 1.5 ± 0.3 | −55% |

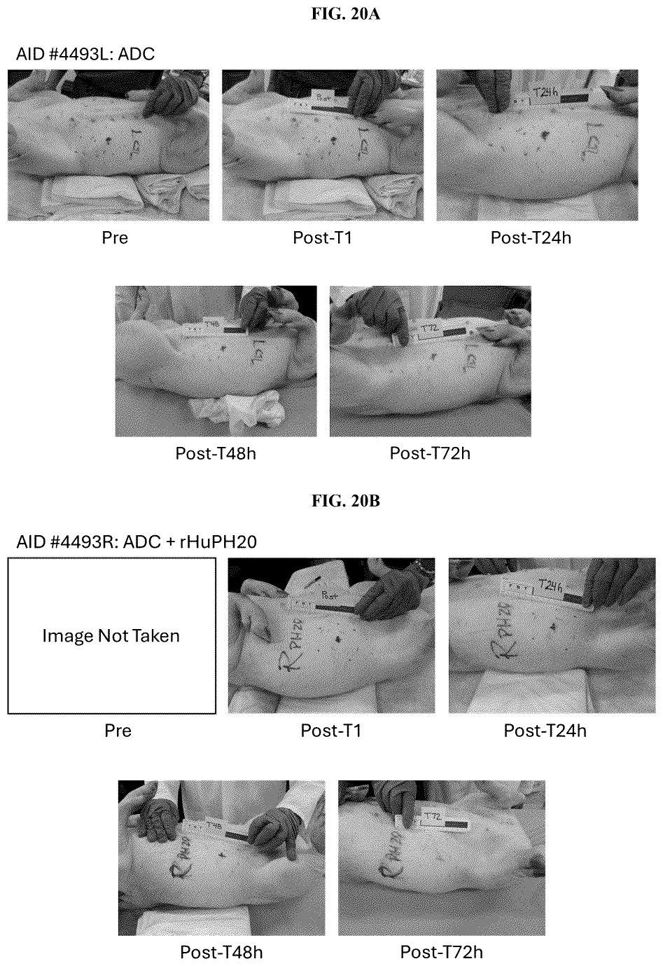

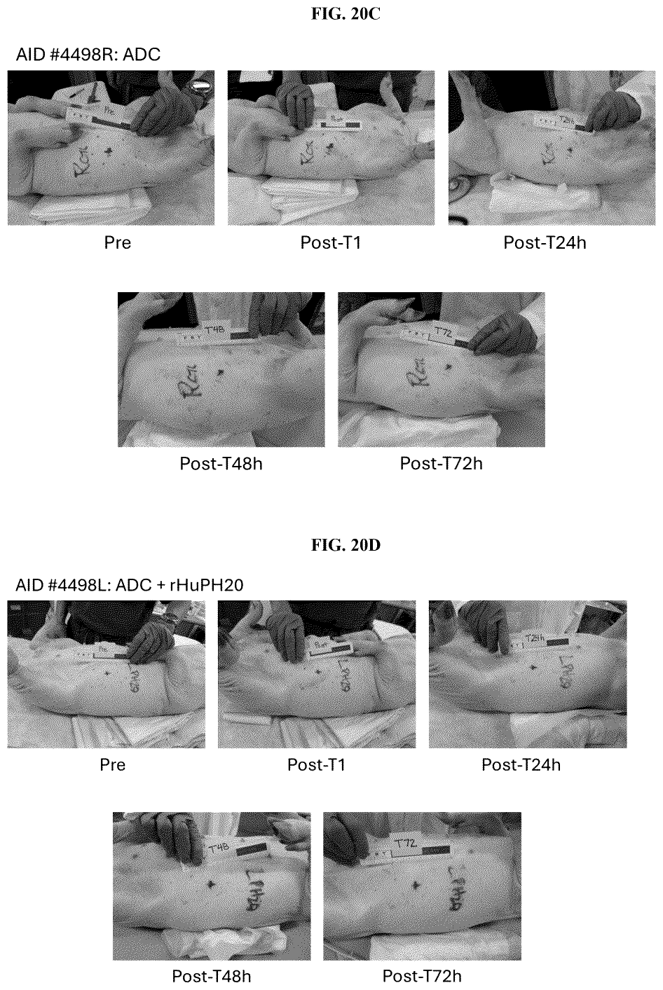

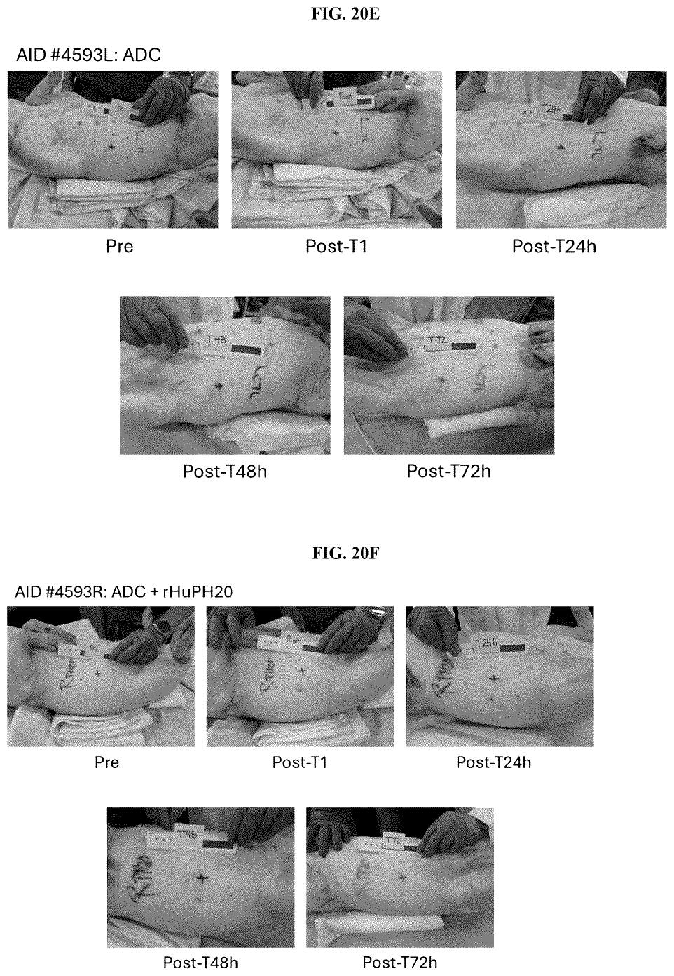

The injection sites were photographed before and immediately post-injection and then at daily intervals up to 72 hours post-injection. Photographic images are shown in FIGS. 20A-20F. Histological Analysis of Post-Injection Tissue Samples. After fixation the samples were processed and embedded into paraffin to make formalin fixed paraffin embedded (FFPE) tissues sections Each tissue section was evaluated at three different tissue depths (1-2-3; low-medium-high). In addition to tissue samples from the injection site, a sample was taken from naïve untreated tissue for comparison. A summary of the histological findings is shown in Table 21.

TABLE 21
Summary of Histological Findings

| Sample | | | Cell | |
|---|---|---|---|---|
| ID | Level | Necrosis | Infiltrates | Other/Comments |
| 4493L | 1 | 0 | 0 | 0 |
| ADC | 2 | 0 | 0 | 0 |
| | 3 | 0 | 0 | 0 |
| 4493 | 1 | 0 | 0 | 0 |
| Naïve | 2 | 0 | 0 | 0 |
| | 3 | 0 | 0 | 0 |
| 4493R | 1 | 0 | 0 | 0 |
| ADC + | 2 | 0 | 0 | 0 |
| rHuPH20 | 3 | 0 | 0 | 0 |
| 4498R | 1 | 0 | 2 focal SC | Multiple foci in SC; |
| ADC | | | | mildfocal epithelial keratosis |
| | 2 | 0 | 2 focal SC | Multiple foci in SC; |
| | | | | mildfocal epithelial keratosis |
| | 3 | 0 | 2 focal SC | Multiple foci in SC; |
| | | | | mildfocal epithelial keratosis |
| 4498 | 1 | 0 | 0 | 0 |
| Naïve | 2 | 0 | 0 | 0 |
| | 3 | 0 | 0 | 0 |
| 4498L | 1 | 0 | 0 | 0 |
| ADC + | 2 | 0 | 0 | 0 |
| rHuPH20 | 3 | 0 | 0 | 0 |
| 4593L | 1 | 0 | 0 | 0 |
| ADC | 2 | 0 | 0 | 0 |
| | 3 | 0 | 0 | 0 |
| 4593 | 1 | 0 | 0 | 0 |
| Naïve | 2 | 0 | 0 | 0 |
| | 3 | 0 | 0 | 0 |
| 4583R | 1 | 0 | 0 | 0 |
| ADC + | 2 | 0 | 0 | 0 |
| rHuPH20 | 3 | 0 | 0 | 0 |

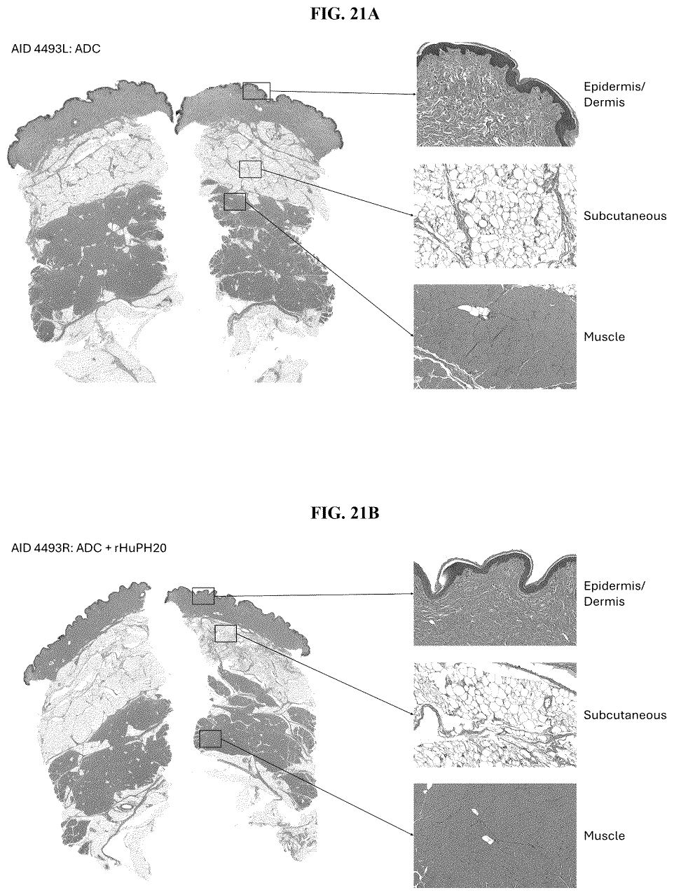

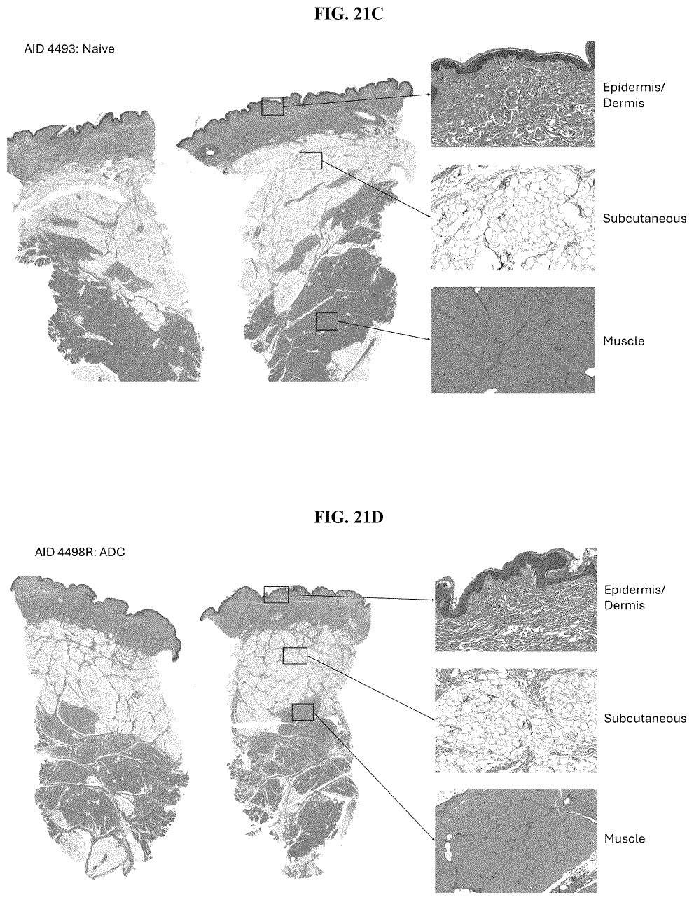

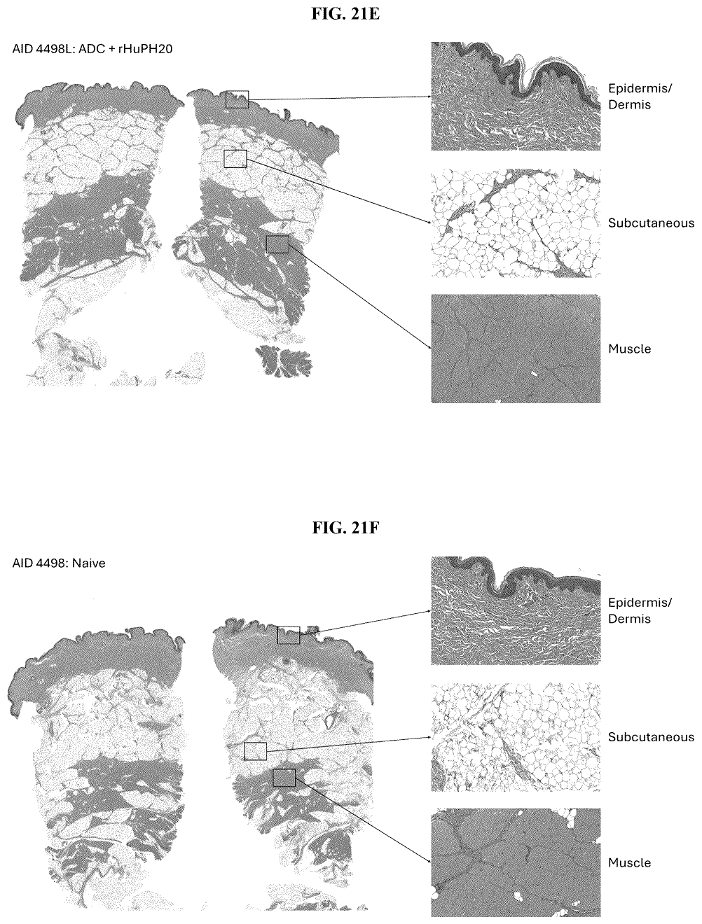

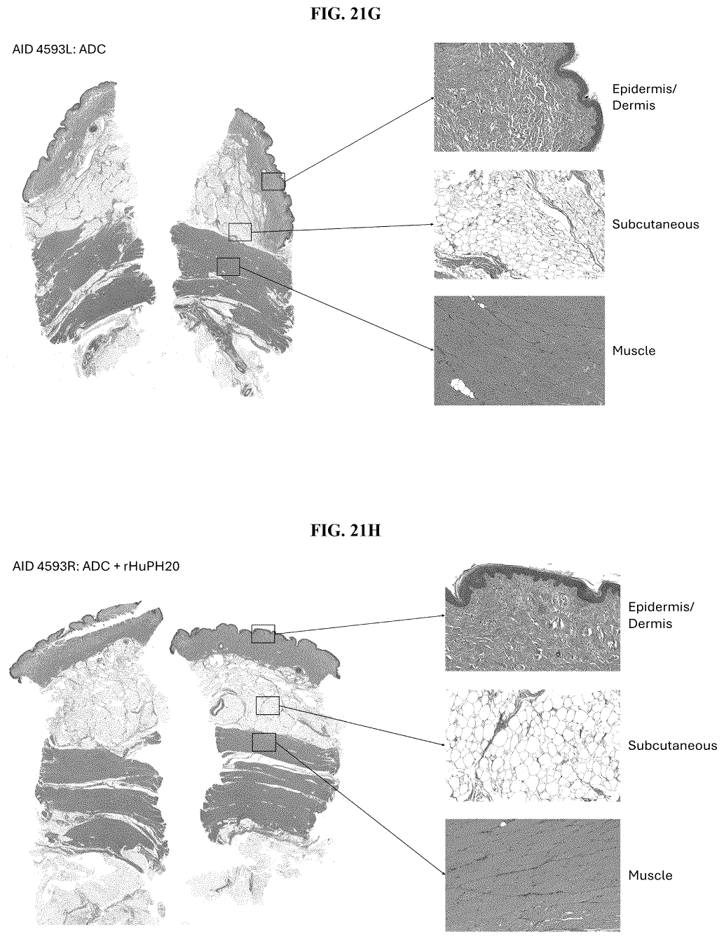

Notably no findings occurred with the injections of ADC+rHuPH20 whereas 1/3 of the injections of ADC alone showed mild focal epithelial keratosis in all levels of the tissue sample. In addition, there were 2 focal instances of [immune] cell infiltrates in the SC space. Histological images of tissue sections stained with hematoxylin and eosin (H&E) together with magnified images of the dermis, subcutaneous and muscle tissues (5×) of biopsies taken from the ADC±rHuPH20 injection sites and naïve skin are shown in FIG. 21A-21I.

The addition of rHuPH20 to the 10 mL dose of ADC was well tolerated when delivered subcutaneously. In addition to the tolerability this study showed:
HVAI devices with ADC+rHuPH20 had a reduced mean time of delivery (−10%) compared to injection times for ADC alone.
Back-leakage was extremely low and showed significantly less variability for HVAI injections with ADC+rHuPH20, whereas injections of ADC alone had substantial and highly variable amounts of back-leakage.
Post-injection bleb volume and height using caliper measurements were found to be significantly less for injections of ADC+rHuPH20 compared to ADC alone.
Post-injection bleb height showed significantly less variability between injection sites that contained rHuPH20 compared to those without.
Qualitative scoring of post-injection swelling size using a 5-point modified Draize scale showed that ADC+rHuPH20 blebs were −42% compared to the size of blebs of ADC alone.
Qualitative scoring of post-injection induration showed that the ADC+rHuPH20 blebs were consistently softer (−55%) than the blebs that were assessed after injection of ADC alone.
Histological analysis showed that one of the injection sites with ADC alone resulted in histopathological findings that included mild focal keratosis and immune cell infiltrates in the subcutaneous space which may be indicative of a wound response to an acute buildup of injection pressure in the extracellular matrix.
No signs of tissue necrosis were observed suggesting that the payload of the ADC was intact and dispersed from the injection site.

### Example 3: Evaluation of Subcutaneous Administration of an Antibody Drug Conjugate (Trodelvy®) with and without Recombinant Human Hyaluronidase PH20 (RhuPH20)

The objective of this study was to determine the local injection site tolerability following a subcutaneous administration of an antibody drug conjugate (ADC). The ADC was administered either alone or co-mixed with recombinant human hyaluronidase PH20 (rHuPH20). For this study the ADC Sacituzumab govitecan was chosen as a representative test solution. This study utilized all the same protocols and parameters as set forth in Example 1 unless disclosed otherwise.

In this initial study one animal was used to assess the local tolerability of subcutaneous administration of the ADC delivered either alone or co-mixed with rHuPH20. For comparison, the animal also received SC administration of the antibody solution alone. Eight SC injections were administered on the abdomen of the animal; four injections were the ADC solution alone and four injections were the ADC co-mixed with rHuPH20. Each injection was one mL and was delivered using a pre-filled 3-cc syringe using a 26G×⅝-inch needle. Injection times were staggered in order to result in various exposure times to the test solutions and resulted in exposures of 0.5, 1, 2 and 4 hours. After the last timepoint, the animal was humanely euthanized. Following euthanasia, punch biopsies of each injection site were obtained and preserved in 10% formalin for histological analyses. Formalin-fixed paraffin sections (FFPE) were prepared of each tissue sample and used to prepare slides that were examined for any morphological changes that occurred as a result of the exposure to ADC alone or ADC+rHuPH20 co-mix.

### Formulation

Prior to study start, the enzyme activity of the test solution was measured using an in vitro activity assay (VV-QWUAL-006751 formerly TM010) to confirm the concentration of rHuPH20 in the co-mix drug solution.

Pre-study Enzymatic Activity Testing of rHuPH20 in Test Solutions. A sample of the ADC+rHuPH20 was tested for hyaluronidase activity and the enzyme activity is shown in Table 22.

TABLE 22
Pre-study enzymatic activity testing of rHuPH20 in test solution.

| Test Solution | Pre-study Enzyme Activity (U/mL ± SD) |
|---|---|
| ADC + rHuPH20 | 2237 ± 42 |

This exploratory study assessed the local tolerability of an approved antibody-drug conjugate (Sacituzumab govitecan) with and without rHuPH20. In this initial study one animal was treated and received four 1-mL injections of the ADC alone and four 1-mL injections of the co-mix of ADC+rHuPH20. Injection times were staggered in order to result in various exposure times to the test solution and resulted in exposures of approximately 0.5, 1, 2 and 4 hours. The ADC was co-mixed with rHuPH20 so that the final concentration was approximately 2000 U/mL (Table 23).

TABLE 23
Description of Treatments.

| | | Dose | | | |
|---|---|---|---|---|---|
| | Test | Volume | # of | [rHuPH20] | Exposure |
| Cohort | Solution | (mL) | Injections | (U/mL) | Time (h) |
| 1 | ADC | 1.0 | 4 | 0 | 0.5, 1, 2, 4 |
| 2 | ADC + | 1.0 | 4 | 2000 | 0.5, 1, 2, 4 |
| | rHuPH20 | | | | |

At each timepoint two injections were given to the animal: one of the ADC solution alone and one of the ADC solution co-mixed with rHuPH20 on the diagonal contralateral side of the animal.

Each injection was one mL and was delivered using a pre-filled 1-cc syringe. After the last timepoint, the animal was humanely euthanized. After euthanasia, punch biopsies of each injection site were obtained and preserved in 10% formalin for histological analyses.

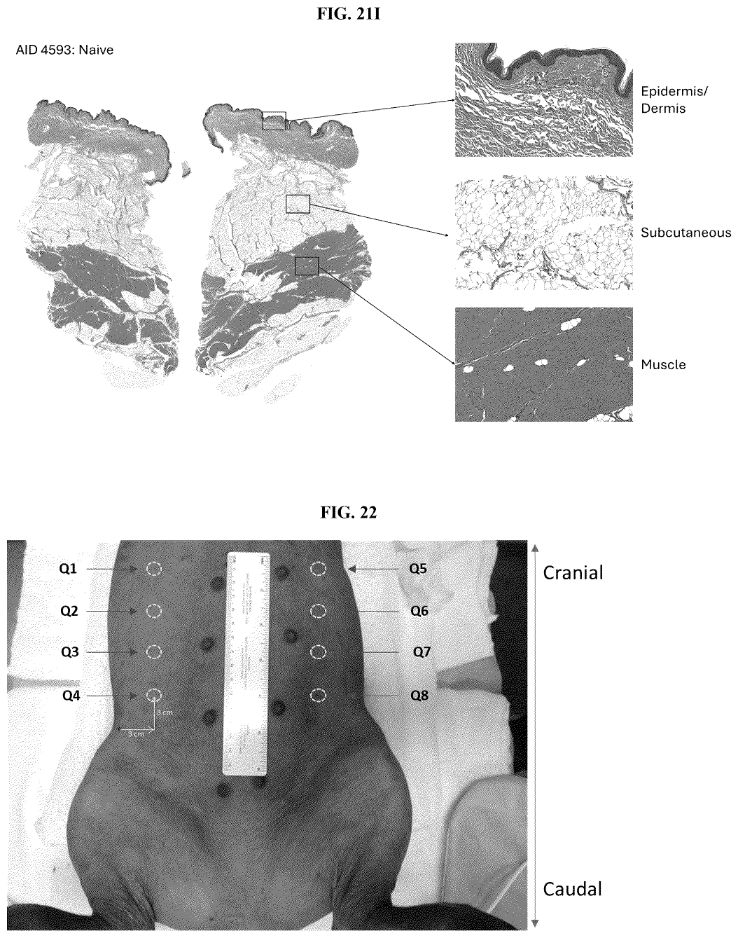

Following anesthetization, the abdominal region was cleaned with Nolvasan followed by wiping the injection site with gauze containing 70% isopropanol and wiped dry with sterile gauze. After drying the eight injection sites were marked (Q1-Q8) and are shown in FIG. 22.

The SC injections were administered on the abdomen of the animal. At each timepoint two injections were given to the animal—one injection of Sacituzumab govitecan alone followed by one injection on the diagonal contralateral side of the animal of Sacituzumab govitecan+rHuPH20.

Four injection sites were located on each side of the animal (Q1-Q4: right abdomen; Q5-Q8: left abdomen) with the lowest sites located 3 cm toward the midline of the animal from the midpoint of the inguinal fold and approximately 3 cm cranially (see below; sites Q7 & Q8). Injection sites (Q1-Q4 & Q5-Q8) were spaced approximately 4 cm apart. The test article and injection time associated for each of the injection sites are described in Table 24.

TABLE 24
Summary of Injection Sites.

| Cohort | Test Solution | Injection Site | Total Exposure Time | |
|---|---|---|---|---|
| 1 | ADC | Q1 | 4 | h |
| | | Q5 | 2 | h |
| | | Q3 | 1 | h |
| | | Q7 | 0.5 | h |
| 2 | ADC + rHuPH20 | Q8 | 4 | h |
| | | Q4 | 2 | h |
| | | Q6 | 1 | h |
| | | Q2 | 0.5 | h |

Each of the injection sites were marked with a permanent marker and then photographed prior to needle insertion. The procedure began with the injection at Injection Site Q1 with the injection of 1 mL SC injection of ADC followed by an injection on the diagonal contralateral side (Q8) which received a 1 mL SC injection of ADC+rHuPH20. Additional injections followed at sites Q5-Q4, Q3-Q7 and Q7-Q2 for exposure times of 2, 1 and 0.5 hours, respectively. Injection sites were photographed at each timepoint. At the end of study, the animal was humanely euthanized. After euthanasia 12 mm punch biopsies were obtained from each injection site and preserved in 10% formalin for tissue processing, paraffin embedding and eventual histological analyses by a licensed pathologist. After obtaining the punch biopsies the animal carcass was removed and disposed.

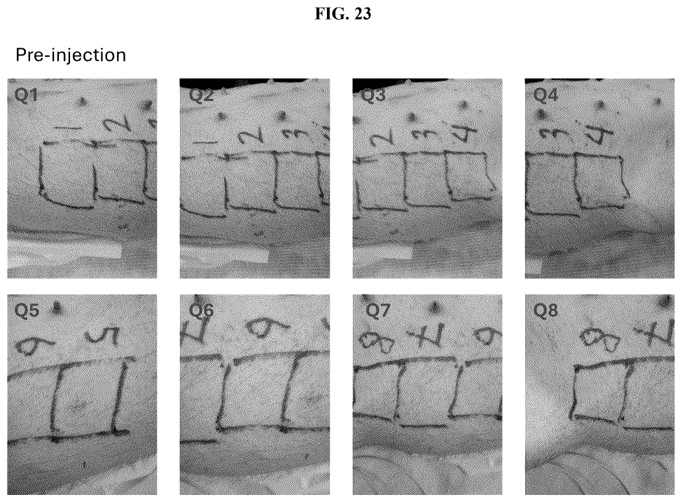

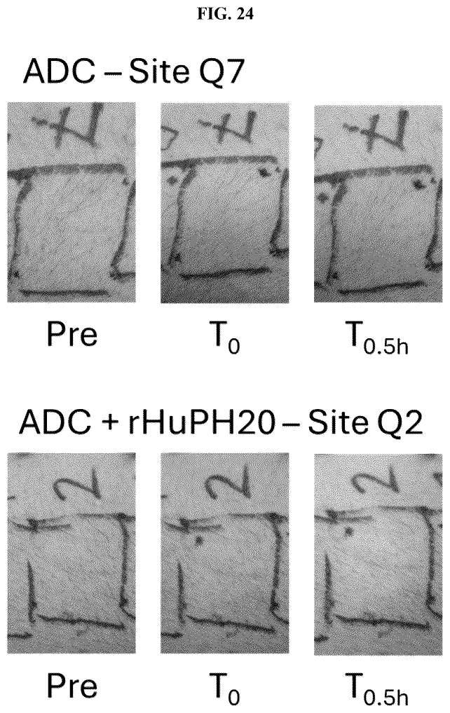

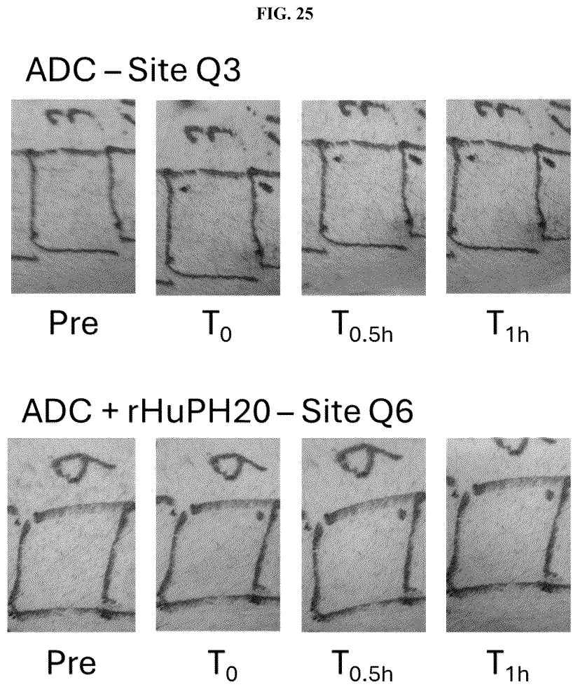

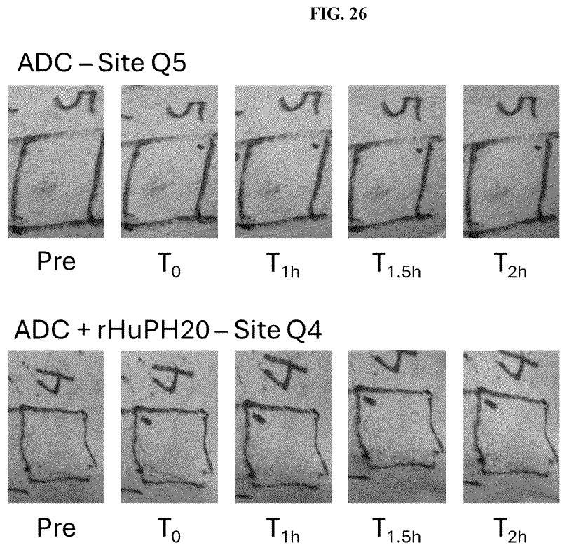

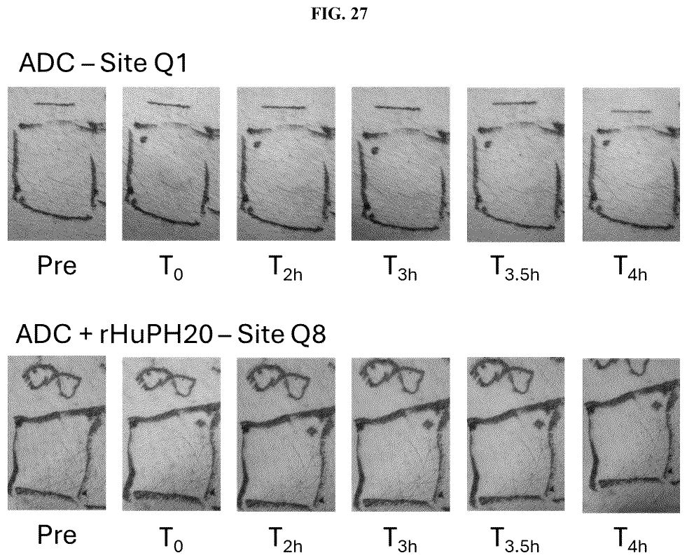

The injection sites were photographed at each timepoint. The pre-injection photos are shown in FIG. 23. The shortest exposure was 0.5 hours and images of the injection site at T₀ and T_{0.5h} are shown in FIG. 24. Images of injection site Q3 (ADC) and Q6 (ADC+rHuPH20) taken at T₀ and T₁ₕ are shown in FIG. 25. Images of injection site Q5 (ADC) and Q4 (ADC+rHuPH20) taken at T₀, T₁ₕ and T₂ₕ are shown in FIG. 26 and images of injection site Q1 (ADC) and Q8 (ADC+rHuPH20 taken at T₀, T₁ₕ, T₂ₕ and T₄ₕ are shown in FIG. 27.

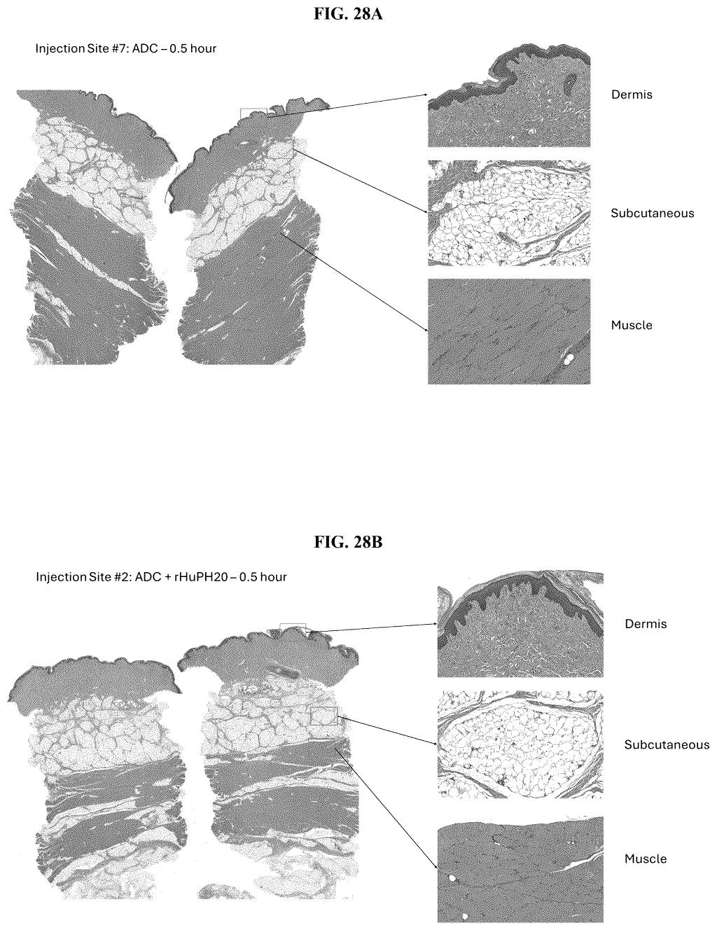

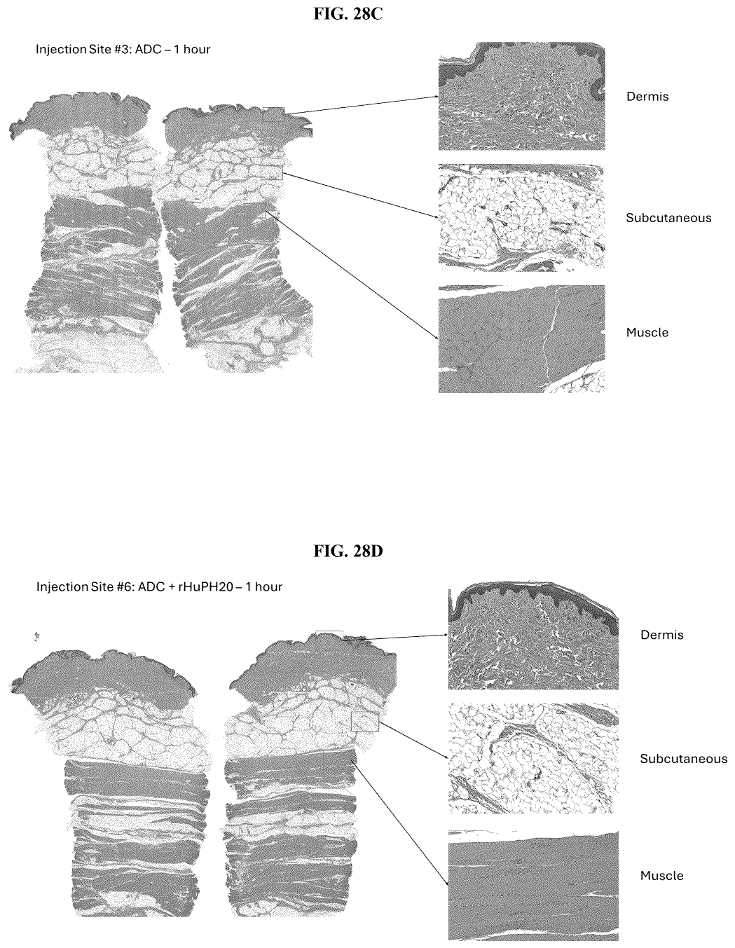

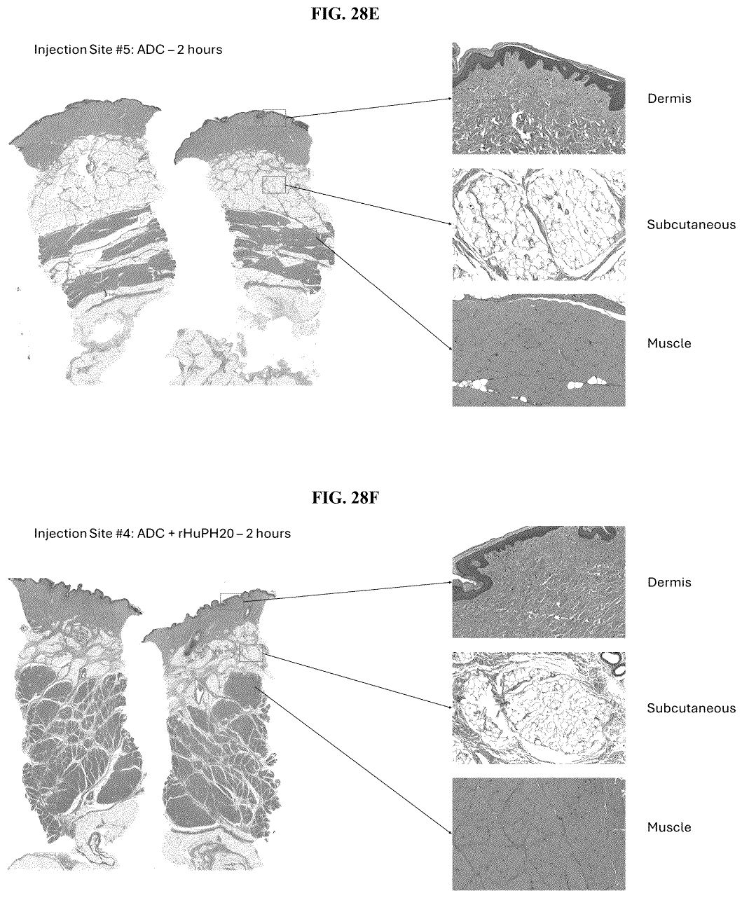

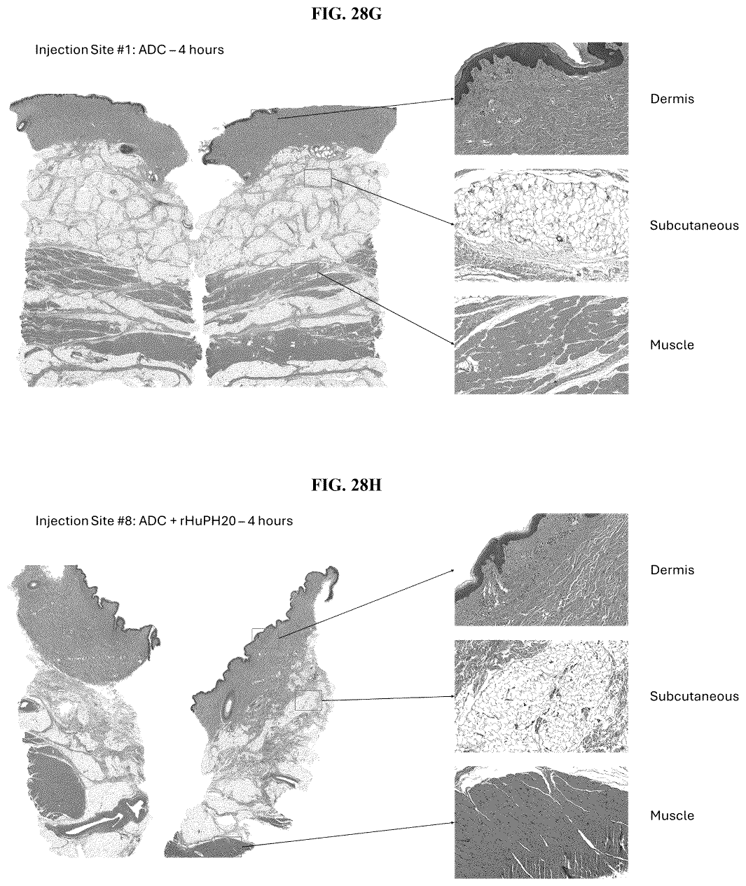

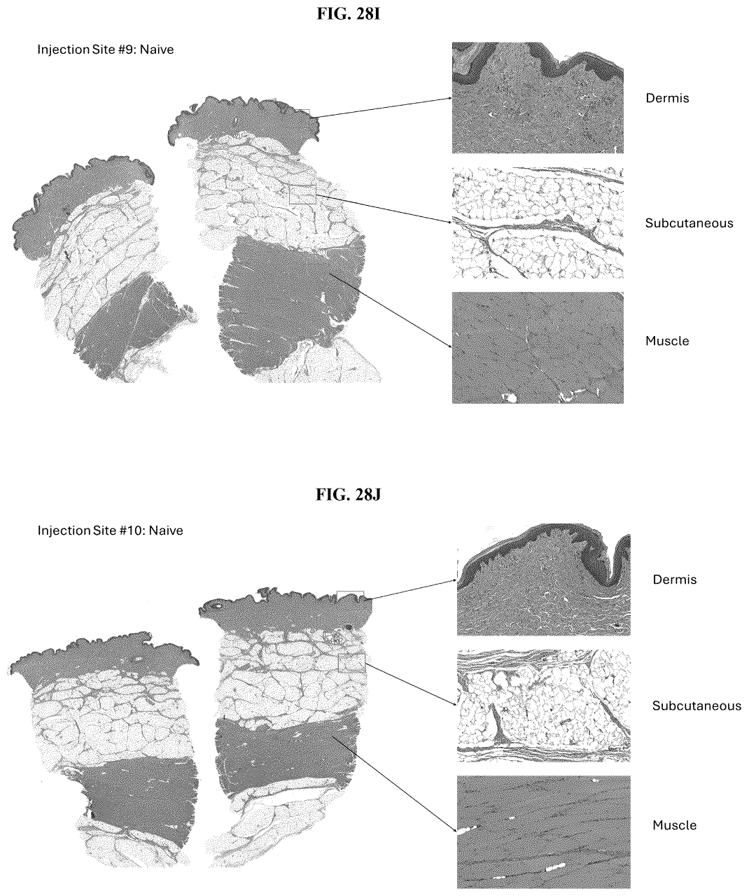

After fixation, the samples were processed and embedded into paraffin to make formalin fixed paraffin embedded (FFPE) tissues sections Each tissue section was evaluated at three different tissue depths (1-2-3; low-medium-high). In addition to tissue samples from the injection site, a sample was taken from two naïve untreated tissue sites for comparison. Tissue sections were prepared and stained with hematoxylin and eosin for pathohistological evaluation. Notably no findings occurred with the injections of either Sacituzumab govitecan or Sacituzumab govitecan+rHuPH20. Histological images of tissue sections stained with hematoxylin and eosin (H&E) together with magnified images of the dermis, subcutaneous and muscle tissue (5×) of biopsies taken from the Trodelvy±rHuPH20 injection sites and naïve skin are shown in FIGS. 28A-28J.

Both injections of the ADC alone and the ADC co-mixed with rHuPH20 demonstrated local skin tolerability when exposure times were 4 hours or less. In addition to this tolerability, this study showed
No pathophysiological findings in any skin component (epidermis-dermis, subcutaneous space or muscle layer).
Histological comparison of skin sections from both the ADC and the ADC+rHuPH20 appear comparable.
Longer exposures may be required to demonstrate if differences can be detected between treatment groups.

### Example 4: Summary of Large Volume ADC Studies

This study utilized all the same protocols and parameters as set forth in Example 1 unless disclosed otherwise.

### Study #1

### Objectives

Assess local tolerability of high dose ADC vs. ADC+rHuPH20
20 mL injection volume; 7.5 mm injection depth; 23G B-D needle
Delivery: 10 mg/mL; 5 mL/min (˜10 mg/kg)

### Endpoints

Back-Leakage measured post-injection (30 sec)
Bleb Size (Volume, Area, Height) measured at T0, T15, T30
Qualitative assessment of Erythema, Swelling and Induration over time (TO, T15, T30, T2h and T24h); Daily observations through 72h

Test Articles: ADC (10 mg/mL)+rHuPH20 (2000 U/mL)

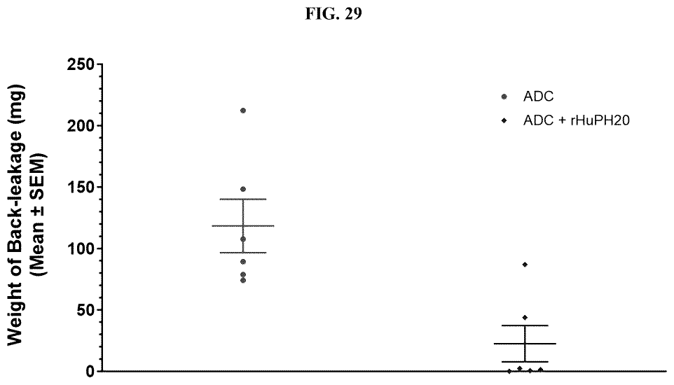

Results: Post-injection Back-leakage is significantly reduced with rHuPH20 (Table 25, FIG. 29).

TABLE 25

| Test Solution | Back-leakage (mg ± SEM) | % Decrease |
|---|---|---|
| ADC | 118.4 ± 21.8 | ~81%* |
| ADC + rHuPH20 | 22.5 ± 14.7 | |

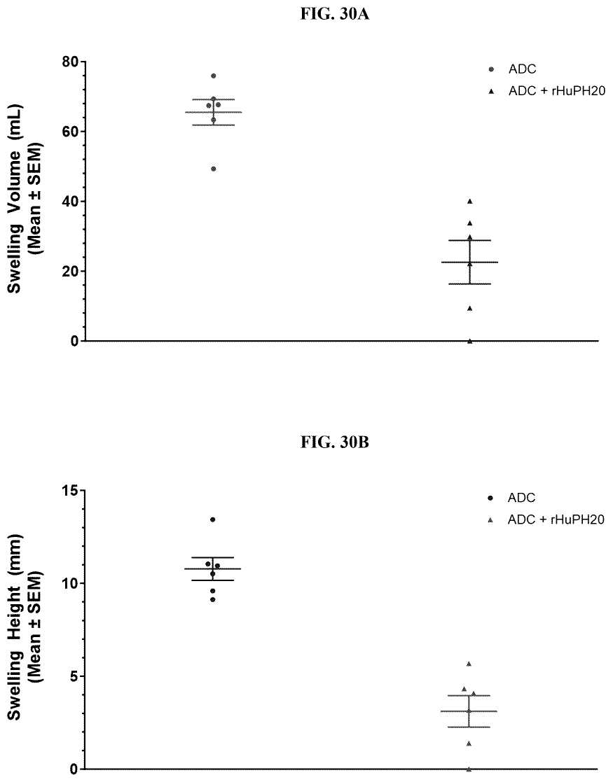

Bleb volume (FIG. 30A) and height (FIG. 30B) are significantly reduced with rHuPH20 (Table 26). Reduced bleb height largest contributing factor to reduced bleb volume.

TABLE 26

| | Volume (cc) | | Height (mm) | |
|---|---|---|---|---|
| Test | (Mean ± | % Decrease | (Mean ± | % Decrease |
| Solution | SEM) | Bleb Volume | SEM) | Bleb Height |
| ADC | 65.5 ± 3.6 | ~66% | 10.8 ± 0.6 | ~70% |
| ADC + | 22.5 ± 6.2 | | 3.1 ± 0.8 | |
| rHuPH20 | | | | |

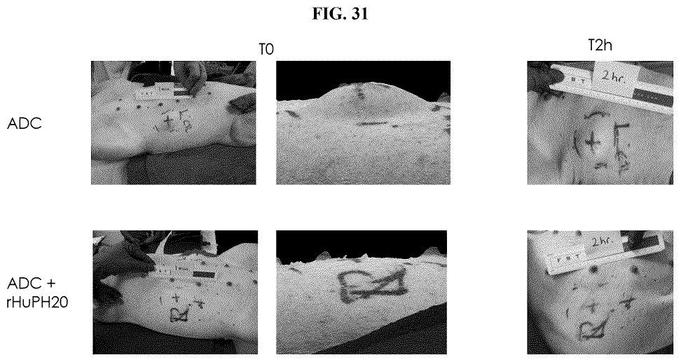

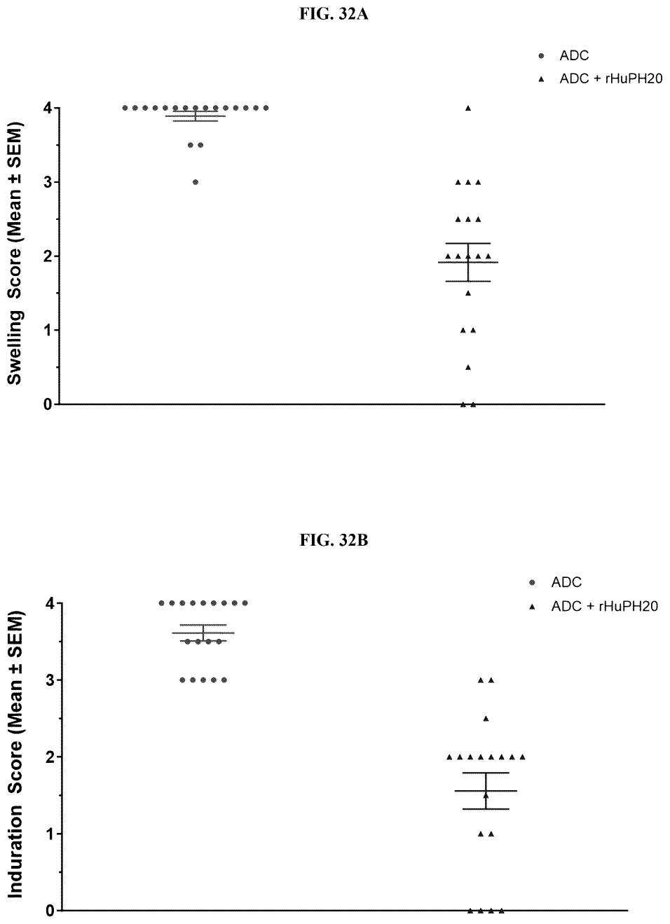

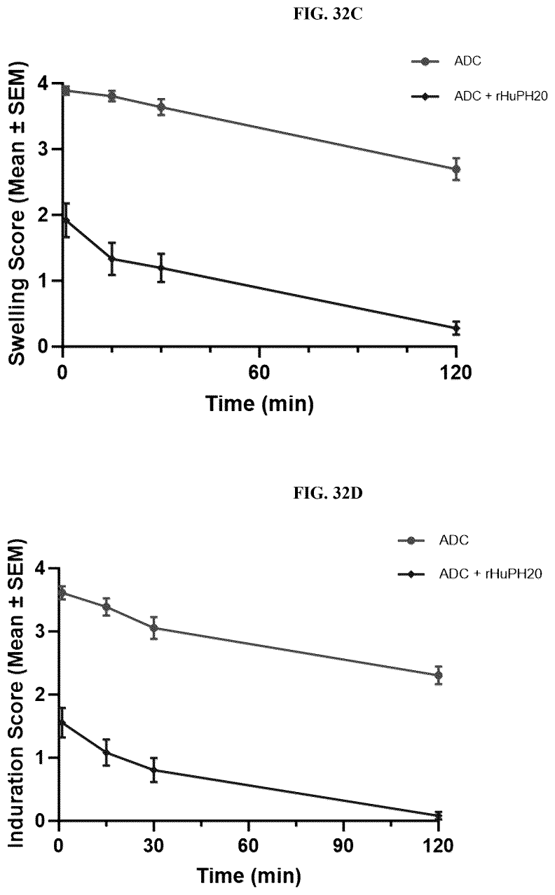

Large volume SC delivery of ADC+rHuPH20 shows minimal post-injection swelling in photographs in FIG. 31. Post-injection site swelling and induration are rapidly reduced with rHuPH20. For injections of ADC+rHuPH20, swelling (FIG. 32A) and induration (FIG. 32B) were lower than for control injections at T0, swelling was ≤1 for injections containing rHuPH20 by 2h (FIG. 32C), and induration was significantly reduced by 30 min and <1 by 2h (FIG. 32D). Score scales for swelling and induration are in Table 27 and 28, respectively.

TABLE 27
Swelling Score Scale

| Scale | Description |
|---|---|
| 0 | No swelling |
| 1 | Very slight swelling |
| 2 | Slight swelling |
| 3 | Moderate swelling |
| 4 | Severe swelling |

TABLE 28
Induration Score Scale

| Scale | Description |
|---|---|
| 0 | No perceptible difference in firmness |
| 1 | Very slightly firm (barely perceptible) |
| 2 | Mildly firm |
| 3 | Moderately firm |
| 4 | Very firm |

### Summary

Back-leakage was significantly reduced for all ADC+rHuPH20 injections
Bleb volume and height were reduced for rHuPH20-mediated injections
Qualitative assessment of post-injection swelling and induration demonstrated improvements for injections containing rHuPH20
Initial bleb size was smaller for rHuPH20-mediated injections with reduced firmness
rHuPH20 increased the rate of bleb resolution over time

### Study #2

### Objective

Assess local tolerability of high dose ADC vs. ADC+rHuPH20
20 mL injection volume
10 mg/mL; 5 mL/min (˜10 mg/kg)

### Endpoints

Back-Leakage measured post-injection (30 sec)
Bleb Size (Volume, Area, Height) measured at T0, T15, T30
Qualitative assessment of Erythema, Swelling and Induration over time (TO, T15, T30, T2h and T24h)

Test Articles: ADC (50 mg/mL)+rHuPH20 (2000 U/mL)

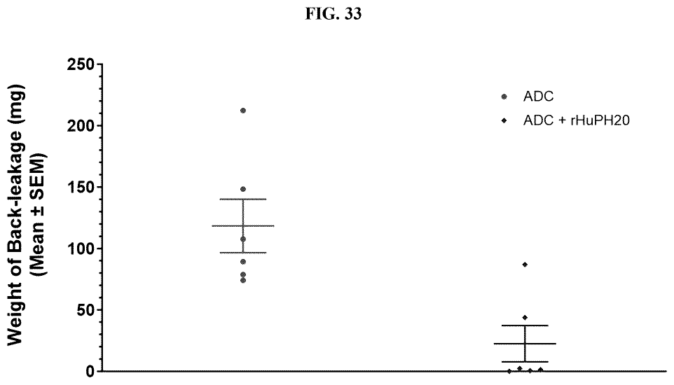

Results: Post-injection Back-leakage is significantly reduced with rHuPH20 (FIG. 33, Table 29).

TABLE 29
Post-injection back-leakage

| Test Solution | Back-leakage (mg ± SEM) | % Decrease |
|---|---|---|
| ADC | 118.4 ± 21.8 | ~81%* |
| ADC + rHuPH20 | 22.5 ± 14.7 | |

*p < 0.01

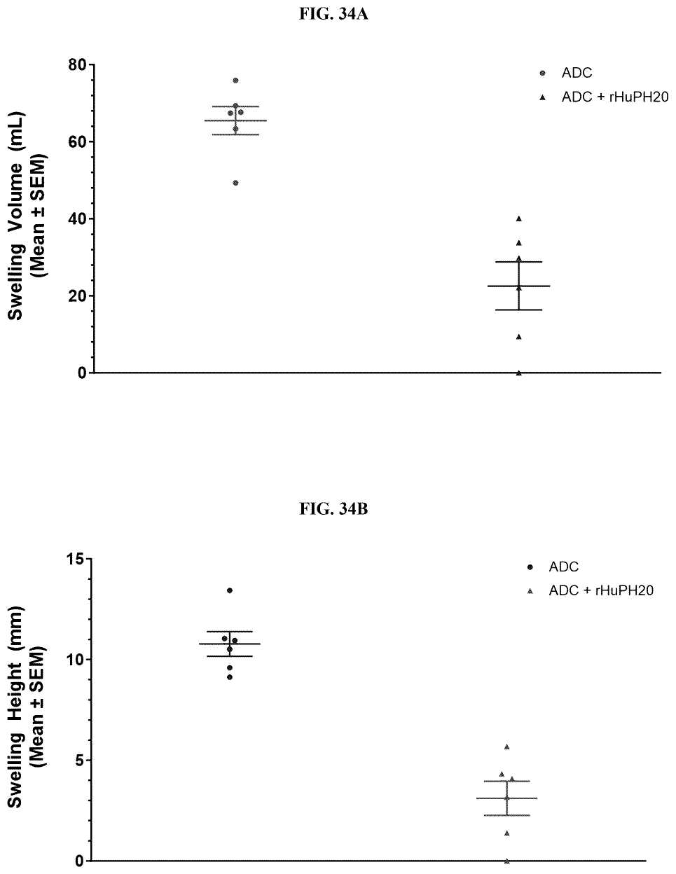

Bleb volume and height are significantly reduced with rHuPH20 (FIG. 34A, Table 30). Reduced bleb height largest contributing factor to reduced bleb volume (FIG. 34B, Table 30).

TABLE 30
Bleb volume and height

| Test | Volume (cc) | % | Height (mm) | % |
|---|---|---|---|---|
| Solution | (Mean ± SEM) | Decrease | (Mean ± SEM) | Decrease |
| ADC | 65.5 ± 3.6 | ~66% | 10.8 ± 0.6 | ~70% |
| ADC + | 22.5 ± 6.2 | | 3.1 ± 0.8 | |
| rHuPH20 | | | | |

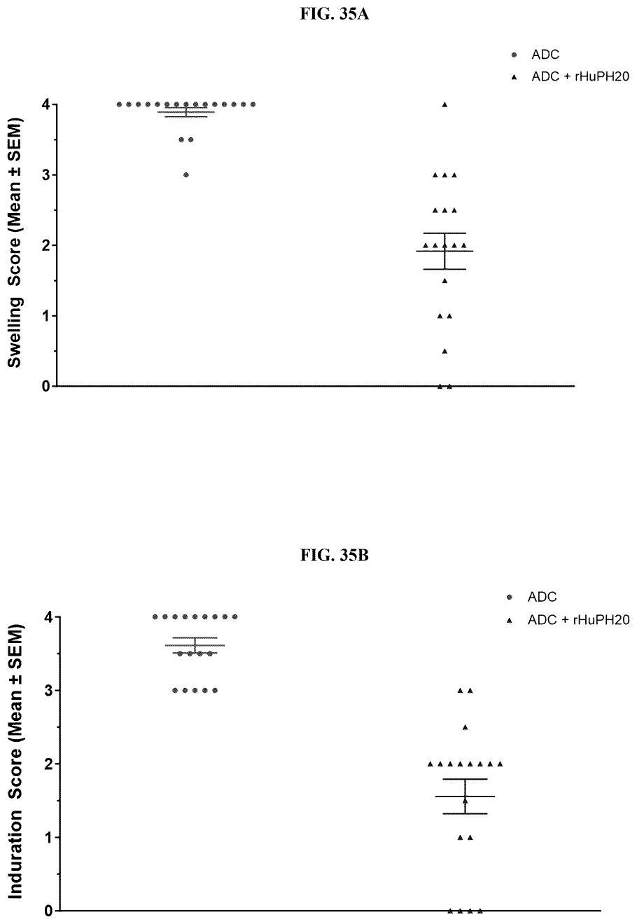

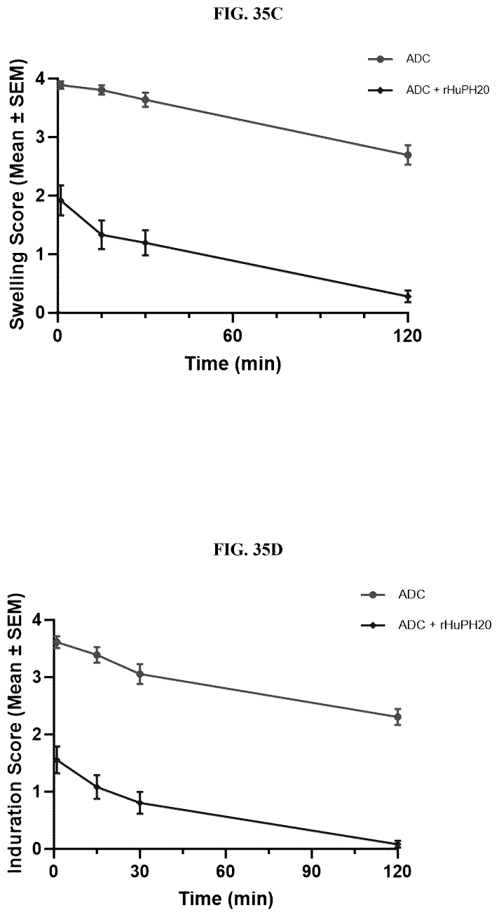

Post-injection site swelling and induration are rapidly reduced with rHuPH20. For injections of ADC+rHuPH20, swelling (FIG. 35A) and induration (FIG. 35B) were lower than for control injections at T0, swelling was ≤1 for injections containing rHuPH20 by 2h (FIG. 35C), and induration was significantly reduced by 30 min and <1 by 2h (FIG. 35D). Summary
Back-leakage was significantly reduced for all ADC+rHuPH20 injections
Bleb volume and height were reduced for rHuPH20-mediated injections
Qualitative assessment of post-injection swelling and induration demonstrated improvements for injections containing rHuPH20
Initial bleb size was smaller for rHuPH20-mediated injections with reduced firmness
rHuPH20 increased the rate of bleb resolution over time

### Example 5: Tolerability of an ADC+rHuPH20 Over Time Following SC Administration

This study utilized all the same protocols and parameters as set forth in Example 1 unless disclosed otherwise.

### Objective

Assess local injection site tolerability following SC administration of an ADC+rHuPH20 over 72h
10 mL injection volume; 2000 U/mL rHuPH20
HVAI used for injections

### Endpoints

Time to deliver measured for each injection
Back-Leakage measured immediately post-injection
Bleb Size (Volume, Area, Height) measured at T0
Qualitative assessment of Erythema, Swelling and Induration (TO, T24h, T48h, T72h)

Test Solutions: Sacituzumab govitecan (Trodelvy®)±rHuPH20 @ 2000 U/mL

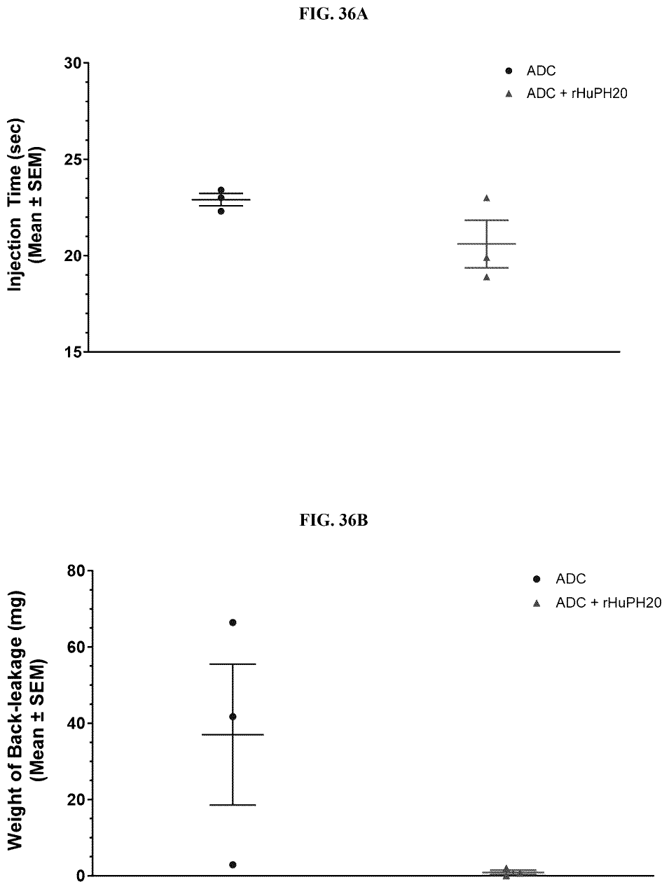

Results: rHuPH20 reduced delivery time and back-leakage (FIGS. 36A and 36B, Table 31).

TABLE 31
Delivery time and back-leakage.

| | Delivery | % | Back-leakage | % |
|---|---|---|---|---|
| Test Solution | Time (sec) | Decrease | (mg) | Decrease |
| ADC* | 22.9 ± 0.6 | −10% | 37.0 ± 32.0 | −98% |
| ADC + rHuPH20 | 20.6 ± 2.1 | | 0.9 ± 1.0 | |

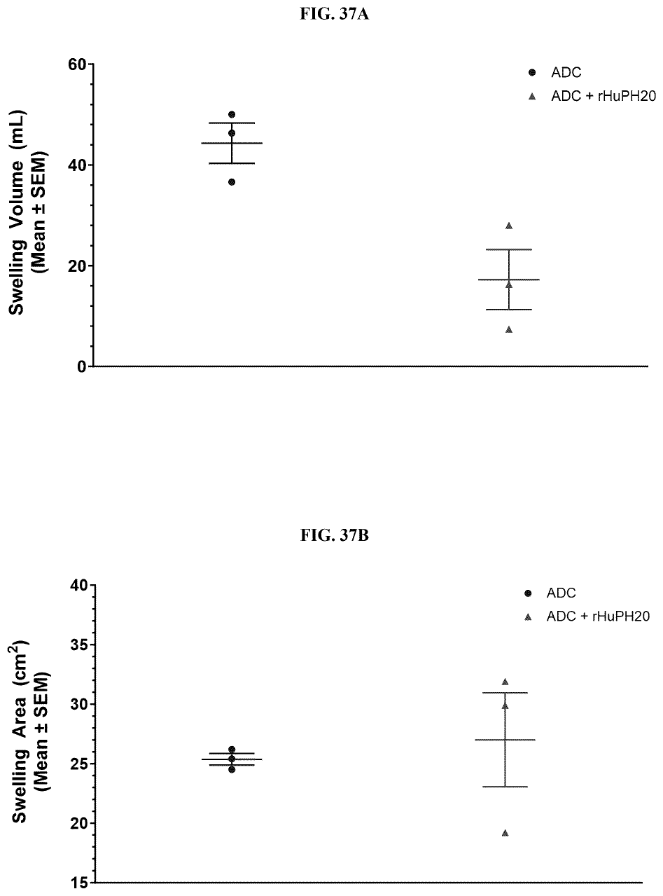

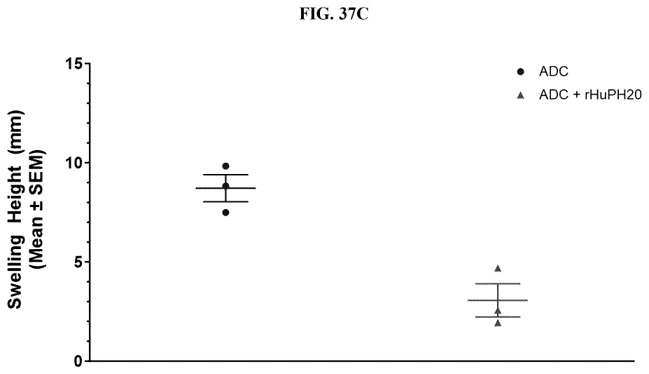

Bleb volume (FIG. 37A), area (FIG. 37B), and height (FIG. 37C) are reduced with rHuPH20 (Table 32). Reduced bleb height largest contributing factor to reduced bleb volume.

TABLE 32
Bleb size

| Test Solution | Volume | Area | Height |
|---|---|---|---|
| ADC | 44.3 ± 4.0 | 25.4 ± 0.5 | 8.7 ± 0.7 |
| ADC + rHuPH20 | 17.3 ± 6.0 | 27.0 ± 3.9 | 3.1 ± 0.8 |
| % Increase/Decrease | −61% | +6% | −64% |

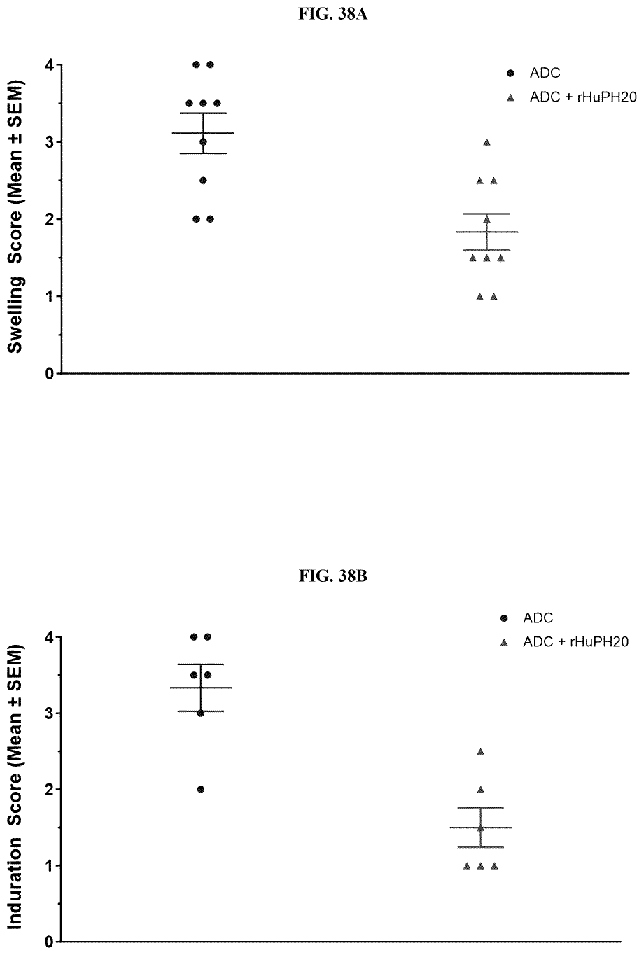

Post-injection site swelling and induration are reduced with rHuPH20. Swelling (FIG. 38A) and induration (FIG. 38B) were lower for ADC+rHuPH20 injections compared to ADC alone injections (Table 33). Score scales for swelling and induration are in Table 43 and 44, respectively.

TABLE 33
Swelling and induration post-injection

| Test Solution | Swelling Size | Induration |
|---|---|---|
| ADC | 3.1 ± 0.3 | 3.3 ± 0.3 |
| ADC + rHuPH20 | 1.8 ± 0.2 | 1.5 ± 0.3 |
| Induration Score | −42% | −55% |

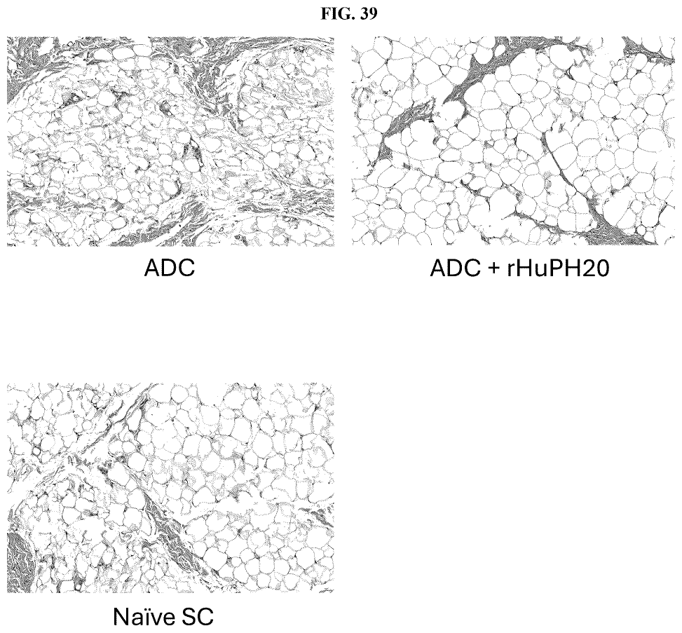

Histological Evaluation of Skin After SC Administration of ADC±rHuPH20. Method: After 72h, animals were euthanized and full-thickness punch biopsies taken (12 mm). FFPE sections were prepared from each injection site. Samples were evaluated at three different tissue depths. A sample of naïve skin was also included for comparison. Results: 1/3 tissue samples from ADC alone had multiple focal infiltrates in SC (all levels). 3/3 tissue samples from ADC+rHuPH20 were normal (no findings) (FIG. 39).

### Summary

Injection time for ADC was reduced ˜10% with addition of rHuPH20. Back-leakage was significantly reduced for all ADC+rHuPH20 injections (˜98%) with less variability. Bleb volume and height were reduced for rHuPH20-facilitate injections. Qualitative assessment of post-injection swelling and induration demonstrated improvements for injections with rHuPH20. Bleb sizes were smaller for rHuPH20-facilitated injections with reduced firmness. No erythema was observed for any injection. Histology demonstrated no findings for ADC+rHuPH20 injections whereas 1/3 injections of ADC alone had SC immune cell infiltrates.

### Example 6: Toxicopathology Data of Porcine Skin Samples

This study utilized all the same protocols and parameters as set forth in Example 1 unless disclosed otherwise. A routine toxicopathology review was performed with emphasis on tissue integrity. Results of the review are in Table 34.

TABLE 34
Toxicopathology review

| Sample | | |
|---|---|---|
| ID | Level | Microscopic Findings |
| 4081 | L Control | focal epithelial hyperplasia/hyperkeratosis minimal |
| | | dermis, leukocyte infiltrates, focal, minimal |
| | | subcutis, leukocytes, focal, minimal |
| | N Naïve | focal epithelial hyperplasia/hyperkeratosis minimal |
| | | dermis, leukocyte infiltrates, focal, minimal |
| | | subcutis, leukocytes, focal, minimal |
| | R PH20 | focal epithelial hyperplasia/hyperkeratosis minimal |
| | | dermis, leukocyte infiltrates, focal, minimal |
| | | subcutis, leukocytes, focal, minimal |
| 5055 | L PH20 | focal epithelial hyperplasia/hyperkeratosis minimal |
| | | dermis, leukocyte infiltrates, focal, minimal |
| | | subcutis, leukocytes, focal, minimal |
| | N Naïve | focal epithelial hyperplasia/hyperkeratosis minimal |
| | | dermis, leukocyte infiltrates, focal, minimal |
| | | subcutis, leukocytes, focal, minimal |
| | R Control | focal epithelial hyperplasia/hyperkeratosis minimal |
| | | dermis, leukocyte infiltrates, focal, minimal |
| | | subcutis, leukocytes, focal, minimal |
| 5061 | L Control | focal epithelial hyperplasia/hyperkeratosis minimal |
| | | dermis, leukocyte infiltrates, focal, minimal |
| | | subcutis, leukocytes, focal, minimal |
| | N Naïve | focal epithelial hyperplasia/hyperkeratosis minimal |
| | | dermis, leukocyte infiltrates, focal, minimal |
| | | subcutis, leukocytes, focal, minimal |
| | R PH20 | focal epithelial hyperplasia/hyperkeratosis minimal |
| | | dermis, leukocyte infiltrates, focal, minimal |
| | | subcutis, leukocytes, focal, minimal |
| 5069 | L PH20 | focal epithelial hyperplasia/hyperkeratosis minimal |
| | | dermis, leukocyte infiltrates, focal, minimal |
| | | subcutis, leukocytes, focal, minimal |
| | n Naïve | focal epithelial hyperplasia/hyperkeratosis minimal |
| | | dermis, leukocyte infiltrates, focal, minimal |
| | | subcutis, leukocytes, focal, minimal |
| | R Control | focal epithelial hyperplasia/hyperkeratosis minimal |
| | | dermis, leukocyte infiltrates, focal, minimal |
| | | subcutis, leukocytes, focal, minimal |
| 5083 | L Control | focal epithelial hyperplasia/hyperkeratosis minimal |
| | | dermis, leukocyte infiltrates, focal, minimal |
| | | subcutis, leukocytes, focal, minimal |
| | N Naïve | focal epithelial hyperplasia/hyperkeratosis minimal |
| | | dermis, leukocyte infiltrates, focal, minimal |
| | | subcutis, leukocytes, focal, minimal |
| | R PH20 | focal epithelial hyperplasia/hyperkeratosis minimal |
| | | dermis, leukocyte infiltrates, focal, minimal |
| | | subcutis, leukocytes, focal, minimal |
| 5085 | L PH20 | focal epithelial hyperplasia/hyperkeratosis minimal |
| | | dermis, leukocyte infiltrates, focal, minimal |
| | | subcutis, leukocytes, focal, minimal |
| | N Naïve | focal epithelial hyperplasia/hyperkeratosis minimal |
| | | dermis, leukocyte infiltrates, focal, minimal |
| | | subcutis, leukocytes, focal, minimal |
| | R Control | focal epithelial hyperplasia/hyperkeratosis minimal |
| | | dermis, leukocyte infiltrates, focal, minimal |
| | | subcutis, leukocytes, focal, minimal |

### Example 7: Tolerability of Full Dose Sacituzumab Govitecan Following Subcutaneous Administration with and without Recombinant Human Hyaluronidase PH20 (rHuPH20)

This study utilized all the same protocols and parameters as set forth in Example 1 unless disclosed otherwise. The objective of this study is to investigate the local tolerability of subcutaneous (SC) administration of an antibody drug conjugate (ADC) both with and without recombinant human hyaluronidase PH20 (rHuPH20). Minipigs are used in this study due to the similarity of the SC skin architecture to humans. Each animal receives two 20 mL SC injections into the lower abdominal region using a high-speed syringe pump. The needle is inserted vertically into the SC space using a custom 3D printed adapter to maintain the needle at a target needle depth of 7.5 mm.

The first injection is the ADC alone on the lower abdominal region of the animal. Following the injection of the ADC on one side of the animal, the animal receives a second SC injection of ADC+rHuPH20 on the contralateral side of the abdomen. Endpoints include measurement of applied force during the injection, as well as measurement of post-injection back-leakage, local injection site measurements (bleb area and volume), qualitative scoring assessment of the local tissue for erythema, swelling size and induration, and 3D imaging to quantitate post-injection changes of the skin. Qualitative assessments are taken at approximately one, two and three days post-injection. After the final timepoint the animals are humanely euthanized and full skin thickness punch biopsies are taken of the injection site for subsequent histopathological analyses.

This study utilizes an FDA approved antibody drug conjugate as a representative ADC. This antibody was used in prior studies that demonstrated the local tolerability both when administered in combination with rHuPH20 after SC administration. The objective of this follow-on study is to evaluate the local tolerability of full dose volumes.

Each animal receives two subcutaneous injections-one of the ADC alone and one of the ADC co-mixed with rHuPH20. The ADC alone injection precedes the ADC+rHuPH20 injection. A summary of the Description of Treatments for each cohort is shown in Table 35.

TABLE 35
Description of Treatments

| | | | Dose | | |
|---|---|---|---|---|---|
| Cohort | Test | | Volume | rHuPH20 | Evaluation |
| # | Solution | N/group | (mL) | (U/mL) | Times (h/d) |
| 1 | ADC | 6 | 20 | 0 | 2 h, 1 d, |
| | | | | | 2 d, 3 d |
| 2 | ADC + | 6 | 20 | 2000 | 2 h, 1 d, |
| | rHuPH20 | | | | 2 d, 3 d |

Each injection is administered using a syringe pump at a flow rate of 5 mL/min. The applied force is measured during the injection via a load cell attached to the end of the syringe barrel. Following administration any back-leakage is collected for a period of 30 seconds and weighed using an analytical balance with a sensitivity of 0.1 mg. The animal is monitored & photographed at approximately 2h post dose, and 1d, 2d and 3d. After the final timepoint, the animals are humanely euthanized and punch biopsies (12 mm) of each injection site are obtained and preserved in 10% formalin for future histological analyses. For comparison, a full thickness punch biopsy is taken from a non-treated portion of the abdomen after the animal is euthanized.

One day prior to the study, syringes are filled with ˜22 mL of the appropriate test solutions, capped, and stored at 2-8° C. Syringes are allowed to acclimate to room temperature for at least 30 minutes prior to use and used within 2 hours. Once the animal has received the two injections, two additional syringes are brought to room temperature as described above for use with the next animal.

After anesthetization the animal is placed in dorsal recumbence on a heated surgical table and is maintained under isoflurane gas for the entire duration of the procedure. Following anesthetization the abdominal region is cleaned with Nolvasan followed by wiping the injection site with gauze containing 70% isopropanol and wiped dry with sterile gauze. Injection sites are located on the left and right abdominal regions, ˜5 cm cranially from the inguinal fold and ˜3 cm towards the midline of the animal. Each of the injection sites are marked with a permanent marker and then photographed with the standard and 3D cameras prior to needle insertion.

Immediately prior to use the syringe containing the test solution is mounted to a load cell in an adapter and then placed on the end of the syringe barrel. The syringe cap is then removed and replaced with a 23G×12-inch BD Vacutainer Blood Collection Set. The infusion set is primed to the needle tip. The syringe is then loaded into a high-pressure syringe pump. The load cell is then zeroed and the pump block then pushed to abut the end of the syringe barrel and locked in place. Load cell measurements are taken at a sampling rate of 2 Hz.

A custom 3D printed adapter is used to hold the needle at a precise depth of 7.5 mm. Approximate needle depths are recorded. The skin at the injection site is then pinched and the needle inserted vertically into the SC space and the skin is then allowed to relax into normal position. The 3D printed adapter is held in place during the injection flush against the skin but without exerting excessive downward pressure.

Upon completion of the injection the needle is removed from the skin and the syringe with infusion set discarded. Test solution samples from at least 3 out of 6 ADC+rHuPH20 syringes will be retained for post-study enzymatic testing. Test solution back-leakage is then absorbed to a tared eye-spear for 30 seconds on the injection site. The weight of the eye spear is then recorded using an analytical balance. The margins of the injection site bleb are marked with a permanent marker and measured for length, width, and height using a digital calliper and recorded then photographed with the standard and 3D cameras. The margins of the injection site blebs are also measured using a digital calliper at T15 and T30 timepoints. Local injection sites are then qualitatively assessed and graded for erythema severity, swelling size appearance, and firmness, using a scoring system described in Example 1.

Approximately 24 hours post-dosing of the first injection, injection sites for each animal are inspected, photographed, and assessed for erythema, induration, and swelling. The scoring and evaluation continue daily for up to approximately three days (72h). Following the assessment after day three, the animal is humanely euthanized using an injectable euthanasia drug provided by the vivarium staff. Following euthanasia punch biopsies of the injection site are taken (12 mm). For comparison, a punch biopsy from an untreated area of the abdomen is obtained for each animal. After obtaining the punch biopsies the animal carcass is removed and disposed of as biohazardous waste.

### Example 8: Pharmacokinetics of Dupilumab Administration Following Subcutaneous Administration with and without Recombinant Human Hyaluronidase PH20 (rHuPH20)

The objective of this study was to investigate the pharmacokinetics (PK) of a monoclonal antibody dupilumab (Dupixent®) following subcutaneous (SC) administration alone or co-administered with recombinant human hyaluronidase PH20 (rHuPH20; 5000 U/mL). This study compared the PK of a standard clinical dose of 2 mL of dupilumab alone versus the PK of a 5 mL bolus of dupilumab co-mixed with rHuPH20. The 2 mL doses were administered using a pre-filled syringe (PFS) while the larger dose volume was administered using a prototype HVAI with a 5 mL fill. Blood was collected prior to the start of the study (pre-dose), and at 6 hours (h), 1 day (d), 2d, 3d, 4d, 5d and 7d post-injection. Blood was collected via jugular vein stick into serum separation tubes (SSTs) and allowed to remain at room temperature for thirty minutes whereupon they were then placed on ice until processed for serum collection. Samples were divided into duplicates and stored at −80° C. until used for bioanalysis. The concentration of functional Dupixent® of individual serum sample was quantitatively detected using Dupilumab ELISA kit, abx395100, Abbexa LLC.

Ten animals were administered a single injection of either dupilumab alone (5 animals) or with dupilumab+rHuPH20 (5 animals). Prior to injection pre-treatment blood samples were obtained from each animal. After test solution injection blood samples were obtained at 6 h, 1d, 2d, 3d, 4d, 5d and 7d post-injection. Blood was collected into serum separator tubes, centrifuged, and stored at −80° C. for bioanalytical analysis.

The concentration of functional Dupixent® in serum sample was quantitatively detected using a competitive Dupilumab ELISA kit (abx395100, Abbexa LLC). Briefly, Dupixent® in serum sample was bound to its antigen immobilized on 96-well plate, which competes the binding of detection reagent, biotin labeled Dupilumab. After incubation and washing to remove unbound Dupilumab from 96-well plate, the bound biotin labeled Dupilumab was visualized by horseradish peroxidase substate and Dupilumab concentration was calculated from a 4PL fitted standard curve.

The two test solutions administered in this study were dupilumab alone and dupilumab co-mixed with rHuPH20. Dupilumab alone was administered as commercially packaged in a 2 mL PFS. To prepare the co-mix, the antibody solution was pooled from dupilumab syringes (28 mL). To the pooled dupilumab, 0.157 mL of rHuPH20 (10 mg/mL) was added to yield 28.157 mL of test solution. A sample of the co-mix was used for enzymatic activity testing.

All device syringes were filled in a sterile fill and finish hood under aseptic conditions. Cyclic olefin copolymer (COC) syringes were filled with 5.1 mL of the co-mix of dupilumab+rHuPH20. To fill the syringes a sterile rubber plunger was initially inserted into the sterile syringe barrel and pushed to a preset depth for filling, The co-mix of dupilumab+rHuPH20 was then pumped into the syringe at a flow rate of 2 mL/min. After adding the test solution to the syringe, the rubber plunger was brought to the final loading position (to remove residual air in the syringe). Syringes were then capped and stored at 2-8° C. until removed and brought to room temperature prior to use in device assembly.

Assembly of all devices occurred in a sterile fill and finish hood under aseptic conditions. The syringe cap was removed and replaced using a 25G×1-inch Becton Dickinson (BD) needle. The needle remained capped until immediately prior to use. The needle and syringe were placed in a proprietary assembly jig with the HVAI for final assembly, which included the placement of a needle guard that brought the exposed needle depth to 10 mm. The length of each needle in the completed device was measured prior to use and all were found to be 10.0 mm. Devices were discarded after use.

This study measured the pharmacokinetics of dupilumab alone or dupilumab co-mixed with rHuPH20 following subcutaneous administration. The dose volume for dupilumab alone was 2 mL (300 mg) while the dose volume for dupilumab+rHuPH20 was 5 mL (750 mg). Five animals were injected with dupilumab alone and five animals were injected with dupilumab+rHuPH20. The treatment groups are shown in Table 36.

TABLE 36
Description of Treatments

| | | | Dose | | |
|---|---|---|---|---|---|
| | | | Volume | rHuPH20 | Blood Sampling |
| Cohort | Test Solution | N | (mL) | (U/mL) | Times (h/d) |
| 1 | dupilumab alone | 5 | 2 | 0 | 6 h, 1 d, 2 d, 3 d, |
| | | | | | 4 d, 5 d, 7 d |
| 2 | dupilumab + | 5 | 5 | 5000 | 6 h, 1 d, 2 d, 3 d, |
| | rHuPH20 | | | | 4 d, 5 d, 7 d |

Each animal received a single SC injection to their left lower abdominal region. For Cohort #1, injections were administered using the manufacturer's pre-filled syringe (2 mL). For Cohort #2, injections of dupilumab+rHuPH20 were delivered using an HVAI device (5 mL). The duration of all injections was timed using a hand-held stopwatch. Following administration any back-leakage was collected for a period of 30 seconds and weighed using an analytical balance. Approximately 3 mL of blood was collected from each animal at intervals of 6 h, 1d, 2d, 3d, 4d, 5d, and 7d via jugular venipuncture. After the final blood collection timepoint, the animal was humanely euthanized.

Prior to the start of study, animals were assessed for general health, body weight recorded, and a pre-treatment baseline blood sample obtained and collected into serum separating tubes (SSTs). Blood samples were processed according to manufacturer's instructions (centrifugation for 10 min @ 1250 rcf,) and serum removed and stored at −80° C. until used for bioanalysis. Additionally, blood collections were obtained from each animal at 6h post injection, and on day 1, 2, 3, 4, 5 and 7.

One day prior to the study, syringes were filled with appropriate test solutions and assembled into HVAIs then stored at 2-8° C. Devices were allowed to acclimate to room temperature for at least 30 minutes prior to use and used within 2 hours.

On the day of the procedure, animals were anesthetized and placed in dorsal recumbence on a heated surgical table and maintained under isoflurane gas for the entire duration of the procedure. Following anesthetization, the abdominal region was cleaned with Nolvasan followed by wiping the injection site with gauze containing 70% isopropanol and wiped dry with sterile gauze. Injection sites were located on the lower left abdominal regions, ˜5 cm cranially from the inguinal fold towards the midline and ˜3 cm towards the midline of the animal. Each injection site was marked with a permanent marker and then photographed prior to needle insertion and immediately post-injection.

Animals in Cohort #1 received a single SC injection of dupilumab alone administered via a PFS using a standard skin pinch method. Animals in Cohort #2 received a single SC injection of dupilumab+rHuPH20 administered via an HVAI. For HVAI injections, the skin was briefly tented for vertical needle insertion and allowed to relax while holding the device in place against the skin prior to activating device.

Injections were timed using a stopwatch. Upon completion of the injection, the needle and HVAI were removed and discarded. Test solution back-leakage was then absorbed to a tared eye-spear for 30 seconds on the injection site and the weight of the eye spear was measured using an analytical balance. The margins of the injection site bleb were marked with a permanent marker and measured for length, width, and height using a digital caliper and recorded then photographed with the standard camera. Caliper measurements and photographs were taken only immediately post-injection. Local injection sites were qualitatively assessed and graded immediately post-injection (T0) for erythema severity, swelling size appearance, and firmness, using a scoring system described in Example 1. Following the blood sampling obtained on d7, the animal was humanely euthanized using an injectable euthanasia drug provided by the vivarium staff.

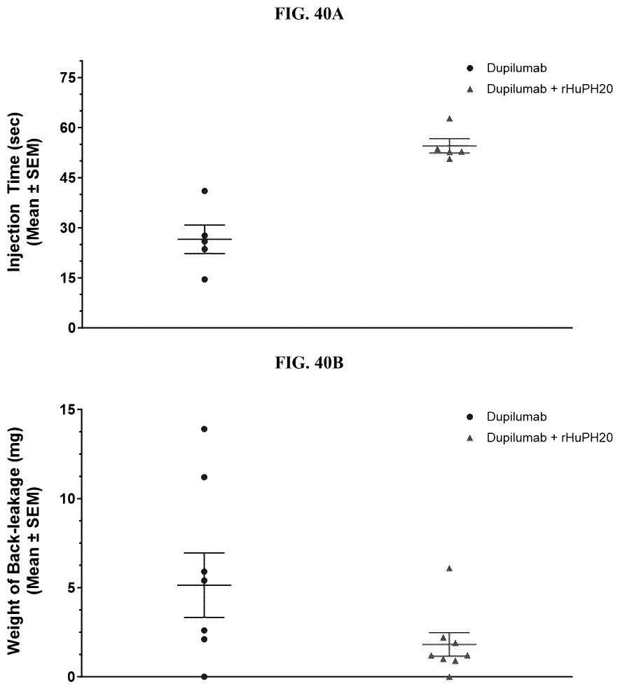

Pre-study hyaluronidase activity testing of dupilumab+rHuPH20 Co-mix. The enzymatic activity of the co-mix of dupilumab+rHuPh20 was tested using VV-QWUAL-006751 formerly TM010) The pre-study testing results are shown in Table 37 and individual animal data is shown in FIG. 40A.

TABLE 37
Summary of Pre-study Enzymatic Activity Testing

| Test Solution | Pre-study Concentration (U/mL ± SD) |
|---|---|
| Dupilumab + rHuPH20 | 5637 ± 22 |

The duration of each injection was measured using a hand-held stopwatch with a precision of 0.1 seconds. The pre-study testing results are shown in Table 38 and individual animal data is shown in FIG. 40B.

TABLE 38
Measurement of Injection Time

| Test Solution | Injection Time (sec ± SEM) |
|---|---|
| Dupilumab | 27.3 ± 4.3 |
| Dupilumab + rHuPH20 | 54.5 ± 2.1 |

The back-leakage was collected using a pre-weighed eye spear for 30 seconds post-injection. The eye spear was then re-weighed, and the weight of the back-leakage recorded. The amount of back-leakage for each test solution is shown in Table 39.

TABLE 39
Measurement of Back-Leakage

| Test Solution | Back-Leakage (mg ± SEM) |
|---|---|
| Dupilumab | 1.9 ± 1.0 |
| Dupilumab + rHuPH20 | 5.4 ± 2.6 |

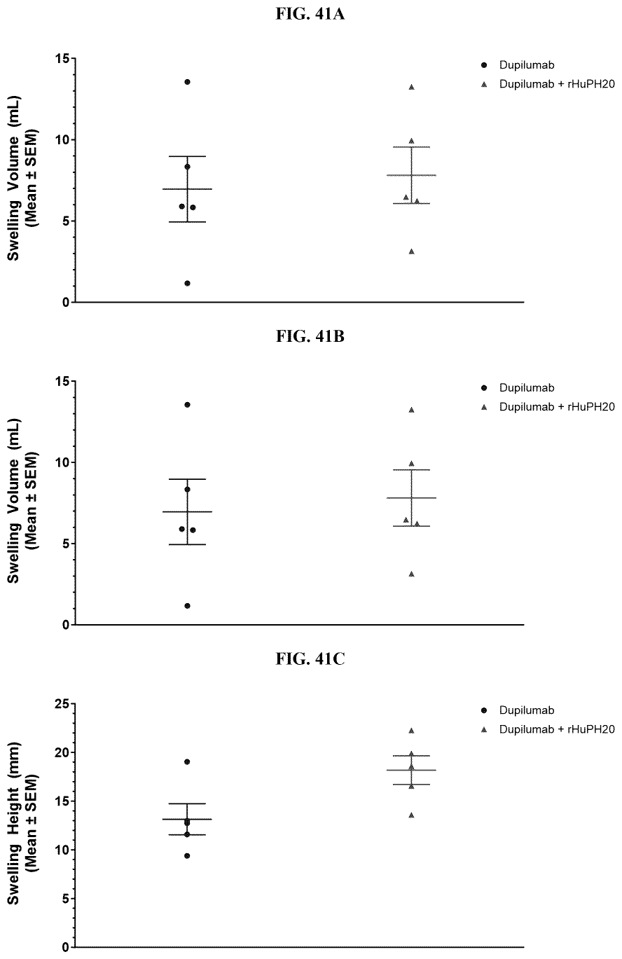

The local injection site swelling (bleb) was marked and measured using a digital caliper. Bleb volume dispersion area and swelling height of each bleb was determined as described above. Mean and individual post-injection bleb volume, area and height values are shown in FIG. 41A-C, respectively.

TABLE 40
Post-Injection Bleb Volume, Area, Height - Caliper Measurements

| | Test | Mean ± SEM | | |
|---|---|---|---|---|
| Cohort # | Solution | Volume | Area | Height |
| 1 | Dupilumab | 7.0 ± 1.8 | 13.1 ± 1.4 | 2.5 ± 0.5 |
| 2 | Dupilumab + | 7.8 ± 1.6 | 18.2 ± 1.3 | 2.3 ± 0.6 |
| | rHuPH20 | | | |

Qualitative assessments were taken immediately post-injection (T0) and daily thereafter. No erythema, swelling or induration was detected on D1, D2 or D3 post-injection (data not shown). No post-injection erythema was observed for any injection.

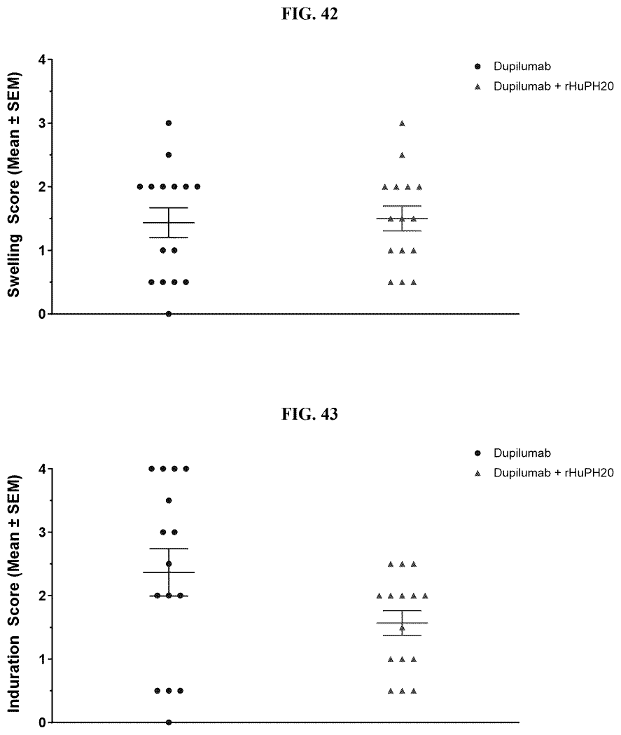

Scoring by three evaluators for swelling size using the modified Draize scoring system are summarized in Table 41 and individual animal data shown in FIG. 42. No swelling was detected at the 24 h, 48h or 72h timepoints (data not shown).

TABLE 41
Post-Injection Swelling of Dupilumab and Dupilumab + rHuPH20

| Test Solution | Swelling Score (Mean ± SEM) |
|---|---|
| Dupilumab | 1.4 ± 0.2 |
| Dupilumab + | 1.5 ± 0.2 |
| rHuPH20 | |

Post-Injection Induration. Scoring by three evaluators for bleb induration was performed and is summarized in Table 42 and shown in FIG. 43.

TABLE 42
Post-Injection Induration After SC Administration
of Dupilumab and Dupilumab + rHuPH20

| | Induration Score |
|---|---|
| Test Solution | (Mean ± SEM) |
| Dupilumab | 2.4 ± 0.4 |
| Dupilumab + rHuPH20 | 1.6 ± 0.2 |

Notably, despite dupilumab+rHuPH20 having injection volumes 2.5× that of the dupilumab alone, the induration of the larger volume appeared to be markedly reduced. Five animals received a 2 mL dose of dupilumab (Cohort 1) and five animals received a 5 mL dose of dupilumab+rHuPH20 (Cohort 2). The serum concentrations of dupilumab were measured using Dupilumab ELISA, Abx395100, Lot E2402922A, Exp. September 2024. The Concentration was calculated using a 4-PL model, SoftMax Pro, Molecule Device.

TABLE 43
Concentrations of Dupixent in Individual Pig Serum

| | Time | | | | | | | | |
|---|---|---|---|---|---|---|---|---|---|
| | Points | | | | | | | | |
| | Post | Dupixent Concentrations (μg/mL) | | | | | Dupixent Concentrations | | |
| | Injection | Animal ID | | | | | (μg/mL) | | |
| Cohort | (hr) | 4497 | 4499 | 4500 | 4501 | 4590 | Mean | SD | CV % |
| 1 | 0 | 0 | 0 | 0 | 0 | 0 | 0.0 | 0.0 | n/a |
| 1 | 6 | 84.7 | 130 | 10.9 | 62.8 | 105.6 | 78.8 | 45.4 | 57.6 |
| 1 | 24 | 162.9 | 203.2 | 88.1 | 129.3 | 178.9 | 152.5 | 44.9 | 29.4 |
| 1 | 48 | 154.7 | 198.9 | 138.5 | 156.3 | 158.2 | 161.3 | 22.4 | 13.9 |
| 1 | 72 | 141.7 | 192.9 | 172.9 | 159.1 | 142.2 | 161.8 | 21.7 | 13.4 |
| 1 | 96 | 134.6 | 208 | 150.2 | 152.9 | 138.4 | 156.8 | 29.6 | 18.9 |
| 1 | 120 | 133 | 210.9 | 152.3 | 162.6 | 147.4 | 161.2 | 29.7 | 18.4 |
| | Post | | | | | | Dupixent Concentrations | | |
| | Injection | Animal ID | | | | | (μg/mL) | | |
| | (hr) | 4483 | 4485 | 4494 | 4495 | 4496 | Mean | SD | CV % |
| 2 | 0 | 0 | 0 | 0 | 0 | 0 | 0 | 0.0 | n/a |
| 2 | 6 | 113.7 | 118.9 | 77.5 | 195.5 | 43.9 | 109.9 | 56.6 | 51.5 |
| 2 | 24 | 332.3 | 366.8 | 301.5 | 448.7 | 316.3 | 353.12 | 58.7 | 16.6 |
| 2 | 48 | 318.7 | 403.2 | 345.7 | 492.4 | 431.8 | 398.36 | 69.1 | 17.3 |
| 2 | 72 | 332.7 | 363.1 | 373.6 | 474.5 | 403.2 | 389.42 | 53.8 | 13.8 |
| 2 | 96 | 348.7 | 406.5 | 353.1 | 486.4 | 386.8 | 396.3 | 55.8 | 14.1 |
| 2 | 120 | 320.8 | 355.6 | 341.4 | 466.7 | 404 | 377.7 | 58.4 | 15.5 |
| 2 | 168 | 340.7 | 392.5 | 326.5 | 404.5 | 357.9 | 364.42 | 33.3 | 9.1 |

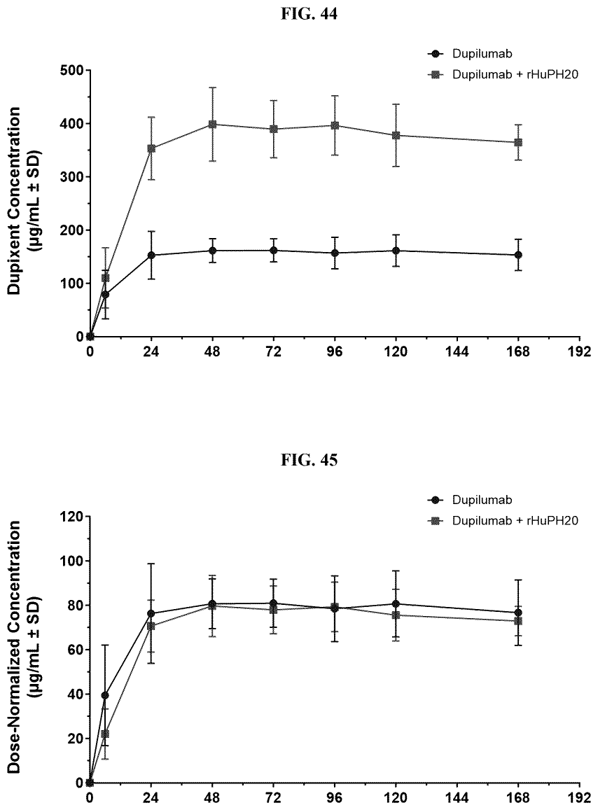

The concentration-time profiles of dupilumab and dupilumab+rHuPH20 are shown in FIG. 44. The concentration of dupilumab at each timepoint was dose-normalized for both treatment groups. To obtain dose-normalized values, the serum values for dupilumab concentration were divided by 2 (dose volume for Cohort #1) or by 5 (dose volume for Cohort #2). A similar calculation was performed for standard deviation values. The dose-normalized concentration versus time profiles for the two treatment groups is shown in FIG. 45. Individual pharmacokinetic parameters were determined by non-compartmental analyses in Excel and are presented in Table 44.

TABLE 44
Pharmacokinetic Parameters for Individual Animals

| | | | Dose | | Dose |
|---|---|---|---|---|---|
| | | | Normalized | | Normalized |
| | Animal | AUC₀₋₁₆₈ | AUC₀₋₁₆₈ | Cₘₐₓ | Cₘₐₓ |
| Group | ID | (μg*hr/mL) | (μg*hr/mL) | (μg/mL) | (μg/mL) |
| 1 | 4497 | 22473 | 11237 | 163 | 81 |
| 1 | 4499 | 32651 | 16325 | 211 | 105 |
| 1 | 4500 | 22164 | 11082 | 173 | 86 |
| 1 | 4501 | 24181 | 12090 | 163 | 81 |
| 1 | 4590 | 24279 | 12140 | 179 | 89 |
| Group Mean | | 25150 | 12575 | 178 | 89 |
| Standard Deviation | | 4302 | 2151 | 20 | 10 |
| Coefficient of | | 17 | 17 | 11 | 11 |
| Variation % | | | | | |
| 2 | 4483 | 52071 | 10414 | 349 | 70 |
| 2 | 4485 | 59498 | 11900 | 407 | 81 |
| 2 | 4494 | 53126 | 10625 | 374 | 75 |
| 2 | 4495 | 73157 | 14631 | 492 | 98 |
| 2 | 4496 | 59626 | 11925 | 432 | 86 |

| | | | | |
|---|---|---|---|---|
| Group Mean | 59496 | 11899 | 411 | 82 |
| Standard Deviation | 8402 | 1680 | 56 | 11 |
| Coefficient of | 14 | 14 | 14 | 14 |
| Variation % | | | | |

A prototype HVAI was able to successfully deliver 5 mL of dupilumab in approximately 55 seconds using rHuPH20. Despite having a dose volume 2.5× greater than that of dupilumab alone, dupilumab+rHuPH20 resulted in similar post-injection bleb size. Post-injection induration was reduced by the addition of rHuPH20 compared to dupilumab alone. Basic pharmacokinetic parameters derived from the two dose groups indicate similar dose-normalized PK profiles, with no significant differences in dose-normalized AUC₀₋₁₆₈ or Cₘₐₓ observed.

### Example 9: Pharmacokinetic Models

The following simulations were conducted for TRODELVY and eight examples (see Table 45 below). Commercially-approved or clinically-evaluated IV dosing regimens were defined for each ADC, along with PK parameters (e.g. volume of distribution and clearance), based on publicly available information (e.g. from package inserts or peer-reviewed publications). Two-compartment PK models were constructed for each ADC based on this information. Simulations were then generated for each ADC to compare the PK profiles of the current IV regimen and SC regimen(s) when co-administered with rHuPH20. Doses of the ADC administered SC with rHuPH20 were chosen to match the AUC (the typical driver of efficacy) of the current IV regimen and simulations of ADCs SC with rHuPH20 were generated for the current schedule (Q3W), as well as less (e.g., Q4W) and more (e.g, Q2W) frequent schedules.

TABLE 45
PK simulations for ADC dosing regimens

| Example # | Generic Name | Trade Name | FIG. |
|---|---|---|---|
| i | patritumab deruxtecan | N/A | 47 |
| ii | datopotamab deruxtecan | N/A | 48 |
| iii | brentuximab vedotin | ADCETRIS | 49 |
| iv | trastuzumab emtansine | KADCYLA | 50 |
| v | trastuzumab deruxtecan | ENHERTU | 51 |
| vi | tisotumab vedotin | TIVDAK | 52 |
| vii | mirvetuximab | ELAHERE | 53 |
| | soravtansine | | |
| viii | loncastuximab tesirine | ZYNLONTA | 54 |

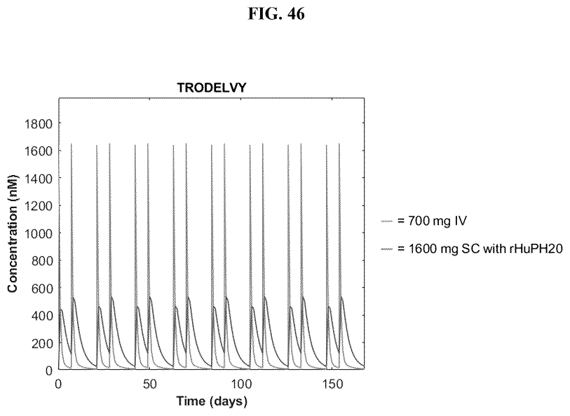

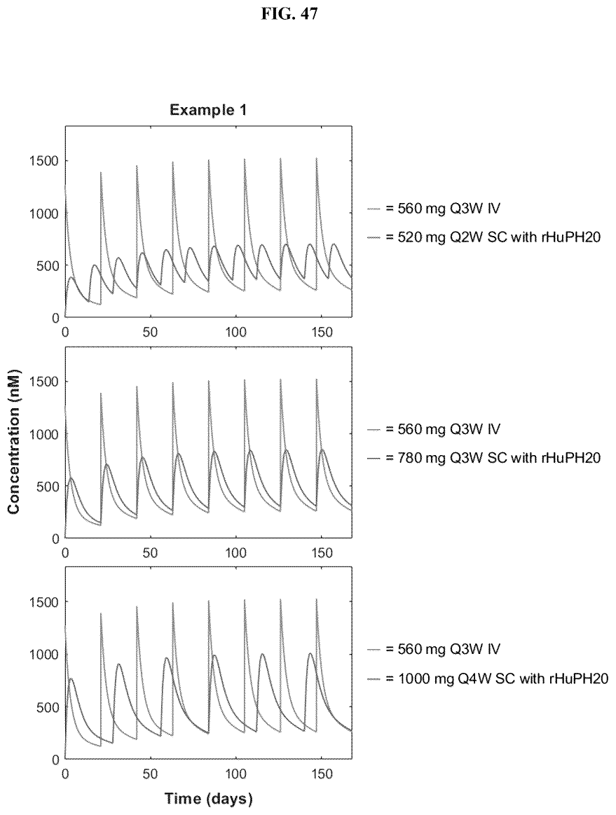

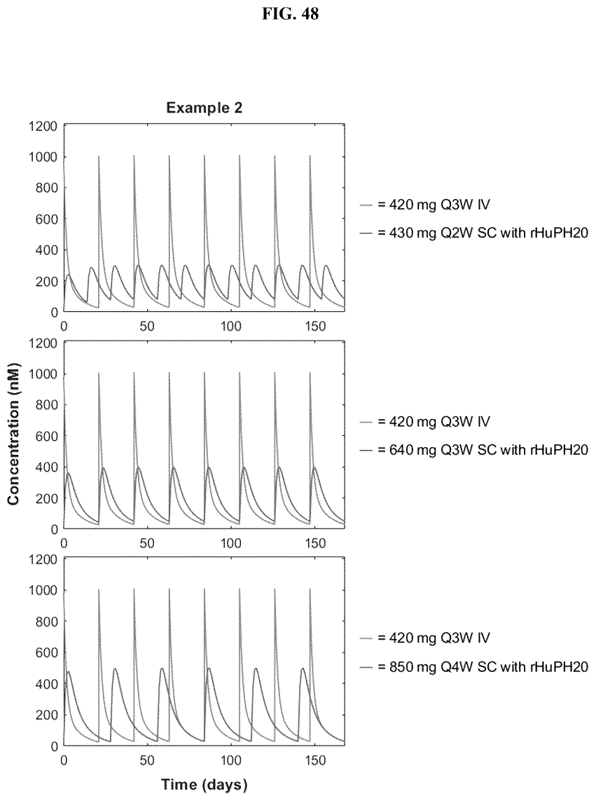

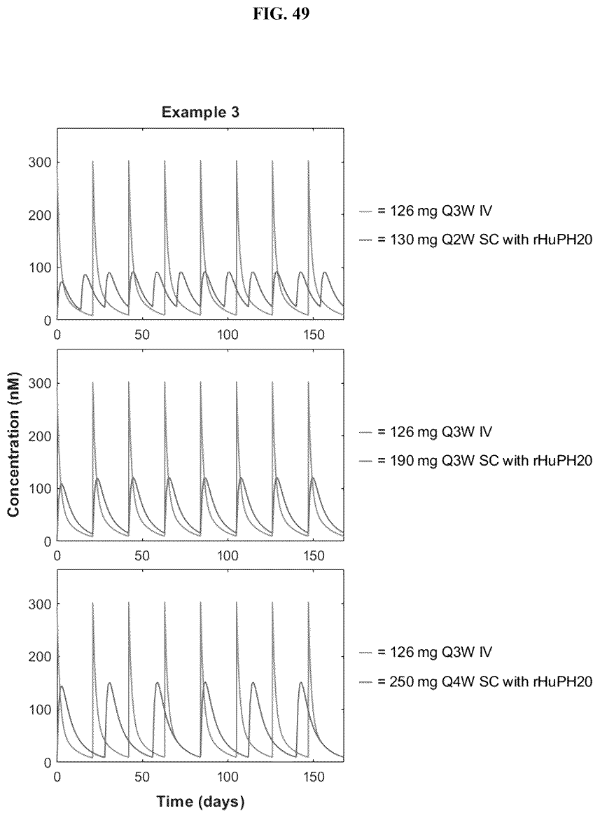

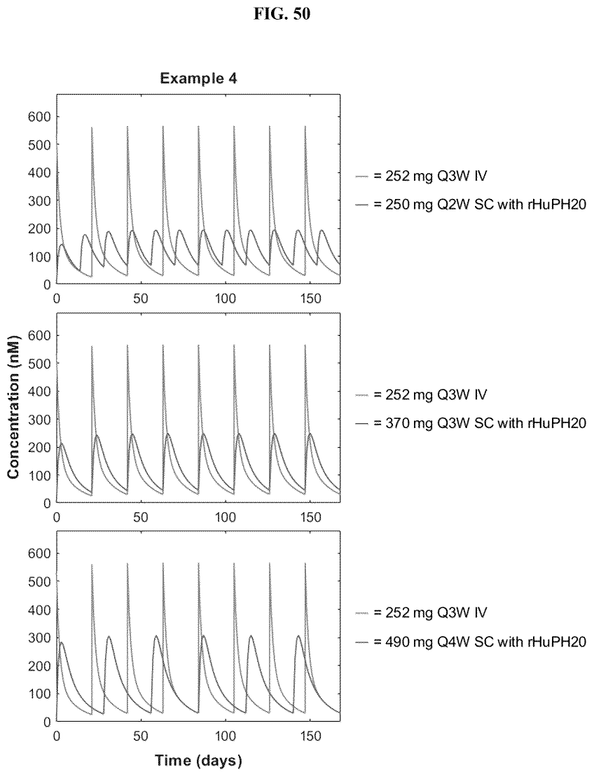

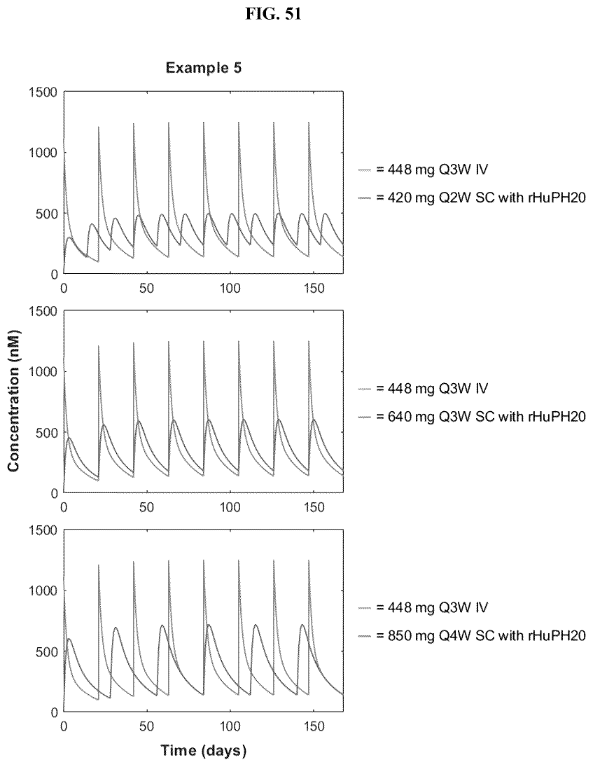

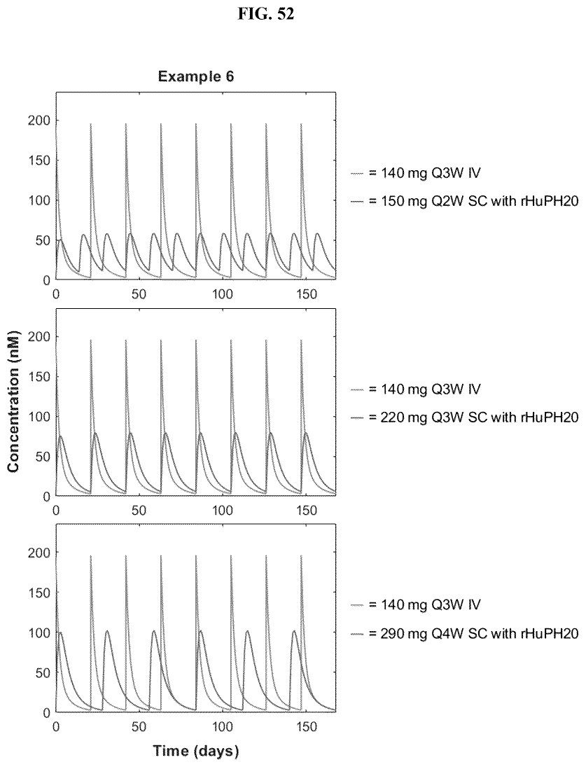

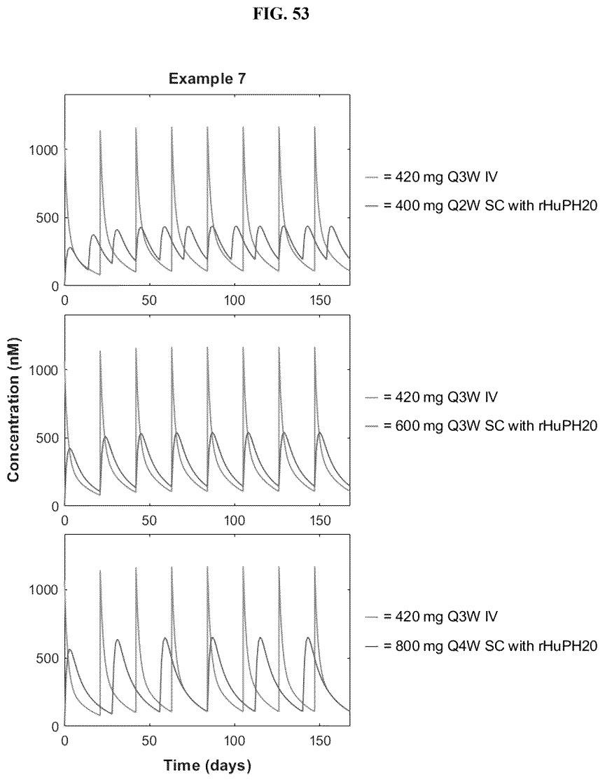

All simulations, for TRODELVY (FIG. 46) and eight others (FIGS. 47-54), demonstrated the ability to convert the current IV dosing regimen to a SC regimen with rHuPH20, with equivalent AUC and lower Cₘₐₓ. Since Cₘₐₓ is typically a driver of systemic toxicities for ADCs, this suggests the regimen of SC with rHuPH20 could provide an improved safety profile. Additionally, all eight simulated examples demonstrated the ability of a SC regimen with rHuPH20 to maintain this equivalent AUC and lower Cₘₐₓ compared to IV, at less and more frequent dosing schedules, enabling more options for optimizing the safety and/or efficacy profile of the ADC.

### Example 10: Pharmacokinetics of an ADC, Trodelvy, Following Subcutaneous Administration with and without rHuPH20

The objective of this study was to investigate the pharmacokinetics (PK) of a monoclonal antibody drug conjugate (ADC), sacituzumab govitecan, following subcutaneous (SC) administration alone or co-administered with recombinant human hyaluronidase PH20 (rHuPH20). This study determined the impact of rHuPH20 on the PK of this antibody, and the impact on the absorption and bioavailability of the antibody following SC injection.

Minipigs were used in this study due to the similarity of the SC skin architecture to humans. Three cohorts were included in this study. Cohort 1 received the ADC administered intravenously. Cohort 2 received the ADC alone delivered via SC injection, while Cohort 3 received the ADC co-mixed with rHuPH20 (2,000 U/mL) also delivered via SC injection. All injections were 5 mL. Blood collection timepoints were pre-treatment, 6 h, 24 h, 2d, 72h, 96h, and 168h post-injection. Blood was drawn via jugular vein stick and collected into serum separator tubes (SSTs), centrifuged, and serum stored at ≤−70° C. until bioanalytical analysis. Punch biopsies of SC injection sites were collected for bioanalytical and histopathological evaluation at the end of study (Day 7). This report includes data from a follow-on study with SC dosing mirroring cohorts 2 and 3, but had punch biopsies of each SC injection site collected on Days 1 and 2.

The results indicate that rHuPH20 enables faster absorption and reduced SC tissue residence time, which can result in improved local safety profile via less payload existing at the SC site of administration. Additionally, rHuPH20 improves SC bioavailability, resulting in higher concentrations of intact, i.e., payload-bound ADC, in systemic circulation.

### Introduction and Objectives

Subcutaneous administration of large volumes of antibodies has been shown to be feasible when the antibody solution is co-formulated with recombinant human hyaluronidase PH20 (rHuPH20). rHuPH20 has been shown to facilitate SC administration of fluids and drugs by transiently and locally depolymerizing hyaluronan (HA) in the extracellular matrix thereby reducing tissue backpressure in the SC space permitting rapid, large volume administration of fluid. Using this technology, subcutaneous administration of large volumes of antibody has become possible and is replacing intravenous administration as a treatment paradigm. However, new treatment paradigms using monoclonal antibodies are focusing on the preparation of higher concentration solutions that may provide higher systemic exposure for longer durations, potentially supporting longer clinical dosing intervals.

The minipig model has been selected due to the high degree of similarity of the subcutaneous space to that of humans. Previous studies using a minipig model have demonstrated the translatability of the model for use in pre-clinical studies. This study determined if recombinant human hyaluronidase (rHuPH20) can improve the PK and absorption of an ADC by enhancing the local dispersion.

### Experimental Design and Methods

### Test and Control Articles

1) Sacituzumab govitecan (Trodelvy)
Lot number: 841845689249; 841912850403 (present study—Example 10)
842135097067; 842828428634; 843806752996 (follow on study—Example 11)
Expiration date: December 2026
Concentration: 10 mg/mL after rehydration in 0.9% saline
Storage Conditions: 2-8° C.
Handling Conditions: Standard laboratory precautions
Manufacturer: Gilead Sciences Ireland UC
Supplier: Tanner Pharma Group

Trodelvy is an antibody drug conjugate (molecular weight ˜150 kDa) consisting of an IgG1 antibody conjugated to a payload of SN-38 via a cleavable pH-sensitive linker.

2) Recombinant Human Hyaluronidase PH20 (rHuPH20)
Description: Clear colorless liquid
Lot number: 1-FIN-3928
Concentration: 1,039,763 U/mL; 10 mg/mL
Formulation: 10 mM Histidine, 130 mM sodium chloride, pH 6.5
Storage Conditions: ≤70° C.
Handling Conditions: Standard laboratory precautions
Supplier: Halozyme, Inc.

### 3) 0.9% Sodium Chloride

Description: Clear colorless liquid
Lot number: LP5732
Expiration date: 2026 Jul. 31
Storage Conditions: Room temperature
Handling Conditions: Standard laboratory precautions
Supplier: Hospira

### Formulation

Two test solutions were used in this study: ADC (sacituzumab govitecan) alone and the ADC co-mixed with rHuPH20. Two vials of the antibody drug conjugate were rehydrated by the addition of 20.0 mL of 0.9% Sodium Chloride to 200 mg of the lyophilized product (40 mL total) yielding a final concentration of 10 mg/mL. 0.035 mL of 10 mg/mL rHuPH20 was added to 18 mL of the rehydrated ADC to yield a test solution of the ADC+rHuPH20 at a target concentration of 2000 U/mL. Each dose of the ADC was 50 mg (5 mL).

### Syringe Preparation.

All solutions were prepared in a Biosafety cabinet using standard laboratory safety protocol. The co-mix of ADC+rHuPH20 was used to prepare (3) 10-cc syringes each containing 5.00 mL of the test solution. The remaining test solution of the ADC alone was then used to fill (4) 10-cc syringes each containing 5.00 mL. After filling the syringe, it was capped and stored at 2-8° C. until removed prior to use and brought to room temperature for at least 30 minutes. The syringe cap was removed and replaced with a 25G×1″ needle immediately prior to use for administration.

### Dose Concentration Analysis.

Prior to study start, enzyme activity of the test solution was measured using an in vitro activity assay to confirm the concentration of rHuPH20 in the co-mix drug solution. The results are shown in Table 46.

TABLE 46
Summary of pre-study activity testing

| Test Solution | Activity (U/mL ± SD) |
|---|---|
| ADC + rHuPH20 | 2206 ± 28 |

### Animal Description

Species: Pig (Sus scrofa domestica)
Strain: Yucatan Miniature
Sex: Female
Age: ˜3-4 months
Weight: ˜15-18 kg
Quantity: 7 animals
Source: Premier BioSource (Ramona, CA)

### Summary of Bodyweights.

The bodyweights and date of birth for each animal is provided in Table 47.

TABLE 47
Summary of bodyweights and dates of birth

| Animal ID# | Bodyweight (kg) | Date of Birth |
|---|---|---|
| 8540 | 17 | Jan. 28, 1925 |
| 8543 | 16 | Jan. 28, 1925 |
| 8546 | 16 | Jan. 29, 1925 |
| 8547 | 15 | Jan. 28, 1925 |
| 8550 | 18 | Jan. 29, 1925 |
| 8551 | 15 | Jan. 29, 1925 |
| 8552 | 16 | Jan. 28, 1925 |

### Husbandry.

The animals were housed in a steel pen with automatic water provided ad libitum. The animals were fed twice daily (AM and PM) but were kept NPO after midnight on the day of the study to prevent anesthesia complications. The room environment was set to maintain a temperature of ˜19-23° C. and a relative humidity of 40-70%, with a 12-hour light/12-hour dark time cycle. The animals were acclimated to the vivarium for 3 days prior to study start.

### Test Materials

The test materials shown in Table 48 were used in the study.

TABLE 48
Test materials

| | | | Expiration |
|---|---|---|---|
| Test Material | Supplier | Catalog # | Date |
| 10-cc syringe | Becton Dickinson | 302995 | NA |
| Syringe caps | Becton Dickinson | 305819 | NA |
| 25 G × 1 inch Precision | Becton Dickinson | 305125 | NA |
| Glide needle | | | |
| 20 G × 1 inch thin | Mckesson | 16-N201 | 2026 Jul. 31 |
| wall needle | | | |
| 0.9% sodium chloride | Mckesson | 239940 | NA |
| for injection | | | |
| Vacutainer Blood | Becton Dickinson | 367977 | NA |
| Collection Tubes | | | |
| (Serum Separator Tubes) | | | |
| Cryogenic Vials | Fischer Scientific | 10-500-26 | NA |
| O.C.T. freezing | Fisher Scientific | 23-730-571 | NA |
| compound | | | |
| Epredid ® Embedding | Fisher Scientific | 22-19 | NA |
| Molds | | | |

### Bioanalytical Methods

Serum concentrations of total ADC, total antibody and Free Payload (unconjugated SN38) were determined in three separate assays conducted by PPD. Method qualification in pig serum and study sample analysis results are reported in Tables 64 and 65.

### Analysis of Total Antibody and Total ADC in Serum and Skin.

A 20.0-μL sample aliquot was diluted 10-fold. A portion of the diluted sample was taken and processed using an immunoaffinity approach. SMART Digest IA magnetic beads coated with anti-human Fc antibody were used to enrich Trodelvy from matrix. The bound proteins were subjected to “on-bead” proteolysis with trypsin. As a result of the trypsin digestion, characteristic peptide fragments originating from the antibody were produced and used for Total Antibody quantitation. After trypsin digestion, the proteins were further subjected to acidic conditions to release SN-38 from the antibody, which was used for Total ADC quantitation. The following sequences in Table 49 were selected for monitoring in the assay:

TABLE 49
Amino acid sequences used for bioanalytical analysis of samples

| Name | Amino Acid Sequence | Purpose |
|---|---|---|
| VVSV | VVSVLTVLHQDWLNGK | Quantitation of Total Antibody |
| | (SEQ ID NO: 18) | (Fc, Heavy Chain) |
| DSTY | DSTYSLSSTLTLSK | Intact Antibody (Qualitative) |
| | (SEQ ID NO: 19) | (Kappa Light Chain) |
| VVSV-IS | VVSVLTVLHQDWLNGK*^{a} | Internal Standard |
| | (SEQ ID NO: 20) | |
| DSTY-IS | DSTYSLSSTLTLSK*^{a} | Internal Standard |
| | (SEQ ID NO: 21) | |
| ADC | SN-38 | Quantitation of Total ADC (SN-38) |
| ADC-IS | SN-38-d3 | Internal Standard |

^{a} K* denotes amino acid containing stable isotope labeling-*Lys = 13C6, 15N2-Lys

Calibration curves and quality controls are prepared by fortifying Trodelvy in blank porcine serum at concentrations ranging from 0.500 to 250 μg/mL. VVSV was used for the quantitation of Total Antibody concentrations, DSTY was used for the quantitation of Intact Antibody (trouble shooting purposes only), and SN-38 was used for quantitating Total ADC concentrations.

Porcine serum was used as surrogate matrix to prepare calibration standards and quality controls for skin sample quantitation. Untreated blank porcine skin was provided for preparation of monitoring quality controls. The porcine skin was homogenized at an approximate ratio of 0.5 mL of buffer for every 100 mg of skin. The homogenized porcine skin (primary matrix) was used to prepare a low (1.50 μg/mL) and high (190 μg/mL) monitoring quality controls.

### Analysis of Free Payload (Unconjugated SN-38) in Serum and Skin Background.

A nominal unconjugated SN-38, concentration range of 10.0 to 5000 μg/mL was chosen to quantitate samples. This assay requires a 50.0 μL porcine serum aliquot. Samples were kept frozen at −80° C. prior to analysis.

A matrix aliquot was fortified with internal standard. Analytes were isolated through protein precipitation. The eluate was evaporated under a nitrogen stream, and the remaining residue reconstituted. Final extracts were analyzed via HPLC MS/MS detection using positive ion electrospray.

Porcine serum was used as surrogate matrix to prepare calibration standards and quality controls for the quantitation of skin samples. Supported liquid-liquid extraction was used for sample clean up. Untreated blank porcine skin was provided to be able to homogenize and prepare monitoring quality controls. The porcine skin was homogenized at an approximate ratio of 0.5 mL of buffer for every 100 mg of skin. The homogenized porcine skin (primary matrix) was used to prepare a low (27.5 pg/mL) and high (3750 pg/mL) monitoring quality control.

### Pharmacokinetic Analysis Methods

Pharmacokinetic data were generated from serum (present study) and skin samples (present and follow-on studies) collected over a 7-day (168h) period. Serum concentrations were measured at nominal time points of 0 (Pre-injection), 6 h, 24 h, 48 h, 72h, 96h, and 168h while skin concentrations were available at 24h. 48h, and 168h. For PK analysis of serum samples, actual time points versus nominal time points (reported in Table 51 through Table 56) varied by less than 2%. Therefore, nominal time points were used for PK analyses. To estimate the serum concentration at time zero for intravenous (IV) data, a second-order polynomial regression was applied to the early serum time points (0.25, 1, and 2 hours). The intercept of the fitted polynomial curve was used as the extrapolated concentration at time zero. Skin concentration data (μg/mL) was normalized to total protein (mg/mL) per sample, resulting in values represented as μg/mg protein.

Pharmacokinetic parameters were calculated using noncompartmental methods performed in Microsoft Excel. The area under the concentration-time curve (AUC) was determined using the linear trapezoidal rule. For serum data, AUC₀₋₇ was calculated using all available time points. For skin data, AUC₁₋₇ was computed based on the three available time point measurements. The maximum observed concentration (Cmax) was identified directly from the dataset, and the time at which it occurred (Tmax) was recorded. All calculations were performed using Microsoft Excel. Raw data for serum concentrations of total antibody, total ADC and free payload as well as the calculated PK metrics are reported in Tables 64 and 65.

### Experimental Design

This study measured the pharmacokinetics (PK) of subcutaneous administration of sacituzumab govitecan alone or co-mixed with rHuPH20 and compared it to the IV PK of sacituzumab govitecan. Each animal received a single 5 mL injection of the ADC or the ADC+rHuPH20. Cohort received the ADC administered IV, while cohort 2 and cohort 3 received the ADC and ADC+rHuPH20 test solutions injected SC, respectively. The treatment groups are shown in Table 50.

TABLE 50
Description of treatments

| | | | | | Injection | | |
|---|---|---|---|---|---|---|---|
| Cohort | | | | Dose | Volume | rHuPH20 | Blood Sampling |
| # | Test Solution | N | Route | (mg) | (mL) | (Units) | Times (h/d) |
| 1 | sacituzumab | 1 | IV | 50 | 5 | 0 | Pre, 6 h, 24 h, 48 h, |
| | govitecan | | | | | | 72 h, 96 h, 168 h |
| 2 | sacituzumab | 3 | SC | 50 | 5 | 0 | Pre, 6 h, 24 h, 48 h, |
| | govitecan | | | | | | 72 h, 96 h, 168 h |
| 3 | sacituzumab | 3 | SC | 50 | 5 | 2,000 | Pre, 6 h, 24 h, 48 h, |
| | govitecan + | | | | | | 72 h, 96 h, 168 h |
| | rHuPH20 | | | | | | |

Animals were anesthetized for all blood collection timepoints. On the day of the procedure, the animals were anesthetized twice (once for the initial procedure and a second time for the 6h blood collection timepoint). AccuLab staff monitored animals to mitigate any possible hypoglycemia that could occur during the interval between the treatment and the 6h timepoint.

A pre-treatment blood sample (Pre) was obtained from each animal prior to the initial injection. The animal had approximately a 3 mL blood sample collected at Predose, 6 h, 24 h, 48h, 72h, 96h, and 168h via jugular vein stick which was transferred to Halozyme staff and processed for serum. After the final blood collection timepoint, the animals were humanely euthanized. Punch biopsies of SC injection sites were collected for bioanalytical and histopathological evaluation.

### Study Design

Prior to the start of study, the animal was assessed for general health, its body weight recorded, and a pre-treatment baseline blood sample obtained. Additionally, blood collections were obtained from each animal at 6h post injection, and at 24 h, 48 h, 72h, 96h, and 168h. All blood samples were collected into serum separating tubes (SSTs), allowed to clot at room temperature for a minimum of 30 minutes, then centrifuged in a swinging bucket centrifuge (1200×g; 10 minutes; 20-23° C.). After centrifugation, the serum was aliquoted into two cryogenic vials (primary and backup samples) and stored in a freezer set to maintain ≤−70° C. until analysis. After euthanasia, 12 mm punch biopsies were collected of the SC injection sites. The tissue was divided into two parts (sectioned vertically) with one-half placed in a histological cassette then frozen in OCT. The other half was placed into cryovials, the weight recorded and then frozen on dry ice for bioanalytical evaluation.

On the day of the procedure, animals were anesthetized and placed on a heated surgical table and maintained under isoflurane gas for the entire duration of the procedure. An intravenous (IV) catheter was placed into the ear of the animal in cohort 1 to facilitate IV dosing. For cohort 2 and cohort 3, the test solution was delivered to the SC space located on the lower left abdomen, mid-flank, approximately 3 cm toward the midline of the animal and 5 cm cranially. Animals in cohort 2 received a single SC injection of sacituzumab govitecan, and animals in cohort 3 received a single SC injection of sacituzumab govitecan co-mixed with rHuPH20 at a concentration of 2000 U/mL.

### Results

### Bioanalytical and Pharmacokinetic Analysis—Serum

### Nominal and Actual Blood Collection Time.

Following dosing, blood was collected at approximately 6 h, 24 h, 48 h, 72h, 96h, and 168h. The time that each blood collection occurred was recorded and the nominal versus the actual blood collection timepoints are shown in Table 51 through Table 56.

TABLE 51
Nominal blood collection times and actual blood
collection times - 6 hours (360 minutes)

| | | Nominal | Actual | | |
|---|---|---|---|---|---|
| | | Blood | Blood | Time | Time |
| Animal | Injection | Collection | Collection | Deviation | Deviation |
| ID# | Time | Time - 6 h | Time - 6 h | (min) | (%) |
| 8540 | 8:59 | 14:59 | 14:55 | 4 | 1.11% |
| 8543 | 9:10 | 15:10 | 15:12 | 2 | 0.56% |
| 8546 | 9:20 | 15:20 | 15:18 | 2 | 0.56% |
| 8547 | 9:29 | 15:29 | 15:27 | 2 | 0.56% |
| 8550 | 9:40 | 15:40 | 15:38 | 2 | 0.56% |
| 8551 | 9:50 | 15:50 | 15:46 | 4 | 1.11% |
| 8552 | 10:00 | 16:00 | 15:53 | 7 | 1.94% |

TABLE 52
Nominal blood collection times and actual blood
collection times - 24 hours (1440 minutes)

| | | Nominal | Actual | | |
|---|---|---|---|---|---|
| | | Blood | Blood | Time | Time |
| Animal | Injection | Collection | Collection | Deviation | Deviation |
| ID# | Time | Time - 24 h | Time - 24 h | (min) | (%) |
| 8540 | 8:59 | 8:59 | 8:50 | 9 | 0.63% |
| 8543 | 9:10 | 9:10 | 9:03 | 7 | 0.49% |
| 8546 | 9:20 | 9:20 | 9:14 | 6 | 0.42% |
| 8547 | 9:29 | 9:29 | 9:23 | 6 | 0.42% |
| 8550 | 9:40 | 9:40 | 9:37 | 3 | 0.21% |
| 8551 | 9:50 | 9:50 | 9:46 | 4 | 0.28% |
| 8552 | 10:00 | 10:00 | 9:59 | 1 | 0.07% |

TABLE 53
Nominal blood collection times and actual blood
collection times - 48 hours (2880 minutes)

| | Injec- | Nominal Blood | Actual Blood | Time | Time |
|---|---|---|---|---|---|
| Animal | tion | Collection | Collection | Deviation | Deviation |
| ID# | Time | Time - 48 h | Time - 48 h | (min) | (%) |
| 8540 | 8:59 | 8:59 | 8:57 | 2 | 0.07% |
| 8543 | 9:10 | 9:10 | 9:02 | 8 | 0.28% |
| 8546 | 9:20 | 9:20 | 9:11 | 9 | 0.31% |
| 8547 | 9:29 | 9:29 | 9:21 | 8 | 0.28% |
| 8550 | 9:40 | 9:40 | 9:32 | 8 | 0.28% |
| 8551 | 9:50 | 9:50 | 9:44 | 6 | 0.21% |
| 8552 | 10:00 | 10:00 | 9:53 | 7 | 0.24% |

TABLE 54
Nominal blood collection times and actual blood
collection times - 72 hours (4320 minutes)

| | Injec- | Nominal Blood | Actual Blood | Time | Time |
|---|---|---|---|---|---|
| Animal | tion | Collection | Collection | Deviation | Deviation |
| ID# | Time | Time - 72 h | Time - 72 h | (min) | (%) |
| 8540 | 8:59 | 8:59 | 8:50 | 9 | 0.21% |
| 8543 | 9:10 | 9:10 | 9:04 | 6 | 0.14% |
| 8546 | 9:20 | 9:20 | 9:12 | 8 | 0.19% |
| 8547 | 9:29 | 9:29 | 9:24 | 5 | 0.12% |
| 8550 | 9:40 | 9:40 | 9:36 | 4 | 0.09% |
| 8551 | 9:50 | 9:50 | 9:44 | 6 | 0.14% |
| 8552 | 10:00 | 10:00 | 9:55 | 5 | 0.12% |

TABLE 55
Nominal Blood Collection Times and Actual Blood
Collection Times - 96 hours (5760 minutes)

| | Injec- | Nominal Blood | Actual Blood | Time | Time |
|---|---|---|---|---|---|
| Animal | tion | Collection | Collection | Deviation | Deviation |
| ID# | Time | Time - 96 h | Time - 96 h | (min) | (%) |
| 8540 | 8:59 | 8:59 | 8:54 | 5 | 0.09% |
| 8543 | 9:10 | 9:10 | 9:04 | 6 | 0.10% |
| 8546 | 9:20 | 9:20 | 9:14 | 6 | 0.10% |
| 8547 | 9:29 | 9:29 | 9:26 | 3 | 0.05% |
| 8550 | 9:40 | 9:40 | 9:35 | 5 | 0.09% |
| 8551 | 9:50 | 9:50 | 9:44 | 6 | 0.10% |
| 8552 | 10:00 | 10:00 | 9:54 | 6 | 0.10% |

TABLE 56
Nominal blood collection times and actual blood
collection times - 168 hours (10080 minutes)

| | Injec- | Nominal Blood | Actual Blood | Time | Time |
|---|---|---|---|---|---|
| Animal | tion | Collection | Collection | Deviation | Deviation |
| ID# | Time | Time - 168 h | Time - 168 h | (min) | (%) |
| 8540 | 8:59 | 8:59 | 9:03 | 4 | 0.04% |
| 8543 | 9:10 | 9:10 | 9:14 | 4 | 0.04% |
| 8546 | 9:20 | 9:20 | 9:30 | 10 | 0.10% |
| 8547 | 9:29 | 9:29 | 9:38 | 9 | 0.09% |
| 8550 | 9:40 | 9:40 | 9:49 | 9 | 0.09% |
| 8551 | 9:50 | 9:50 | 10:01 | 11 | 0.11% |
| 8552 | 10:00 | 10:00 | 10:11 | 11 | 0.11% |

### Pharmacokinetic Analysis

### Measurement of Serum ADC Values—Total ADC (TADC), Total Antibody (TAB) and Free Payload.

Quantitation of total antibody, total ADC, and free payload (SN-38) from Trodelvy in porcine serum and tissue samples was performed via HPLC with MS/MS detection. The serum values for Total Antibody (TAB) for cohort 1 (ADC-IV), cohort 2 (ADC-SC), and cohort 3 (ADC+rHuPH20-SC) at each blood collection timepoint are listed in Table 57 and shown graphically in FIG. 56.

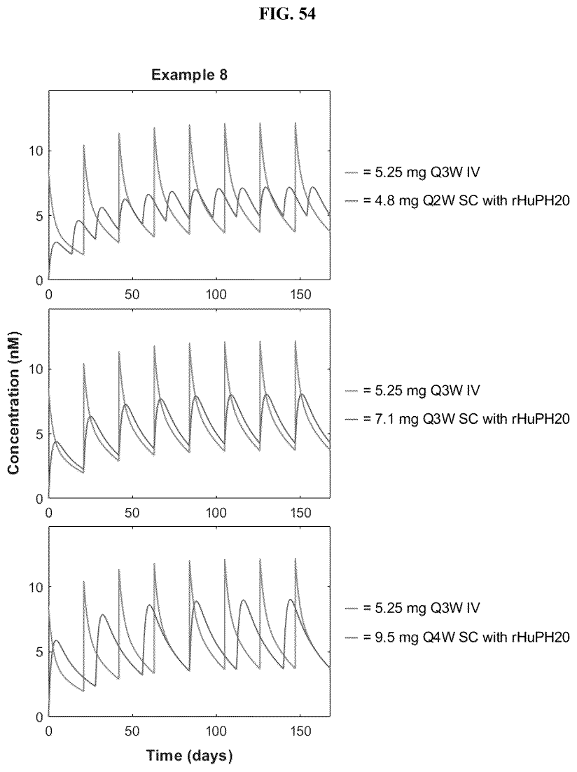

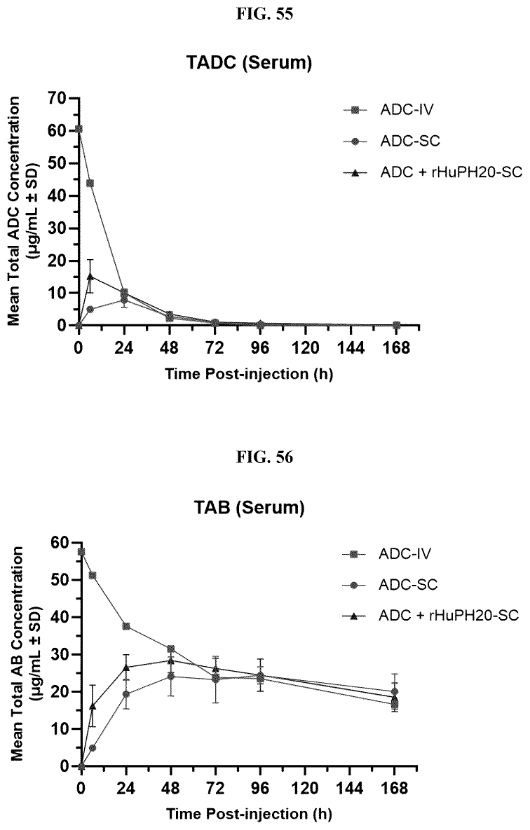

Total ADC (TADC) concentration was also measured in the serum at each blood collection time point for cohort 1 (ADC-IV), cohort 2 (ADC-SC), and cohort 3 (ADC+rHuPH20-SC) and is described in Table 58 and shown graphically in FIG. 55.

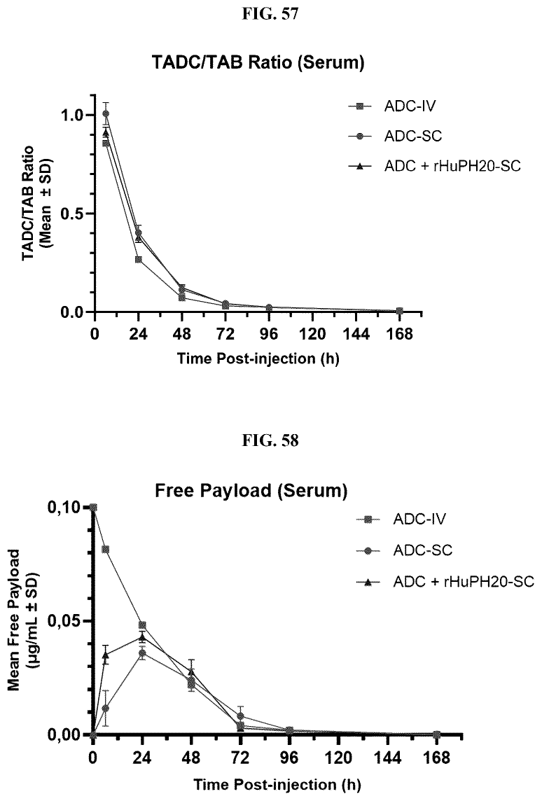

The total free payload (unconjugated SN-38) was measured in serum samples to determine how long it remained conjugated to the antibody component of the ADC. The hydrolyzable linker used to conjugate SN38 to sacituzumab is relatively instable, even at neutral pH. As such, the sample preparation method for the LC/MS free payload assay is known to cause some hydrolysis of the linker, leading to an overestimation of the free payload in each sample. The measured quantity of free payload for cohort 1 (ADC-IV), cohort 2 (ADC-SC), and cohort 3 (ADC+rHuPH20-SC) are described in Table 59 and shown graphically in FIG. 58.

Tables 57, 58, 59, and 61 describe the differences in AUC₀₋₇, Cmax, and Tmax for cohort 1 (ADC-IV), cohort 2 (ADC-SC), and SC with rHuPH20 (ADC+rHuPH20-SC). In general, rHuPH20 improves the SC absorption and distribution of each analyte, i.e., total antibody (TAB), total ADC (TADC) and free payload (Payload), as demonstrated by the increases in AUC₀₋₇ and Cmax, and decrease in Tmax, relative to SC alone. Relative to IV, AUC₀₋₇ is lower for SC and rHuPH20, but with a greater-than-proportional decrease in Cmax. For example, the ratio of (ADC+rHuPH20-SC)/(ADC-IV) AUC₀₋₇ is 52%, while that for Cmax is 25% (see Table 58).

The concentration of total ADC (TADC) concentration was compared to the total antibody (TAB) concentrations and is expressed as a ratio in Table 60 and shown graphically in FIG. 57.

TABLE 57
Individual and mean serum concentrations of total antibody (TAB) - (μg/mL)

| | | | | | | | | ADC + | ADC + | ADC + | | | |
|---|---|---|---|---|---|---|---|---|---|---|---|---|---|
| | | | | | | | | rHuPH20 | rHuPH20 | rHuPH20 | | | |
| | ADC IV | ADC SC | ADC SC | ADC SC | | | | SC | SC | SC | Mean | SD | % CV |
| Time Post- | (Animal | (Animal | (Animal | (Animal | Mean | SD | % CV | (Animal | (Animal | (Animal | SC + | SC + | SC + |
| Injection (h) | 8540) | 8543) | 8546) | 8547) | SC | SC | SC | 8550) | 8551) | 8552) | PH20 | PH20 | PH20 |
| 0 | 57.60^{a} | <0.10^{b} | <0.10^{b} | <0.10^{b} | 0.00^{b} | NA | NA | <0.10^{b} | <0.10^{b} | <0.10^{b} | 0.000 | NA | NA |
| 6 | 51.30 | 5.00 | 4.88 | 4.93 | 4.94 | 0.06 | 0.01 | 13.20 | 22.70 | 12.80 | 16.23 | 5.60 | 0.35 |
| 24 | 37.60 | 16.30 | 18.00 | 23.90 | 19.40 | 3.99 | 0.21 | 24.80 | 30.50 | 24.40 | 26.57 | 3.41 | 0.13 |
| 48 | 31.50 | 19.90 | 22.50 | 30.00 | 24.13 | 5.24 | 0.22 | 24.70 | 30.80 | 29.80 | 28.43 | 3.27 | 0.12 |
| 72 | 23.90 | 18.40 | 21.10 | 30.30 | 23.27 | 6.24 | 0.27 | 23.50 | 29.00 | 26.30 | 26.27 | 2.75 | 0.10 |
| 96 | 23.50 | 23.80 | 22.50 | 27.00 | 24.43 | 2.32 | 0.09 | 19.60 | 25.80 | 28.00 | 24.47 | 4.36 | 0.18 |
| 168 | 16.60 | 16.20 | 18.60 | 25.40 | 20.07 | 4.77 | 0.24 | 16.20 | 16.30 | 23.00 | 18.50 | 3.90 | 0.21 |
| AUC₀₋₇ | 193.05 | 126.96 | 134.69 | 175.78 | 145.81 | 26.24 | 0.18 | 140.00 | 173.89 | 174.35 | 162.75 | 19.70 | 0.12 |
| (μg*day/mL) | | | | | | | | | | | | | |
| Cmax | 57.60 | 23.80 | 22.50 | 30.30 | 25.53 | 4.18 | 0.16 | 24.80 | 30.80 | 29.80 | 28.47 | 3.21 | 0.11 |
| (μg/mL) | | | | | | | | | | | | | |
| Tmax (day) | 0.00 | 4.00 | 2.00 | 3.00 | 3.00 | 1.00 | 0.33 | 1.00 | 2.00 | 2.00 | 1.67 | 0.58 | 0.35 |

^{a}Extrapolated
^{b}Values <LLOQ set to ‘0’ for PK parameter calculations

TABLE 58
Individual and mean serum concentrations of total ADC (TADC) - (μg/mL)

| | Animal ID# | | | | | | | | | | | | |
|---|---|---|---|---|---|---|---|---|---|---|---|---|---|
| | | | | | | | | 8550 | 8551 | 8552 | | | |
| | 8540 | 8543 | 8546 | 8547 | | | | ADC + | ADC + | ADC + | Mean | SD | % CV |
| Time Post- | ADC | ADCS | ADC | ADC | Mean | SD | % CV | rHuPH20 | rHuPH20 | rHuPH20 | SC + | SC + | SC + |
| Injection (h) | IV | C | SC | SC | SC | SC | SC | SC | SC | SC | PH20 | PH20 | PH20 |
| 0 | 60.55^{a} | <0.10^{b} | <0.10^{b} | <0.10^{b} | 0.00 | N/A | N/A | <0.10^{b} | <0.10^{b} | <0.10^{b} | 0.00 | N/A | N/A |
| 6 | 43.90 | 4.71 | 5.03 | 5.15 | 4.96 | 0.23 | 0.05 | 11.80 | 21.10 | 12.60 | 15.17 | 5.15 | 0.34 |
| 24 | 10.00 | 6.60 | 6.50 | 10.50 | 7.87 | 2.28 | 0.29 | 8.92 | 11.30 | 10.00 | 10.07 | 1.19 | 0.12 |
| 48 | 2.27 | 2.31 | 2.12 | 3.89 | 2.77 | 0.97 | 0.35 | 2.73 | 3.78 | 4.11 | 3.54 | 0.72 | 0.20 |
| 72 | 0.75 | 0.84 | 0.92 | 1.23 | 1.00 | 0.20 | 0.21 | 0.79 | 1.24 | 1.28 | 1.10 | 0.27 | 0.25 |
| 96 | <0.50^{b} | 0.53 | <0.50^{b} | 0.65 | 0.39 | 0.35 | 0.88 | <0.50^{b} | <0.50^{b} | 0.71 | 0.24 | 0.41 | 1.73 |
| 168 | 0.10 | 0.13 | 0.10 | 0.16 | 0.13 | 0.03 | 0.22 | 0.11 | 0.13 | 0.15 | 0.13 | 0.02 | 0.18 |
| AUC₀₋₇ | 41.44 | 12.55 | 11.40 | 18.42 | 14.12 | 3.77 | 0.27 | 17.38 | 25.66 | 22.09 | 21.71 | 4.15 | 0.19 |
| (ug*day/mL) | | | | | | | | | | | | | |
| Cmax | 60.55 | 6.60 | 6.50 | 10.50 | 7.87 | 2.28 | 0.29 | 11.80 | 21.10 | 12.60 | 15.17 | 5.15 | 0.34 |
| (ug/mL) | | | | | | | | | | | | | |
| Tmax (day) | 0.00 | 1.00 | 1.00 | 1.00 | 1.0 | 0.00 | 0.00 | 0.25 | 0.25 | 0.25 | 0.25 | 0.00 | 0.00 |

^{a}Extrapolated
^{b}Values <LLOQ set to ‘0.00’ for PK parameter calculations

TABLE 59
Individual and mean serum concentrations of free payload - (μg/mL)

| | Animal ID# | | | | | | | | | | | | |
|---|---|---|---|---|---|---|---|---|---|---|---|---|---|
| Time | | | | | | | | 8550 | 8551 | 8552 | | | % |
| Post- | 8540 | 8543 | 8546 | 8547 | | | % | ADC + | ADC + | ADC + | Mean | SD | CV |
| Injection | ADC | ADC | ADC | ADC | Mean | SD | CV | rHuPH20 | rHuPH20 | rHuPH20 | SC + | SC + | SC + |
| (h) | IV | SC | SC | SC | SC | SC | SC | SC | SC | SC | PH20 | PH20 | PH20 |
| 0 | 0.10^{a} | <0.0001^{b} | <0.0001^{b} | <0.0001^{b} | 0.0000 | N/A | N/A | <0.0001^{b} | <0.0001^{b} | <0.0001^{b} | 0.0000 | N/A | N/A |
| 6 | 0.0816 | 0.0189 | 0.0127 | 0.0034 | 0.0117 | 0.0078 | 67% | 0.0331 | 0.0400 | 0.0326 | 0.0352 | 0.0041 | 12% |
| 24 | 0.0483 | 0.0367 | 0.0328 | 0.0385 | 0.0360 | 0.0029 | 8% | 0.0445 | 0.0401 | 0.0445 | 0.0430 | 0.0025 | 6% |
| 48 | 0.0221 | 0.0237 | 0.0193 | 0.0292 | 0.0241 | 0.0050 | 21% | 0.0216 | 0.0311 | 0.0306 | 0.0278 | 0.0053 | 19% |
| 72 | 0.0041 | 0.0088 | 0.0038 | 0.0122 | 0.0083 | 0.0042 | 51% | 0.0018 | 0.0038 | 0.0033 | 0.0030 | 0.0010 | 35% |
| 96 | 0.0020 | 0.0027 | 0.0016 | 0.0020 | 0.0021 | 0.0005 | 26% | 0.0010 | 0.0021 | 0.0019 | 0.0016 | 0.0006 | 36% |
| 148 | 0.0001 | 0.0002 | 0.0001 | 0.0002 | 0.0002 | 0.0001 | 47% | 0.0001 | 0.0001 | 0.0002 | 0.0001 | 0.0001 | 37% |
| AUC₀₋₇ | 0.1252 | 0.0796 | 0.0614 | 0.0811 | 0.0740 | 0.0109 | 15% | 0.0810 | 0.0944 | 0.0931 | 0.0895 | 0.0074 | 8% |
| (μg*day/mL) | | | | | | | | | | | | | |
| Cmax | 0.0953 | 0.0367 | 0.0328 | 0.0385 | 0.0360 | 0.0029 | 8% | 0.0445 | 0.0401 | 0.0445 | 0.0430 | 0.0025 | 6% |
| (μg/mL) | | | | | | | | | | | | | |
| Tmax (day) | 0 | 1 | 1 | 1 | 1 | 0 | 0% | 1 | 1 | 1 | 1 | 0 | 0% |

^{0.012a}Extrapolated
^{b}Values <LLOQ set to ‘0.0000’ for PK parameter calculations

TABLE 60
TADC/TAB ratio - serum

| | Animal ID# | | | | | | | | | | | | |
|---|---|---|---|---|---|---|---|---|---|---|---|---|---|
| | | | | | | | | 8550 | 8551 | 8552 | | | |
| Time Post | 8540 | 8543 | 8546 | 8547 | | | | ADC + | ADC + | ADC + | | | |
| Injection | ADC | ADC | ADC | ADC | Mean | SD | % CV | rHuPH20 | rHuPH20 | rHuPH20 | Mean | SD | % CV |
| (h) | IV | SC | SC | SC | SC | SC | SC | SC | SC | SC | SC | SC | SC |
| 6 | 0.86 | 0.942 | 1.031 | 1.045 | 1.006 | 0.056 | 6% | 0.894 | 0.930 | 0.984 | 0.936 | 0.046 | 5% |
| 24 | 0.27 | 0.405 | 0.361 | 0.439 | 0.27 | 0.039 | 14% | 0.360 | 0.370 | 0.410 | 0.380 | 0.026 | 7% |
| 48 | 0.07 | 0.116 | 0.094 | 0.130 | 0.113 | 0.018 | 16% | 0.111 | 0.123 | 0.138 | 0.124 | 0.014 | 11% |
| 72 | 0.03 | 0.046 | 0.044 | 0.041 | 0.043 | 0.003 | 7% | 0.034 | 0.043 | 0.049 | 0.042 | 0.008 | 19% |
| 96 | NA | 0.022 | — | 0.024 | 0.023 | 0.001 | 4% | NA | NA | 0.025 | 0.025 | — | NA |
| 168 | 0.01 | 0.008 | 0.006 | 0.006 | 0.007 | 0.001 | 14% | 0.007 | 0.008 | 0.007 | 0.007 | 0.001 | 14% |

TABLE 61
Relative differences in serum PK metrics for ADC
analytes comparing routes of administration

| | | ADC-SC | ADC + | ADC + |
|---|---|---|---|---|
| | | vs. | rHuPH20- | rHuPH20- |
| | | ADC-IV | SC vs. ADC-IV | SC vs. ADC-SC |
| AUC₀₋₇ | TAB | 76% | 84% | 112% |
| | TADC | 34% | 52% | 154% |
| | Free Payload | 59% | 71% | 121% |
| Cmax | TAB | 44% | 49% | 111% |
| | TADC | 13% | 25% | 193% |
| | Free Payload | 38% | 45% | 120% |
| Tmax | TAB | NA | NA | 56% |
| | TADC | NA | NA | 25% |
| | Free Payload | NA | NA | 100% |

In Table 62, the ADC/Ab and ADC/Free Payload ratios reflect the extent of intact ADC in circulation. While IV exhibits the highest values for AUC₀₋₇ and Cmax, ADC+rHuPH20-SC provides an improvement relative to ADC-SC alone, as reflected in both ratios.

TABLE 62
Relative differences in serum PK metrics for routes
of administration comparing analyte compositions

| | | TADC/TAB | TADC/Free Payload |
|---|---|---|---|
| AUC₀₋₇ | ADC (IV) | 21% | 33088% |
| | ADC (SC) | 10% | 19072% |
| | ADC + rHuPH20 (SC) | 13% | 24255% |
| Cmax | ADC (IV) | 105% | 63539% |
| | ADC (SC) | 31% | 21852% |
| | ADC + rHuPH20 (SC) | 53% | 35244% |
| Tmax | ADC (IV) | NA | NA |
| | ADC (SC) | 33% | 100% |
| | ADC + rHuPH20 (SC) | 15% | 25% |

### Bioanalytical Analysis of Post-Injection Tissue Samples

Tissue Samples from Injection Site—Present Study (Example 10)—D7 Skin Samples.

In the present study, tissue samples of the injection site were taken at 168h (D7) by taking a full thickness 12 mm tissue biopsies of the injection site. This sample was divided vertically into two identical pieces. One skin sample was placed into a pre-weighed cryogenic tube and its weight was recorded then stored on dry ice prior to transfer to ≤−70° C. The other skin sample was processed for potential future histological assessment into OCT freezing media and frozen on dry ice. Only the animals that were dosed SC had tissue biopsies taken: (3) animals were dosed SC with the ADC and (3) animals were dosed SC with the ADC+rHuPH20. The source of each tissue sample is provided in Table 63.

TABLE 63
Source of skin tissue samples - animal ID# -
time point - tissue weight

| Animal | Study | ‘Skin Tissue | Time | | Tissue Weight |
|---|---|---|---|---|---|
| ID# | Protocol | Sample Treatment | Point | | (mg) |
| 8544L | 25042 | ADC | 24 | h | 185.3 |
| 8544R | 25042 | ADC + rHuPH20 | 24 | h | 350.3 |
| 8545L | 25042 | ADC | 24 | h | 215.9 |
| 8545R | 25042 | ADC + rHuPH20 | 24 | h | 258.5 |
| 8554L | 25042 | ADC | 24 | h | 235.3 |
| 8554R | 25042 | ADC + rHuPH20 | 24 | h | 278.5 |
| 8557L | 25042 | ADC | 48 | h | 245.2 |
| 8557R | 25042 | ADC + rHuPH20 | 48 | h | 291.4 |
| 8562L | 25042 | ADC | 48 | h | 355.3 |
| 8562R | 25042 | ADC + rHuPH20 | 48 | h | 500.5 |
| 8567L | 25042 | ADC | 48 | h | 291.4 |
| 8567R | 25042 | ADC + rHuPH20 | 48 | h | 280.7 |
| 8543 | 25041 | ADC | 168 | h | 254.0 |
| 8546 | 25041 | ADC | 168 | h | 328.1 |
| 8547 | 25041 | ADC | 168 | h | 270.6 |
| 8550 | 25041 | ADC + rHuPH20 | 168 | h | 318.3 |
| 8551 | 25041 | ADC + rHuPH20 | 168 | h | 366.4 |
| 8552 | 25041 | ADC + rHuPH20 | 168 | h | 299.5 |

Tissue Samples from Injection Site—Follow-on Study (Example 11)—D1 and D2 Skin Samples.

A follow-on study repeated the subcutaneous dosing of the ADC+rHuPH20 that was conducted in the present study. The objective of this study was to identically mirror the SC dosing performed in the present study except that the tissue harvest occurred at 24 h and 48h. Animals received two SC doses on their lower abdominal region. The first SC injection was the ADC alone (5 mL; 50 mg), followed by a SC dose of the ADC+rHuPH20 (5 mL; 10 mg/mL; 2000 U/mL) on the contralateral side of the abdomen.

The test solutions used for the follow-on study were prepared in the same manner as the present study and dose volumes mirrored those used in the present study. The data from the follow-on study is presented in conjunction with the data from the present study in this report because these studies were designed to complement each other with identical dosing solutions and volumes to enable a combined readout from both studies. Specific details about the dosing of animals in the follow-on study can be found in Example 11.

Two cohorts were included in the follow-on study. Cohort 1 was euthanized 24 h after injection and cohort 2 was euthanized 48h after injection. Each animal had two full thickness skin biopsies taken post-mortem using a 12 mm punch. This sample was divided vertically into two identical pieces. One skin sample was placed into a pre-weighed cryogenic tube and its weight was recorded, then stored on dry ice prior to transfer to ≤−70° C. The other skin sample was processed into OCT molds for potential future histological assessment.

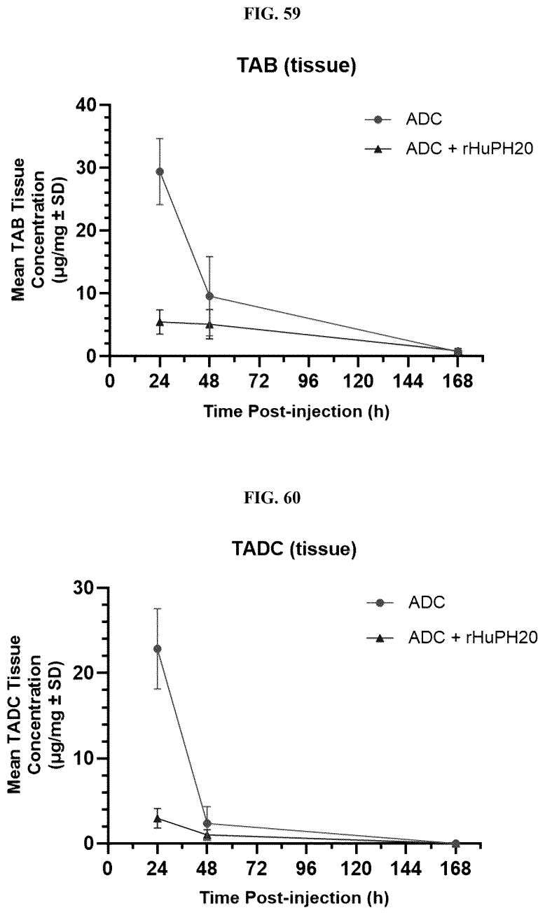

Tissue lysates were prepared based upon the weight of the sample at a ratio of 0.2 g/mL homogenization buffer (Tables 64 and 65). The total protein concentration of each homogenate was determined by BCA assay. The concentration of the total ADC (TADC), total antibody (TAB) and free payload (SN38) in each sample was then normalized to the protein concentration and reported in μg analyte per mg total protein. The source of each tissue sample is provided in Table 63. The concentration of total antibody over time in tissue homogenates is provided in Table 66 and FIG. 59 The total ADC (TADC) concentrations in tissue homogenates is shown in Table 67 and FIG. 60.

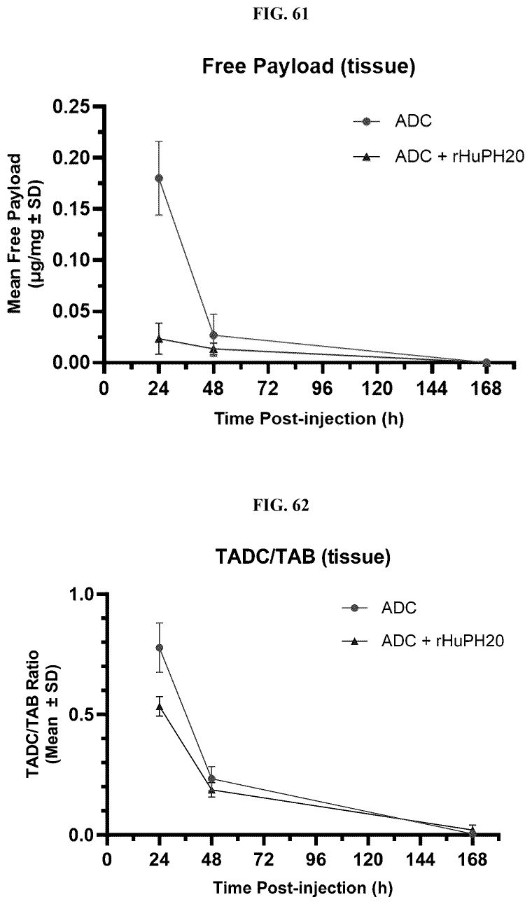

The total Free Payload concentrations in tissue homogenates are shown in Table 68 and FIG. 61. Linker stability during skin tissue sample preparation was assessed by spiking 100 μg/mL Trodelvy into skin tissue, followed by protein precipitation and subsequent sample preparation for LC-MS/MS analysis. Free SN-38 was detected at approximately 10% of the total, which may be attributable to residual impurities in Trodelvy and/or linker hydrolysis occurring during the skin tissue processing steps. FIG. 62 provides the ratio of total ADC to total antibody (TADC/TAB) in skin tissue over time. Table 69 depicts the ratio of total ADC to total antibody in skin tissue homogenates, providing an indication of the extent of intact ADC that is remaining in the SC space over time.

The liquid-liquid extraction method used for the free payload sample processing method for skin tissue homogenates was shown to hydrolyze a significant proportion of payload from intact ADC (Tables 64 and 65), therefore, the measured “free” payload values likely results are known to significantly overestimate the true quantity of unbound payload present in the tissue samples.

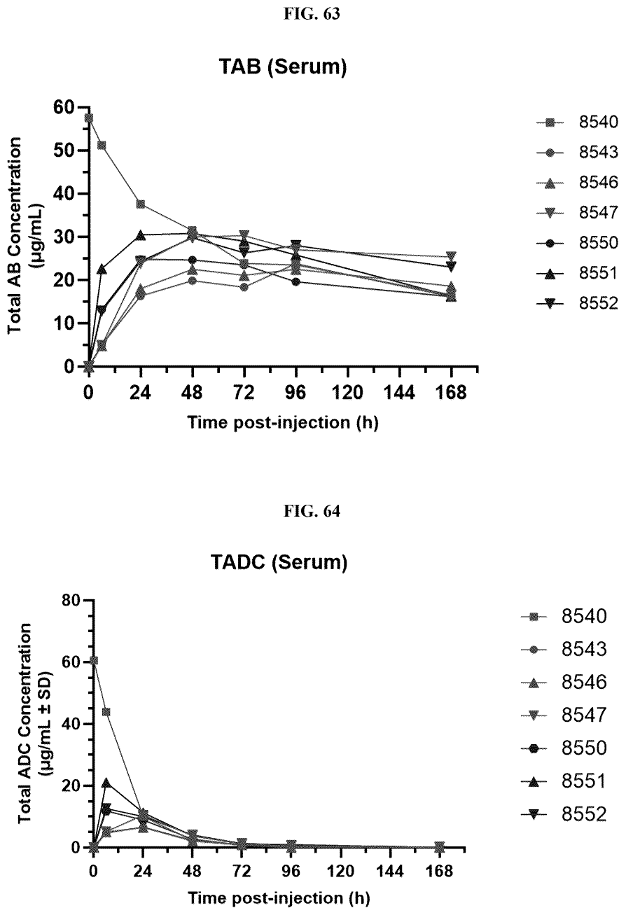

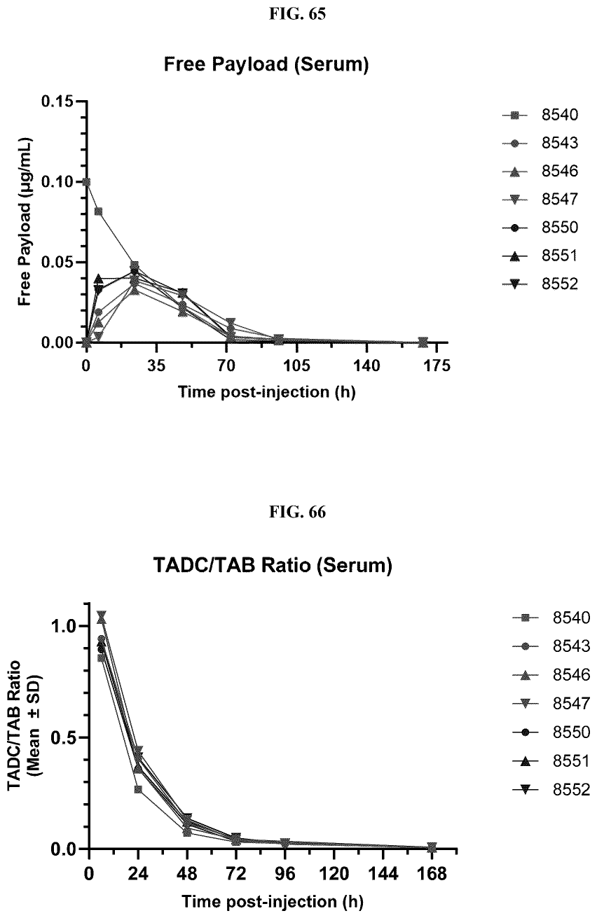

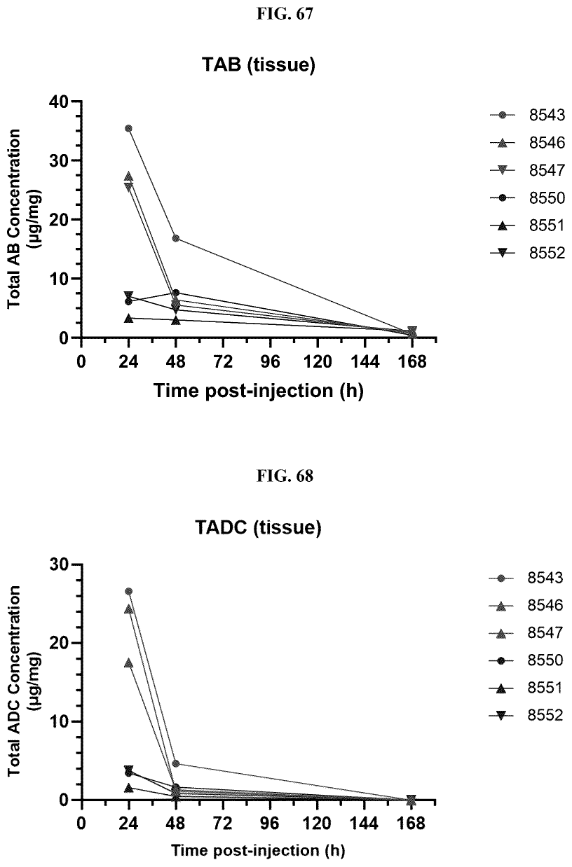

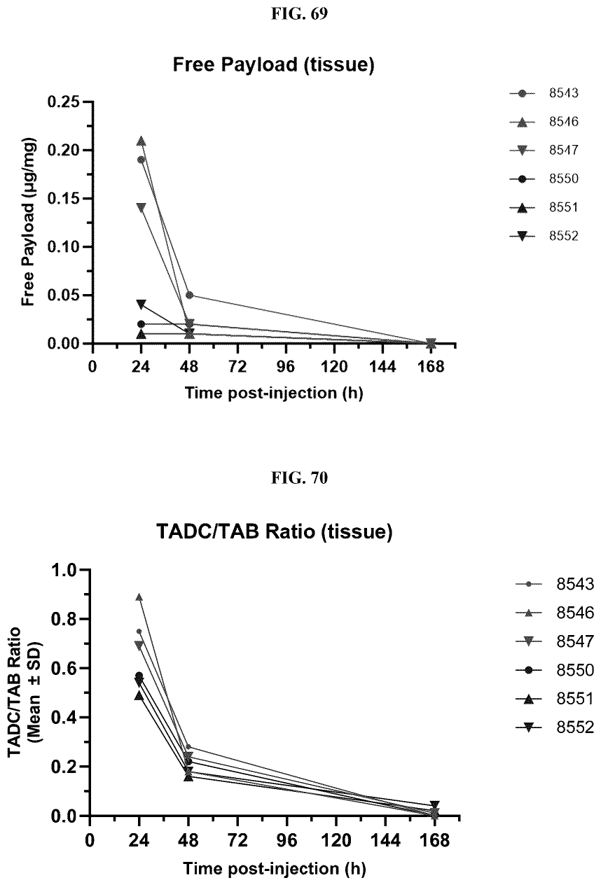

The measurement of serum ADC values for Total Antibody (TAB), Total ADC (TADC), Free Payload and TADC/TAB ratios for individual animals is shown in FIG. 63 through FIG. 66. The measurement of TAB, TADC, Free Payload and TADC/TAB ratios for tissue homogenates is shown in FIG. 67 through FIG. 70.

TABLE 64
Quantitation of Total Antibody, Total ADC and
Free Payload from Trodelvy ® in Minipig
Serum Samples (present study - Example 10)

| | | | Total | Total | | Free |
|---|---|---|---|---|---|---|
| Animal | | | ADC | Antibody | | Payload |
| ID | | Time | Result | Result | TADC/TAB | Result |
| # | Treatment | Point | (μg/mL) | (μg/mL) | Ratio | (pg/mL) |
| 8540 | ADC IV | Pre | <0.100 | <0.100 | NA | <10.0 |
| | | 6 | 43.9 | 51.3 | 0.86 | 81600 |
| | | 24 | 10 | 37.6 | 0.27 | 48300 |
| | | 48 | 2.27 | 31.5 | 0.07 | 22100 |
| | | 72 | 0.751 | 23.9 | 0.03 | 4080 |
| | | 96 | <0.500 | 23.5 | NA | 1980 |
| | | 168 | 0.1 | 16.6 | 0.01 | 85.2 |
| 8543 | ADC SC | Pre | <0.100 | <0.100 | NA | <10.0 |
| | | 6 | 4.71 | 5 | 0.94 | 18900 |
| | | 24 | 6.6 | 16.3 | 0.4 | 36700 |
| | | 48 | 2.31 | 19.9 | 0.12 | 23700 |
| | | 72 | 0.843 | 18.4 | 0.05 | 8750 |
| | | 96 | 0.532 | 23.8 | 0.02 | 2660 |
| | | 168 | 0.133 | 16.2 | 0.01 | 169 |
| 8546 | ADC SC | Pre | <0.100 | <0.100 | NA | <10.0 |
| | | 6 | 5.03 | 4.88 | 1.03 | 12700 |
| | | 24 | 6.5 | 18 | 0.36 | 32800 |
| | | 48 | 2.12 | 22.5 | 0.09 | 19300 |
| | | 72 | 0.921 | 21.1 | 0.04 | 3800 |
| | | 96 | <0.500 | 22.5 | NA | 1580 |
| | | 168 | 0.103 | 18.6 | 0.01 | 84.8 |
| 8547 | ADC SC | Pre | <0.100 | <0.100 | NA | <10.0 |
| | | 6 | 5.15 | 4.93 | 1.04 | 3440 |
| | | 24 | 10.5 | 23.9 | 0.44 | 38500 |
| | | 48 | 3.89 | 30 | 0.13 | 29200 |
| | | 72 | 1.23 | 30.3 | 0.04 | 12200 |
| | | 96 | 0.649 | 27 | 0.02 | 1980 |
| | | 168 | 0.162 | 25.4 | 0.01 | 239 |
| 8550 | ADC + | Pre | <0.100 | <0.100 | NA | <10.0 |
| | rHuPH20 | 6 | 11.8 | 13.2 | 0.89 | 33100 |
| | SC | 24 | 8.92 | 24.8 | 0.36 | 44500 |
| | | 48 | 2.73 | 24.7 | 0.11 | 21600 |
| | | 72 | 0.789 | 23.5 | 0.03 | 1830 |
| | | 96 | <0.500 | 19.6 | NA | 963 |
| | | 168 | 0.107 | 16.2 | 0.01 | 90.9 |
| 8551 | ADC + | Pre | <0.100 | <0.100 | NA | <10.0 |
| | rHuPH20 | 6 | 21.1 | 22.7 | 0.93 | 40000 |
| | SC | 24 | 11.3 | 30.5 | 0.37 | 40100 |
| | | 48 | 3.78 | 30.8 | 0.12 | 31100 |
| | | 72 | 1.24 | 29 | 0.04 | 3830 |
| | | 96 | <0.500 | 25.8 | NA | 2090 |
| | | 168 | 0.132 | 16.3 | 0.01 | 128 |
| 8552 | ADC + | Pre | <0.100 | <0.100 | NA | <10.0 |
| | rHuPH20 | 6 | 12.6 | 12.8 | 0.98 | 32600 |
| | SC | 24 | 10 | 24.4 | 0.41 | 44500 |
| | | 48 | 4.11 | 29.8 | 0.14 | 30600 |
| | | 72 | 1.28 | 26.3 | 0.05 | 3250 |
| | | 96 | 0.707 | 28 | 0.03 | 1880 |
| | | 168 | 0.154 | 23 | 0.01 | 192 |

TABLE 65
Quantitation of total antibody, total ADC and free payload from TrodeIvy in minipig
skin samples (present study (168 h) and follow-on study (24 h & 48 h))

| | | | | Total | Total | | Free |
|---|---|---|---|---|---|---|---|
| | | | | ADC | Antibody | | Payload |
| Animal ID | | | Time | Result | Result | TADC/TAB | Result |
| # | Treatment | Route | Point | (μg/mL) | (μg/mL) | Ratio | (pg/mL) |
| 8544-Left | ADC | SC | 24 | 236 | 314 | 0.75 | 1,670,000 |
| 8544-Right | ADC + | SC | 24 | 29.2 | 51.4 | 0.57 | 181,000 |
| | rHuPH20 | | | | | | |
| 8545-Left | ADC | SC | 24 | 227 | 255 | 0.89 | 1,910,000 |
| 8545-Right | ADC + | SC | 24 | 20.6 | 42.3 | 0.49 | 172,000 |
| | rHuPH20 | | | | | | |
| 8554-Left | ADC | SC | 24 | 152 | 220 | 0.69 | 1,230,000 |
| 8554-Right | ADC + | SC | 24 | 28.5 | 53 | 0.54 | 265,000 |
| | rHuPH20 | | | | | | |
| 8557-Left | ADC | SC | 48 | 40.2 | 145 | 0.28 | 461,000 |
| 8557-Right | ADC + | SC | 48 | 13.3 | 60.8 | 0.22 | 177,000 |
| | rHuPH20 | | | | | | |
| 8562-Left | ADC | SC | 48 | 12.2 | 68.8 | 0.18 | 156,000 |
| 8562-Right | ADC + | SC | 48 | 3.96 | 24.6 | 0.16 | 84,800 |
| | rHuPH20 | | | | | | |
| 8565-Left | ADC | SC | 48 | 11.9 | 50 | 0.24 | 142,000 |
| 8565-Right | ADC + | SC | 48 | 7.27 | 39.4 | 0.18 | 98,600 |
| | rHuPH20 | | | | | | |
| 8543-Left | ADC | SC | 168 | <0.100 | 7.04 | NA | 1,360 |
| 8546-Left | ADC | SC | 168 | <0.100 | 5.72 | NA | 1,010 |
| 8547-Left | ADC | SC | 168 | 0.112 | 7.85 | 0.01 | 1,680 |
| 8550-Left | ADC + | SC | 168 | <0.100 | 3.37 | NA | 1,050 |
| | rHuPH20 | | | | | | |
| 8551-Left | ADC + | SC | 168 | 0.105 | 7.98 | 0.01 | 2,550 |
| | rHuPH20 | | | | | | |
| 8552-Left | ADC + | SC | 168 | 0.308 | 8.53 | 0.04 | 2,650 |
| | rHuPH20 | | | | | | |

TABLE 66
Concentration of total antibody (TAB) in tissue homogenates of injection site (μg/mg protein ± SD)

| | | | | Mean | SD | CV | ADC + | ADC + | ADC + | Mean ADC + | | |
|---|---|---|---|---|---|---|---|---|---|---|---|---|
| Time Post- | ADC | ADC | ADC | ADC | ADC | ADC | rHuPH20 | rHuPH20 | rHuPH20 | rHuPH20 | SD ADC + | CV ADC + |
| injection (h) | SC | SC | SC | SC | SC | SC | SC | SC | SC | SC | rHuPH20 SC | rHuPH20 SC |
| 24 | 35.40 | 27.43 | 25.40 | 29.41 | 5.29 | 18% | 6.10 | 3.32 | 7.02 | 5.48 | 1.92 | 35% |
| 48 | 16.83 | 6.38 | 5.51 | 9.57 | 6.30 | 66% | 7.63 | 3.01 | 4.72 | 5.12 | 2.34 | 46% |
| 168 | 0.63 | 0.71 | 0.92 | 0.75 | 0.15 | 20% | 0.43 | 1.16 | 1.09 | 0.89 | 0.40 | 45% |
| AUC₍₁₋₇₎ | 69.75 | 34.63 | 31.53 | 45.30 | 21.23 | 47% | 27.02 | 13.59 | 20.38 | 20.33 | 6.72 | 33% |
| (μg*day/mL) | | | | | | | | | | | | |
| Cₘₐₓ | 35.40 | 27.43 | 25.40 | 29.41 | 5.29 | 18% | 7.63 | 3.32 | 7.02 | 5.99 | 2.33 | 39% |
| (μg/mL) | | | | | | | | | | | | |

TABLE 67
Concentration of total ADC (TADC) in tissue homogenates of injection site (μg/mg protein ± SD)

| | | | | Mean | SD | CV | ADC + | ADC + | ADC + | Mean ADC + | SD ADC + | CV ADC + |
|---|---|---|---|---|---|---|---|---|---|---|---|---|
| Time Post- | ADC | ADC | ADC | ADC | ADC | ADC | rHuPH20 | rHuPH20 | rHuPH20 | rHuPH20 | rHuPH20 | rHuPH20 |
| injection (h) | SC | SC | SC | SC | SC | SC | SC | SC | SC | SC | SC | SC |
| 24 | 26.61 | 24.42 | 17.55 | 22.86 | 4.73 | 21% | 3.46 | 1.62 | 3.78 | 2.95 | 1.17 | 39% |
| 48 | 4.67 | 1.13 | 1.31 | 2.37 | 1.99 | 84% | 1.67 | 0.48 | 0.87 | 1.01 | 0.60 | 60% |
| 168 | 0.00 | 0.00 | 0.01 | 0.00 | 0.01 | 173% | 0.00 | 0.02 | 0.04 | 0.02 | 0.02 | 109% |
| AUC₍₁₋₇₎ | 27.30 | 15.61 | 12.74 | 18.55 | 7.71 | 42% | 6.74 | 2.30 | 4.60 | 4.55 | 2.22 | 49% |
| (μg*day/mL) | | | | | | | | | | | | |
| Cₘₐₓ | 26.61 | 24.42 | 17.55 | 22.86 | 4.73 | 21% | 3.46 | 1.62 | 3.78 | 2.95 | 1.17 | 39% |
| (μg/mL) | | | | | | | | | | | | |

TABLE 68
Concentration of free payload in tissue homogenates of injection site (μg/mg protein ± SD)

| | | | | | | | | | | Mean | SD | |
|---|---|---|---|---|---|---|---|---|---|---|---|---|
| | | | | Mean | | | ADC + | ADC + | ADC + | ADC + | ADC + | CV ADC + |
| Time Post-injection | ADC- | ADC- | ADC- | ADC- | SD | CV | rHuPH20 | rHuPH20 | rHuPH20 | rHuPH20 | rHuPH20 | rHuPH20 |
| (h) | SC | SC | SC | SC | SC | SC | SC | SC | SC | SC | SC | SC |
| 24 | 0.19 | 0.21 | 0.14 | 0.18 | 0.03 | 18% | 0.02 | 0.01 | 0.04 | 0.02 | 0.01 | 47% |
| 48 | 0.05 | 0.01 | 0.02 | 0.03 | 0.02 | 80% | 0.02 | 0.01 | 0.01 | 0.01 | 0.01 | 44% |
| 168 | 0.00 | 0.00 | 0.00 | 0.00 | 0.00 | 29% | 0.00 | 0.00 | 0.00 | 0.00 | 0.00 | 46% |
| AUC₍₁₋₇₎ | 0.25 | 0.15 | 0.12 | 0.17 | 0.07 | 42% | 0.08 | 0.04 | 0.05 | 0.06 | 0.02 | 35% |
| (ug*day/mL) | | | | | | | | | | | | |
| Cₘₐₓ (ug/mL) | 0.19 | 0.21 | 0.14 | 0.18 | 0.03 | 18% | 0.02 | 0.01 | 0.04 | 0.02 | 0.01 | 46% |

TABLE 69
TADC/TAB ratios in tissue homogenates (Mean SD)

| | | | | | | | | | | Mean | SD | |
|---|---|---|---|---|---|---|---|---|---|---|---|---|
| Time Post | | | | Mean | SD | | ADC + | ADC + | ADC + | ADC + | ADC + | |
| Injection | ADC | ADC | ADC | ADC | ADC | % CV | rHuPH20 | rHuPH20 | rHuPH20 | rHuPH20 | rHuPH20 | |
| (h) | SC | SC | SC | SC | SC | SC | SC | SC | SC | SC | SC | % CV SC |
| 24 | 0.75 | 0.89 | 0.69 | 0.78 | 0.89 | 114% | 0.57 | 0.49 | 0.54 | 0.53 | 0.04 | 8% |
| 48 | 0.28 | 0.18 | 0.24 | 0.25 | 0.32 | 128% | 0.22 | 0.16 | 0.18 | 0.19 | 0.03 | 16% |
| 168 | 0.00 | 0.00 | 0.01 | 0.00 | 0.04 | NA | 0.00 | 0.02 | 0.04 | 0.02 | 0.02 | 100% |

Table 70 supports these concepts via perspective from the skin. The AUC₁₋₇ and Cmax for all analytes are substantially lower for ADC+rHuPH20 relative to ADC alone when delivered via SC administration.

TABLE 70
Relative difference in skin pharmacokinetic metrics for ADC analytes
after SC administration (ADC + rHuPH20 versus ADC alone)

| | Metric | (ADC + rHuPH20)/ADC |
|---|---|---|
| AUC₁₋₇ | Total Antibody (TAB) | 45% |
| | Total ADC (TADC) | 25% |
| | Free Payload (Payload) | 33% |
| Cmax | Total Antibody (TAB) | 20% |
| | Total ADC (TADC) | 13% |
| | Free Payload (Payload) | 13% |

Table 71 further demonstrates that, at the 24 and 48 hour time points, the addition of rHuPH20 greatly reduces the concentration of all analytes in the skin relative to SC alone.

TABLE 71
Comparison of mean skin concentration (μg/mg protein)
for ADC analytes comparing routes of administration

| Time Post- | | | | (ADC + |
|---|---|---|---|---|
| injection | | | ADC + | rHuPH20)/ |
| (h) | Metric | ADC | rHuPH20 | ADC |
| 24 | Total Antibody (TAB) | 29.41 | 5.48 | 19% |
| | Total ADC (TADC) | 22.86 | 2.95 | 13% |
| | Free Payload | 0.18 | 0.02 | 13% |
| 48 | Total Antibody (TAB) | 9.57 | 5.12 | 54% |
| | Total ADC (TADC) | 2.37 | 1.01 | 43% |
| | Free Payload | 0.03 | 0.01 | 53% |
| 168 | Total Antibody (TAB) | 0.75 | 0.89 | 118% |
| | Total ADC (TADC) | 0.00 | 0.02 | 413% |
| | Free Payload | 0.00 | 0.00 | 0% |

### Conclusions

rHuPH20 enables faster absorption and reduced residence time, which can result in improved local safety profile via less payload existing at the SC site of administration. Additionally, rHuPH20 improves SC bioavailability, resulting in higher concentrations of intact, i.e., payload-bound ADC, in systemic circulation.

The PK improvements afforded by rHuPH20 (greater dispersion, higher bioavailability and reduced residence time), combined with the ability to accommodate greater doses and volumes, enable potential improvements to safety and/or efficacy of this ADC (Trodelvy®).

### Example 11: Trodelvy Follow-on Study: Assessment of Local Injection Site Retention of an ADC after Subcutaneous Administration with and without rHuPH20

### Summary

The objective of this study was to determine the local injection site tissue retention of an antibody-drug conjugate (ADC), Todelvy™, after subcutaneous (SC) administration either alone or co-mixed with recombinant human hyaluronidase PH20 (rHuPH20). Each animal received two SC injections on the lower abdomen; the first injection of ADC alone followed by a second SC injection of the ADC co-mixed with rHuPH20 at a concentration of 2000 U/mL. Dose volume was 5 mL (5 mg ADC) for each injection.

The Yucatan miniature pig model was selected for this study due to the high degree of similarity of the subcutaneous (SC) space to that of humans. Previous studies using a mini-pig model have demonstrated the translatability of the model for use in pre-clinical studies. To determine the amount of ADC that remains present after SC injection, tissue samples of each injection site were obtained from each animal post-mortem. Timepoints for tissue collection were 24 and 48 hours post-injection. The tissue samples were preserved for later bioanalytical analysis. Bioanalysis included assays on skin tissue homogenates for intact ADC, total antibody (TAB) and free payload (SN38).

### Introduction and Objectives

Rapid subcutaneous administration of large volumes of antibodies has been shown to be feasible with recombinant human hyaluronidase PH20 (rHuPH20). rHuPH20 facilitates SC administration of fluids and drugs by transiently and locally depolymerizing hyaluronan (HA) in the extracellular matrix (ECM) thereby reducing tissue backpressure in the SC space permitting rapid, large volume administration of fluid.

A novel class of antibody-based therapeutics has been developed and recently approved by the FDA for the management or treatment of cancer. These therapeutics combine monoclonal antibodies specific to surface antigens present on particular tumor cells with highly potent anti-cancer agents linked via a chemical linker to form an antibody drug conjugate (ADC). It is important to assess the distribution from the injection site following SC administration, and to determine if rHuPH20 is able to enhance the disperse of the ADC more effectively away from the site, potentially reducing local skin toxicity and improving absorption. The swine model has been chosen for this assessment due to the high similarity of the skin and subcutaneous space between humans and swine.

In this study each animal received two SC injections-one injection of the ADC alone followed by a second SC injection of the ADC co-mixed with rHuPH20. Each injection was five mL and was delivered using a pre-filled 10-cc syringe connected to a 20G×1-inch needle. Two cohorts were evaluated in this study. The first was euthanized 24 hours post-injection and biopsies were collected from each site for analysis post-mortem. The second cohort was euthanized at 48 hours post-injection and biopsies were similarly taken from the injection sites.

### Experimental Design and Methods

### Test and Control Articles

See Example 10 for a description of the ADC, rHuPH20, and 0.9% sodium chloride for injection used in this follow-on study.

### Formulation

All solutions were prepared in a biosafety cabinet using standard laboratory safety protocol. The antibody drug conjugate (sacituzumab govitecan) was rehydrated by the addition of 20 mL of 0.9% Sodium Chloride to the lyophilized product to yield a test solution at 10 mg/mL. After rehydration of the ADC, 6 syringes were prepared containing 5.05 mL of the antibody solution alone (control syringes). After preparation of the control syringes, the remaining antibody solution had rHuPH20 (10 mg/mL) added to a final concentration of 2000 U/mL. After preparation of the co-mix of ADC and rHuPH20, 5.2 mL of the solution was drawn into 6 additional syringes. After preparation of all syringes, they were stored in a sealed container and stored at 2-8° C. or on ice until removed and brought to room temperature prior to use for a minimum of 30 minutes.

### Test Methods

Prior to study start, the enzyme activity of the test solution was measured using an in vitro activity assay to confirm the concentration of rHuPH20 in the co-mix drug solution. The results of the pre-study testing is provided in Table 72 below.

TABLE 72
Summary of pre-study activity testing

| Test Solution | Activity (U/mL ± SD) |
|---|---|
| ADC + rHuPH20 | 2113 ± 6 |

### Animal Description

Species: Pig (Sus scrofa domestica)
Strain: Yucatan Miniature
Sex: Female
Age: ˜3-6 months
Weight: ˜10-12 kg
Quantity: 6 animals
Source: Premier BioSource (Ramona, CA)

### Summary of Bodyweights and Dates of Birth.

The bodyweights for each animal on the day of the procedure, as well as the date of birth for each animal are provided in Table 73.

TABLE 73
Summary of bodyweights and dates of birth

| Cohort # | Animal ID# | Bodyweight (kg) | Date of Birth |
|---|---|---|---|
| 1 | 8544 | 17.0 | Jan. 29, 2025 |
| 1 | 8545 | 15.1 | Feb. 3, 2025 |
| 1 | 8554 | 15.1 | Feb. 3, 2025 |
| 2 | 8557 | 18.0 | Feb. 6, 2025 |
| 2 | 8562 | 17.1 | Feb. 6, 2025 |
| 2 | 8565 | 17.4 | Feb. 6, 2025 |

### Husbandry.

See Example 10 for a description of husbandry methods used in this follow-on study.

### Test Materials

The following test materials in Table 74 were used in the study.

TABLE 74
Test Materials

| Test Material | Supplier | Catalog # |
|---|---|---|
| 10-cc syringe | Becton Dickinson | 302995 |
| 23 G × ¾-inch × 3½-inch infusion set | Terumo | SV*23BLS |
| Syringe caps | Becton Dickinson | 305819 |
| Digital point and shoot camera | Canon | S120 |
| Accuderm 12 mm biopsy punch | Fisher Scientific | NC9253254 |
| Disposable scissors | Fisher Scientific | 50-109-4247 |
| Disposable forceps | Fisher Scientific | NC0266178 |
| O.C.T. freezing compound | Fisher Scientific | 23-730-571 |
| Epredid ® Embedding Molds | Fisher Scientific | 22-19 |
| 22 × 22 × 20 mm | | |
| Cryogenic vials, 1.2 mL | Fisher Scientific | 10-500-25 |

### Bioanalytical Methods

### Analysis of Total Antibody and Total ADC in Skin.

See Example 10 for a description of the TAB and TADC analysis methods used in this follow-on study.

### Analysis of Free Payload (Unconjugated SN-38) in Skin Background.

See Example 10 for a description of the free payload analysis methods used in this follow-on study.

### Experimental Design

This was an exploratory study to assess the local retention time of an antibody-drug conjugate (sacituzumab govitecan) with and without rHuPH20. In this initial study, six animals were used. Each animal received (2) subcutaneous injections—the first injection was the ADC alone followed by the second injection of the co-mix of ADC+rHuPH20. Two cohorts were included in this study. Cohort 1 was euthanized 24 hours after treatment, while Cohort 2 was euthanized 48 hours after exposure to the two test solutions. A summary of the treatment groups is shown in Table 75.

TABLE 75
Description of treatments

| | | | Dose of | | | |
|---|---|---|---|---|---|---|
| | | | ADC, | | | |
| | | | rHuPH20 | # of Animals | | |
| | | Dose Volume | (mg, U) per | (2 injections / | [rHuPH20] | Exposure |
| Cohort # | Test Solution | (mL) per site | site | animal) | (U/mL) | Time (h) |
| 1 | sacituzumab | 5.0 | 50 mg | 3 | 0 | 24 |
| | govitecan | | 0 U | | | |
| | sacituzumab | 5.0 | 50 mg | 3 | 2000 | 24 |
| | govitecan + | | 10,000 U | | | |
| | rHuPH20 | | | | | |
| 2 | sacituzumab | 5.0 | 50 mg | 3 | 0 | 48 |
| | govitecan | | 0 U | | | |
| | sacituzumab | 5.0 | 50 mg | 3 | 2000 | 48 |
| | govitecan + | | 10,000 U | | | |
| | rHuPH20 | | | | | |

After euthanasia, full thickness biopsies of the skin at each injection site were obtained using a 12 mm punch. The tissue was cut in half and placed into OCT cryogenic molds with the inner cut surface facing the bottom of the mold. The sample was then placed in a dry ice and 70% ethanol bath for freezing. Once frozen the sample was collected and stored on dry ice until transported for long-term storage at ≤70° C. The weight of the other half of the biopsy was recorded and the tissue placed into a cryogenic tube whereupon it was frozen on dry ice until transfer to long-term storage at ≤70° C.

### Study Design

Prior to the start of study, the animals were assessed for general health, and the body weight collected on the day of the study. All anesthesia (isoflurane) was administered and monitored by the staff. After anesthetization, the animal was placed in dorsal recumbence (no wedge) on a heated surgical table and was maintained under isoflurane gas for the entire duration of the procedure. Following anesthetization, the abdominal region was shaved and cleaned with Nolvasan followed by wiping the injection site with gauze containing 70% isopropanol and wiped dry with sterile gauze. Injection sites were located on the left and right abdominal regions, located 3 mm towards the midline of the animal from the middle of the inguinal fold and approximately 5 mm cranially. The infusion set was primed to the needle tip with 0.2 mL of the test solution. For needle insertion a standard pinch method was used. After needle insertion the injection proceeded. The time was recorded for each injection. At 24h post-injection, the animals for Cohort 1 were euthanized (3 animals) and punch biopsies obtained as described above. After obtaining the punch biopsies, the animal carcass was removed and disposed of as biohazardous waste. Animals for Cohort 2 were euthanized at 48 hours post-injection and samples obtained as described above.

Samples were analyzed histologically to detect and quantitate presence of the ADC at the injection site tissue. Furthermore, tissue samples were analyzed for possible detection of the association of the antibody and payload.

### Results

### Bioanalytical Analysis of Post-Injection Tissue Samples

Tissue Samples from Injection Site—Follow-on Study—D1 and D2 Skin Samples.

See Example 10 for a description of the tissue sample methods used in this follow-on study. See also Tables 66-69, 70, and 71 as well as FIGS. 59-62 of Example 10 for the data associated with this follow-on study.

Tables 76 and 77 depict the ratios of analytes and PK metrics in the skin tissue homogenate for the different administrations.

TABLE 76
Comparison of mean AUC₁₋₇ and Cmax for ADC
analytes comparing routes of administration

| | | | | TADC/Free |
|---|---|---|---|---|
| | | | TADC/TAB | Payload |
| AUC₍₁₋₇₎ | ADC | IV | NA | NA |
| | ADC | SC | 41% | 10704% |
| | ADC + rHuPH20 | SC | 22% | 8006% |
| Cmax | ADC | IV | NA | NA |
| | ADC | SC | 78% | 12800% |
| | ADC + rHuPH20 | SC | 49% | 12501% |

TABLE 77
Comparison of mean AUC₁₋₇ and Cmax for TAB, TADC and Free
Payload in SC skin samples: ADC + rHuPH20 versus ADC

| | | ADC + rHuPH20 (SC)/ |
|---|---|---|
| | | ADC (SC) |
| AUC₁₋₇ | Total Antibody (TAB) | 45% |
| | Total ADC (TADC) | 25% |
| | Free Payload | 33% |
| Cmax | Total Antibody (TAB) | 20% |
| | Total ADC (TADC) | 13% |
| | Free Payload | 13% |

### Example 12: Pharmacokinetics of an ADC (Trastuzumab Deruxtecan) after Subcutaneous Administration with and without rHuPH20

### Summary

The objective of this study was to investigate the PK of a monoclonal ADC, fam-trastuzumab deruxtecan-nxki, following SC administration alone or co-administered with rHuPH20. This study determined the impact of rHuPH20 on the PK of this antibody, and the impact on the absorption and bioavailability of the antibody following SC injection.

Minipigs were used in this study due to the similarity of the SC skin architecture to humans. Three cohorts were included in this study. Cohort 1 received the ADC administered intravenously. Cohort 2 received the ADC alone delivered via SC injection, while Cohort 3 received the ADC co-mixed with rHuPH20 (2,000 U/mL) also delivered via SC injection. All injections were 5 mL. Blood collection timepoints were pre-treatment, 6 h, 24 h, 48 h, 72h, 96h, and 168h post-injection. Blood was drawn via jugular vein stick and collected into serum separator tubes (SSTs), centrifuged, and serum stored at ≤−70° C. until bioanalytical analysis.

The results indicate that rHuPH20 enables faster absorption and reduced SC tissue residence time of the ADC, which can result in improved local safety profile via less payload existing at the SC site of administration. Additionally, rHuPH20 improves SC bioavailability, resulting in higher concentrations of intact, i.e., payload-bound ADC, in systemic circulation.

### Introduction and Objectives

Subcutaneous administration of large volumes of antibodies has been shown to be feasible when the antibody solution is co-formulated with recombinant human hyaluronidase PH20 (rHuPH20). rHuPH20 has been shown to facilitate SC administration of fluids and drugs by transiently and locally depolymerizing hyaluronan (HA) in the extracellular matrix thereby reducing tissue backpressure in the SC space permitting rapid, large volume administration of fluid. Using this technology, subcutaneous administration of large volumes of antibody has become possible and is replacing intravenous administration as a treatment paradigm. However, new treatment paradigms using monoclonal antibodies are focusing on the preparation of higher concentration solutions that may provide higher systemic exposure for longer durations, potentially supporting longer clinical dosing intervals.

The minipig model has been selected due to the high degree of similarity of the subcutaneous space to that of humans. Previous studies using a minipig model have demonstrated the translatability of the model for use in pre-clinical studies. This study demonstrated that recombinant human hyaluronidase (rHuPH20) can improve the PK and absorption of an ADC by enhancing the local dispersion.

### Experimental Design and Methods

### Test and Control Articles

### 1) Fam-Trastuzumab Deruxtecan-Nxki (Enhertu®)

Catalog number: 04150172532192
Lot numbers: #1) 28477147910513602152417730747270
   #2) 2517487499669367
   #3) 2232984620808796
Expiration date: April 2028
Concentration: 20 mg/mL after reconstitution in sterile water for injection
Storage Conditions: 2-8° C.
Handling Conditions: Standard laboratory precautions
Supplier: Halozyme Therapeutics, Inc.
   2) Recombinant Human Hyaluronidase PH20 (rHuPH20)
Description: Clear colorless liquid
Lot number: 1-FIN-3928
Concentration: 1,039,763 U/mL; 10 mg/mL
Formulation: 10 mM Histidine, 130 mM sodium chloride, pH 6.5
Storage Conditions: ≤70° C.
Handling Conditions: Standard laboratory precautions
Supplier: Halozyme, Inc.

### 3) Sterile Water for Injection, USP

Description: Clear colorless liquid
Catalog number: NDC 0409-4887-17
Lot number: LT4430
Expiration date: 2027 Aug. 31
Storage Conditions: Room temperature
Handling Conditions: Standard laboratory precautions
Supplier: Hospira

### Formulation

All solutions were prepared in a Biosafety cabinet using standard laboratory safety protocol. Two test solutions were used in this study: ADC (fam-trastuzumab deruxtecan-nxki) alone and the ADC co-mixed with rHuPH20. Three vials of the antibody drug conjugate were rehydrated by the addition of 5.0 mL of sterile water for injection into the lyophilized product (15 mL total; 20 mg/mL). After rehydration the contents of the three vials were combined to prepare the syringes.

### Syringe Preparation.

The rehydrated ADC was used to prepare (4) syringes that each contained 1.3 mL of the test solution (1.25 mL dose volume+0.05 mL needle priming volume). After filling each syringe, it was capped and stored at 2-8° C. until removed prior to use.

The remaining ADC solution was used to prepare the ADC+rHuPH20 test solution. The co-mix of ADC rHuPH0 was prepared by adding 17.3 μL of rHuPH20 to 9 mL of the ADC solution to yield a test solution of the ADC+rHuPH20 at a target concentration of 2000 U/mL. This solution was used to prepare (3) syringes each containing 1.3 mL of the ADC+rHuPH20 test solution. After filling each syringe was capped and stored at 2-8° C.

Syringes were brought to room temperature for at least 30 minutes before use. The syringe cap was removed and replaced with a 25G×1″ needle which was primed immediately before use.

### Test Methods

Prior to study start, enzyme activity of the test solution was measured using an in vitro activity assay to confirm the concentration of rHuPH20 in the co-mix drug solution. The results are shown in Table 78.

TABLE 78
Summary of pre-study activity testing

| Test Solution | Activity (U/mL ± SD) |
|---|---|
| ADC + rHuPH20 | 2192 ± 26 |

### Animal Description

Species: Pig (Sus scrofa domestica)
Strain: Yucatan Miniature
Sex: Female
Age: ˜3-4 months
Weight: ˜14-18 kg
Quantity: 7 animals
Source: Premier BioSource (Ramona, CA)

Summary of Bodyweight and Date of Birth. The bodyweight of each animal on the day of treatment and at the end of study together with the date of birth for each animal is listed in Table 79.

TABLE 79
Summary of bodyweight and date of birth

| Animal ID# | Bodyweight (D 0) | Bodyweight (D 7) | Date of Birth |
|---|---|---|---|
| 8726 | 15.8 | 15.0 | 3 Mar. 2025 |
| 8730 | 17.4 | 16.7 | 4 Mar. 2025 |
| 8732 | 15.4 | 15.0 | 5 Mar. 2025 |
| 8734 | 15.1 | 14.3 | 7 Mar. 2025 |
| 8737 | 14.4 | 14.5 | 6 Mar. 2025 |
| 8739 | 14.7 | 14.3 | 6 Mar. 2025 |
| 8743 | 15.7 | 14.4 | 3 Mar. 2025 |

### Husbandry.

The animals were housed in a steel pen with automatic water provided ad libitum. The animals were fed twice daily (AM and PM) but were kept NPO after midnight on the day of the study to prevent anesthesia complications. The room environment was set to maintain a temperature of ˜19-23° C. and a relative humidity of 40-70%, with a 12-hour light/12-hour dark time cycle. The animals were acclimated to the vivarium for a minimum of 3 days prior to study start.

### Test Materials

The following test materials were used in the study (Table 80):

TABLE 80
Test materials

| Test Material | Supplier | Catalog # | Expiration Date |
|---|---|---|---|
| 10-cc syringe | Becton Dickinson | 302995 | NA |
| Syringe caps | Becton Dickinson | 305819 | NA |
| 25G x 1 inch Precision Glide needle | Becton Dickinson | 305125 | NA |
| 20G x 1 inch thin wall needle | Mckesson | 16-N201 | NA |
| Accuderm 12 mm biopsy punch | Fisher Scientific | NC9253254 | NA |
| 0.9% sodium chloride for injection | Mckesson | 239940 | 2026 Jul. 31 |
| Vacutainer Blood Collection Tubes | Becton Dickinson | 367977 | Mar. 31 2027 |
| (Serum Separator Tubes) | | | |
| Cryogenic Vials | Fischer Scientific | 10-500-26 | NA |
| O.C.T. freezing compound | Fisher Scientific | 23-730-571 | NA |
| Epredid ® Embedding Molds | Fisher Scientific | 22-19 | NA |

### Bioanalytical Methods

Serum concentrations of total ADC, total antibody and Free Payload (unconjugated deruxtecan) were determined in three separate assays using HPLC with MS/MS detection. Method qualification in pig serum and study sample analysis results are reported in Tables 81 and 82.

TABLE 81
Quantitation of total antibody, total ADC and free
payload from Enhertu in minipig serum samples

| | | | Total | Total | | Free |
|---|---|---|---|---|---|---|
| | | | ADC | Antibody | | Payload |
| Animal | | Time | Result | Result | TADC/TAB | Result |
| ID # | Treatment | Point | (μg/mL) | (μg/mL) | Ratio | (pg/mL) |
| 8726 | ADC IV | Pre | <0.100 | <0.100 | NA | <20.0 |
| | | 6 | 21.4 | 23.3 | 0.92 | 10600 |
| | | 24 | 12.8 | 14.7 | 0.87 | 6110 |
| | | 48 | 7.28 | 9.06 | 0.80 | 2900 |
| | | 72 | 5.04 | 5.75 | 0.88 | 9470 |
| | | 96 | 3.91 | 5.06 | 0.77 | 6690 |
| | | 168 | 1.70 | 1.71 | 0.99 | 2110 |
| 8730 | ADC SC | Pre | <0.100 | <0.100 | NA | <20.0 |
| | | 6 | 1.82 | 1.81 | 1.01 | 3220 |
| | | 24 | 4.70 | 3.04 | 1.55 | 8310 |
| | | 48 | 3.54 | 4.38 | 0.81 | 5960 |
| | | 72 | 3.02 | 3.96 | 0.76 | 5400 |
| | | 96 | 2.76 | 3.71 | 0.74 | 3440 |
| | | 168 | 1.83 | 1.52 | 1.20 | 2940 |
| 8732 | ADC SC | Pre | <0.100 | <0.100 | NA | <20.0 |
| | | 6 | 4.20 | 3.72 | 1.13 | 5970 |
| | | 24 | 8.87 | 10.5 | 0.84 | 3820 |
| | | 48 | 5.98 | 7.27 | 0.82 | 3410 |
| | | 72 | 5.40 | 7.49 | 0.72 | 6740 |
| | | 96 | 4.93 | 6.74 | 0.73 | 8180 |
| | | 168 | 2.44 | 2.74 | 0.89 | 3500 |
| 8734 | ADC SC | Pre | <0.100 | <0.100 | NA | <20.0 |
| | | 6 | 6.23 | 6.07 | 1.03 | 9650 |
| | | 24 | 8.25 | 6.91 | 1.19 | 3930 |
| | | 48 | 5.99 | 7.92 | 0.76 | 3050 |
| | | 72 | 4.64 | 4.97 | 0.93 | 7990 |
| | | 96 | 3.53 | 4.31 | 0.82 | 4890 |
| | | 168 | 1.86 | 1.88 | 0.99 | 2390 |
| 8737 | ADC + | Pre | <0.100 | <0.100 | NA | <20.0 |
| | rHuPH20 | 6 | 3.70 | 3.42 | 1.08 | 5110 |
| | SC | 24 | 9.77 | 13.6 | 0.72 | 5020 |
| | | 48 | 7.09 | 7.45 | 0.95 | 3530 |
| | | 72 | 6.25 | 8.28 | 0.75 | 9490 |
| | | 96 | 5.25 | 5.96 | 0.88 | 6530 |
| | | 168 | 2.79 | 2.76 | 1.01 | 3990 |
| 8739 | ADC + | Pre | <0.100 | <0.100 | NA | <20.0 |
| | rHuPH20 | 6 | 3.97 | 5.23 | 0.76 | 5460 |
| | SC | 24 | 10.8 | 11.7 | 0.92 | 6140 |
| | | 48 | 8.25 | 9.28 | 0.89 | 3810 |
| | | 72 | 7.03 | 8.70 | 0.81 | 2880 |
| | | 96 | 5.68 | 7.41 | 0.77 | 2780 |
| | | 168 | 2.68 | 2.77 | 0.97 | 3850 |
| 8743 | ADC + | Pre | <0.100 | <0.100 | NA | <20.0 |
| | rHuPH20 | 6 | 7.12 | 7.53 | 0.95 | 7320 |
| | SC | 24 | 9.00 | 10.9 | 0.83 | 4710 |
| | | 48 | 6.54 | 6.92 | 0.95 | 3540 |
| | | 72 | 5.22 | 4.91 | 1.06 | 8890 |
| | | 96 | 3.70 | 4.55 | 0.81 | 7440 |
| | | 168 | 1.77 | 1.60 | 1.11 | 2250 |

TABLE 82
Quantitation of total antibody, total ADC and free payload from Enhertu in minipig
skin samples (present study (168 h) and follow-on study (24 h & 48 h))

| | | | | Total | Total | | Free |
|---|---|---|---|---|---|---|---|
| | | | | ADC | Antibody | | Payload |
| Animal ID | | | Time | Result | Result | TADC/TAB | Result |
| # | Treatment | Route | Point | (μg/mL) | (μg/mL) | Ratio | (pg/mL) |
| 8731-Left | ADC | SC | 24 | 207 | 237 | 0.87 | 138000 |
| 8731-Right | ADC + rHuPH20 | SC | 24 | 22.4 | 25.8 | 0.87 | 15700 |
| 8733-Left | ADC | SC | 24 | 139 | 143 | 0.97 | 124000 |
| 8733-Right | ADC + rHuPH20 | SC | 24 | 73.8 | 79.1 | 0.93 | 76200 |
| 8736-Left | ADC | SC | 24 | 73.0 | 97.8 | 0.75 | 67800 |
| 8736-Right | ADC + rHuPH20 | SC | 24 | 131 | 159 | 0.82 | 85500 |
| 8738-Left | ADC | SC | 48 | 38.9 | 50.2 | 0.77 | 21700 |
| 8738-Right | ADC + rHuPH20 | SC | 48 | 5.78 | 6.27 | 0.92 | 3670 |
| 8740-Left | ADC | SC | 48 | 44.7 | 51.7 | 0.86 | 29500 |
| 8740-Right | ADC + rHuPH20 | SC | 48 | 32.6 | 64.1 | 0.51 | 18800 |
| 8742-Left | ADC | SC | 48 | 55.7 | 58.1 | 0.96 | 33800 |
| 8742-Right | ADC + rHuPH20 | SC | 48 | 48.8 | 75.3 | 0.65 | 25800 |
| 8742-Left | ADC | SC | 168 | 4.45 | 4.43 | 1.00 | 3320 |
| 8742-Left | ADC | SC | 168 | 3.26 | 4.86 | 0.67 | 2240 |
| 8547-Left | ADC | SC | 168 | 4.04 | 5.80 | 0.70 | 3520 |
| 8550-Left | ADC + rHuPH20 | SC | 168 | 2.80 | 3.26 | 0.86 | 1850 |
| 8551-Left | ADC + rHuPH20 | SC | 168 | 1.75 | 2.24 | 0.78 | 1310 |
| 8552-Left | ADC + rHuPH20 | SC | 168 | 1.50 | 2.44 | 0.61 | 850 |

### Analysis of Total Antibody and Total ADC in Serum and Skin.

A 20.0-μL sample aliquot was diluted 10-fold. A portion of the diluted sample was taken and processed using an immunoaffinity approach. SMART Digest IA magnetic beads coated with anti-human Fc antibody were used to enrich Enhertu from matrix. The bound proteins were subjected to “on-bead” proteolysis with trypsin. After trypsin digestion, the samples were split into two portions. One portion used the characteristic peptide fragments produced from the trypsin digestion to quantitate total antibody. The second portion was further digested using papain to hydrolyze Dxd from the antibody to quantitate total ADC.

Calibration curves and quality controls were prepared by fortifying Enhertu in blank porcine serum (surrogate matrix) at concentrations ranging from 0.100 to 100 μg/mL. VVSV was used for the quantitation of total antibody concentrations, DSTY was used for the quantitation of intact antibody (troubleshooting purposes only), and Dxd was used for quantitating Total ADC concentrations.

Porcine serum was used as surrogate matrix to prepare calibration standards and quality controls for skin sample quantitation. Untreated blank porcine skin was provided for preparation of monitoring quality controls. The porcine skin was homogenized at an approximate ratio of 0.5 mL of buffer for every 100 mg of skin. The homogenized porcine skin (primary matrix) was used to prepare a low (0.600 μg/mL) and high (76.0 μg/mL) monitoring quality controls. The monitoring controls were analyzed during the method performance evaluation. The following were selected for monitoring in the assay (Table 83).

TABLE 83
Amino acid sequences used for bioanalytical analysis of samples

| Name | Amino Acid Sequence | Purpose |
|---|---|---|
| VVSV | VVSVLTVLHQDWLNGK | Quantitation of Total Antibody (Fc, |
| | (SEQ ID NO: 18) | Heavy Chain) |
| DSTY | DSTYSLSSTLTLSK | Quantitation of Intact Antibody (Kappa |
| | (SEQ ID NO: 19) | Light Chain) |
| VVSV-IS | VVSVLTVLHQDWLNGK* | Internal Standard |
| | (SEQ ID NO: 20) | |
| DSTY-IS | DSTYSLSSTLTLSK* | Internal Standard |
| | (SEQ ID NO: 21) | |
| ADC | Dxd | Quantitation of Total ADC (Dxd) |

Analysis of Free Payload (deruxtecan) in Serum and Skin.

A nominal free Dxd, concentration range of 10.0 to 5000 μg/mL was chosen to quantitate samples. This assay requires a 50.0 μL porcine serum and skin aliquot. Samples were kept frozen at −80° C. prior to analysis.

A matrix aliquot is fortified with internal standard. Analytes are isolated through acidified protein precipitation. The eluate is evaporated under a nitrogen stream, and the remaining residue is reconstituted. Final extracts are analyzed via HPLC MS/MS detection using positive ion electrospray.

Porcine serum was used as surrogate matrix to prepare calibration standards and quality controls for the quantitation of skin samples. Untreated blank porcine skin was provided to be able to homogenize and prepare monitoring quality controls. The porcine skin was homogenized at an approximate ratio of 0.5 mL of buffer for every 100 mg of skin. The homogenized porcine skin (primary matrix) was used prepare a low (55.0 pg/mL) and high (7500 pg/mL) monitoring quality controls. These were analyzed during the method performance evaluation. The reference and internal standards for the Dxd analysis are found in Table 84.

TABLE 84
Reference and internal standards for the Dxd analysis

| Name | Source | Lot | Material |
|---|---|---|---|
| Dxd^{a} | MedChemExpress | 101442 | MA00711345 |
| Dxd-d5^{b} | MedChemExpress | 116882 | MA00937426 |

^{a}Dxd was purchased from MedChemExpress as a powder with a CF of 0.98 and stored at 2-8° C.
^{b}Dxd-d5 was purchased from MedChemExpress as a powder (CF = IS) and stored at 2-8° C.

### Experimental Design

This study measured the pharmacokinetics (PK) of subcutaneous administration of fam-trastuzumab deruxtecan-nxki alone or co-mixed with rHuPH20 and compared it to the IV PK of fam-trastuzumab deruxtecan-nxki. Each animal received a single 5 mL injection of the ADC or the ADC+rHuPH20. Cohort received the ADC administered IV, while cohort 2 and cohort 3 received the ADC and ADC+rHuPH20 test solutions injected SC, respectively. The treatment groups are shown in Table 85.

TABLE 85
Description of treatments

| | | | | | Injection | | Blood |
|---|---|---|---|---|---|---|---|
| Cohort | | | | Dose | Volume | rHuPH20 | Sampling |
| # | Test Solution | N | Route | (mg) | (mL) | (Units) | Times (h/d) |
| 1 | fam-trastuzumab | 1 | IV | 25 | 1.25 | 0 | Pre, 6 h, 24 h, |
| | deruxtecan-nxki | | | | | | 48 h, 72 h, 96 h, |
| | | | | | | | 168 h |
| 2 | fam-trastuzumab | 3 | SC | 25 | 1.25 | 0 | Pre, 6 h, 24 h, |
| | deruxtecan-nxki | | | | | | 48 h, 72 h, 96 h, |
| | | | | | | | 168 h |
| 3 | fam-trastuzumab | 3 | SC | 25 | 1.25 | 2,000 | Pre, 6 h, 24 h, |
| | deruxtecan-nxki + | | | | | | 48 h, 72 h, 96 h, |
| | rHuPH20 | | | | | | 168 h |

Animals were anesthetized for all blood collection timepoints. On the day of the procedure the animals were anesthetized twice (once for the initial procedure and a second time for the 6h blood collection timepoint). Staff monitored animals to mitigate any possible hypoglycemia that could occur during the interval between the treatment and the 6h timepoint. A pre-treatment blood sample (Pre) was obtained from each animal prior to the initial injection. The animal had approximately a 3 mL blood sample collected at Predose, 6 h, 24 h, 48 h, 72h, 96h, and 168h via jugular vein stick which was transferred to staff and processed for serum. After the final blood collection timepoint, the animals were humanely euthanized. Punch biopsies of SC injection sites were collected for bioanalytical and histopathological evaluation.

The tissue samples were analyzed for TADC, TAB, and Free Payload and this data was generated to complement the tissue data from a follow-on study (Example 13) which had analogous dosing regimens but had tissue samples taken at 24 h and 48h. The results from the follow-on study are presented in this report in conjunction with the data from tissue samples from the present study (D1, D2 and D7). Details and data specific to the follow-on study are also presented in Example 13.

### Study Design

Prior to the start of study, the animal was assessed for general health, its body weight recorded, and a pre-treatment baseline blood sample obtained. Additionally, blood collections were obtained from each animal at 6h post injection, and at 24 h, 48 h, 72h, 96h, and 168h. All blood samples were collected into serum separating tubes (SSTs), allowed to clot at room temperature for a minimum of 30 minutes, then centrifuged in a swinging bucket centrifuge (1200×g; 10 minutes; 20-23° C.). After centrifugation, the serum was aliquoted into two cryogenic vials (primary and backup samples) and stored in a freezer set to maintain ≤−70° C. until analysis. After euthanasia, 12 mm punch biopsies were collected of the SC injection sites. The tissue was divided into two parts (sectioned vertically) with one-half placed in a histological cassette then frozen in OCT. The other half was placed into cryovials, the weight recorded and then frozen on dry ice for bioanalytical evaluation.

On the day of the procedure animals were anesthetized and placed on a heated surgical table and maintained under isoflurane gas for the entire duration of the procedure. An intravenous (IV) catheter was placed into the ear of the animal in cohort 1 to facilitate IV dosing. For cohort 2 and cohort 3, the test solution was delivered to the SC space located on the lower left abdomen, mid-flank, approximately 3 cm toward the midline of the animal and 5 cm cranially. Animals in cohort 2 received a single SC injection of trastuzumab deruxtecan, and animals in cohort 3 received a single SC injection trastuzumab deruxtecan co-mixed with rHuPH20 at a concentration of 2000 U/mL.

### Results

### Bioanalytical and Pharmacokinetic Analysis—Serum

### Nominal and Actual Blood Collection Time.

Following dosing, blood was collected at approximately 6 h, 24 h, 48 h, 72h, 96h, and 168h. The time that each blood collection occurred was recorded and the nominal versus the actual blood collection timepoints are shown in Table 86 through Table 91.

TABLE 86
Nominal blood collection times and actual blood
collection times - 6 hours (360 minutes)

| | | Nominal | Actual | | |
|---|---|---|---|---|---|
| | | Blood | Blood | Time | Time |
| Animal | Injection | Collection | Collection | Deviation | Deviation |
| ID# | Time | Time - 6 h | Time - 6 h | (min) | (%) |
| 8726 | 8:50 | 14:50 | 14:51 | 1 | 0.28% |
| 8730 | 9:00 | 15:00 | 15:02 | 2 | 0.56% |
| 8732 | 9:10 | 15:10 | 15:12 | 2 | 0.56% |
| 8734 | 9:20 | 15:20 | 15:21 | 1 | 0.28% |
| 8737 | 9:30 | 15:30 | 15:29 | 1 | 0.28% |
| 8739 | 9:40 | 15:40 | 15:40 | 0 | 0.00% |
| 8743 | 9:50 | 15:50 | 15:49 | 1 | 0.28% |

TABLE 87
Nominal blood collection times and actual blood
collection times - 24 hours (1440 minutes)

| | | Nominal | Actual | | |
|---|---|---|---|---|---|
| | | Blood | Blood | Time | Time |
| Animal | Injection | Collection | Collection | Deviation | Deviation |
| ID# | Time | Time - 24 h | Time - 24 h | (min) | (%) |
| 8726 | 8:50 | 8:50 | 8:54 | 4 | 0.28% |
| 8730 | 9:00 | 9:00 | 9:03 | 3 | 0.21% |
| 8732 | 9:10 | 9:10 | 9:14 | 4 | 0.28% |
| 8734 | 9:20 | 9:20 | 9:23 | 3 | 0.21% |
| 8737 | 9:30 | 9:30 | 9:32 | 2 | 0.14% |
| 8739 | 9:40 | 9:40 | 9:43 | 3 | 0.21% |
| 8743 | 9:50 | 9:50 | 9:49 | 1 | 0.07% |

TABLE 88
Nominal blood collection times and actual blood
collection times - 48 hours (2880 minutes)

| | | Nominal | Actual | | |
|---|---|---|---|---|---|
| | | Blood | Blood | Time | Time |
| Animal | Injection | Collection | Collection | Deviation | Deviation |
| ID# | Time | Time - 48h | Time - 48h | (min) | (%) |
| 8726 | 8:50 | 8:50 | 8:48 | 2 | 0.07% |
| 8730 | 9:00 | 9:00 | 9:00 | 0 | 0.00% |
| 8732 | 9:10 | 9:10 | 9:14 | 4 | 0.14% |
| 8734 | 9:20 | 9:20 | 9:23 | 3 | 0.10% |
| 8737 | 9:30 | 9:30 | 9:31 | 1 | 0.03% |
| 8739 | 9:40 | 9:40 | 9:42 | 2 | 0.07% |
| 8743 | 9:50 | 9:50 | 9:51 | 1 | 0.03% |

TABLE 89
Nominal Blood Collection Times and Actual Blood
Collection Times - 72 hours (4320 minutes)

| | | Nominal | Actual | | |
|---|---|---|---|---|---|
| | | Blood | Blood | Time | Time |
| Animal | Injection | Collection | Collection | Deviation | Deviation |
| ID# | Time | Time - 72 h | Time - 72 h | (min) | (%) |
| 8726 | 8:50 | 8:50 | 8:45 | 5 | 0.12% |
| 8730 | 9:00 | 9:00 | 9:01 | 1 | 0.02% |
| 8732 | 9:10 | 9:10 | 9:08 | 2 | 0.05% |
| 8734 | 9:20 | 9:20 | 9:18 | 2 | 0.05% |
| 8737 | 9:30 | 9:30 | 9:26 | 4 | 0.09% |
| 8739 | 9:40 | 9:40 | 9:38 | 2 | 0.05% |
| 8743 | 9:50 | 9:50 | 9:49 | 1 | 0.02% |

TABLE 90
Nominal Blood Collection Times and Actual Blood
Collection Times - 96 hours (5760 minutes)

| | | Nominal | Actual | | |
|---|---|---|---|---|---|
| | | Blood | Blood | Time | Time |
| Animal | Injection | Collection | Collection | Deviation | Deviation |
| ID# | Time | Time - 96 h | Time - 96 h | (min) | (%) |
| 8726 | 8:50 | 8:50 | 8:55 | 5 | 0.09% |
| 8730 | 9:00 | 9:00 | 9:07 | 7 | 0.12% |
| 8732 | 9:10 | 9:10 | 9:15 | 5 | 0.09% |
| 8734 | 9:20 | 9:20 | 9:23 | 3 | 0.05% |
| 8737 | 9:30 | 9:30 | 9:33 | 3 | 0.05% |
| 8739 | 9:40 | 9:40 | 9:40 | 0 | 0.00% |
| 8743 | 9:50 | 9:50 | 9:48 | 2 | 0.03% |

TABLE 91
Nominal Blood Collection Times and Actual Blood
Collection Times - 168 hours (10080 minutes)

| | | Nominal | Actual | | |
|---|---|---|---|---|---|
| | | Blood | Blood | Time | Time |
| Animal | Injection | Collection | Collection | Deviation | Deviation |
| ID# | Time | Time - 168 h | Time - 168 h | (min) | (%) |
| 8726 | 8:50 | 8:59 | 8:47 | 3 | 0.03% |
| 8730 | 9:00 | 9:10 | 8:58 | 2 | 0.02% |
| 8732 | 9:10 | 9:20 | 9:05 | 5 | 0.05% |
| 8734 | 9:20 | 9:29 | 9:18 | 2 | 0.02% |
| 8737 | 9:30 | 9:40 | 9:26 | 4 | 0.04% |
| 8739 | 9:40 | 9:50 | 9:36 | 4 | 0.04% |
| 8743 | 9:50 | 10:00 | 9:45 | 5 | 0.05% |

### Measurement of Serum ADC Values—Total Antibody (TAB).

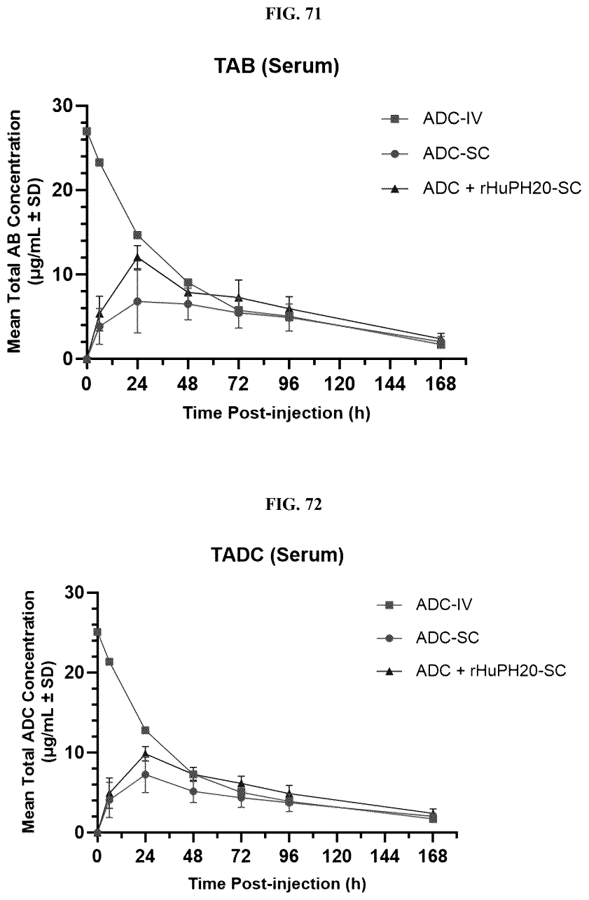

Quantitation of total antibody, total ADC, and free payload (SN-38) from Enhertu in porcine serum and tissue samples was performed via HPLC with MS/MS detection. The serum values for Total Antibody (TAB) for cohort 1 (ADC-IV), cohort 2 (ADC-SC) and cohort 3 (ADC+rHuPH20-SC) at each blood collection timepoint are listed in Table 92 and shown graphically in FIG. 71. Total ADC (TADC) concentration was also measured in the serum at each blood collection time point for cohort 1 (ADC-IV), cohort 2 (ADC-SC) and cohort 3 (ADC+rHuPH20-SC) and is described in Table 93 and shown graphically in FIG. 72.

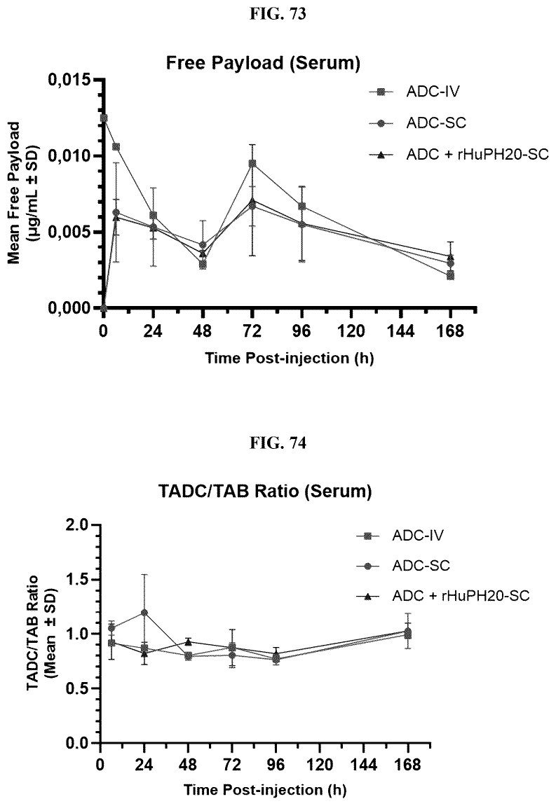

The total free payload (unconjugated SN-38) was measured in serum samples to determine how long it remained conjugated to the antibody component of the ADC. The hydrolyzable linker used to conjugate deruxtecan to trastuzumab is relatively instable, even at neutral pH. As such, the sample preparation method for the LC/MS free payload assay is known to cause some hydrolysis of the linker, leading to an overestimation of the free payload in each sample. The measured quantity of free payload for cohort 1 (ADC-IV), cohort 2 (ADC-SC) and cohort 3 (ADC+rHuPH20-SC) are described in Table 94 and shown graphically in FIG. 73. The concentration of total ADC (TADC) concentration was compared to the total antibody (TAB) concentrations and is expressed as a ratio in Table 95 and shown graphically in FIG. 74.

Tables 92, 93, 94, and 96 describe the differences in AUC₀₋₇, Cmax, and Tmax for cohort 1 (ADC-IV), cohort 2 (ADC-SC), and SC with rHuPH20 (ADC+rHuPH20-SC). In general, rHuPH20 improves the SC absorption and distribution of each analyte, i.e., total antibody (TAB), total ADC (TADC) and free payload (Payload), as demonstrated by the increases in AUC₀₋₇ relative to SC alone. rHuPH20 also increased Cmax for TAB and TADC but not Free Payload. Similarly, rHuPH20 decreased Tmax for TAB and TADC but not Free Payload relative to SC alone. Relative to IV, AUC₀₋₇ is lower for SC and rHuPH20, but with a greater-than-proportional decrease in Cmax. For example, the ratio of (ADC+rHuPH20-SC)/(ADC-IV) AUC₀₋₇ is 79%, while that for Cmax is 39% (see Table 93).

TABLE 92
Individual and mean serum concentrations of total antibody (TAB) - (μg/mL)

| | Animal ID# | | | | | | | | | | | | |
|---|---|---|---|---|---|---|---|---|---|---|---|---|---|
| | | | | | | | | 8737 | 8739 | 8743 | | | |
| | | | | | | | | ADC + | ADC + | ADC + | | | |
| | 8726 | 8730 | 8732 | 8734 | | | | rHuPH20 | rHuPH20 | rHuPH20 | | | |
| | ADC IV | ADC SC | ADC SC | ADC SC | | | | SC | SC | SC | Mean | SD | % CV |
| Time Post- | (Animal | (Animal | (Animal | (Animal | Mean | SD | % CV | (Animal | (Animal | (Animal | SC + | SC + | SC + |
| Injection (h) | 8726) | 8730) | 8732) | 8734) | SC | SC | SC | 8737) | 8739) | 8743) | PH20 | PH20 | PH20 |
| 0 | 27.00^{a} | <0.10^{b} | <0.10^{b} | <0.10^{b} | 0.00^{b} | NA | NA | <0.10^{b} | <0.10^{b} | <0.10^{b} | 0.00^{b} | NA | NA |
| 6 | 23.30 | 1.81 | 3.72 | 6.07 | 3.87 | 2.13 | 55% | 3.42 | 5.23 | 7.53 | 5.39 | 2.06 | 38% |
| 24 | 14.70 | 3.04 | 10.50 | 6.91 | 6.82 | 3.73 | 55% | 13.60 | 11.70 | 10.90 | 12.07 | 1.39 | 11% |
| 48 | 9.06 | 4.38 | 7.27 | 7.92 | 6.52 | 1.88 | 29% | 7.45 | 9.28 | 6.92 | 7.88 | 1.24 | 16% |
| 72 | 5.75 | 3.96 | 7.49 | 4.97 | 5.47 | 1.82 | 33% | 8.28 | 8.70 | 4.91 | 7.30 | 2.08 | 28% |
| 96 | 5.06 | 3.71 | 6.74 | 4.31 | 4.92 | 1.60 | 33% | 5.96 | 7.41 | 4.55 | 5.97 | 1.43 | 24% |
| 168 | 1.71 | 1.52 | 2.74 | 1.88 | 2.05 | 0.63 | 31% | 2.76 | 2.77 | 1.60 | 2.38 | 0.67 | 28% |
| AUC₀₋₇ | 55.38 | 21.61 | 43.40 | 33.41 | 32.80 | 10.91 | 33% | 45.40 | 49.81 | 36.63 | 43.95 | 6.71 | 15% |
| (μg*day/mL) | | | | | | | | | | | | | |
| Cmax | 27.00 | 4.38 | 10.50 | 7.92 | 7.60 | 3.07 | 40% | 13.60 | 11.70 | 10.90 | 12.07 | 1.39 | 11% |
| (μg/mL) | | | | | | | | | | | | | |
| Tmax (day) | 0 | 2 | 1 | 2 | 2 | 1 | 35% | 1 | 1 | 1 | 1 | 0 | 0% |

^{a}Extrapolated
^{b}Values <LLOQ set to ‘0’ for PK parameter calculations

TABLE 93
Individual and mean serum concentrations of total ADC (TADC) - (μg/mL)

| | Animal ID# | | | | | | | | | | | | |
|---|---|---|---|---|---|---|---|---|---|---|---|---|---|
| | | | | | | | | 8737 | 8739 | 8743 | | | |
| | 8726 | 8730 | 8732 | 8734 | | | | ADC + | ADC + | ADC + | Mean | SD | % CV |
| Time Post- | ADC | ADCS | ADC | ADC | Mean | SD | % CV | rHuPH20 | rHuPH20 | rHuPH20 | SC + | SC + | SC + |
| Injection (h) | IV | C | SC | SC | SC | SC | SC | SC | SC | SC | PH20 | PH20 | PH20 |
| 0 | 25.12^{a} | <0.10^{b} | <0.10^{b} | <0.10^{b} | 0.00 | N/A | N/A | <0.10^{b} | <0.10^{b} | <0.10^{b} | 0.00 | N/A | N/A |
| 6 | 21.40 | 1.82 | 4.20 | 6.23 | 4.08 | 2.21 | 54% | 3.70 | 3.97 | 7.12 | 4.93 | 1.90 | 39% |
| 24 | 12.80 | 4.70 | 8.87 | 8.25 | 7.27 | 2.25 | 31% | 9.77 | 10.80 | 9.00 | 9.86 | 0.90 | 9% |
| 48 | 7.28 | 3.54 | 5.98 | 5.99 | 5.17 | 1.41 | 27% | 7.09 | 8.25 | 6.54 | 7.29 | 0.87 | 12% |
| 72 | 5.04 | 3.02 | 5.40 | 4.64 | 4.35 | 1.22 | 28% | 6.25 | 7.03 | 5.22 | 6.17 | 0.91 | 15% |
| 96 | 3.91 | 2.76 | 4.93 | 3.53 | 3.74 | 1.10 | 29% | 5.25 | 5.68 | 3.70 | 4.88 | 1.04 | 21% |
| 168 | 1.70 | 1.83 | 2.44 | 1.86 | 2.04 | 0.34 | 17% | 2.79 | 2.68 | 1.77 | 2.41 | 0.56 | 23% |
| AUC₀₋₇ | 47.73 | 19.85 | 34.76 | 30.81 | 28.47 | 7.73 | 27% | 38.42 | 42.10 | 33.25 | 37.92 | 4.44 | 12% |
| (ug*day/mL) | | | | | | | | | | | | | |
| Cmax | 25.12 | 4.70 | 8.87 | 8.25 | 7.27 | 2.25 | 31% | 9.77 | 10.80 | 9.00 | 9.86 | 0.90 | 9% |
| (ug/mL) | | | | | | | | | | | | | |
| Tmax (day) | 0 | 1 | 1 | 1 | 1 | 0 | 0% | 1.00 | 1.00 | 1.00 | 1 | 0 | 0% |

^{a}Extrapolated
^{b}Values <LLOQ set to ‘0.00’ for PK parameter calculations

TABLE 94
Individual and mean serum concentrations of free payload - (μg/mL)

| | Animal ID# | | | | | | | | | | | | |
|---|---|---|---|---|---|---|---|---|---|---|---|---|---|
| | | | | | | | | 8737 | 8739 | 8743 | | | |
| Time Post- | 8726 | 8730 | 8732 | 8734 | | | | ADC + | ADC + | ADC + | Mean | SD | % CV |
| Injection | ADC | ADC | ADC | ADC | Mean | SD | % CV | rHuPH20 | rHuPH20 | rHuPH20 | SC + | SC + | SC + |
| (h) | IV | SC | SC | SC | SC | SC | SC | SC | SC | SC | PH20 | PH20 | PH20 |
| 0 | 0.012^{a} | | | | | | | | | | | | |
| 6 | 0.0106 | 0.0032 | 0.0060 | 0.0097 | 0.0063 | 0.0032 | 51% | 0.0051 | 0.0055 | 0.0073 | 0.0060 | 0.0012 | 20% |
| 24 | 0.0061 | 0.0083 | 0.0038 | 0.0039 | 0.0054 | 0.0026 | 48% | 0.0050 | 0.0061 | 0.0047 | 0.0053 | 0.0008 | 14% |
| 48 | 0.0029 | 0.0060 | 0.0034 | 0.0031 | 0.0041 | 0.0016 | 38% | 0.0035 | 0.0038 | 0.0035 | 0.0036 | 0.0002 | 4% |
| 72 | 0.0095 | 0.0054 | 0.0067 | 0.0080 | 0.0067 | 0.0013 | 19% | 0.0095 | 0.0029 | 0.0089 | 0.0071 | 0.0037 | 52% |
| 96 | 0.0067 | 0.0034 | 0.0082 | 0.0049 | 0.0055 | 0.0024 | 44% | 0.0065 | 0.0028 | 0.0074 | 0.0056 | 0.0025 | 44% |
| 148 | 0.0021 | 0.0029 | 0.0035 | 0.0024 | 0.0029 | 0.0006 | 19% | 0.0040 | 0.0039 | 0.0023 | 0.0034 | 0.0010 | 29% |
| AUC₀₋₇ | 0.04 | 0.03 | 0.04 | 0.03 | 0.0341 | 0.0035 | 10% | 0.04 | 0.03 | 0.04 | 0.0345 | 0.0073 | 21% |
| (μg*day/mL) | | | | | | | | | | | | | |
| Cmax | 0.01 | 0.01 | 0.01 | 0.01 | 0.0087 | 0.0008 | 9% | 0.01 | 0.01 | 0.01 | 0.0082 | 0.0018 | 22% |
| (μg/mL) | | | | | | | | | | | | | |
| Tmax (day) | 0 | 1 | 4 | 0 | 2 | 2 | 113% | 3 | 1 | 3 | 2 | 1 | 49% |

0.012^{a}Extrapolated
^{b}Values <LLOQ set to ‘0.0000’ for PK parameter calculations

TABLE 95
TADC/TAB ratio - serum

| | Animal ID# | | | | | | | | | | | | |
|---|---|---|---|---|---|---|---|---|---|---|---|---|---|
| | | | | | | | | 8737 | 8739 | 8743 | | | |
| Time Post | 8726 | 8730 | 8732 | 8734 | | | | ADC + | ADC + | ADC + | | | |
| Injection | ADC | ADC | ADC | ADC | Mean | SD | % CV | rHuPH20 | rHuPH20 | rHuPH20 | Mean | SD | % CV |
| (h) | IV | SC | SC | SC | SC | SC | SC | SC | SC | SC | SC | SC | SC |
| 6 | 0.918 | 1.006 | 1.129 | 1.026 | 1.054 | 0.066 | 6.3% | 1.082 | 0.759 | 0.946 | 0.929 | 0.162 | 17.4% |
| 24 | 0.871 | 1.546 | 0.845 | 1.194 | 1.195 | 0.351 | 29.3% | 0.718 | 0.923 | 0.826 | 0.822 | 0.102 | 12.5% |
| 48 | 0.804 | 0.808 | 0.823 | 0.756 | 0.796 | 0.035 | 4.4% | 0.952 | 0.889 | 0.945 | 0.929 | 0.034 | 3.7% |
| 72 | 0.877 | 0.763 | 0.721 | 0.934 | 0.806 | 0.113 | 14.0% | 0.755 | 0.808 | 1.063 | 0.875 | 0.165 | 18.8% |
| 96 | 0.773 | 0.744 | 0.731 | 0.819 | 0.765 | 0.047 | 6.2% | 0.881 | 0.767 | 0.813 | 0.820 | 0.057 | 7.0% |
| 168 | 0.994 | 1.204 | 0.891 | 0.989 | 1.028 | 0.160 | 15.6% | 1.011 | 0.968 | 1.106 | 1.028 | 0.071 | 6.9% |

TABLE 96
Relative differences in serum PK metrics for ADC
analytes comparing routes of administration

| | | | ADC + | ADC + |
|---|---|---|---|---|
| | | ADC-SC vs. | rHuPH20-SC | rHuPH20-SC |
| | | ADC-IV | vs. ADC-IV | vs. ADC-SC |
| AUC₀₋₇ | TAB | 59% | 79% | 134% |
| | TADC | 60% | 79% | 133% |
| | Free Payload | 83% | 84% | 101% |
| Cmax | TAB | 28% | 45% | 159% |
| | TADC | 29% | 39% | 136% |
| | Free Payload | 70% | 65% | 94% |
| Tmax | TAB | NA | NA | 60% |
| | TADC | NA | NA | 100% |
| | Free Payload | NA | NA | 133% |

In Table 97, the ADC/Ab and ADC/Free Payload ratios reflect the extent of intact ADC in circulation. While IV exhibits the highest values for AUC₀₋₇ and Cₘₐₓ, ADC+rHuPH20-SC provides an improvement relative to ADC-SC alone, as reflected in both ratios.

TABLE 97
Relative differences in serum PK metrics for routes
of administration comparing analyte compositions

| | | | TADC/Free |
|---|---|---|---|
| | | TADC/TAB | Payload |
| AUC₀₋₇ | ADC (IV) | 86% | 116066% |
| | ADC (SC) | 87% | 83512% |
| | ADC + rHuPH20 (SC) | 86% | 109809% |
| Cmax | ADC (IV) | 93% | 201037% |
| | ADC (SC) | 96% | 83474% |
| | ADC + rHuPH20 (SC) | 82% | 120595% |
| Tmax | ADC (IV) | NA | NA |
| | ADC (SC) | 60% | 57% |
| | ADC + rHuPH20 (SC) | 100% | 43% |

### Bioanalytical Analysis of Post-Injection Tissue Samples

Tissue Samples from Injection Site—Present Study (Example 12)—D7 Skin Samples.

Tissue samples of the injection site were taken at 168h (D7) by taking a full thickness 12 mm tissue biopsies of the injection site. This sample was divided vertically into two identical pieces. One skin sample was placed into a pre-weighed cryogenic tube and its weight was recorded then stored on dry ice prior to transfer to ≤−70° C. The other skin sample was processed for potential future histological assessment into OCT freezing media and frozen on dry ice. Only the animals that were dosed SC had tissue biopsies taken: (3) animals were dosed SC with the ADC and (3) animals were dosed SC with the ADC+rHuPH20. The source of each tissue sample is provided in Table 98.

TABLE 98
Source of skin tissue samples, time points, and tissue weights

| Animal | Study | ‘Skin Tissue | Time | | Tissue Weight |
|---|---|---|---|---|---|
| ID# | Protocol | Sample Treatment | Point | | (mg) |
| 8731L | 25048 | ADC | 24 | h | 294.8 |
| 8731R | 25048 | ADC + rHuPH20 | 24 | h | 188.6 |
| 8733L | 25048 | ADC | 24 | h | 299.4 |
| 8733R | 25048 | ADC + rHuPH20 | 24 | h | 249.9 |
| 8536L | 25048 | ADC | 24 | h | 214.5 |
| 8536R | 25042 | ADC + rHuPH20 | 24 | h | 333.0 |
| 8738L | 25048 | ADC | 48 | h | 258.8 |
| 8738R | 25048 | ADC + rHuPH20 | 48 | h | 224.1 |
| 8740L | 25048 | ADC | 48 | h | 226.1 |
| 8740R | 25048 | ADC + rHuPH20 | 48 | h | 310.1 |
| 8742L | 25048 | ADC | 48 | h | 169.4 |
| 8742R | 25048 | ADC + rHuPH20 | 48 | h | 252.0 |
| 8730 | 25047 | ADC | 168 | h | 253.1 |
| 8732 | 25047 | ADC | 168 | h | 279.1 |
| 8734 | 25047 | ADC | 168 | h | 242.9 |
| 8737 | 25047 | ADC + rHuPH20 | 168 | h | 224.7 |
| 8739 | 25047 | ADC + rHuPH20 | 168 | h | 222.2 |
| 8743 | 25047 | ADC + rHuPH20 | 168 | h | 242.5 |

Tissue Samples from Injection Site—Follow-on Study (Example 13)—D1 and D2 Skin Samples.

A follow-on study repeated the subcutaneous dosing of the ADC+rHuPH20 that was conducted in the present study. The objective of this study was to identically mirror the SC dosing performed in the above study except that the tissue harvest occurred at 24 h and 48h. Animals received two SC doses on their lower abdominal region. The first SC injection was the ADC alone (5 mL; 50 mg), followed by a SC dose of the ADC+rHuPH20 (5 mL; 10 mg/mL; 2000 U/mL) on the contralateral side of the abdomen.

The test solutions used for the follow-on study were prepared in the same manner described previously (see above) and dose volumes mirrored those used in the present study. The data from the follow-on study is presented in conjunction with the data from the present study because these studies were designed to complement each other with identical dosing solutions and volumes to enable a combined readout from both studies.

Two cohorts were included in the follow-on study. Cohort 1 was euthanized 24 h after injection and cohort 2 was euthanized 48h after injection. Each animal had two full thickness skin biopsies taken post-mortem using a 12 mm punch. This sample was divided vertically into two identical pieces. One skin sample was placed into a pre-weighed cryogenic tube and its weight was recorded, then stored on dry ice prior to transfer to ≤−70° C. The other skin sample was processed into OCT molds for potential future histological assessment.

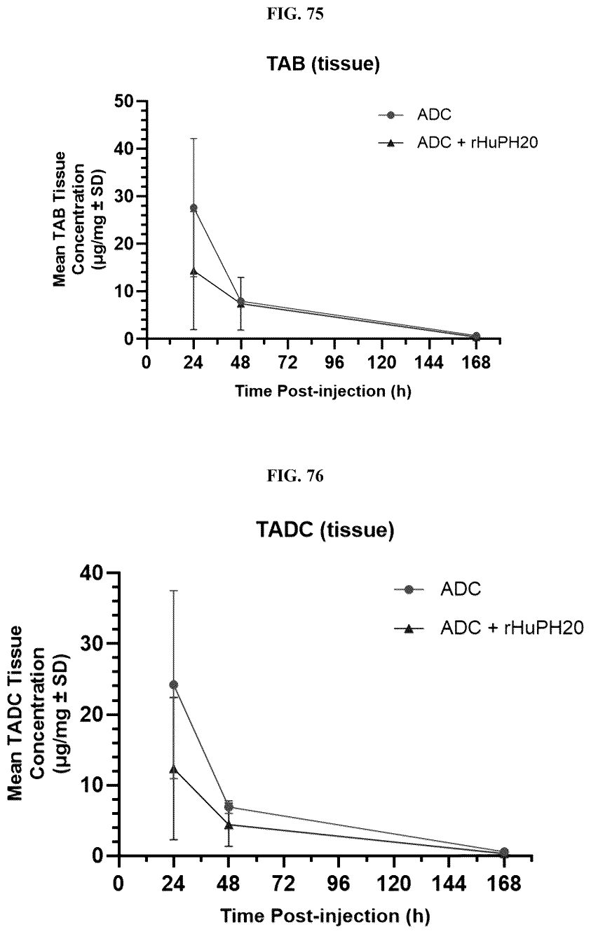

Tissue lysates were prepared based upon the weight of the sample at a ratio of 0.2 g/mL homogenization buffer (Tables 81 and 82). The total protein concentration of each homogenate was determined by BCA assay. The concentration of the total ADC (TADC), total antibody (TAB) and free payload (deruxtecan) in each sample was then normalized to the protein concentration and reported in μg analyte per mg total protein. The source of each tissue sample is provided in Table 98. The concentration of total antibody over time in tissue homogenates is provided in Table 99 and FIG. 75. The total ADC (TADC) concentrations in tissue homogenates are shown in Table 100 and FIG. 76.

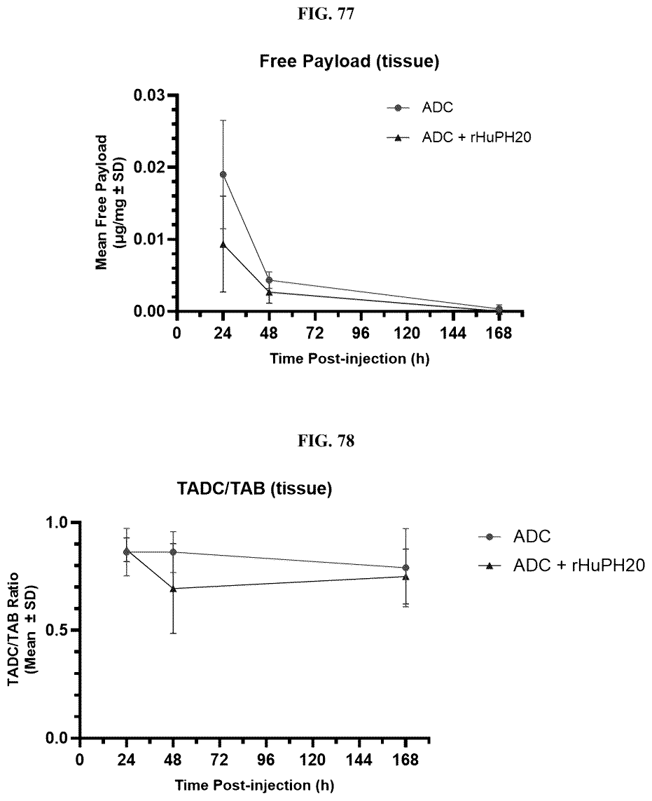

The total Free Payload concentrations in tissue homogenates are shown in Table 101 and FIG. 77. FIG. 78 provides the ratio of total ADC to total antibody (TADC/TAB) in skin tissue over time. Table 102 depicts the ratio of total ADC to total antibody in skin tissue homogenates, providing an indication of the extent of intact ADC that is remaining in the SC space over time.

The liquid-liquid extraction method used for the free payload sample processing method for skin tissue homogenates was shown to hydrolyze a significant proportion of payload from intact ADC (see Tables 81 and 82), therefore, the “free” payload results are known to significantly overestimate the quantity of unbound payload in the tissue samples.

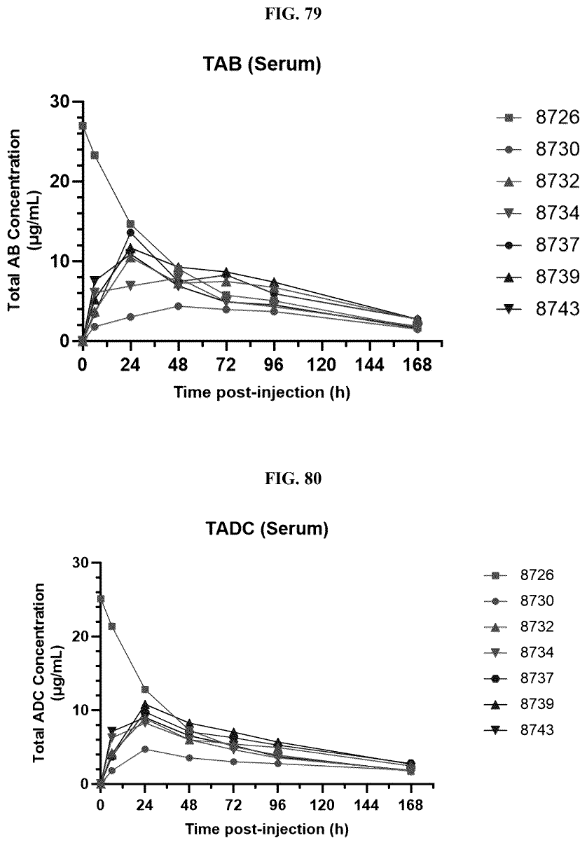

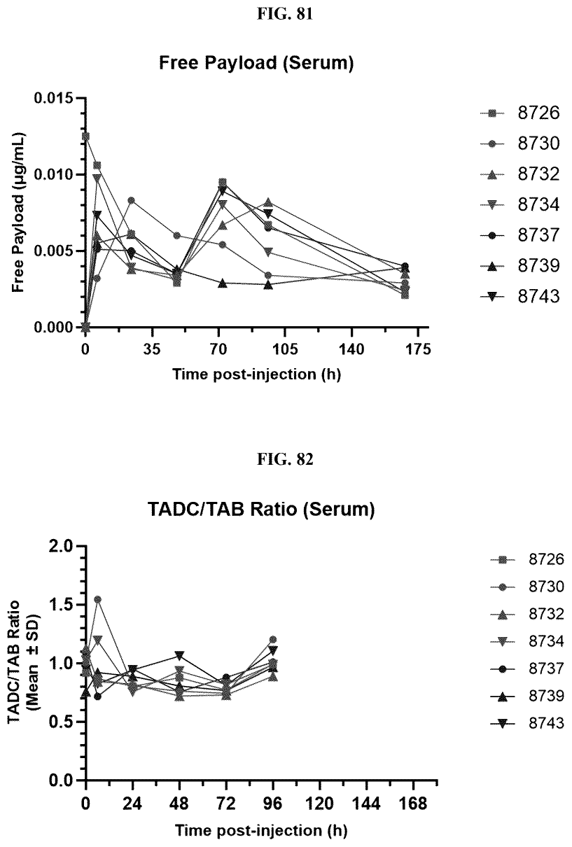

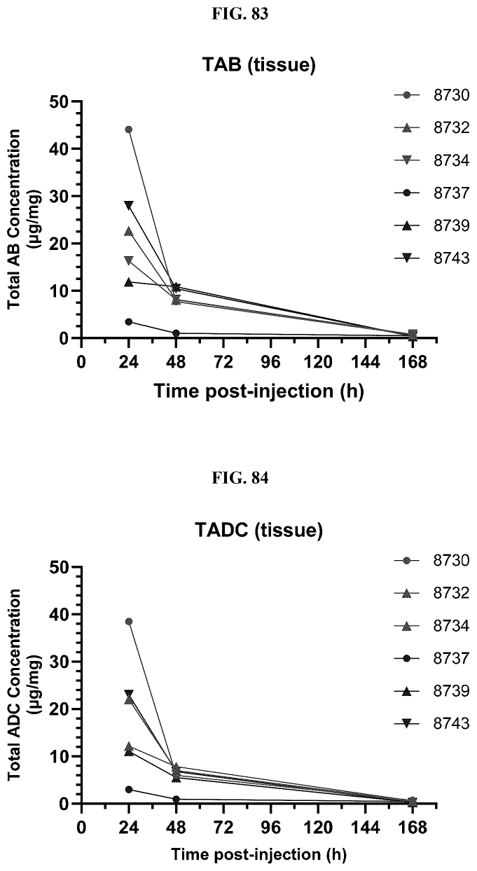

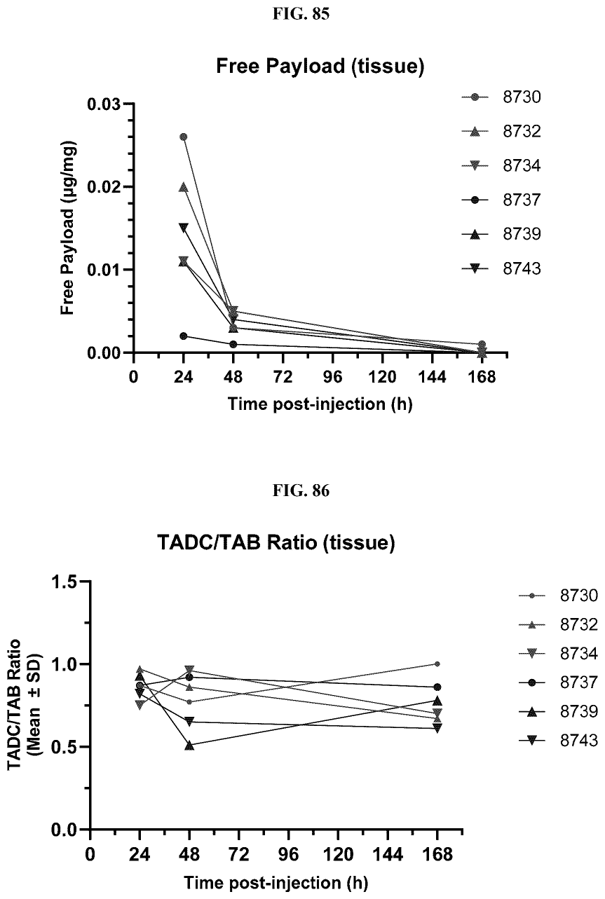

The measurement of serum ADC values for Total Antibody (TAB), Total ADC (TADC), Free Payload and TADC/TAB ratios for individual animals is shown in FIG. 79 through FIG. 82. The measurement of TAB, TADC, Free Payload and TADC/TAB ratios for tissue homogenates is shown in FIG. 83 through FIG. 86.

TABLE 99
Concentration of total antibody (TAB) in tissue homogenates of injection site (μg/mg protein ± SD)

| | | | | | | | | | | Mean | SD | CV |
|---|---|---|---|---|---|---|---|---|---|---|---|---|
| | | | | Mean | SD | CV | | | | ADC + | ADC + | ADC + |
| Time Post- | ADC | ADC | ADC | ADC | ADC | ADC | ADC + | ADC + | ADC + | rHuPH20 | rHuPH20 | rHuPH20 |
| injection (h) | SC | SC | SC | SC | SC | SC | rHuPH20SC | rHuPH20SC | rHuPH20SC | SC | SC | SC |
| 24 | 44.05 | 22.63 | 16.28 | 27.65 | 14.55 | 53% | 3.43 | 11.87 | 27.94 | 14.41 | 12.45 | 86% |
| 48 | 7.70 | 8.07 | 8.14 | 7.97 | 0.23 | 3% | 1.03 | 10.86 | 10.42 | 7.44 | 5.55 | 75% |
| 168 | 0.68 | 0.72 | 0.79 | 0.73 | 0.05 | 7% | 0.49 | 0.36 | 0.35 | 0.40 | 0.08 | 19% |
| AUC₍₁₋₇₎ | 46.85 | 37.33 | 34.52 | 39.57 | 6.46 | 16% | 6.03 | 39.42 | 46.11 | 30.52 | 21.47 | 70% |
| (μg*day/mL) | | | | | | | | | | | | |
| Cₘₐₓ | 44.05 | 22.63 | 16.28 | 27.65 | 14.55 | 53% | 3.43 | 11.87 | 27.94 | 14.41 | 12.45 | 86% |
| (μg/mL) | | | | | | | | | | | | |

TABLE 100
Concentration of total ADC (TADC) in tissue homogenates of injection site (μg/mg protein ± SD)

| | | | | | | | | | | Mean | SD | CV |
|---|---|---|---|---|---|---|---|---|---|---|---|---|
| | | | | Mean | SD | CV | ADC + | ADC + | ADC + | ADC + | ADC + | ADC + |
| Time Post- | ADC | ADC | ADC | ADC | ADC | ADC | rHuPH20 | rHuPH20 | rHuPH20 | rHuPH20 | rHuPH20 | rHuPH20 |
| injection (h) | SC | SC | SC | SC | SC | SC | SC | SC | SC | SC | SC | SC |
| 24 | 38.48 | 22.00 | 12.15 | 24.21 | 13.30 | 55% | 2.98 | 11.07 | 23.02 | 12.36 | 10.08 | 82% |
| 48 | 5.97 | 6.98 | 7.80 | 6.92 | 0.92 | 13% | 0.95 | 5.53 | 6.75 | 4.41 | 3.06 | 69% |
| 168 | 0.69 | 0.48 | 0.55 | 0.57 | 0.10 | 18% | 0.42 | 0.28 | 0.22 | 0.30 | 0.10 | 34% |
| AUC₍₁₋₇₎ | 38.87 | 33.14 | 30.85 | 34.29 | 4.13 | 12% | 5.39 | 22.81 | 32.31 | 20.17 | 13.65 | 68% |
| (μg*day/mL) | | | | | | | | | | | | |
| Cₘₐₓ | 38.48 | 22.00 | 12.15 | 24.21 | 13.30 | 55% | 2.98 | 11.07 | 23.02 | 12.36 | 10.08 | 82% |
| (μg/mL) | | | | | | | | | | | | |

TABLE 101
Concentration of free payload in tissue homogenates of injection site (μg/mg protein ± SD)

| | | | | | | | | | | Mean | SD | CV |
|---|---|---|---|---|---|---|---|---|---|---|---|---|
| | | | | Mean | | | ADC + | ADC + | ADC + | ADC + | ADC + | ADC + |
| Time Post- | ADC- | ADC- | ADC- | ADC- | SD | CV | rHuPH20 | rHuPH20 | rHuPH20 | rHuPH20 | rHuPH20 | rHuPH20 |
| injection (h) | SC | SC | SC | SC | SC | SC | SC | SC | SC | SC | SC | SC |
| 24 | 0.026 | 0.020 | 0.011 | 0.02 | 0.01 | 38% | 0.002 | 0.011 | 0.015 | 0.01 | 0.01 | 70% |
| 48 | 0.003 | 0.005 | 0.005 | 0.00 | 0.00 | 18% | 0.001 | 0.003 | 0.004 | 0.00 | 0.00 | 66% |
| 168 | 0.001 | 0.000 | 0.000 | 0.00 | 0.00 | 22% | 0.000 | 0.000 | 0.000 | 0.00 | 0.00 | 38% |
| AUC₍₁₋₇₎ | 0.024 | 0.024 | 0.021 | 0.02 | 0.00 | 8% | 0.004 | 0.016 | 0.019 | 0.01 | 0.01 | 63% |
| (ug*day/mL) | | | | | | | | | | | | |
| Cₘₐₓ (ug/mL) | 0.026 | 0.020 | 0.011 | 0.02 | 0.01 | 38% | 0.002 | 0.011 | 0.015 | 0.01 | 0.01 | 70% |

TABLE 102
TADC/TAB ratios in tissue homogenates (Mean SD)

| | | | | | | | | | | Mean | SD | % CV |
|---|---|---|---|---|---|---|---|---|---|---|---|---|
| Time Post | | | | Mean | SD | % CV | ADC + | ADC + | ADC + | ADC + | ADC + | ADC + |
| Injection | ADC | ADC | ADC | ADC | ADC | ADC | rHuPH20 | rHuPH20 | rHuPH20 | rHuPH20 | rHuPH20 | rHuPH20 |
| (h) | SC | SC | SC | SC | SC | SC | SC | SC | SC | SC | SC | SC |
| 24 | 0.87 | 0.97 | 0.75 | 1.14 | 0.11 | 9.9% | 0.87 | 0.93 | 0.82 | 1.17 | 0.05 | 4.7% |
| 48 | 0.78 | 0.86 | 0.96 | 1.15 | 0.09 | 7.9% | 0.92 | 0.51 | 0.65 | 1.69 | 0.21 | 12.5% |
| 168 | 1.01 | 0.67 | 0.70 | 1.27 | 0.19 | 15.2% | 0.86 | 0.78 | 0.63 | 1.30 | 0.12 | 8.9% |

Table 103 demonstrates the AUC₁₋₇ and Cmax for all analytes are substantially lower for ADC+rHuPH20 relative to ADC alone when delivered via SC administration.

TABLE 103
Relative difference in skin pharmacokinetic metrics for ADC analytes
after SC administration (ADC + rHuPH20 versus ADC alone)

| | Metric | (ADC + rHuPH20)/ADC |
|---|---|---|
| AUC₁₋₇ | Total Antibody (TAB) | 77% |
| | Total ADC (TADC) | 59% |
| | Free Payload (Payload) | 54% |
| Cmax | Total Antibody (TAB) | 52% |
| | Total ADC (TADC) | 51% |
| | Free Payload (Payload) | 50% |

Table 104 further demonstrates that at the 24 and 48 hour time points, the addition of rHuPH20 greatly reduces the concentration of all analytes in the skin relative to SC alone.

TABLE 104
Comparison of mean skin concentration (μg/mg protein)
for ADC analytes comparing routes of administration

| | | | | (ADC + |
|---|---|---|---|---|
| Time Post- | | | ADC + | rHuPH20)/ |
| injection (h) | Metric | ADC | rHuPH20 | ADC |
| 24 | Total Antibody (TAB) | 27.65 | 14.41 | 52% |
| | Total ADC (TADC) | 24.21 | 12.36 | 51% |
| | Free Payload | 0.02 | 0.01 | 50% |
| 48 | Total Antibody (TAB) | 7.97 | 7.44 | 93% |
| | Total ADC (TADC) | 6.92 | 4.41 | 64% |
| | Free Payload | 0.0042 | 0.0025 | 58% |
| 168 | Total Antibody (TAB) | 0.73 | 0.40 | 55% |
| | Total ADC (TADC) | 0.57 | 0.30 | 53% |
| | Free Payload | 0.0004 | 0.0002 | 46% |

### Summary

rHuPH20 enables faster absorption and reduced residence time, which resulted in less payload existing at the SC site of administration and higher payload-bound ADC, in systemic circulation.

The PK improvements afforded by rHuPH20 (greater dispersion, higher bioavailability and reduced residence time), combined with the ability to accommodate greater doses and volumes, enable potential improvements to safety and/or efficacy of this ADC.

### Example 13: Follow-on Study-Assessment of Local Injection Site Retention of an ADC (Trastuzumab Deruxtecan) after Subcutaneous Administration with and without rHuPH20

### Summary

The objective of this study was to determine the local injection site tissue retention of an ADC after SC administration either alone or co-mixed with rHuPH20. Each animal received two SC injections on the lower abdomen; the first injection of ADC alone followed by a second SC injection of the ADC co-mixed with rHuPH20 at a concentration of 2000 U/mL. Dose volume was 1.25 mL for each injection. The ADC used for this study was trastuzumab deruxtecan.

The Yucatan miniature pig model has been selected for this study due to the high degree of similarity of the subcutaneous (SC) space to that of humans. Previous studies using a mini-pig model have demonstrated the translatability of the model for use in pre-clinical studies. To determine the amount of ADC that remains present after SC injection, tissue samples of each injection site were obtained from each animal post-mortem. Timepoints for tissue collection were 24 and 48 hours post-injection. The tissue samples were snap frozen on dry ice for bioanalytical analysis.

### Introduction and Objectives

Rapid subcutaneous administration of large volumes of antibodies has been shown to be feasible with rHuPH20. rHuPH20 facilitates SC administration of fluids and drugs by transiently and locally depolymerizing hyaluronan (HA) in the extracellular matrix thereby reducing tissue backpressure in the SC space permitting rapid, large volume administration of fluid.

A novel class of antibody-based therapeutics has been developed and recently approved by the FDA for the management or treatment of cancer (Ducry et al., 2010). These therapeutics combine monoclonal antibodies specific to surface antigens present on particular tumor cells with highly potent anti-cancer agents linked via a chemical linker to form an ADC. It is important to assess the distribution from the injection site following SC administration, and to determine if rHuPH20 is able to enhance the disperse of the ADC more effectively away from the site, potentially reducing local skin toxicity and improving absorption. The swine model was chosen for this assessment due to the high similarity of the skin and subcutaneous space between humans and swine.

In this study, each animal received two SC injections—one injection of the ADC alone followed by a second SC injection of the ADC co-mixed with rHuPH20. Each injection was 1.25 mL and was delivered using a pre-filled 3-cc syringe connected to a 25G×1-inch needle. Two cohorts were evaluated in this study. The first was euthanized at 24 hours post-injection and biopsies were collected from each site for analysis post-mortem. The second cohort was euthanized at 48 hours post-injection and biopsies were similarly be taken from the injection sites.

### Experimental Design and Methods

### Test and Control Articles

### 1) Antibody Drug Conjugate (ADC)—Fam-Trastuzumab Deruxtecan-Nxki

Description: Clear colorless liquid
Lot numbers: #1) 2152417730747270
   #2) 2504552804161651
   #3) 2867837734739503
Vial Contents: 100 mg (lyophilized)
Final Concentration: 20 mg/mL
Formulation: Reconstituted in Sterile Water for Injection, USP
Storage Conditions: 2-8° C.
Handling Conditions: Standard laboratory precautions
   2) Recombinant Human Hyaluronidase (rHuPH20)
Description: Clear colorless liquid
Lot number: 1-FIN-3928
Concentration: 1,039,763 U/mL
Formulation: 10 mM Histidine, 130 mM sodium chloride, pH 6.5
Storage Conditions: ≤70° C.
Handling Conditions: Standard laboratory precautions
Supplier: Halozyme, Inc.

### 3) Sterile Water for Injection

Description: Clear colorless liquid
Catalog #: NDC 0409-4887-17
Lot number: LT4430
Expiration date: 2027 Aug. 31
Storage Conditions: Room temperature
Handling Conditions: Standard laboratory precautions
Supplier: Hospira

### Formulation

All solutions were prepared in a biosafety cabinet using standard laboratory safety protocol. The antibody drug conjugate (fam-trastuzumab deruxtecan-nxki) was rehydrated by the addition of 5 mL of Sterile Water for Injection to the lyophilized product to yield a test solution at 20 mg/mL. After rehydration of the ADC, 6 syringes were prepared containing 1.25 mL of the antibody solution alone (control syringes). After preparation of the control syringes, the remaining antibody solution had concentrated rHuPH20 added to a final concentration of 2000 U/mL. After preparation of the co-mix of antibody and rHuPH20, 1.25 mL of the solution was drawn into 6 additional syringes. After filling each syringe, it was capped and stored at 2-8° C. or on ice until removed and brought to room temperature prior to use. On the day of the study the syringes were brought to room temperature for a minimum of 45 minutes prior to use. The syringe cap was removed and replaced with a 25G×1-inch needle.

### Test Methods

Prior to study start, the enzyme activity of the test solution was measured using an activity assay to confirm the concentration of rHuPH20 in the co-mix drug solution. The result of the pre-study activity testing is shown in Table 105.

TABLE 105
Summary of Pre-study Activity Testing

| Test Solution | Hyaluronidase Activity (U/mL ± SD) |
|---|---|
| ADC + rHuPH20 | 2097 ± 26 |

### Animal Description

Species: Pig (Sus scrofa domestica)
Strain: Yucatan Miniature
Sex: Female
Age: ˜3-6 months
Weight: ˜14-16 kg
Quantity: 6 animals
Source: Premier BioSource (Ramona, CA)

### Husbandry.

The animals were housed in a steel pen with automatic water provided ad libitum. The animals were fed twice daily (AM and PM) but was kept NPO after midnight on the day of the study to prevent anesthesia complications. The room environment was set to maintain a temperature of ˜19-23° C. and a relative humidity of 40-70%, with a 12 hour light/12 hour dark time cycle. The animals were acclimated to the vivarium for 3 days prior to study start. The bodyweights and date of birth for each animal is provided in Table 106.

TABLE 106
Summary of bodyweights and dates of birth

| Animal ID# | Bodyweight (kg) | Date of Birth |
|---|---|---|
| 8731 | 18 | Mar. 18, 2025 |
| 8733 | 17 | Mar. 5, 2025 |
| 8736 | 17 | Mar. 3, 2025 |
| 8738 | 15 | Mar. 5, 2025 |
| 8740 | 17 | Mar. 5, 2025 |
| 8742 | 16 | Mar. 3, 2025 |

### Test Materials

The following test materials shown in Table 107 were used in the study:

TABLE 107
Test materials

| Test Material | Supplier | Catalog # |
|---|---|---|
| 3-cc syringe | Becton Dickinson | 309657 |
| Syringe caps | Becton Dickinson | 305819 |
| 25G × 1-inch needle | Becton Dickinson | 305125 |
| Digital point and shoot camera | Canon | S120 |
| Accuderm 12 mm biopsy punch | Fisher Scientific | NC9253254 |
| Disposable scissors | Fisher Scientific | 50-109-4247 |
| Disposable forceps | Fisher Scientific | NC0266178 |
| O.C.T. freezing compound | Fisher Scientific | 23-730-571 |
| Epredid ® Embedding Molds | Fisher Scientific | 22-19 |
| 22 × 22 × 20 mm | | |
| Cryogenic vials, 1.2 mL | Fisher Scientific | 10-500-25 |

### Experimental Design

This was an exploratory study to assess the local retention time of an antibody-drug conjugate (fam-trastuzumab deruxtecan-nxki) with and without rHuPH20. In this study, six animals were used. Each animal received (2) subcutaneous injections—the first injection was the ADC alone followed by the second injection of ADC+rHuPH20 on the contralateral side of the abdomen of the animal. Two cohorts were included in this study. Cohort 1 was euthanized 24 hours after treatment, while Cohort 2 was euthanized 48 hours after exposure to the two test solutions. A summary of the treatment groups is shown in Table 108.

TABLE 108
Description of treatments

| | | Dose | | | |
|---|---|---|---|---|---|
| | | Volume | # of | [rHuPH20] | Exposure |
| Cohort # | Test Solution | (mL) | Injections | (U/mL) | Time (h) |
| 1 | trastuzumab deruxtecan | 1.25 | 3 | 0 | 24 |
| | trastuzumab deruxtecan + rHuPH20 | 1.25 | 3 | 2000 | 24 |
| 2 | trastuzumab deruxtecan | 1.25 | 3 | 0 | 48 |
| | trastuzumab deruxtecan + rHuPH20 | 1.25 | 3 | 2000 | 48 |

After euthanasia, 12 mm punch biopsies of each injection site were obtained. The punch biopsy was cut in half (sectioned vertically) and placed into OCT cryo molds with the inner cut surface facing the bottom of the cryo mold. The sample was then placed in a dry ice/2-methylbutane bath for freezing. Once frozen the sample was stored on dry ice until transported for long-term storage at ≤70° C.

### Study Design

Prior to the start of study, the animal was assessed for general health, and its body weight collected. All anesthesia (isoflurane) was administered and monitored by the AccuLab staff. After anesthetization, the animal was placed in dorsal recumbence (no wedge) on a heated surgical table and was maintained under isoflurane gas for the entire duration of the procedure. Following anesthetization, the abdominal region was shaved and cleaned with Nolvasan followed by wiping the injection site with gauze containing 70% isopropanol and wiped dry with sterile gauze. Injection sites were located on the left and right abdominal regions, located 3 mm towards the midline of the animal from the middle of the inguinal fold and approximately 5 mm cranially.

Immediately prior to injection, the cap was removed from the syringe and replaced with a 25G×1-inch needle. For needle insertion, a standard pinch method was used. After needle insertion the injection proceeded. At 24h post-injection, the animals for cohort 1 were euthanized (3 animals) and punch biopsies obtained as described above. After obtaining the punch biopsies, the animal carcass was removed and disposed of as biohazardous waste. Animals for cohort 2 were euthanized at 48 hours post-injection and samples obtained as described above.

Bioanalytical testing of the samples was conducted to quantitate presence of the ADC at the injection site tissue. Tissue samples were analyzed for the presence of the free antibody (no payload) as well as free payload.

### Results

See Example 12 for a description of the tissue sample methods used in this follow-on study. See also Tables 99-102 as well as FIGS. 75-78 of Example 12 for the data associated with this follow-on study.

TABLE 112
Total antibody concentrations (μg/mL) in the serum of minipigs

| | Serum | | | | | | | | | | | | |
|---|---|---|---|---|---|---|---|---|---|---|---|---|---|
| | Total Ab (ug/mL) | | | | | | | | | | | | |
| | | | | | Mean | SD | CV | | | | Mean | SD | CV |
| days | IV | SC | SC | SC | SC | SC | SC | PH20 | PH20 | PH20 | PH20 | PH20 | PH20 |
| 0 | 27.00 | 0.00 | 0.00 | 0.00 | 0.00 | 0.00 | NA | 0.00 | 0.00 | 0.00 | 0.00 | 0.00 | NA |
| 0.25 | 23.30 | 1.81 | 3.72 | 6.07 | 3.87 | 2.13 | 55% | 3.42 | 5.23 | 7.53 | 5.39 | 2.06 | 38% |
| 1 | 14.70 | 3.04 | 10.50 | 6.91 | 6.82 | 3.73 | 55% | 13.60 | 11.70 | 10.90 | 12.07 | 1.39 | 11% |
| 2 | 9.06 | 4.38 | 7.27 | 7.92 | 6.52 | 1.88 | 29% | 7.45 | 9.28 | 6.92 | 7.88 | 1.24 | 16% |
| 3 | 5.75 | 3.96 | 7.49 | 4.97 | 5.47 | 1.82 | 33% | 8.28 | 8.70 | 4.91 | 7.30 | 2.08 | 28% |
| 4 | 5.06 | 3.71 | 6.74 | 4.31 | 4.92 | 1.60 | 33% | 5.96 | 7.41 | 4.55 | 5.97 | 1.43 | 24% |
| 7 | 1.71 | 1.52 | 2.74 | 1.88 | 2.05 | 0.63 | 31% | 2.76 | 2.77 | 1.60 | 2.38 | 0.67 | 28% |
| AUC₀₋₇ | 55.38 | 21.61 | 43.40 | 33.41 | 32.80 | 10.91 | 33% | 45.40 | 49.81 | 36.63 | 43.95 | 6.71 | 15% |
| (ug*day/mL) | | | | | | | | | | | | | |
| Cmax (ug/mL) | 27.00 | 4.38 | 10.50 | 7.92 | 7.60 | 3.07 | 40% | 13.60 | 11.70 | 10.90 | 12.07 | 1.39 | 11% |
| Tmax (day) | 0 | 2 | 1 | 2 | 2 | 1 | 35% | 1 | 1 | 1 | 1 | 0 | 0% |

TABLE 113
Total ADC concentrations (ug/mL) in the serum of minipigs

| | Total ADC (μg/mL) | | | | | | | | | | | | |
|---|---|---|---|---|---|---|---|---|---|---|---|---|---|
| | | | | | Mean | SD | CV | | | | Mean | SD | CV |
| days | IV | SC | SC | SC | SC | SC | SC | PH20 | PH20 | PH20 | PH20 | PH20 | PH20 |
| 0 | 25.12 | 0.00 | 0.00 | 0.00 | 0.00 | 0.00 | NaN | 0.00 | 0.00 | 0.00 | 0.00 | 0.00 | NaN |
| 0.25 | 21.40 | 1.82 | 4.20 | 6.23 | 4.08 | 2.21 | 54% | 3.70 | 3.97 | 7.12 | 4.93 | 1.90 | 39% |
| 1 | 12.80 | 4.70 | 8.87 | 8.25 | 7.27 | 2.25 | 31% | 9.77 | 10.80 | 9.00 | 9.86 | 0.90 | 9% |
| 2 | 7.28 | 3.54 | 5.98 | 5.99 | 5.17 | 1.41 | 27% | 7.09 | 8.25 | 6.54 | 7.29 | 0.87 | 12% |
| 3 | 5.04 | 3.02 | 5.40 | 4.64 | 4.35 | 1.22 | 28% | 6.25 | 7.03 | 5.22 | 6.17 | 0.91 | 15% |
| 4 | 3.91 | 2.76 | 4.93 | 3.53 | 3.74 | 1.10 | 29% | 5.25 | 5.68 | 3.70 | 4.88 | 1.04 | 21% |
| 7 | 1.70 | 1.83 | 2.44 | 1.86 | 2.04 | 0.34 | 17% | 2.79 | 2.68 | 1.77 | 2.41 | 0.56 | 23% |
| AUC₀₋₇ | 47.73 | 19.85 | 34.76 | 30.81 | 28.47 | 7.73 | 27% | 38.42 | 42.10 | 33.25 | 37.92 | 4.44 | 12% |
| (ug*day/mL) | | | | | | | | | | | | | |
| Cmax (ug/mL) | 25.12 | 4.70 | 8.87 | 8.25 | 7.27 | 2.25 | 31% | 9.77 | 10.80 | 9.00 | 9.86 | 0.90 | 9% |
| Tmax (day) | 0 | 1 | 1 | 1 | 1 | 0 | 0% | 1.00 | 1.00 | 1.00 | 1 | 0 | 0% |

TABLE 114
Total concentrations (μg/mL) of free payload in the serum of minipigs

| | Free Payload (ug/mL) | | | | | | | | | | | | |
|---|---|---|---|---|---|---|---|---|---|---|---|---|---|
| | | | | | Mean | SD | CV | | | | Mean | SD | CV |
| days | IV | SC | SC | SC | SC | SC | SC | PH20 | PH20 | PH20 | PH20 | PH20 | PH20 |
| 0 | 0.0125 | 0.0000 | 0.0000 | 0.0000 | 0.0000 | 0.0000 | NaN | 0.0000 | 0.0000 | 0.0000 | 0.0000 | 0.0000 | NaN |
| 0.25 | 0.0106 | 0.0032 | 0.0060 | 0.0097 | 0.0063 | 0.0032 | 51% | 0.0051 | 0.0055 | 0.0073 | 0.0060 | 0.0012 | 20% |
| 1 | 0.0061 | 0.0083 | 0.0038 | 0.0039 | 0.0054 | 0.0026 | 48% | 0.0050 | 0.0061 | 0.0047 | 0.0053 | 0.0008 | 14% |
| 2 | 0.0029 | 0.0060 | 0.0034 | 0.0031 | 0.0041 | 0.0016 | 38% | 0.0035 | 0.0038 | 0.0035 | 0.0036 | 0.0002 | 4% |
| 3 | 0.0095 | 0.0054 | 0.0067 | 0.0080 | 0.0067 | 0.0013 | 19% | 0.0095 | 0.0029 | 0.0089 | 0.0071 | 0.0037 | 52% |
| 4 | 0.0067 | 0.0034 | 0.0082 | 0.0049 | 0.0055 | 0.0024 | 44% | 0.0065 | 0.0028 | 0.0074 | 0.0056 | 0.0025 | 44% |
| 7 | 0.0021 | 0.0029 | 0.0035 | 0.0024 | 0.0029 | 0.0006 | 19% | 0.0040 | 0.0039 | 0.0023 | 0.0034 | 0.0010 | 29% |
| AUC₀₋₇ | 0.04 | 0.03 | 0.04 | 0.03 | 0.0341 | 0.0035 | 10% | 0.04 | 0.03 | 0.04 | 0.0345 | 0.0073 | 21% |
| (ug*day/mL) | | | | | | | | | | | | | |
| Cmax (ug/mL) | 0.01 | 0.01 | 0.01 | 0.01 | 0.0087 | 0.0008 | 9% | 0.01 | 0.01 | 0.01 | 0.0082 | 0.0018 | 22% |
| Tmax (day) | 0 | 1 | 4 | 0 | 2 | 2 | 113% | 3 | 1 | 3 | 2 | 1 | 49% |

TABLE 115
Total antibody concentrations (μg/mL) in the skin of minipigs

| | Total Ab (ug/mg protein) | | | | | | | | | | | | |
|---|---|---|---|---|---|---|---|---|---|---|---|---|---|
| | | | | | Mean | SD | CV | | | | Mean | SD | CV |
| days | IV | SC | SC | SC | SC | SC | SC | PH20 | PH20 | PH20 | PH20 | PH20 | PH20 |
| 0 | NaN | NaN | NaN | NaN | NaN | NaN | NaN | NaN | NaN | NaN | NaN | NaN | NaN |
| 1 | NaN | 44.05 | 22.63 | 16.28 | 27.65 | 14.55 | 53% | 3.43 | 11.87 | 27.94 | 14.41 | 12.45 | 86% |
| 2 | NaN | 7.70 | 8.07 | 8.14 | 7.97 | 0.23 | 3% | 1.03 | 10.86 | 10.42 | 7.44 | 5.55 | 75% |
| 7 | NaN | 0.68 | 0.72 | 0.79 | 0.73 | 0.05 | 7% | 0.49 | 0.36 | 0.35 | 0.40 | 0.08 | 19% |
| AUC₁₋₇ | NaN | 46.85 | 37.33 | 34.52 | 39.57 | 6.46 | 16% | 6.03 | 39.42 | 46.11 | 30.52 | 21.47 | 70% |
| (ug*day/mL) | | | | | | | | | | | | | |
| Cmax (ug/mL) | NaN | 44.05 | 22.63 | 16.28 | 27.65 | 14.55 | 53% | 3.43 | 11.87 | 27.94 | 14.41 | 12.45 | 86% |

TABLE 116
Total ADC concentrations (μg/mL) in the skin of minipigs

| | Total ADC (ug/mg protein) | | | | | | | | | | | | |
|---|---|---|---|---|---|---|---|---|---|---|---|---|---|
| | | | | | Mean | SD | CV | | | | Mean | SD | CV |
| days | IV | SC | SC | SC | SC | SC | SC | PH20 | PH20 | PH20 | PH20 | PH20 | PH20 |
| 0 | NaN | NaN | NaN | NaN | NaN | NaN | NaN | NaN | NaN | NaN | NaN | NaN | NaN |
| 1 | NaN | 38.48 | 22.00 | 12.15 | 24.21 | 13.30 | 55% | 2.98 | 11.07 | 23.02 | 12.36 | 10.08 | 82% |
| 2 | NaN | 5.97 | 6.98 | 7.80 | 6.92 | 0.92 | 13% | 0.95 | 5.53 | 6.75 | 4.41 | 3.06 | 69% |
| 7 | NaN | 0.69 | 0.48 | 0.55 | 0.57 | 0.10 | 18% | 0.42 | 0.28 | 0.22 | 0.30 | 0.10 | 34% |
| AUC₁₋₇ | NaN | 38.87 | 33.14 | 30.85 | 34.29 | 4.13 | 12% | 5.39 | 22.81 | 32.31 | 20.17 | 13.65 | 68% |
| (ug*day/mL) | | | | | | | | | | | | | |
| Cmax (ug/mL) | NaN | 38.48 | 22.00 | 12.15 | 24.21 | 13.30 | 55% | 2.98 | 11.07 | 23.02 | 12.36 | 10.08 | 82% |

TABLE 117
Total concentrations (μg/mL) of free payload in the skin of minipigs

| | Free Payload (ug/mg protein) | | | | | | | | | | | | |
|---|---|---|---|---|---|---|---|---|---|---|---|---|---|
| | | | | | Mean | SD | CV | | | | Mean | SD | CV |
| days | IV | SC | SC | SC | SC | SC | SC | PH20 | PH20 | PH20 | PH20 | PH20 | PH20 |
| 0 | NaN | NaN | NaN | NaN | NaN | NaN | NaN | NaN | NaN | NaN | NaN | NaN | NaN |
| 1 | NaN | 0.026 | 0.020 | 0.011 | 0.02 | 0.01 | 38% | 0.002 | 0.011 | 0.015 | 0.01 | 0.01 | 70% |
| 2 | NaN | 0.003 | 0.005 | 0.005 | 0.00 | 0.00 | 18% | 0.001 | 0.003 | 0.004 | 0.00 | 0.00 | 66% |
| 7 | NaN | 0.001 | 0.000 | 0.000 | 0.00 | 0.00 | 22% | 0.000 | 0.000 | 0.000 | 0.00 | 0.00 | 38% |
| AUC₁₋₇ | NaN | 0.024 | 0.024 | 0.021 | 0.02 | 0.00 | 8% | 0.004 | 0.016 | 0.019 | 0.01 | 0.01 | 63% |
| (ug*day/mL) | | | | | | | | | | | | | |
| Cmax (ug/mL) | NaN | 0.026 | 0.020 | 0.011 | 0.02 | 0.01 | 38% | 0.002 | 0.011 | 0.015 | 0.01 | 0.01 | 70% |

TABLE 118
Mean AUC₀₋₇, Cmax, and Tmax for antibody,
ADC, and payload analytes in minipig serum
comparing routes of administration

| | | SC/IV | PH20/IV | PH20/SC |
|---|---|---|---|---|
| AUC₀₋₇ | Ab | 59% | 79% | 134% |
| | ADC | 60% | 79% | 133% |
| | Payload | 83% | 84% | 101% |
| Cmax | Ab | 28% | 45% | 159% |
| | ADC | 29% | 39% | 136% |
| | Payload | 70% | 65% | 94% |
| Tmax | Ab | NaN | NaN | 60% |
| | ADC | NaN | NaN | 100% |
| | Payload | NaN | NaN | 133% |

TABLE 119
Mean AUC₀₋₇, Cmax, and Tmax for ADC/Ab ratio and ADC/Payload
ratio in minipig serum comparing routes of administration

| | | ADC/Ab | ADC/Payload |
|---|---|---|---|
| AUC₀₋₇ | IV | 86% | 116066% |
| | SC | 87% | 83512% |
| | PH20 | 86% | 109809% |
| Cmax | IV | 93% | 201037% |
| | SC | 96% | 83474% |
| | PH20 | 82% | 120595% |
| Tmax | IV | NaN | NaN |
| | SC | 60% | 57% |
| | PH20 | 100% | 43% |

TABLE 120
Mean AUC₁₋₇ and Cmax for antibody, ADC, and payload analytes
in minipig skin comparing routes of administration

| | | SC/IV | PH20/IV | PH20/SC |
|---|---|---|---|---|
| AUC₁₋₇ | Ab | NaN | NaN | 77% |
| | ADC | NaN | NaN | 59% |
| | Payload | NaN | NaN | 54% |
| Cmax | Ab | NaN | NaN | 52% |
| | ADC | NaN | NaN | 51% |
| | Payload | NaN | NaN | 50% |

TABLE 121
Mean AUC₁₋₇ and Cmax for ADC/Ab ratio and ADC/Payload ratio
in minipig skin comparing routes of administration

| | | ADC/Ab | ADC/Payload |
|---|---|---|---|
| AUC₁₋₇ | IV | NaN | NaN |
| | SC | 87% | 147791% |
| | PH20 | 66% | 159762% |
| Cmax | IV | NaN | NaN |
| | SC | 88% | 128404% |
| | PH20 | 86% | 129869% |

TABLE 122
Mean percentage of antibody, ADC, and payload
in minipig skin 24 hours and 48 hours after
SC administration with and without rHuPH20

| Hours | Comp | SC | PH20 | PH20/SC |
|---|---|---|---|---|
| 24 | Ab | 27.65 | 14.41 | 52% |
| | ADC | 24.21 | 12.36 | 51% |
| | Payload | 0.02 | 0.01 | 50% |
| 48 | Ab | 7.97 | 7.44 | 93% |
| | ADC | 6.92 | 4.41 | 64% |
| | Payload | 0.00 | 0.00 | 58% |

### Example 14: Total Payload Concentrations in Minipigs Administered Trodelvy and Enhertu

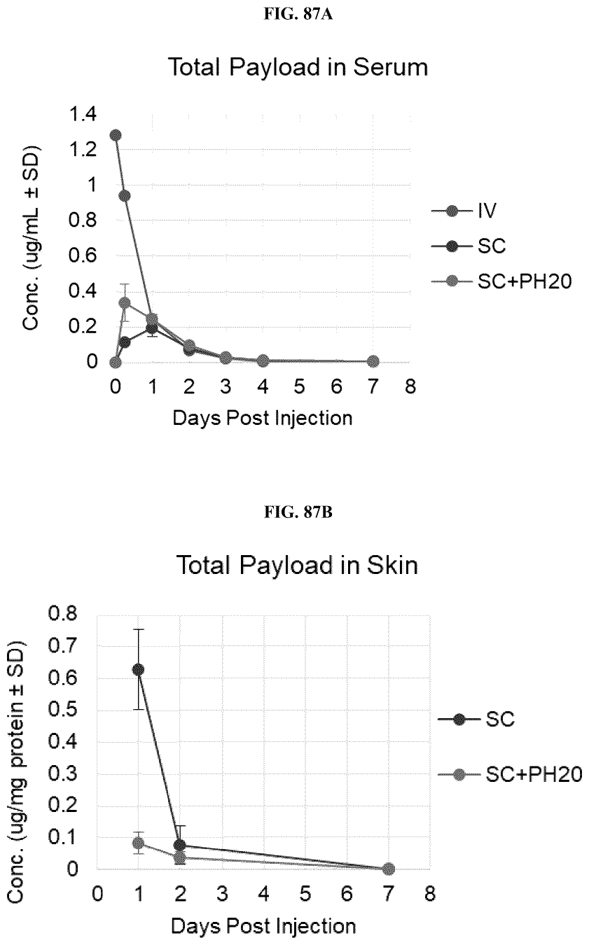

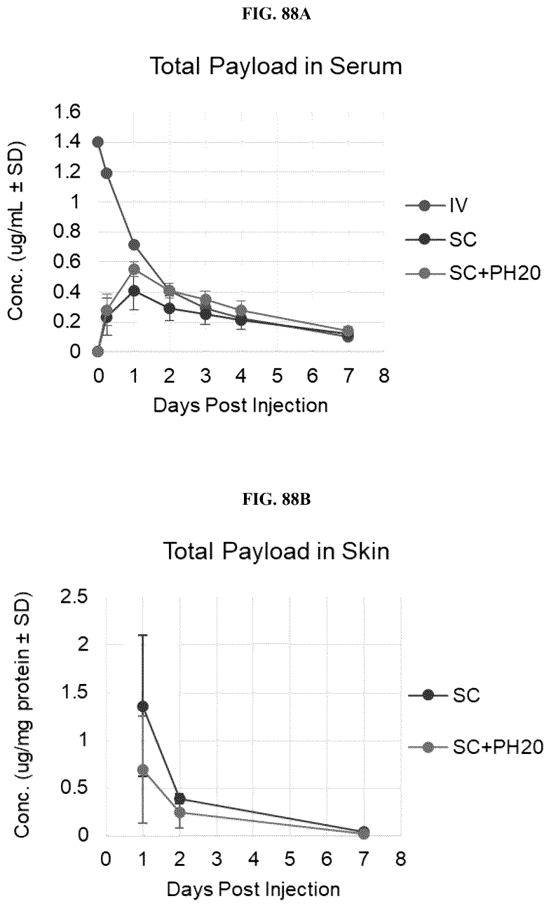

Total payload was calculated on a molar basis from measured total ADC and free payload using the equation: Total payload=(Total ADC×DAR)+Free payload, where DAR (drug-to-antibody ratio) was assumed to be time-invariant and fixed at the average reported value for the ADC. For Trodelvy and Enhertu, DAR was set to 8. The results are shown in FIGS. 87A-88B.

Trends in total payload concentrations in serum indicate that a subcutaneous dose of the ADC and the soluble hyaluronidase achieving equivalent AUC to an IV dose can result in a Cmax approximately 50% lower than that of IV administration. At the site of administration (skin), subcutaneous administration of the ADC and the soluble hyaluronidase shows reduced Cmax and AUC compared to SC alone. These findings suggest that decreased residence time and lower local concentrations of total payload with the subcutaneous administration soluble hyaluronidase may reduce the risk of local toxicity and potentially enhance the therapeutic window.

### Example 15: Pharmacokinetic Data Based on Route of Administration

Percentages provided are for amount of rHuPH20 relative to IV or SC alone, not percent improvement.
1. The higher the ratio of total ADC:free antibody, the better; this means the ADC is comprised of more attached payload
2. The higher the ratio of total ADC:free payload, the better; this means less payload has dissociated and more remains intact on the ADC.

| SC + HuPH20 relative to IV | | | SC + HuPH20 relative to SC alone | | |
|---|---|---|---|---|---|
| Cmin | Cmax | AUC | Cmin | Cmax | AUC |

Systemic

| Total ADC | lower- | lower | lower- | higher (100-200%) |
|---|---|---|---|---|
| Concentration | higher | (25-100%) | higher | |
| | (50-100%) | | (50-200%) | |
| Total ADC | lower-equal (75-100%) | | | higher (100-200%) |
| Free | | | | |
| Antibody¹ | | | | |
| Total ADC | lower-equal (75-100%) | | | higher (100-200%) |
| Free Payload² | | | | |

Subcutaneous

| Total ADC | not applicable | lower (50-100%) |
|---|---|---|
| Concentration | | |
| Total ADC | not applicable | higher (100-200%) |
| Free | | |
| Antibody¹ | | |
| Total ADC | not applicable | higher (100-200%) |
| Free Payload² | | |

Lymphatic

| Total ADC | higher (100-500%) | higher (100-300%) |
|---|---|---|
| Concentration | | |
| Total ADC | higher (100-500%) | higher (100-300%) |
| Free | | |
| Antibody¹ | | |
| Total ADC | higher (100-500%) | higher (100-300%) |
| Free Payload² | | |

| rHuPH20 treated subjects | | | |
|---|---|---|---|
| Day 1 | Day 7 | Day 14 | Day 21 |

Systemic

| | | | | |
|---|---|---|---|---|
| Free antibody Total ADC | 0-45% | 0-190% | 0-300% | 0-425% |
| Free antibody:Total ADC (ADCs with | 20-45% | 85-130% | 150-220% | 225-310% |
| Ab-linker instability) | | | | |
| Total ADC:Free antibody (very | 50-70% | 0-5% | 0-5% | 0-5% |
| unstable linker, such as carbonate- | | | | |
| based) | | | | |
| Free payload:Total ADC (mass | 0-1.3% | 0-4% | 0-4% | 0-4% |
| concentrations) | | | | |

### Example 16: Calculation of Free Antibody Following Antibody Drug Conjugate Administration

Free Ab: ADC is measured because for completely stable antibody, ADC:free antibody would be infinity. Since total Ab-total ADC is measured, for the free antibody measure, it does not matter if the linker detached from the antibody or the payload from the linker. Data is for percentage drug remaining conjugated.

| Day | 1 | 7 | 14 | 21 |
|---|---|---|---|---|
| ADC/total Ab | 83.9% | 54.0% | 39.4% | 30.7% |
| free Ab:ADC | 19.1% | 85.1% | 153.7% | 226.2% |
| ADC:free AB | 522.7% | 117.5% | 65.1% | 44.2% |
| Adjustment factor to allow for | 80.0% | 80.0% | 80.0% | 80.0% |
| variability-lower end | | | | |
| | 67.2% | 43.2% | 31.5% | 24.5% |
| free Ab:ADC | 48.9% | 131.4% | 217.1% | 307.7% |
| ADC:free AB | 204.4% | 76.1% | 46.1% | 32.5% |

### Example 17: Calculation of Free Payload Following Antibody Drug Conjugate Administration

Antibody drug conjugates with thiol-malimide conjugation undergo deconjugation following administration. To assess this deconjugation, free payload: ADC was calculated rather than reverse, because for completely stable antibody, ADC:free payload would be infinity. From below examples, highest release of payload was from Polivy, though no certain that their measure excluded detached linker-payload.

| Day | 1 | 7 | 14 | 21 |
|---|---|---|---|---|
| | 0.9% | 2.8% | 2.8% | 2.8% |
| Adjustment factor to allow for | 1.5 | 1.5 | 1.5 | 1.5 |
| variability | | | | |
| | 1.3% | 4.2% | 4.2% | 4.2% |

### Example 18: Calculation of Free Payload with Specific Antibody Drug Conjugates

Antibody drug conjugates with thiol-malimide conjugation undergo deconjugation following administration. To assess this deconjugation, free payload: ADC was calculated for specific antibody drug conjugates including Trastuzumab, Polatuzumab, Enfortumab and Tisotumab.

| | Concentration | | | | |
|---|---|---|---|---|---|
| | (weight/mL) basis | | Molar basis | | |
| | Cmax | AUC | Cmax | AUC | |
| Trastuzumab | 0.00413% | 0.00444% | 1.5% | 1.6% | |
| deruxtecan (Enhertu) | | | | | |
| Polatuzumab vedotin | 0.46% | 1.52% | 96.9% | 317.5% | unconjugated |
| (Polivy) | | | | | MMAE/antibody |
| | | | | | conjugated MMAE |
| | | | | | over 21 days |
| | | | 87.2% | 257.1% | different tumor |
| | | | 52.8% | 173.1% | Using molecular |
| | | | | | weight for the entire |
| | | | | | linker |
| | | | 47.5% | 140.2% | different tumor |
| Enfortumab vedotin | 0.02% | 0.08% | 4.2% | 16.4% | Unconjugated |
| (Padcev) | | | | | MMAE/ADC over 28 |
| | | | | | days |
| | | | 2.3% | 8.9% | Using molecular |
| | | | | | weight for the entire |
| | | | | | linker |
| Tisotumab vedotin | 0.01% | 0.09% | 3.1% | 18.5% | |
| (Tivdak) | | | | | |
| | | | 1.7% | 10.1% | Unconjugated |
| | | | | | MMAE/ADC over 21 |
| | | | | | days |

### Example 19: Calculation of Drug to Antibody Ratio (DAR)

The Drug-to-Antibody Ratio (DAR) is a critical parameter in antibody drug conjugate development, representing the average number of drug molecules attached to each antibody molecule. DAR significantly impacts the efficacy, pharmacokinetics, and safety profile of each antibody drug conjugate. The DAR was calculated for different antibody drug conjugates as set forth in the tables below.

MCC-DM1 Linker in Sacituzumab govitecan

| Day | 1 | 7 | 14 | 21 |
|---|---|---|---|---|
| % DAR remaining (which may | 76.2% | 42.9% | 31.0% | 23.8% |
| be ~ADC/ total Ab) | | | | |
| Adjustment factor to allow | 90.0% | 80.0% | 80.0% | 80.0% |
| for variability | | | | |
| | 68.6% | 34.3% | 24.8% | 19.0% |
| free Ab:ADC | 45.8% | 191.7% | 303.8% | 425.0% |
| ADC:free Ab | 218.2% | 52.2% | 32.9% | 23.5% |

SPDB-DM4 linker in Mirvetuximab soravtansine

| Day | 1 | 7 |
|---|---|---|
| % DAR remaining (which may be ~ADC/total Ab) | 88.4% | 32.6% |
| Adjustment factor to allow for variability | 80.0% | 80.0% |
| | 70.7% | 26.0% |
| free Ab:ADC | 41.4% | 283.9% |
| ADC:free Ab | 241.3% | 35.2% |

### REFERENCES

Ducry L and Stump B., “Antibody-drug conjugates: linking cytotoxic payloads to monoclonal antibodies.” Bioconjug. Chem. (2010) 21(1): 5-13.

1-128. (canceled)

129. A pharmaceutical composition for subcutaneous administration, the pharmaceutical composition comprising:
an antibody-drug conjugate (ADC) comprising an antibody and a payload conjugated to the antibody via a cleavable linker; and
a soluble hyaluronidase.

130. The pharmaceutical composition of claim 129, wherein the antibody binds to at least one antigen selected from Trop-2, HER-2, B7-H3, EGFR, DLL3, HER-3, CDH17, folate receptor alpha, Nectin-4, CLDN18.2, c-MET, NaPI2b, CEACAM5, PSMA, CLDN6, FGFR2b, ROR1, CD33, CD30, CD22, CD79b, CD19, integrin beta-6, or Tissue Factor.

131. The pharmaceutical composition of claim 129, wherein the payload is a topoisomerase I inhibitor payload.

132. The pharmaceutical composition of claim 131, wherein the topoisomerase I inhibitor payload is selected from A-1743332 (Adizutecan), AMDCPT, ATI020, AZ14170132 (AZ′0132) (Samrotecan), AZ14170133, BCPT02, Belotecan, BLD1102, Bultecan, C24, Camptothecin, CPT-113, CPT116, CPT2, D2102, Deruxtecan, DDDXd, DXd/DX8951 (MAAA-1181a), Dxh, Ed-4, Exatecan, FL-118, GS-P-000, HC74, HS-9265/SHR9265/Rezetecan, Irinotecan (CPT-11), JS-1, KL610023, LD-38, LDX2, Masetecan, MH30010008, Mtoxin (MF-6), NT1, P1003, P1021 (Drozuntecan), PBX-7, PBX-7016, PY-4car2, PY-4car2, QLS6916, SC3386, SN-38, T01, Tavatecan, Topotecan, VIP126, YL0010014, YL0014, ZD06519, or a derivative or analogue of any one thereof.

133. The pharmaceutical composition of claim 131, wherein the topoisomerase I inhibitor payload is a chemotherapy drug.

134. The pharmaceutical composition of claim 133, wherein the antibody specifically targets Trop 2.

135. The pharmaceutical composition of claim 134, wherein the chemotherapy drug is SN-38.

136. The pharmaceutical composition of claim 135, wherein the ADC is sacituzumab govitecan.

137. The pharmaceutical composition of claim 133, wherein the antibody specifically targets HER2.

138. The pharmaceutical composition of claim 137, wherein the chemotherapy drug is exatecan derivative DXd.

139. The pharmaceutical composition of claim 138, wherein the ADC is trastuzumab deruxtecan.

140. The pharmaceutical composition of claim 129, wherein the cleavable linker is a chemically cleavable linker.

141. The pharmaceutical composition of claim 129, wherein the cleavable linker is selected from an acid labile linker, an enzyme cleavable linker, a reducible disulfide linker, a glutathione-sensitive linker, an Fe (II)-responsive linker, an oxidation labile/ROS (reactive oxygen species) sensitive linker, a photo-responsive linker, a bioorthogonal linker, or a combination thereof.

142. The pharmaceutical composition of claim 141, wherein the acid labile linker is a hydrazone linker or a CL2A linker.

143. The pharmaceutical composition of claim 129, wherein the ADC has a drug antibody ratio of 2-16.

144. The pharmaceutical composition of claim 143, wherein the ADC has a drug-antibody ratio of 2-8.

145. The pharmaceutical composition of claim 144, wherein the ADC has a drug-antibody ratio of 2-4.

146. The pharmaceutical composition of claim 129, further comprising one or more excipients selected from 2-(N-morpholino) ethane sulfonic acid (MES), citric acid monohydrate, dextran, d-mannitol, glacial acetic acid, histidine, histidine hydrochloride monohydrate, L-histidine, L-histidine hydrochloride monohydrate, L-histidine monohydrochloride, polysorbate, sodium acetate, sodium chloride, sodium citrate dihydrate, sodium hydroxide, sodium phosphate dibasic anhydrous, sodium phosphate monobasic monohydrate, sodium succinate, succinic acid, sucrose, trehalose, trehalose dihydrate, or tromethamine.

147. The pharmaceutical composition of claim 129, further comprising one or more buffers selected from histidine, MES, citrate, acetate, phosphate, or TRIS.

148. The pharmaceutical composition of claim 129, further comprising one or more stabilizers selected from trehalose, sucrose, mannitol, sorbitol, glycine, or arginine.

149. The pharmaceutical composition of claim 129, further comprising one or more surfactants selected from polysorbate 20, polysorbate 80, poloxamer 188, or sodium deoxycholate.

150. The pharmaceutical composition of claim 129, further comprising one or more tonicity-adjusting agents selected from sodium chloride, potassium chloride, calcium chloride, or glycerol.

151. The pharmaceutical composition of claim 129, further comprising one or more antioxidants selected from methionine, cysteine, ascorbic acid, a tocopherol, or BHT.

152. The pharmaceutical composition of claim 129, further comprising one or more preservatives selected from benzyl alcohol, phenol, m-cresol, or a paraben.

153. The pharmaceutical composition of claim 129, wherein the soluble hyaluronidase comprises a soluble human hyaluronidase.

154. The pharmaceutical composition of claim 129, wherein the soluble hyaluronidase comprises a recombinant soluble human hyaluronidase.

155. The pharmaceutical composition of claim 129, wherein the soluble hyaluronidase comprises a sequence of amino acids that has at least 85%, 86%, 87%, 88%, 89%, 90%, 91%, 92%, 93%, 94%, 95%, 96%, 97%, 98% or 99% sequence identity to a sequence of amino acids that contains at least amino acids 36-464 of SEQ ID NO:1 and retains hyaluronidase activity.

156. A combination dosing regimen, comprising:
subcutaneously administering to a patient in need thereof an antibody-drug conjugate (ADC) and a soluble hyaluronidase, wherein the subcutaneous administration of the soluble hyaluronidase is subcutaneously administered to the patient in an amount sufficient to obtain at least 50% bioavailability of the ADC compared to the bioavailability obtained when the ADC is administered intravenously.

157. The combination dosing regimen of claim 156, wherein the ADC and the soluble hyaluronidase are subcutaneously administered to the patient in a composition comprising the soluble hyaluronidase and the ADC.

158. The combination dosing regimen of claim 157, wherein the soluble hyaluronidase comprises a sequence of amino acids that has at least 85%, 86%, 87%, 88%, 89%, 90%, 91%, 92%, 93%, 94%, 95%, 96%, 97%, 98% or 99% sequence identity to a sequence of amino acids that contains at least amino acids 36-464 of SEQ ID NO:1 and retains hyaluronidase activity.

159. The combination dosing regimen of claim 156, wherein the soluble hyaluronidase is subcutaneously administered to the patient in a first composition comprising the soluble hyaluronidase and the ADC is subcutaneously administered to the patient in a second composition comprising the ADC.

160. The combination dosing regimen of claim 156, wherein the subcutaneous administration of the ADC with the soluble hyaluronidase provides:
a maximum blood concentration (Cmax) of ADC that is about 20% to about 60% of the Cmax obtained via intravenous (IV) administration of an equivalent dose of the ADC; and
an area under the concentration-time curve (AUC) in blood of ADC that is about 50% to about 90% of the AUC obtained via IV administration of an equivalent dose of the ADC.

161. The combination dosing regimen of claim 160, wherein the subcutaneous administration of the ADC with the soluble hyaluronidase achieves a weekly average area under the concentration-time curve (AUC) in blood of the ADC that is about 100 μg/mL*day to about 230 μg/mL*day.

162. The combination dosing regimen of claim 160, wherein the subcutaneous administration of the ADC with the soluble hyaluronidase achieves a Cmax of the ADC that is about 20 μg/mL to about 150 μg/mL.

163. The combination dosing regimen of claim 156, wherein the dose of the ADC administered subcutaneously is 25% to 400% of the dose of the ADC administered intravenously.

164. The combination dosing regimen of claim 156, wherein the ADC comprises an antibody and a payload conjugated to the antibody via a cleavable linker, and wherein free antibody and free payload are released upon cleavage of the cleavable linker.

165. The combination dosing regimen of claim 164, wherein the subcutaneous administration of the ADC with the soluble hyaluronidase provides a Cmax of the free antibody that is about 20% to 60% of the Cmax achieved by intravenous (IV) administration of an equivalent dose of the ADC.

166. The combination dosing regimen of claim 164, wherein the subcutaneous administration of the ADC with the soluble hyaluronidase provides a Cmax of the free payload that is about 30% to about 80% of the Cmax achieved by intravenous (IV) administration of an equivalent dose of the ADC.

167. The combination dosing regimen of claim 164, wherein the subcutaneous administration of the ADC with the soluble hyaluronidase provides an AUC in blood of total antibody that is about 50% to 90% of the blood area under the concentration-time curve (AUC) achieved by intravenous (IV) administration of an equivalent dose of the ADC.

168. The combination dosing regimen of claim 164, wherein the subcutaneous administration of the ADC with the soluble hyaluronidase provides an AUC in blood of the free payload that is about 50% to 90% of the blood area under the concentration-time curve (AUC) achieved by intravenous (IV) administration of an equivalent dose of the ADC.

169. The combination dosing regimen of claim 156, wherein the combination dosing regimen results in:
a higher dose of the ADC being administered subcutaneously than a therapeutically effective dose of the ADC when administered intravenously;
the subcutaneous administration of the ADC yields an area under the concentration-time curve (AUC) in blood of total antibody that is equal to or higher than the blood AUC obtained from an intravenously administered therapeutically effective dose of the ADC; and
the subcutaneous administration of the ADC yields a Cmax of the ADC that is equal to or lower than the Cmax obtained from an intravenously administered therapeutically effective dose of the ADC.

170. The combination dosing regimen of claim 169, wherein the subcutaneous administration of the ADC with the soluble hyaluronidase achieves a weekly average AUC in blood of the ADC that is about 120 μg/mL*day to 680 μg/mL*day.

171. The combination dosing regimen of claim 169, wherein the subcutaneous administration of the ADC with the soluble hyaluronidase achieves a Cmax of the ADC that is about 25 g/mL to 410 μg/mL.

172. The combination dosing regimen of claim 169, wherein the subcutaneous administration of the ADC with the soluble hyaluronidase achieves a Cmax of the ADC that is about 35 μg/mL to 330 μg/mL.

173. The combination dosing regimen of claim 169, wherein the subcutaneous administration of the ADC in combination with the soluble hyaluronidase results one or more of:
a greater therapeutic efficacy of the ADC relative to intravenous (IV) administration of the ADC;
an increased overall survival of the patient relative to IV administration of the ADC;
a greater complete response of the ADC in the patient relative to IV administration of the ADC;
an increased duration of response of the ADC in the patient relative to IV administration of the ADC;
an increased progression-free survival of the patient relative to IV administration of the ADC;
an increased disease-free survival of the patient relative to IV administration of the ADC; and
a decreased time to treatment failure in the patient relative to IV administration of the ADC.

174. The combination dosing regimen of claim 169, wherein the subcutaneous administration of the ADC in combination with the soluble hyaluronidase results in reduced toxicity of the ADC in the patient relative to intravenous (IV) administration of the ADC.

175. The combination dosing regimen of claim 169, wherein the subcutaneous administration of the ADC in combination with the soluble hyaluronidase results in a reduced frequency or a reduced severity of an adverse event of the ADC in the patient relative to intravenous (IV) administration of the ADC.

176. The combination dosing regimen of claim 164, wherein the combination dosing regimen results in:
a higher dose of the ADC being administered subcutaneously than a therapeutically effective dose of the ADC when administered intravenously;
the subcutaneous administration of the ADC yields an area under the concentration-time curve (AUC) in blood of the free antibody that is equal to or higher than the blood AUC obtained from an intravenously administered therapeutically effective dose of the ADC; and
the subcutaneous administration of the ADC yields a Cmax of the free antibody that is equal to or lower than the Cmax obtained from an intravenously administered therapeutically effective dose of the ADC.

177. The combination dosing regimen of claim 176, wherein the subcutaneous administration of the ADC with the soluble hyaluronidase achieves a weekly average AUC in blood of the ADC that is about 120 μg/mL*day to 680 μg/mL*day.

178. The combination dosing regimen of claim 176, wherein the subcutaneous administration of the ADC with the soluble hyaluronidase achieves a Cmax of the ADC that is about 25 g/mL to 410 μg/mL.

179. The combination dosing regimen of claim 176, wherein the subcutaneous administration of the ADC with the soluble hyaluronidase achieves a Cmax of the ADC that is about 35 μg/mL to 330 μg/mL.

180. The combination dosing regimen of claim 164, wherein the combination dosing regimen results in:
a higher dose of the ADC being administered subcutaneously than a therapeutically effective dose of the ADC when administered intravenously;
the subcutaneous administration of the ADC yields an area under the concentration-time curve (AUC) in blood of total payload that is equal to or higher than the blood AUC obtained from an intravenously administered therapeutically effective dose of the ADC; and
the subcutaneous administration of the ADC yields a Cmax of the total payload that is equal to or lower than the Cmax obtained from an intravenously administered therapeutically effective dose of the ADC.

181. The combination dosing regimen of claim 180, wherein the subcutaneous administration of the ADC with the soluble hyaluronidase achieves a weekly average AUC in blood of the ADC that is about 120 μg/mL*day to 680 μg/mL*day.

182. The combination dosing regimen of claim 180, wherein the subcutaneous administration of the ADC with the soluble hyaluronidase achieves a Cmax of the ADC that is about 25 μg/mL to 410 μg/mL.

183. The combination dosing regimen of claim 180, wherein the subcutaneous administration of the ADC with the soluble hyaluronidase achieves a Cmax of the ADC that is about 35 μg/mL to 330 μg/mL.

184. The combination dosing regimen of claim 164, wherein the combination dosing regimen results in:
a higher dose of the ADC being administered subcutaneously than a therapeutically effective dose of the ADC when administered intravenously;
the subcutaneous administration of the ADC yields an area under the concentration-time curve (AUC) in blood of the free payload that is equal to or higher than the blood AUC obtained from an intravenously administered therapeutically effective dose of the ADC; and
the subcutaneous administration of the ADC yields a Cmax of the free payload that is equal to or lower than the Cmax obtained from an intravenously administered therapeutically effective dose of the ADC.

185. The combination dosing regimen of claim 184, wherein the subcutaneous administration of the ADC with the soluble hyaluronidase achieves a weekly average AUC in blood of the ADC that is about 120 μg/mL*day to 680 μg/mL*day.

186. The combination dosing regimen of claim 184, wherein the subcutaneous administration of the ADC with the soluble hyaluronidase achieves a Cmax of the ADC that is about 25 g/mL to 410 μg/mL.

187. The combination dosing regimen of claim 184, wherein the subcutaneous administration of the ADC with the soluble hyaluronidase achieves a Cmax of the ADC that is about 35 g/mL to 330 μg/mL.

188. The combination dosing regimen of claim 164, wherein the combination dosing regimen results in:
a higher dose of the ADC being administered subcutaneously than a therapeutically effective dose of the ADC when administered intravenously;
the subcutaneous administration of the ADC yields an area under the concentration-time curve (AUC) in blood of the ADC that is equal to or higher than the blood AUC obtained from an intravenously administered therapeutically effective dose of the ADC; and
the subcutaneous administration of the ADC yields a Cmax of a total payload that is equal to or lower than the Cmax obtained from an intravenously administered therapeutically effective dose of the ADC.

189. The combination dosing regimen of claim 188, wherein the subcutaneous administration of the ADC with the soluble hyaluronidase achieves a weekly average AUC in blood of the ADC that is about 120 μg/mL*day to 680 μg/mL*day.

190. The combination dosing regimen of claim 188, wherein the subcutaneous administration of the ADC with the soluble hyaluronidase achieves a Cmax of the ADC that is about 25 μg/mL to 410 μg/mL.

191. The combination dosing regimen of claim 188, wherein the subcutaneous administration of the ADC with the soluble hyaluronidase achieves a Cmax of the ADC that is about 35 μg/mL to 330 μg/mL.

192. The combination dosing regimen of claim 164, wherein the combination dosing regimen results in:
a higher dose of the ADC being administered subcutaneously than a therapeutically effective dose of the ADC when administered intravenously;
the subcutaneous administration of the ADC yields an area under the concentration-time curve (AUC) in blood of a total payload that is equal to or higher than the blood AUC obtained from an intravenously administered therapeutically effective dose of the ADC; and
the subcutaneous administration of the ADC yields a Cmax of the total payload that is equal to or lower than the Cmax obtained from an intravenously administered therapeutically effective dose of the ADC.

193. The combination dosing regimen of claim 192, wherein the subcutaneous administration of the ADC with the soluble hyaluronidase achieves a weekly average AUC in blood of the ADC that is about 120 μg/mL*day to 680 μg/mL*day.

194. The combination dosing regimen of claim 192, wherein the subcutaneous administration of the ADC with the soluble hyaluronidase achieves a Cmax of the ADC that is about 25 μg/mL to 410 μg/mL.

195. The combination dosing regimen of claim 192, wherein the subcutaneous administration of the ADC with the soluble hyaluronidase achieves a Cmax of the ADC that is about 35 μg/mL to 330 μg/mL.

196. The combination dosing regimen of claim 156, wherein the intravenous (IV) administration of the ADC is administered without soluble hyaluronidase.

197. The combination dosing regimen of claim 156, wherein the subcutaneous administration of the ADC in combination with the soluble hyaluronidase results one or more of:
a greater therapeutic efficacy of the ADC relative to intravenous (IV) administration of the ADC;
an increased overall survival of the patient relative to IV administration of the ADC;
a greater complete response of the ADC in the patient relative to IV administration of the ADC;
an increased duration of response of the ADC in the patient relative to IV administration of the ADC;
an increased progression-free survival of the patient relative to IV administration of the ADC;
an increased disease-free survival of the patient relative to IV administration of the ADC; and
a decreased time to treatment failure in the patient relative to IV administration of the ADC.

198. The combination dosing regimen of claim 156, wherein the subcutaneous administration of the ADC in combination with the soluble hyaluronidase results in reduced toxicity of the ADC in the patient relative to intravenous (IV) administration of the ADC.

199. The combination dosing regimen of claim 156, wherein the subcutaneous administration of the ADC in combination with the soluble hyaluronidase results in a reduced frequency or a reduced severity of an adverse event of the ADC in the patient relative to intravenous (IV) administration of the ADC.

200. The combination dosing regimen of claim 199, wherein the adverse event is selected from hypersensitivity and infusion-related reactions.

201. The combination dosing regimen of claim 199, wherein the adverse event is selected from itching, redness, rash, hives, fever, chills, back pain, belly pain, muscle pain, joint pain, arthralgia, neuropathy, interstitial lung disease, increased heart rate, irregular heartbeat, nausea, vomiting, diarrhea, constipation, abdominal pain, gastroenteritis, anorexia, mucositis, stomatitis, rash, pruritic, edema, dry skin, alopecia, and severe anaphylactic reactions, and wherein severe anaphylactic reactions can include signs and symptoms of cardiac arrest, hypotension, wheezing, angioedema, swelling, pneumonitis, or skin reactions.

202. The combination dosing regimen of claim 199, wherein the adverse event is selected from cytopenia, neutropenia, thrombocytopenia, anemia, leukopenia and lymphocytopenia.

203. The combination dosing regimen of claim 156, wherein the subcutaneous administration of the ADC in combination with the soluble hyaluronidase results in one or more of:
an increased blood hemoglobin in the patient relative to intravenous (IV) administration of the ADC;
an increased blood albumin in the patient relative to IV administration of the ADC;
an increased creatinine clearance in the patient relative to IV administration of the ADC;
a decreased blood alkaline phosphatase in the patient relative to IV administration of the ADC;
an increased blood magnesium in the patient relative to IV administration of the ADC;
an increased blood potassium in the patient relative to IV administration of the ADC; and
an increased blood sodium in the patient relative to IV administration of the ADC.


SEQUENCE LISTING (21 sequences)

SEQ ID NO: 1 - protein, 509 aa, synthetic construct
  REGION 1..509: note = precursor human PH20
MGVLKFKHIFFRSFVKSSGVSQIVFTFLLIPCCLTLNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLSATMFIVSILFLIISSVASL

SEQ ID NO: 2 - protein, 474 aa, synthetic construct
  REGION 1..474: note = Mature PH20
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLSATMFIVSILFLIISSVASL

SEQ ID NO: 3 - protein, 482 aa, synthetic construct
  REGION 1..482: note = precursor soluble rHuPH20
MGVLKFKHIFFRSFVKSSGVSQIVFTFLLIPCCLTLNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFY

SEQ ID NO: 4 - protein, 447 aa, synthetic construct [sequence omitted]

SEQ ID NO: 5 - protein, 446 aa, synthetic construct
  REGION 1..446: note = soluble rHuPH20 1-446
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIF

SEQ ID NO: 6 - protein, 445 aa, synthetic construct
  REGION 1..445: note = soluble rHuPH20 1-445
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQI

SEQ ID NO: 7 - protein, 444 aa, synthetic construct
  REGION 1..444: note = soluble rHuPH20 1-444
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQ

SEQ ID NO: 8 - protein, 443 aa, synthetic construct
  REGION 1..443: note = soluble rHuPH20 1-443
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEP

SEQ ID NO: 9 - protein, 442 aa, synthetic construct
  REGION 1..442: note = soluble rHuPH20 1-442
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEE

SEQ ID NO: 10 - protein, 414 aa, synthetic construct
  REGION 1..414: note = hyaluronidase 3
MTTRLGPALVLGVALCLGCGQPLPQVPERPFSVLWNVPSAHCKSRFGVHLPLNALGIIANRGQHFHGQNMTIFYKNQLGLYPYFGPKGTAHNGGIPQALPLDRHLALAAYQIHHSLRPGFAGPAVLDWEEWCPLWAGNWGRRRAYQAASWAWAQQVFPDLDPQEQLYKAYTGFEQAARALMEDTLRVAQALRPHGLWGFYHYPACGNGWHSMASNYTGRCHAATLARNTQLHWLWAASSALFPSIYLPPRLPPAHHQAFVRHRLEEAFRVALVGHLPVLAYVRLTHRRSGRFLSQDDLVQTIGVSAALGAAGVVLWGDLSLSSSEEECWHLHDYLVDTLGPYGINVTRAAMACSHQRCHGHGRCARRDPGQMEAFLHLWPDGSLGDWKSFSCHCYWGWAGPTCQEPRLGPKEAV

SEQ ID NO: 11 - protein, 477 aa, synthetic construct
  REGION 1..477: note = sHuPH20 precursor 1-477
MGVLKFKHIFFRSFVKSSGVSQIVFTFLLIPCCLTLNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEE

SEQ ID NO: 12 - protein, 478 aa, synthetic construct
  REGION 1..478: note = sHuPH20 precursor 1-478
MGVLKFKHIFFRSFVKSSGVSQIVFTFLLIPCCLTLNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEP

SEQ ID NO: 13 - protein, 479 aa, synthetic construct
  REGION 1..479: note = sHuPH20 precursor 1-479
MGVLKFKHIFFRSFVKSSGVSQIVFTFLLIPCCLTLNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQ

SEQ ID NO: 14 - protein, 480 aa, synthetic construct
  REGION 1..480: note = sHuPH20 precursor 1-480
MGVLKFKHIFFRSFVKSSGVSQIVFTFLLIPCCLTLNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQI

SEQ ID NO: 15 - protein, 481 aa, synthetic construct
  REGION 1..481: note = sHuPH20 precursor 1-481
MGVLKFKHIFFRSFVKSSGVSQIVFTFLLIPCCLTLNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIF

SEQ ID NO: 16 - protein, 483 aa, synthetic construct
  REGION 1..483: note = sHuPH20 precursor 1-483
MGVLKFKHIFFRSFVKSSGVSQIVFTFLLIPCCLTLNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYN

SEQ ID NO: 17 - protein, 448 aa, synthetic construct
  REGION 1..448: note = sHuPH20 mature 36-483
LNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYN

SEQ ID NO: 18 - protein, 16 aa, synthetic construct
VVSVLTVLHQDWLNGK

SEQ ID NO: 19 - protein, 14 aa, synthetic construct
DSTYSLSSTLTLSK

SEQ ID NO: 20 - protein, 16 aa, synthetic construct
VVSVLTVLHQDWLNGK

SEQ ID NO: 21 - protein, 14 aa, synthetic construct
DSTYSLSSTLTLSK